(19) United States
(12) Patent Application Publication
(10) Pub. No.: US 2026/0206412 A1
(43) Pub. Date: Jul. 16, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Eun-Joung CHOI, Gyeonggi-do (KR); HaeYeon KIM, Gyeonggi-do (KR); So-Mi PARK, Gyeonggi-do (KR); Seung-Hyun YOON, Gyeonggi-do (KR)

(21) Appl. No.: 19/440,779

(22) Filed: Jan. 6, 2026

(30) Foreign Application Priority Data
Jan. 13, 2025 (KR) 10-2025-0004583
Sep. 1, 2025 (KR) 10-2025-0123077
Dec. 2, 2025 (KR) 10-2025-0187924

(51) Int. Cl. H10K 50/17; C07B 59/00; C07D 471/04; C09K 11/06; H10K 85/60

(52) U.S. Cl. CPC H10K 50/171; C07B 59/004; C07D 471/04; C09K 11/06; H10K 85/615; H10K 85/633; H10K 85/636; H10K 85/654; H10K 85/6572; H10K 85/6574; H10K 85/658; C07B 2200/05; C09K 2211/1062; C09K 2211/1066; C09K 2211/1077; C09K 2211/1081

(57) ABSTRACT

The present disclosure relates to an organic electroluminescent compound represented by Formula 1, and an organic electroluminescent material and an organic electroluminescent device comprising the same. By comprising the organic electroluminescent compound according to the present disclosure, an organic electroluminescent device can provide improved lifespan characteristics as compared with conventional devices.

### TECHNICAL FIELD

The present disclosure relates to an organic electroluminescent compound, and an organic electroluminescent device comprising the same.

### BACKGROUND ART

Since Tang et al. of Eastman Kodak first developed in 1987 a low-molecular-weight green organic electroluminescent device (OLED) of a TPD/Alq3 bilayer composed of a light-emitting layer and a charge transport layer, research on organic electroluminescent devices has rapidly progressed and has now reached commercialization. At present, organic electroluminescent devices mainly use phosphorescent materials having excellent luminous efficiency in panel implementation. For long-time use of displays and high resolution, OLEDs having high luminous efficiency are required.

Korean Patent Application Laid-Open No. 10-2022-0076881 discloses a compound comprising a heteroaryl derivative; however, it does not specifically disclose the particular compound claimed herein. In addition, there is a continuing demand to develop emission materials having improved performance, for example, improved lifetime characteristics, compared with the compounds disclosed in the prior art.

### DISCLOSURE OF INVENTION

### Technical Problem

An object of the present disclosure is to provide an organic electroluminescent compound of a new structure suitable for application to an organic electroluminescent device. Another object of the present disclosure is to provide an organic electroluminescent material and an organic electroluminescent device having improved lifetime characteristics.

### Solution to Problem

As a result of extensive studies to solve the above technical problems, the present inventors have found that an organic electroluminescent compound represented by the following Formula 1 and an organic electroluminescent device comprising the same achieves the above-described objects, thereby completing the present invention.

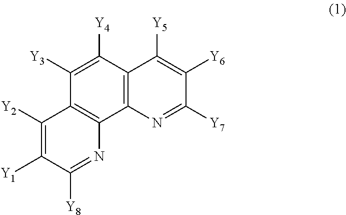

In Formula 1,
Y₁ to Y₆ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀)aliphatic ring and a (C₆-C₃₀)aromatic ring; or may be linked to an adjacent substituent to form a ring; and
Y₇ and Y₈ each independently represent hydrogen, deuterium, halogen, cyano, a substituted or unsubstituted phenyl, a substituted or unsubstituted biphenyl, a substituted or unsubstituted terphenyl, a substituted or unsubstituted (C₁-C₃₀)alkyl, a substituted or unsubstituted (C₂-C₃₀)alkynyl, or a substituted or unsubstituted (3- to 30-membered)heteroaryl;
provided that at least one of Y₁ to Y₈ is represented by the following Formula 1-a or 1-b, or is HAr, which is a substituted or unsubstituted (3- to 30-membered)heteroaryl:

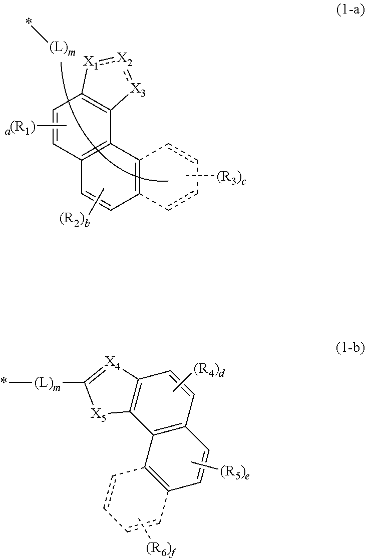

In Formulas 1-a and 1-b,
the dotted line indicates the presence or absence of each ring;
L represents a single bond, a substituted or unsubstituted (C₆-C₃₀)arylene, a substituted or unsubstituted (3- to 30-membered)heteroarylene, or a substituted or unsubstituted (C₂-C₃₀)alkynylene;
X₁ to X₃ each independently represent —N═, —O—, —S—, or —CRₐ—; provided that one of X₁ to X₃ is —N═, another one of X₁ to X₃ is —O— or —S—, and the remaining one of X₁ to X₃ is —O—, —S—, or —CRₐ—;
X₄ and X₅ each independently represent —N═, —O—, or —S—; provided that one of X₄ and X₅ is —N═ and the other is —O— or —S—;
R₁ to R₃ each independently represent a position for bonding to L, or hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, or a substituted or unsubstituted (C₁-C₃₀)alkoxy; or may be linked to an adjacent substituent to form a ring;
Rₐ and R₄ to R₆ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, or a substituted or unsubstituted (C₁-C₃₀)alkoxy; or may be linked to an adjacent substituent to form a ring;
m is an integer of 1 or 2, and when m is 2, each L may be the same or different;
a and d are each independently an integer of 1 or 2, and b, c, e, and f are each independently an integer of 1 to 4; and
when a to f are integers of 2 or more, each of R₁ to each of R₆ may be the same or different.

### Advantageous Effects of Invention

The organic electroluminescent compound according to the present disclosure exhibits performance suitable for use in an organic electroluminescent device. In addition, by comprising the compound according to the present disclosure as an organic electroluminescent material, an organic electroluminescent device exhibiting improved lifetime characteristics as compared with conventional organic electroluminescent devices can be provided, and a display device or a lighting device using the same can be manufactured.

### MODE FOR INVENTION

Hereinafter, the present disclosure will be described in detail. However, the following description is intended to explain the invention, and is not meant in any way to restrict the scope of the invention.

Herein, the term “organic electroluminescent compound” means a compound that may be used in an organic electroluminescent device, and this may be comprised in any material layer constituting an organic electroluminescent device, as necessary.

Herein, the term “organic electroluminescent material” means a material that can be used in an organic electroluminescent device, may comprise one or more kinds of compounds, and, as necessary, may be included in any layer constituting the organic electroluminescent device. For example, the organic electroluminescent material may be a hole injection material, a hole transport material, a hole auxiliary material, a charge generation material, an n-type charge generation material, a p-type charge generation material, a light-emitting auxiliary material, an electron blocking material, a light-emitting material (including a host material and a dopant material), an electron buffer material, a hole blocking material, an electron transport material, or an electron injection material.

Herein, “(C₁-C₃₀)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, “(C₂-C₃₀)alkynyl(ene)” means a straight-chain or branched-chain alkynyl group in which the number of carbons constituting the chain is 2 to 30; herein, it is preferably 2 to 20, and more preferably 2 to 10. The alkynyl group is an unsaturated hydrocarbon group containing one or more triple bonds, and may have a terminal or an internal triple bond. Specific examples include ethynyl (—C≡CH), propynyl (e.g., 1-propynyl, 2-propynyl), butynyl (e.g., 1-butynyl, 3-butynyl), and pentynyl, which may have a straight-chain or a branched structure. Herein, “(C₃-C₃₀)cycloalkyl” or “(C₃-C₃₀)cycloalkylene” means a monocyclic or polycyclic hydrocarbon in which the number of ring framework carbons is 3 to 30; herein, it is preferably 3 to 20, and more preferably 3 to 7. Examples of the cycloalkyl include cyclopropyl, cyclobutyl, cyclopentyl, cyclohexyl, cyclopentylmethyl, and cyclohexylmethyl. Herein, “(3- to 7-membered)heterocycloalkyl” means a ring-shaped hydrocarbon substituent of a saturated or partially unsaturated monocyclic or polycyclic ring having 3 to 7 ring framework atoms; herein, the number of carbons is preferably 5 to 7, and it includes one or more heteroatoms selected from B, N, O, S, Si, P, and Se. The heterocycloalkyl includes, for example, tetrahydrofuran, pyrrolidine, thiolane, and tetrahydropyran, etc. Herein, “(C₆-C₃₀)aryl” or “(C₆-C₃₀)arylene” means a monocyclic or fused-ring radical derived from an aromatic hydrocarbon in which the number of ring framework carbons is 6 to 30, and may be partially saturated; herein, the number of ring framework carbons is preferably 6 to 20, and more preferably 6 to 15. The aryl includes those having a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl” or “(3- to 30-membered)heteroarylene” means an aryl group or an arylene group in which the number of ring framework atoms is 3 to 30 and which includes one or more heteroatoms selected from the group consisting of B, N, O, S, Si, P, and Se. Herein, the number of ring framework atoms is preferably 3 to 30, and more preferably 5 to 20. The number of heteroatoms is preferably 1 to 4, and the ring may be monocyclic or a fused-ring system condensed with one or more benzene rings, and may be partially saturated. In addition, in the present disclosure, the heteroaryl or heteroarylene also includes forms in which one or more heteroaryl or aryl groups are linked to a heteroaryl group by a single bond, and includes those having a spiro structure. Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho[1,2-b]benzofuranyl, 2-naphtho[1,2-b]benzofuranyl, 3-naphtho[1,2-b]benzofuranyl, 4-naphtho[1,2-b]benzofuranyl, 5-naphtho[1,2-b]benzofuranyl, 6-naphtho[1,2-b]benzofuranyl, 7-naphtho[1,2-b]benzofuranyl, 8-naphtho[1,2-b]benzofuranyl, 9-naphtho[1,2-b]benzofuranyl, 10-naphtho[1,2-b]benzofuranyl, 1-naphtho[2,3-b]benzofuranyl, 2-naphtho[2,3-b]benzofuranyl, 3-naphtho[2,3-b]benzofuranyl, 4-naphtho[2,3-b]benzofuranyl, 5-naphtho[2,3-b]benzofuranyl, 6-naphtho[2,3-b]benzofuranyl, 7-naphtho[2,3-b]benzofuranyl, 8-naphtho[2,3-b]benzofuranyl, 9-naphtho[2,3-b]benzofuranyl, 10-naphtho[2,3-b]benzofuranyl, 1-naphtho[2,1-b]benzofuranyl, 2-naphtho[2,1-b]benzofuranyl, 3-naphtho[2,1-b]benzofuranyl, 4-naphtho[2,1-b]benzofuranyl, 5-naphtho[2,1-b]benzofuranyl, 6-naphtho[2,1-b]benzofuranyl, 7-naphtho[2,1-b]benzofuranyl, 8-naphtho[2,1-b]benzofuranyl, 9-naphtho[2,1-b]benzofuranyl, 10-naphtho[2,1-b]benzofuranyl, 1-naphtho[1,2-b]benzothiophenyl, 2-naphtho[1,2-b]benzothiophenyl, 3-naphtho[1,2-b]benzothiophenyl, 4-naphtho[1,2-b]benzothiophenyl, 5-naphtho[1,2-b]benzothiophenyl, 6-naphtho[1,2-b]benzothiophenyl, 7-naphtho[1,2-b]benzothiophenyl, 8-naphtho[1,2-b]benzothiophenyl, 9-naphtho[1,2-b]benzothiophenyl, 10-naphtho[1,2-b]benzothiophenyl, 1-naphtho[2,3-b]benzothiophenyl, 2-naphtho[2,3-b]benzothiophenyl, 3-naphtho[2,3-b]benzothiophenyl, 4-naphtho[2,3-b]benzothiophenyl, 5-naphtho[2,3-b]benzothiophenyl, 1-naphtho[2,1-b]benzothiophenyl, 2-naphtho[2,1-b]benzothiophenyl, 3-naphtho[2,1-b]benzothiophenyl, 4-naphtho[2,1-b]benzothiophenyl, 5-naphtho[2,1-b]benzothiophenyl, 6-naphtho[2,1-b]benzothiophenyl, 7-naphtho[2,1-b]benzothiophenyl, 8-naphtho[2,1-b]benzothiophenyl, 9-naphtho[2,1-b]benzothiophenyl, 10-naphtho[2,1-b]benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent with relatively abundant electrons in the parent nucleus, and for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene), which has hole properties, is a substituent with a relative lack of electrons in the parent nucleus, and for example, it may be a substituted or unsubstituted carbazolyl, a substituted or unsubstituted dibenzofuranyl, or a substituted or unsubstituted dibenzothiophenyl.

Herein, the term “a fused ring of a (C₃-C₃₀)aliphatic ring and a (C₆-C₃₀)aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of a (C₃-C₃₀)aliphatic ring and a (C₆-C₃₀)aromatic ring may be replaced with at least one heteroatom selected from B, N, O, S, Si, P, and Se. The “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, which is formed by two or more adjacent substituents being linked or fused together; preferably, it may be a substituted or unsubstituted (5- to 25-membered) monocyclic or polycyclic alicyclic ring, an aromatic ring, or a combination thereof. The formed ring may include one or more heteroatoms selected from B, N, O, S, Si, P, and Se. According to one embodiment, the number of ring framework atoms is 5- to 20-membered, and according to another embodiment, the number of ring framework atoms is 5- to 15-membered. By way of example, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring.

In addition, “substituted or unsubstituted” as described herein means that “substituted” refers to a case where a hydrogen atom in a functional group is replaced by another atom or another functional group (i.e., a substituent), and also includes replacement by a group in which two or more of the substituents are linked together. Unless otherwise specified, the substituent(s) can, without limitation, replace hydrogens at positions in which substitution by a substituent is possible, and, where two or more hydrogen atoms in a given functional group are respectively replaced by substituents, the respective substituents may be the same as or different from one another. The maximum number of substituents that can be introduced into a given functional group may be the sum of the substitutable valences of the respective atoms constituting the functional group. In Formulas of the present disclosure, the substituted alkyl, the substituted alkynyl(ene), the substituted aryl(ene), the substituted heteroaryl(ene), the substituted cycloalkyl(ene), the substituted alkoxy, the substituted trialkylsilyl, the substituted dialkylarylsilyl, the substituted alkyldiarylsilyl, the substituted triarylsilyl, the substituted fused-ring group of an aliphatic ring and an aromatic ring, the substituted benzene ring, and the substituted naphthalene ring are each independently substituted with one or more selected from the group consisting of: deuterium, halogen, cyano, carboxyl, nitro, hydroxy, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (3- to 30-membered)heteroaryl unsubstituted or substituted with (C₆-C₃₀)aryl, (C₆-C₃₀)aryl unsubstituted or substituted with (3- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused-ring group of a (C₃-C₃₀)aliphatic ring and a (C₆-C₃₀)aromatic ring, amino, mono- or di(C₁-C₃₀)alkylamino, a substituted or unsubstituted mono- or di(C₆-C₃₀)arylamino, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, (C₁-C₃₀)alkyl(C₆-C₃₀)aryl, phosphine oxide, and combinations thereof. According to one embodiment, the group is selected from deuterium, (C₁-C₂₀)alkyl, (C₆-C₂₅)aryl, (3- to 25-membered)heteroaryl, and combinations thereof. According to another embodiment, the group is selected from deuterium, (C₁-C₁₀)alkyl, (C₆-C₁₈)aryl, and combinations thereof. For example, the group may be deuterium, methyl, phenyl, or naphthyl, and these may be further substituted with deuterium.

When a substituent is not shown in the chemical formula or the compound structure of the present disclosure, it may signify that all positions that may be present as substituents are hydrogen or deuterium. That is, in the case of deuterium, an isotope of hydrogen, some of the hydrogen atoms may be deuterium, which is an isotope; and in this case, the content of deuterium may be 0% to 100%. In the case where the substituent is not shown in the chemical formula or the compound structure of the present disclosure, when deuterium is not explicitly excluded, hydrogen and deuterium may be mixed and used in the compound, such as when the content of deuterium is 0%, the content of hydrogen is 100%, and all substituents are hydrogen. The deuterium is an element having a deuteron composed of one proton and one neutron as an atomic nucleus, which is one of the isotopes of hydrogen, and may be represented by hydrogen-2, and the element symbol may be D or ²H. Although the isotope has the same atomic number (Z), an isotope having a different mass number (A) means the same number of protons and the number of neutrons may also be interpreted as an element having different numbers.

Herein, “combinations thereof” signifies that one or more components of the corresponding list are combined to form a known or chemically stable arrangement that a person skilled in the art could conceive of from the corresponding list. For example, alkyl and deuterium may be combined to form partially or entirely deuterated alkyl groups; halogen and alkyl may be combined to form halogenated alkyl substituents; and halogen, alkyl, and aryl may be combined to form halogenated arylalkyl. For example, preferred combinations of substituents may include up to 50 atoms excluding hydrogen and deuterium, or include up to 40 atoms excluding hydrogen and deuterium, or include up to 30 atoms excluding hydrogen and deuterium, or in many cases, preferred combinations of substituents may include up to 20 atoms excluding hydrogen and deuterium.

In the formula of the present disclosure, when multiple substituents are indicated by the same symbol, each of these substituents represented by the same symbol may be the same as or different from one another.

The compound represented by the above Formula 1 is described in more detail as follows.

In Formula 1, Y₁ to Y₆ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀)aliphatic ring and a (C₆-C₃₀)aromatic ring; or may be linked to an adjacent substituent to form a ring. According to one embodiment, Y₁, Y₂, Y₆, and Y₆ each independently may be hydrogen or deuterium. According to another embodiment, Y₃ and Y₄ each independently may be hydrogen, deuterium, or a substituted or unsubstituted (3- to 30-membered)heteroaryl. For example, Y₃ and Y₄ each independently may be hydrogen or deuterium, or a substituted or unsubstituted (5- to 30-membered)heteroaryl represented by Formula 1-a or 1-b.

In Formula 1, Y₇ and Y₈ each independently may be hydrogen, deuterium, halogen, cyano, a substituted or unsubstituted phenyl, a substituted or unsubstituted biphenyl, a substituted or unsubstituted terphenyl, a substituted or unsubstituted (C₁-C₃₀)alkyl, a substituted or unsubstituted (C₂-C₃₀)alkynyl, or a substituted or unsubstituted (3- to 30-membered)heteroaryl. According to one embodiment, Y₇ and Y₈ each independently may be hydrogen, deuterium, halogen, a substituted or unsubstituted phenyl, a substituted or unsubstituted biphenyl, a substituted or unsubstituted terphenyl, a substituted or unsubstituted (C₁-C₃₀)alkyl, a substituted or unsubstituted (C₂-C₃₀)alkynyl, or a substituted or unsubstituted (3- to 30-membered)heteroaryl. According to another embodiment, Y₇ and Y₈ each independently may be hydrogen, deuterium, halogen, a substituted or unsubstituted phenyl, a substituted or unsubstituted biphenyl, a substituted or unsubstituted terphenyl, a substituted or unsubstituted (C₁-C₁₀)alkyl, a substituted or unsubstituted (C₂-C₁₀)alkynyl, or a substituted or unsubstituted (3- to 10-membered)heteroaryl. For example, Y₇ and Y₈ each independently may be hydrogen, deuterium, fluoro, a dimethylphosphine oxide-substituted or unsubstituted phenyl, an unsubstituted pyridinyl, a phenyl-substituted pyridinyl, an unsubstituted pyrimidinyl, a phenyl-substituted pyrimidinyl, an unsubstituted biphenyl, an unsubstituted terphenyl, or a deuterium-substituted or unsubstituted methyl, and these may be further substituted with deuterium. Y₇ and Y₈ may be the same as or different from each other.

At least one of Y₁ to Y₈ is represented by the following Formula 1-a or 1-b, or is HAr, which is a substituted or unsubstituted (3- to 30-membered)heteroaryl.

HAr may be a substituted or unsubstituted (5- to 30-membered)heteroaryl, preferably a substituted or unsubstituted (5- to 30-membered)heteroaryl containing nitrogen. For example, HAr may be an unsubstituted pyridinyl.

In Formulas 1-a and 1-b, the dotted line indicates the presence or absence of each ring; for example, the dotted line denotes either absence or an aromatic ring.

In Formulas 1-a and 1-b, L may be a single bond, a substituted or unsubstituted (C₆-C₃₀)arylene, a substituted or unsubstituted (3- to 30-membered)heteroarylene, or a substituted or unsubstituted (C₂-C₃₀)alkynylene. According to one embodiment, L may be a single bond, a substituted or unsubstituted (C₆-C₃₀)arylene, or a substituted or unsubstituted (C₂-C₁₀)alkynylene. For example, L may be a single bond, un unsubstituted phenylene, an unsubstituted biphenylene, an unsubstituted naphthylene, an unsubstituted ethynylene, or the like.

In Formula 1-a, X₁ to X₃ each independently represent —N═, —O—, —S—, or —CRₐ—; provided that one of X₁ to X₃ is —N═, another one of X₁ to X₃ is —O— or —S—, and the remaining one of X₁ to X₃ is —O—, —S—, or —CRₐ—. For example, one of X₁ and X₃ may be —N═, the other of X₁ and X₃ may be —O— or —S—, and X₂ may be CRₐ.

In Formulas 1-b, X₄ and X₆ each independently represent —N═, —O—, or —S—; provided that one of X₄ and X₆ is —N═ and the other is —O— or —S—;

In Formula 1-a, X₂ may be —CRₐ—.

In Formula 1-a, R₁ to R₃ each independently represent a position for bonding to L, or hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, or a substituted or unsubstituted (C₁-C₃₀)alkoxy; or may be linked to an adjacent substituent to form a ring.

In Formula 1-a, Rₐ represents hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, or a substituted or unsubstituted (C₁-C₃₀)alkoxy; or may be linked to an adjacent substituent to form a ring.

In Formula 1-b, R₄ to R₆ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, or a substituted or unsubstituted (C₁-C₃₀)alkoxy; or may be linked to an adjacent substituent to form a ring.

According to one embodiment, Rₐ and R₁ to R₆ each independently may be hydrogen, deuterium, or a substituted or unsubstituted (C₆-C₃₀)aryl. According to another embodiment, Rₐ and R₁ to R₆ each independently may be hydrogen, deuterium, or a substituted or unsubstituted (C₆-C₁₈)aryl. For example, Rₐ and R₁ to R₆ each independently may be hydrogen; a dimethylphosphine oxide-substituted or unsubstituted phenyl; an unsubstituted naphthyl; an unsubstituted phenanthrenyl; an unsubstituted biphenyl; an unsubstituted terphenyl; a phenyl-substituted naphthyl; or a naphthyl-substituted phenyl. Rₐ and R₁ to R₆ may be the same as or different from each other.

In Formulas 1-a and 1-b, m is an integer of 1 or 2. For example, m may be 2, wherein each L may be the same or different.

In Formulas 1-a and 1-b, a and d are each independently an integer of 1 or 2, and b, c, e, and f are each independently an integer of 1 to 4.

In Formulas 1-a and 1-b, when a to f are integers of 2 or more, each of R₁ to each of R₆ may be the same or different.

Formula 1 may be represented by any one of the following Formulas 1-1 to 1-6.

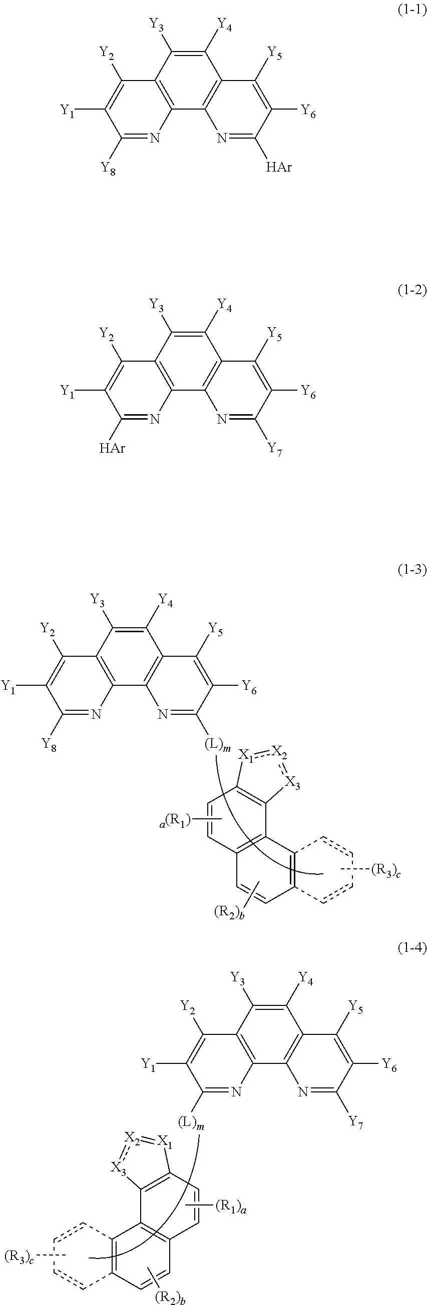

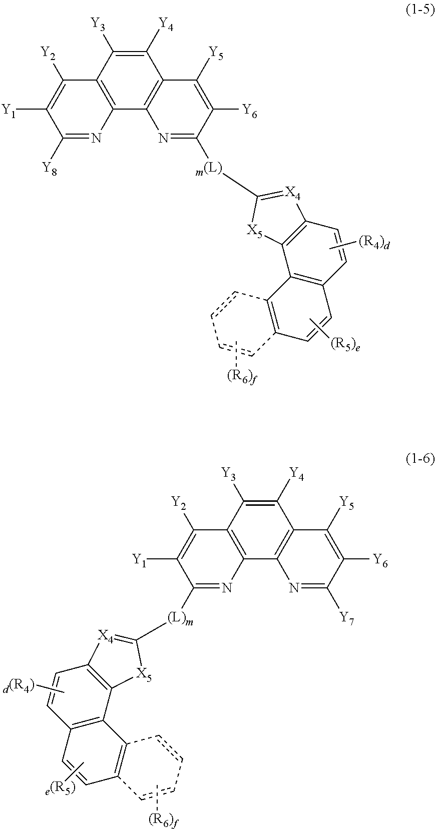

In Formulas 1-1 to 1-6, Y₁ to Y₈, HAr, L, X₁ to X₅, R₁ to R₆, m, and a to f are as defined in Formula 1.

Formula 1 may be represented by any one of the following Formulas 1-7 to 1-12.

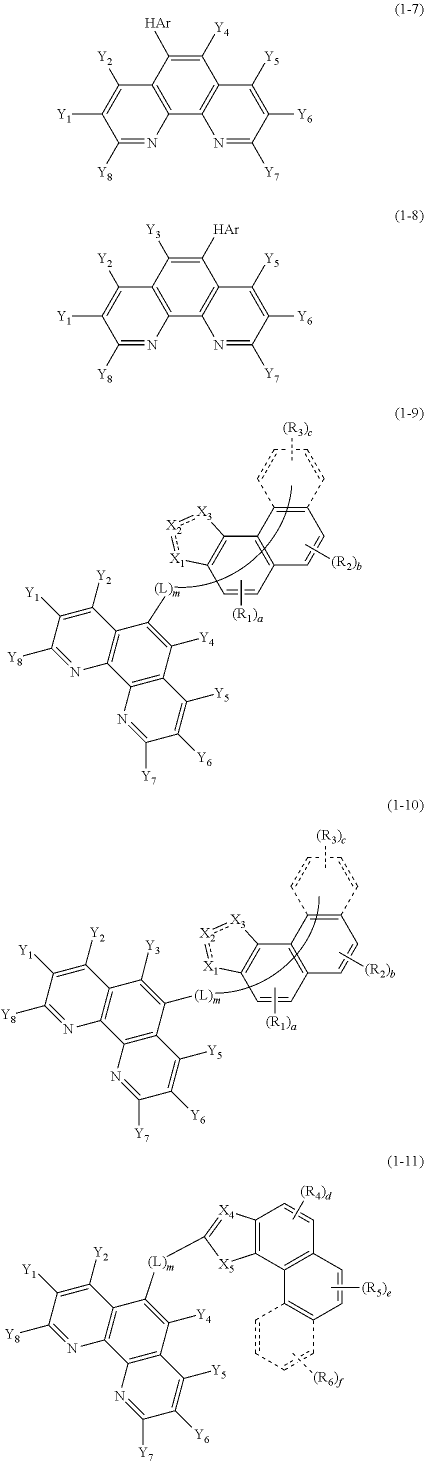

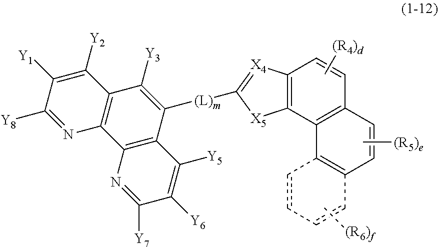

In Formulas 1-7 to 1-12, Y₁ to Y₈, HAr, L, X₁ to X₅, R₁ to R₆, m, and a to f are as defined in Formula 1.

Formula 1-a may be represented by any one of the following Formulas 1-a-1 to 1-a-10, and Formula 1-b may be represented by any one of the following Formulas 1-b-1 and 1-b-2.

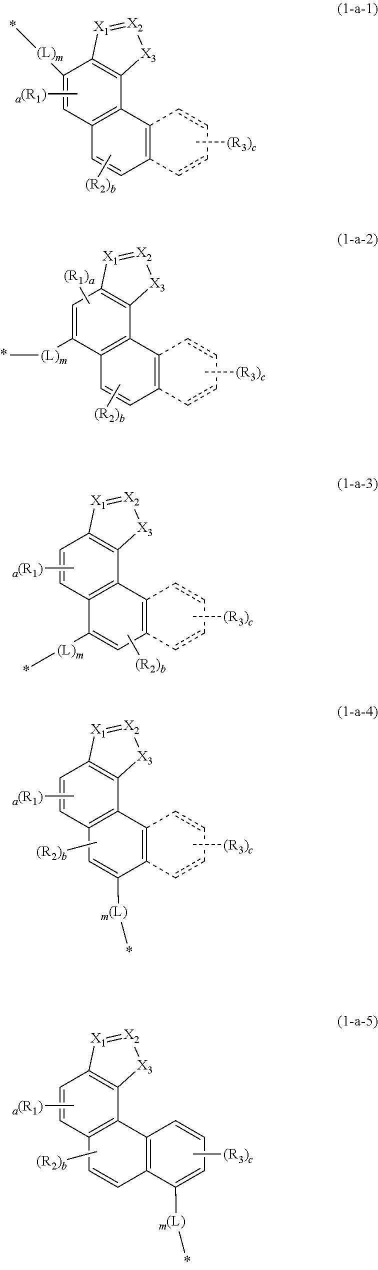

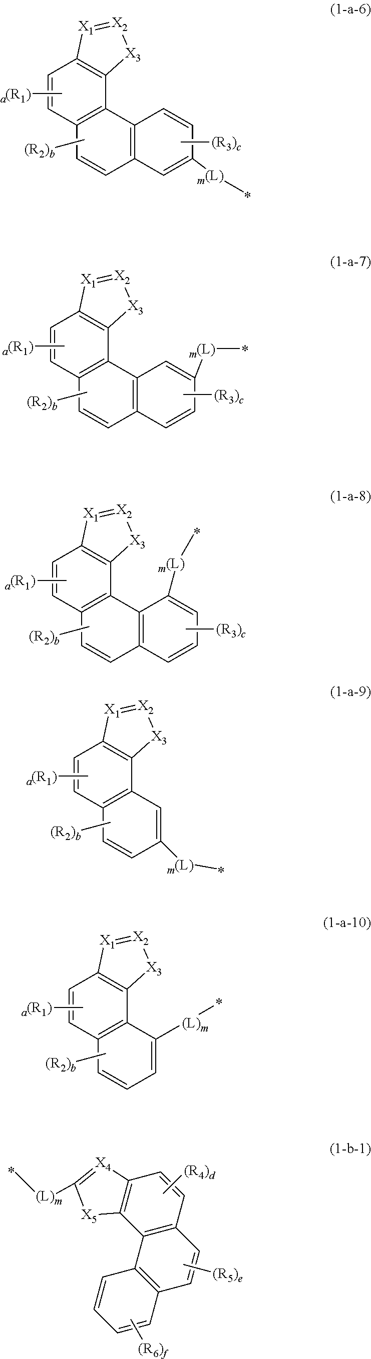

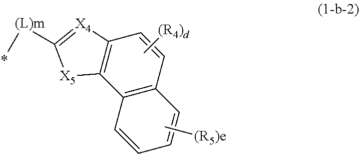

In Formulas 1-a-1 to 1-a-10, 1-b-1, and 1-b-2, R₁ to R₆, X₁ to X₅, L, a to f, and m are as defined in Formula 1.

The compound represented by Formula 1 may be at least one selected from the following compounds, but is not limited thereto.

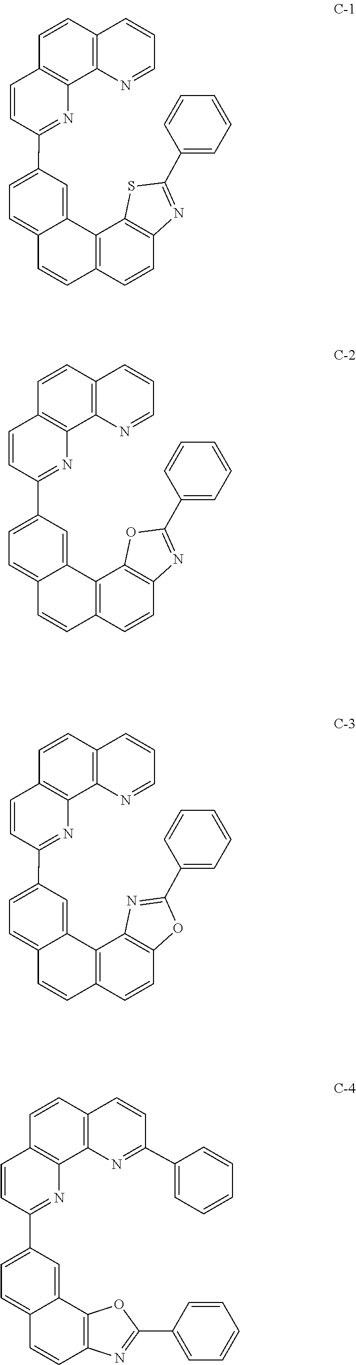 [SMILES: c1ccc(-c2nc3ccc4ccc5ccc(-c6ccc7ccc8cccnc8c7n6)cc5c4c3s2)cc1 ; c1ccc(-c2nc3ccc4ccc5ccc(-c6ccc7ccc8cccnc8c7n6)cc5c4c3o2)cc1 ; c1ccc(-c2nc3c(ccc4ccc5ccc(-c6ccc7ccc8cccnc8c7n6)cc5c43)o2)cc1 ; c1ccc(-c2ccc3ccc4ccc(-c5ccc6ccc7nc(-c8ccccc8)oc7c6c5)nc4c3n2)cc1]

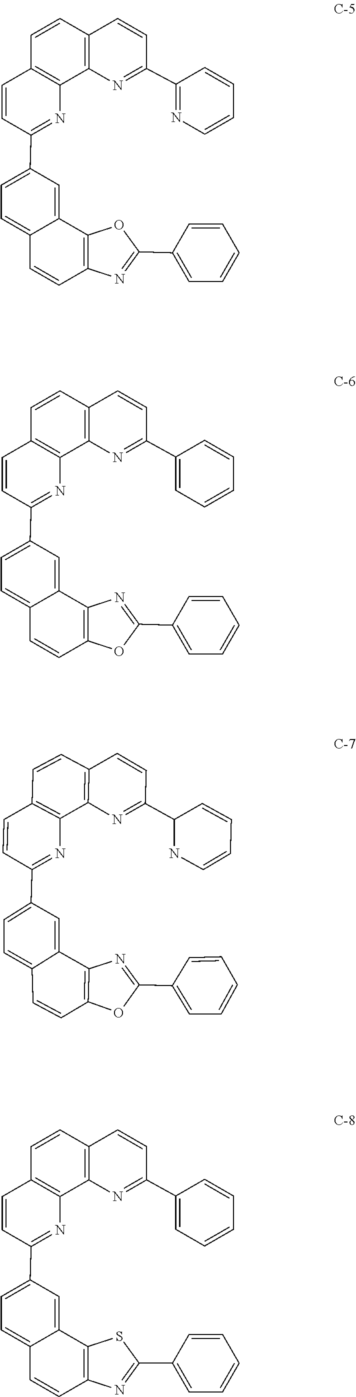 [SMILES: c1ccc(-c2nc3ccc4ccc(-c5ccc6ccc7ccc(-c8ccccn8)nc7c6n5)cc4c3o2)cc1 ; c1ccc(-c2ccc3ccc4ccc(-c5ccc6ccc7oc(-c8ccccc8)nc7c6c5)nc4c3n2)cc1 ; C1=CNC(c2ccc3ccc4ccc(-c5ccc6ccc7oc(-c8ccccc8)nc7c6c5)nc4c3n2)C=C1 ; c1ccc(-c2ccc3ccc4ccc(-c5ccc6ccc7nc(-c8ccccc8)sc7c6c5)nc4c3n2)cc1]

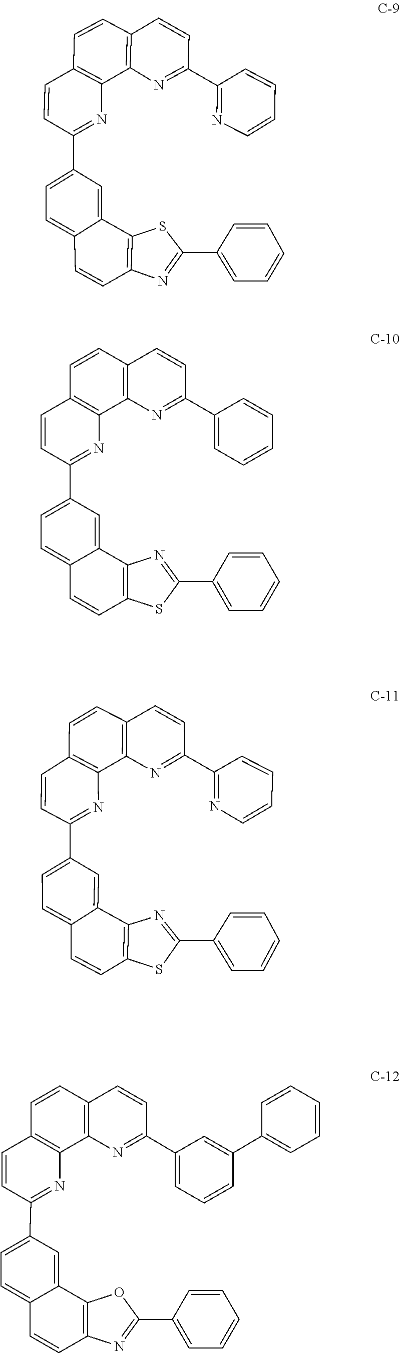 [SMILES: c1ccc(-c2nc3ccc4ccc(-c5ccc6ccc7ccc(-c8ccccn8)nc7c6n5)cc4c3s2)cc1 ; c1ccc(-c2ccc3ccc4ccc(-c5ccc6ccc7sc(-c8ccccc8)nc7c6c5)nc4c3n2)cc1 ; c1ccc(-c2nc3c(ccc4ccc(-c5ccc6ccc7ccc(-c8ccccn8)nc7c6n5)cc43)s2)cc1 ; c1ccc(-c2cccc(-c3ccc4ccc5ccc(-c6ccc7ccc8nc(-c9ccccc9)oc8c7c6)nc5c4n3)c2)cc1]

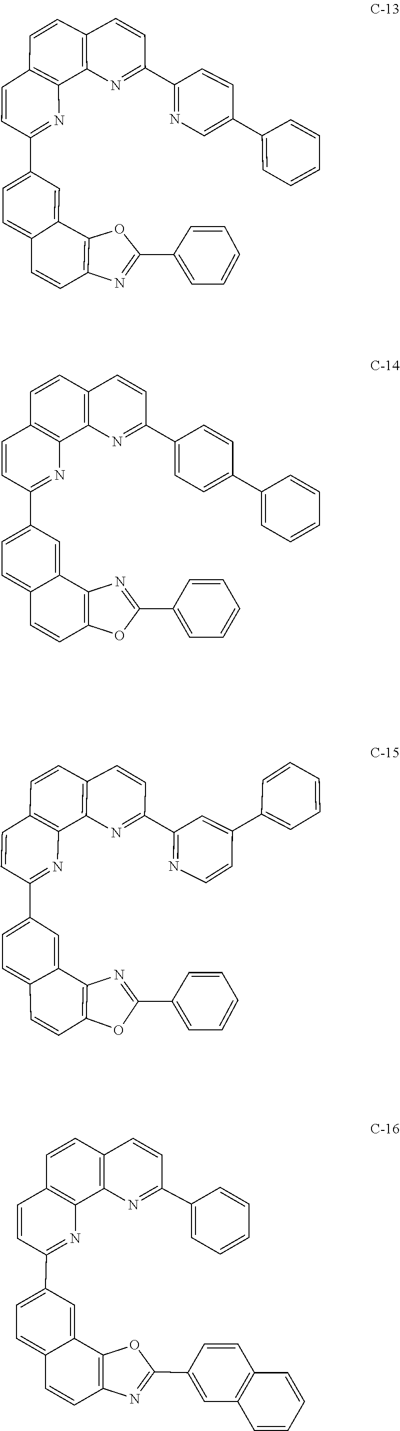 [SMILES: c1ccc(-c2ccc(-c3ccc4ccc5ccc(-c6ccc7ccc8nc(-c9ccccc9)oc8c7c6)nc5c4n3)nc2)cc1 ; c1ccc(-c2ccc(-c3ccc4ccc5ccc(-c6ccc7ccc8oc(-c9ccccc9)nc8c7c6)nc5c4n3)cc2)cc1 ; c1ccc(-c2ccnc(-c3ccc4ccc5ccc(-c6ccc7ccc8oc(-c9ccccc9)nc8c7c6)nc5c4n3)c2)cc1 ; c1ccc(-c2ccc3ccc4ccc(-c5ccc6ccc7nc(-c8ccc9ccccc9c8)oc7c6c5)nc4c3n2)cc1]

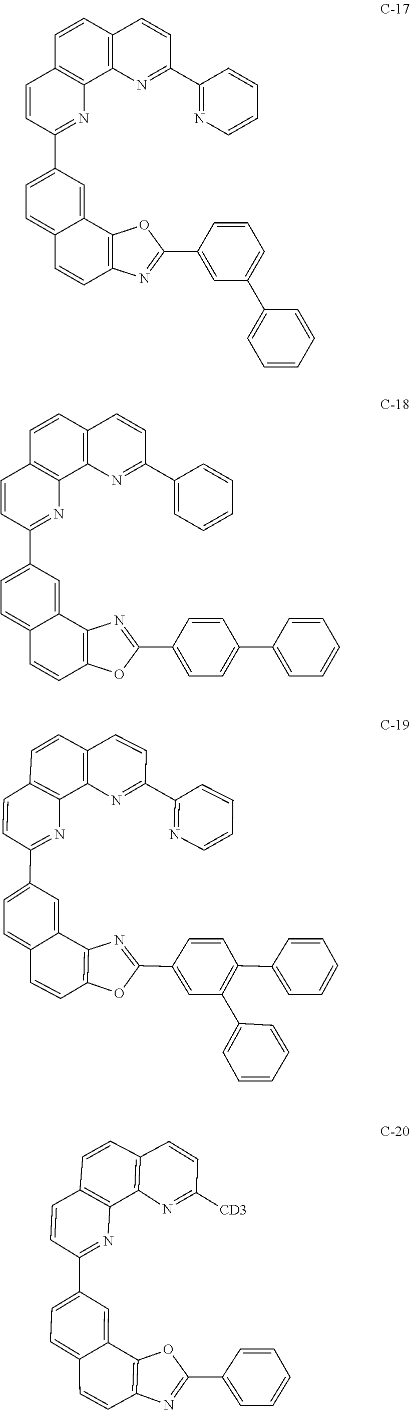 [SMILES: c1ccc(-c2cccc(-c3nc4ccc5ccc(-c6ccc7ccc8ccc(-c9ccccn9)nc8c7n6)cc5c4o3)c2)cc1 ; c1ccc(-c2ccc(-c3nc4c(ccc5ccc(-c6ccc7ccc8ccc(-c9ccccc9)nc8c7n6)cc54)o3)cc2)cc1 ; c1ccc(-c2ccc(-c3nc4c(ccc5ccc(-c6ccc7ccc8ccc(-c9ccccn9)nc8c7n6)cc54)o3)cc2-c2ccccc2)cc1 ; [2H]C([2H])([2H])c1ccc2ccc3ccc(-c4ccc5ccc6nc(-c7ccccc7)oc6c5c4)nc3c2n1]

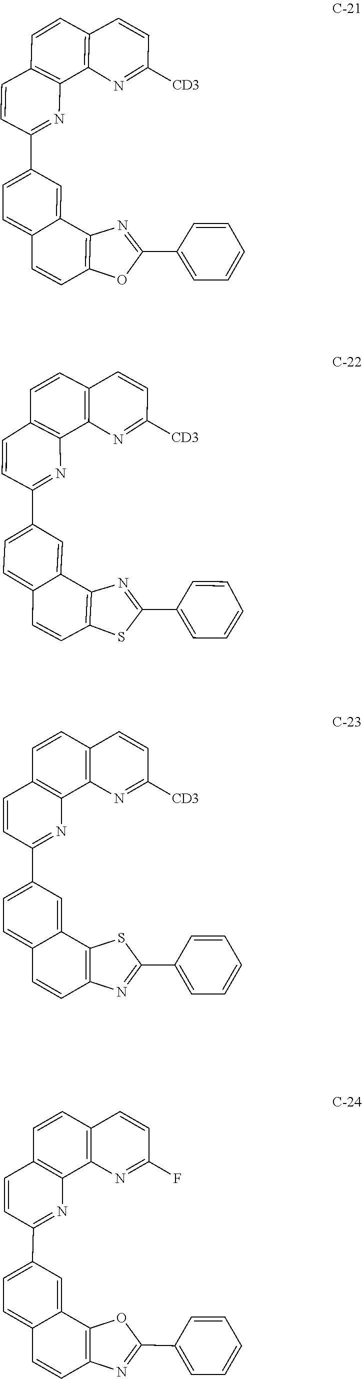 [SMILES: [2H]C([2H])([2H])c1ccc2ccc3ccc(-c4ccc5ccc6oc(-c7ccccc7)nc6c5c4)nc3c2n1 ; [2H]C([2H])([2H])c1ccc2ccc3ccc(-c4ccc5ccc6sc(-c7ccccc7)nc6c5c4)nc3c2n1 ; [2H]C([2H])([2H])c1ccc2ccc3ccc(-c4ccc5ccc6nc(-c7ccccc7)sc6c5c4)nc3c2n1 ; Fc1ccc2ccc3ccc(-c4ccc5ccc6nc(-c7ccccc7)oc6c5c4)nc3c2n1]

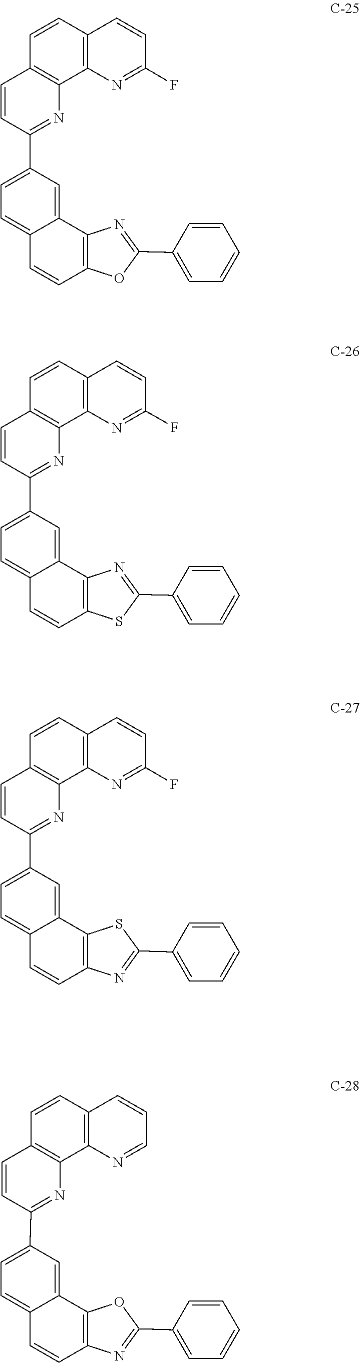 [SMILES: Fc1ccc2ccc3ccc(-c4ccc5ccc6oc(-c7ccccc7)nc6c5c4)nc3c2n1 ; Fc1ccc2ccc3ccc(-c4ccc5ccc6sc(-c7ccccc7)nc6c5c4)nc3c2n1 ; Fc1ccc2ccc3ccc(-c4ccc5ccc6nc(-c7ccccc7)sc6c5c4)nc3c2n1 ; c1ccc(-c2nc3ccc4ccc(-c5ccc6ccc7cccnc7c6n5)cc4c3o2)cc1]

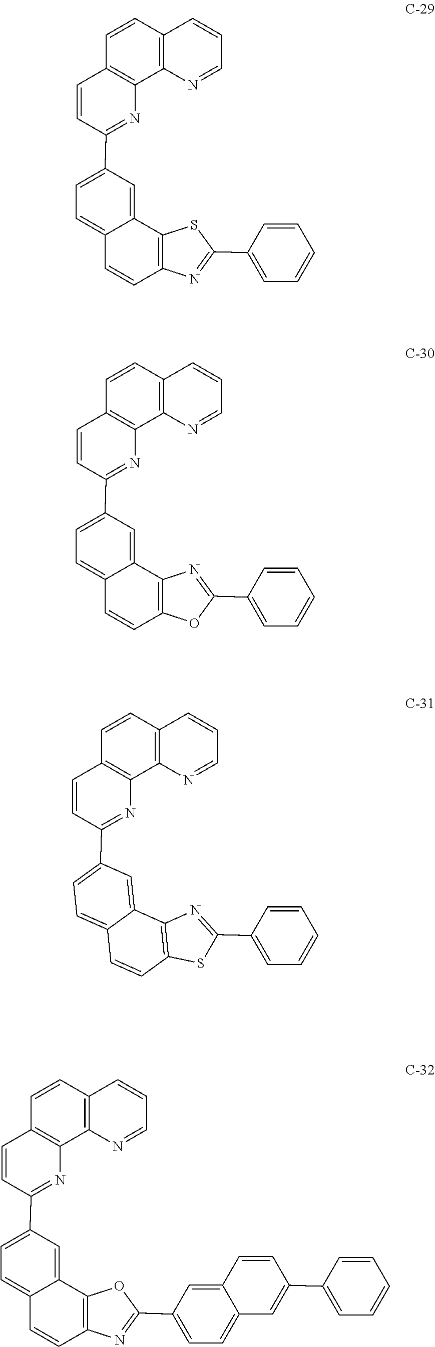 [SMILES: c1ccc(-c2nc3ccc4ccc(-c5ccc6ccc7cccnc7c6n5)cc4c3s2)cc1 ; c1ccc(-c2nc3c(ccc4ccc(-c5ccc6ccc7cccnc7c6n5)cc43)o2)cc1 ; c1ccc(-c2nc3c(ccc4ccc(-c5ccc6ccc7cccnc7c6n5)cc43)s2)cc1 ; c1ccc(-c2ccc3cc(-c4nc5ccc6ccc(-c7ccc8ccc9cccnc9c8n7)cc6c5o4)ccc3c2)cc1]

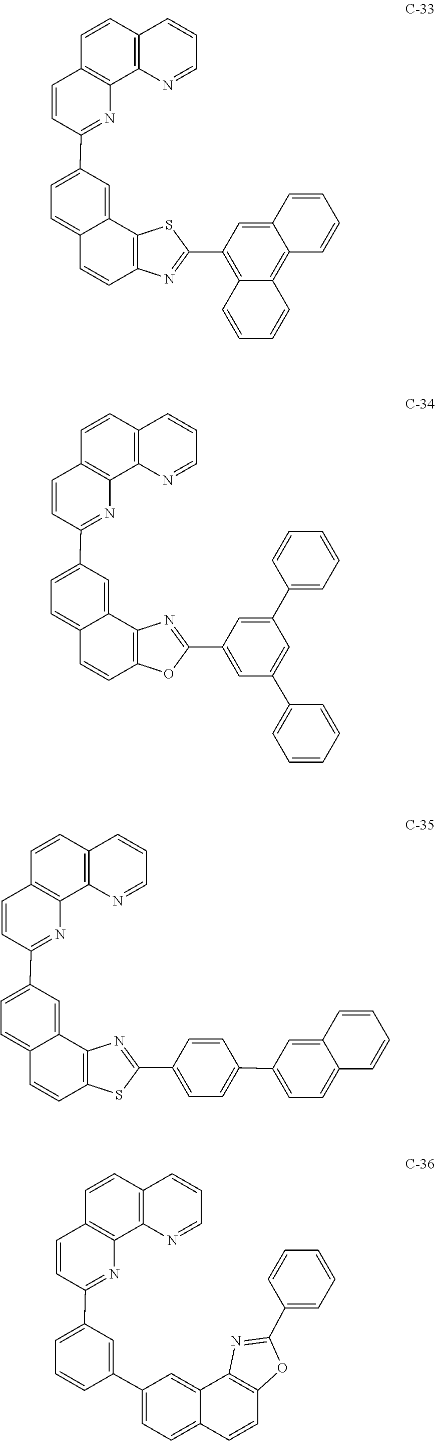 [SMILES: c1ccc2c(c1)cc(-c1nc3ccc4ccc(-c5ccc6ccc7cccnc7c6n5)cc4c3s1)c1ccccc12 ; c1ccc(-c2cc(-c3ccccc3)cc(-c3nc4c(ccc5ccc(-c6ccc7ccc8cccnc8c7n6)cc54)o3)c2)cc1 ; c1ccc2cc(-c3ccc(-c4nc5c(ccc6ccc(-c7ccc8ccc9cccnc9c8n7)cc65)s4)cc3)ccc2c1 ; c1ccc(-c2nc3c(ccc4ccc(-c5cccc(-c6ccc7ccc8cccnc8c7n6)c5)cc43)o2)cc1]

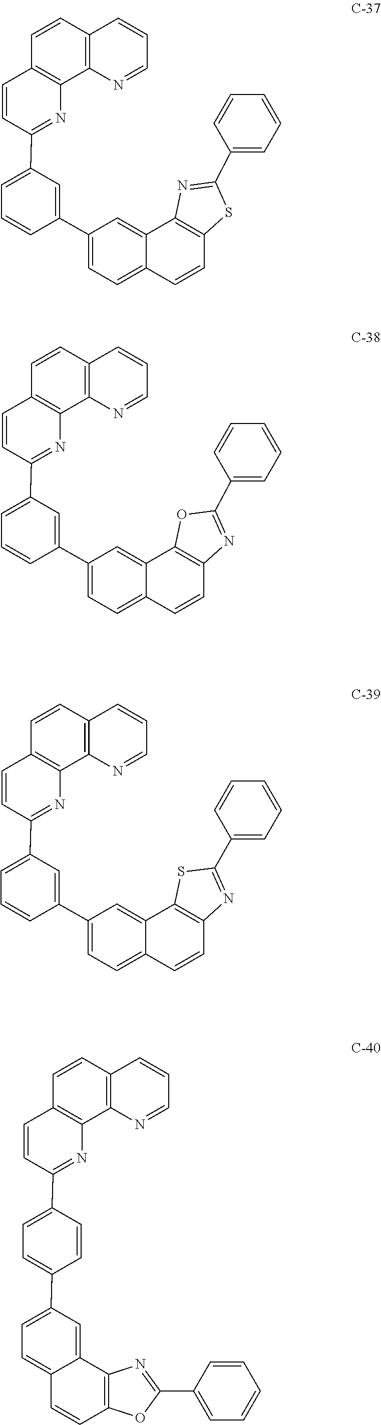 [SMILES: c1ccc(-c2nc3c(ccc4ccc(-c5cccc(-c6ccc7ccc8cccnc8c7n6)c5)cc43)s2)cc1 ; c1ccc(-c2nc3ccc4ccc(-c5cccc(-c6ccc7ccc8cccnc8c7n6)c5)cc4c3o2)cc1 ; c1ccc(-c2nc3ccc4ccc(-c5cccc(-c6ccc7ccc8cccnc8c7n6)c5)cc4c3s2)cc1 ; c1ccc(-c2nc3c(ccc4ccc(-c5ccc(-c6ccc7ccc8cccnc8c7n6)cc5)cc43)o2)cc1]

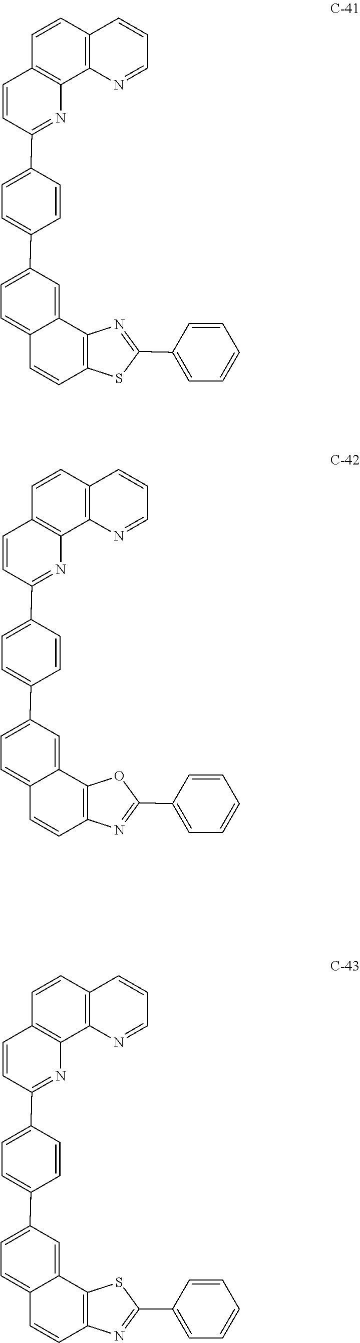 [SMILES: c1ccc(-c2nc3c(ccc4ccc(-c5ccc(-c6ccc7ccc8cccnc8c7n6)cc5)cc43)s2)cc1 ; c1ccc(-c2nc3ccc4ccc(-c5ccc(-c6ccc7ccc8cccnc8c7n6)cc5)cc4c3o2)cc1 ; c1ccc(-c2nc3ccc4ccc(-c5ccc(-c6ccc7ccc8cccnc8c7n6)cc5)cc4c3s2)cc1]

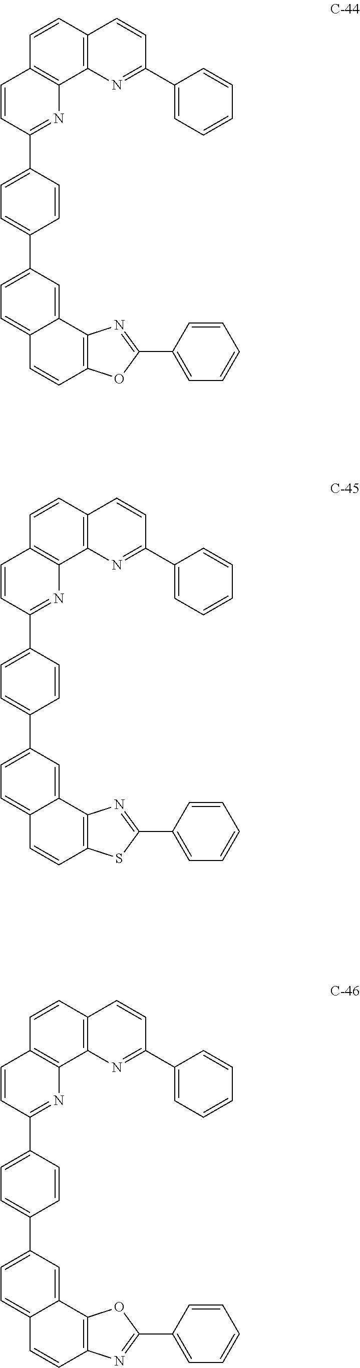 [SMILES: c1ccc(-c2ccc3ccc4ccc(-c5ccc(-c6ccc7ccc8oc(-c9ccccc9)nc8c7c6)cc5)nc4c3n2)cc1 ; c1ccc(-c2ccc3ccc4ccc(-c5ccc(-c6ccc7ccc8sc(-c9ccccc9)nc8c7c6)cc5)nc4c3n2)cc1 ; c1ccc(-c2ccc3ccc4ccc(-c5ccc(-c6ccc7ccc8nc(-c9ccccc9)oc8c7c6)cc5)nc4c3n2)cc1]

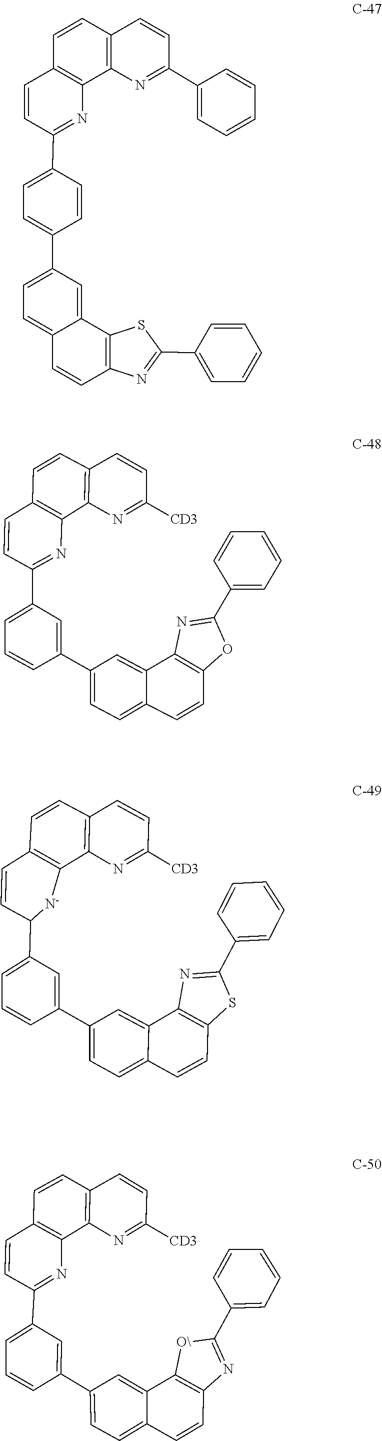

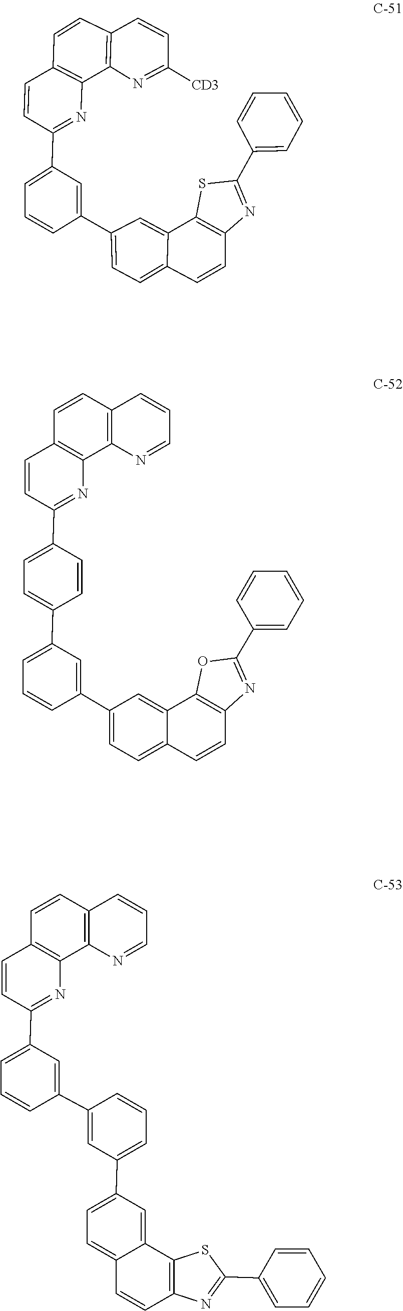 [SMILES: [2H]C([2H])([2H])c1ccc2ccc3ccc(-c4cccc(-c5ccc6ccc7nc(-c8ccccc8)sc7c6c5)c4)nc3c2n1 ; c1ccc(-c2nc3ccc4ccc(-c5cccc(-c6ccc(-c7ccc8ccc9cccnc9c8n7)cc6)c5)cc4c3o2)cc1 ; c1ccc(-c2nc3ccc4ccc(-c5cccc(-c6cccc(-c7ccc8ccc9cccnc9c8n7)c6)c5)cc4c3s2)cc1]

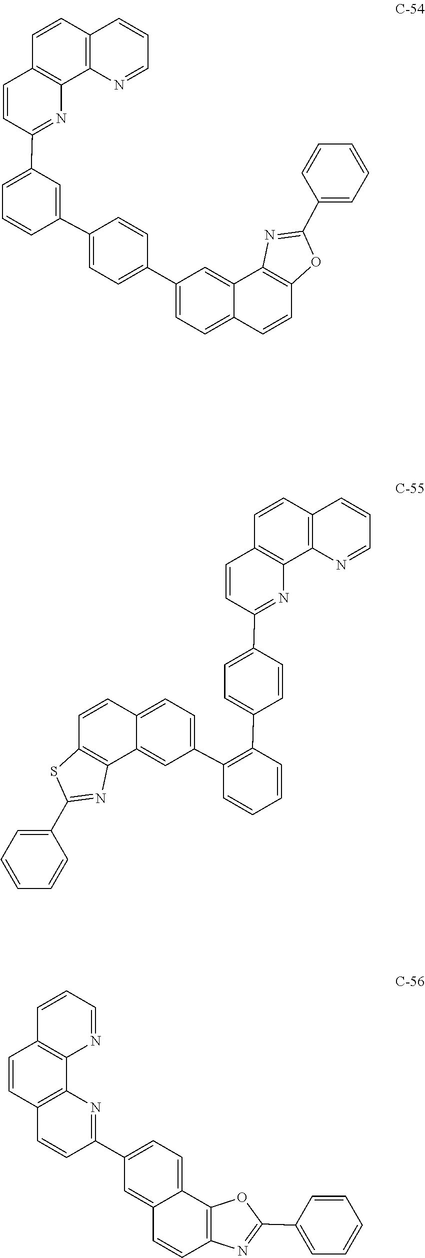 [SMILES: c1ccc(-c2nc3c(ccc4ccc(-c5ccc(-c6cccc(-c7ccc8ccc9cccnc9c8n7)c6)cc5)cc43)o2)cc1 ; c1ccc(-c2nc3c(ccc4ccc(-c5ccccc5-c5ccc(-c6ccc7ccc8cccnc8c7n6)cc5)cc43)s2)cc1 ; c1ccc(-c2nc3ccc4cc(-c5ccc6ccc7cccnc7c6n5)ccc4c3o2)cc1]

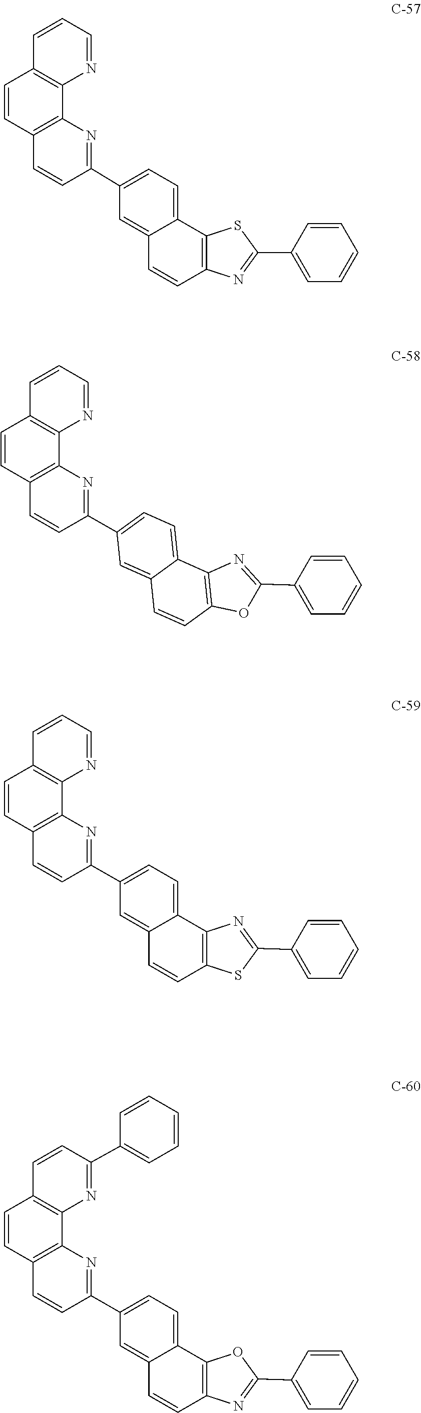 [SMILES: c1ccc(-c2nc3ccc4cc(-c5ccc6ccc7cccnc7c6n5)ccc4c3s2)cc1 ; c1ccc(-c2nc3c(ccc4cc(-c5ccc6ccc7cccnc7c6n5)ccc43)o2)cc1 ; c1ccc(-c2nc3c(ccc4cc(-c5ccc6ccc7cccnc7c6n5)ccc43)s2)cc1 ; c1ccc(-c2ccc3ccc4ccc(-c5ccc6c(ccc7nc(-c8ccccc8)oc76)c5)nc4c3n2)cc1]

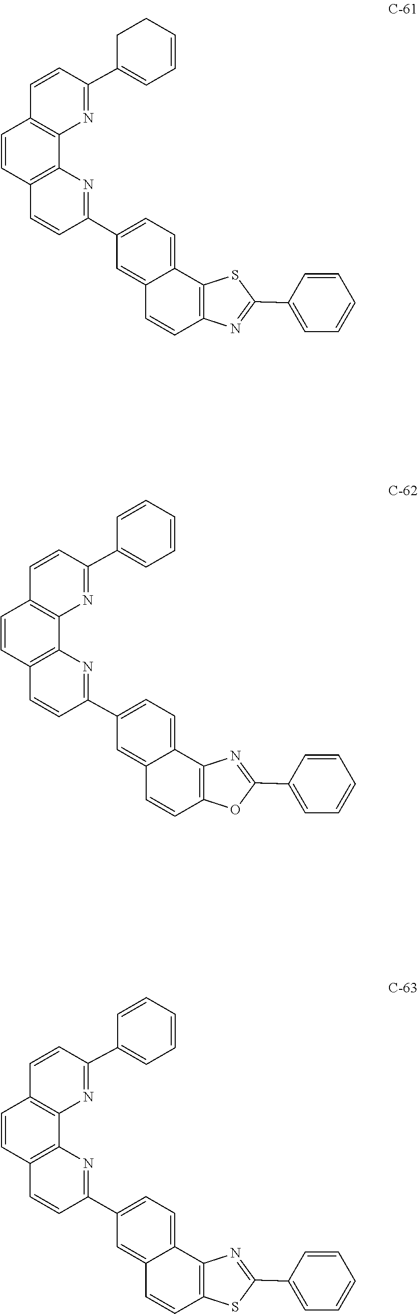 [SMILES: C1=CCCC(c2ccc3ccc4ccc(-c5ccc6c(ccc7nc(-c8ccccc8)sc76)c5)nc4c3n2)=C1 ; c1ccc(-c2ccc3ccc4ccc(-c5ccc6c(ccc7oc(-c8ccccc8)nc76)c5)nc4c3n2)cc1 ; c1ccc(-c2ccc3ccc4ccc(-c5ccc6c(ccc7sc(-c8ccccc8)nc76)c5)nc4c3n2)cc1]

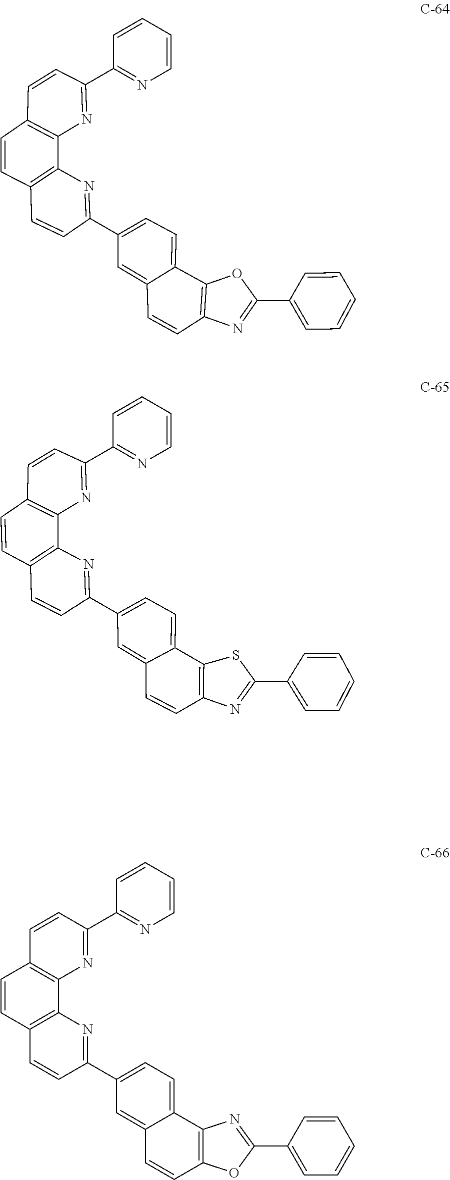 [SMILES: c1ccc(-c2nc3ccc4cc(-c5ccc6ccc7ccc(-c8ccccn8)nc7c6n5)ccc4c3o2)cc1 ; c1ccc(-c2nc3ccc4cc(-c5ccc6ccc7ccc(-c8ccccn8)nc7c6n5)ccc4c3s2)cc1 ; c1ccc(-c2nc3c(ccc4cc(-c5ccc6ccc7ccc(-c8ccccn8)nc7c6n5)ccc43)o2)cc1]

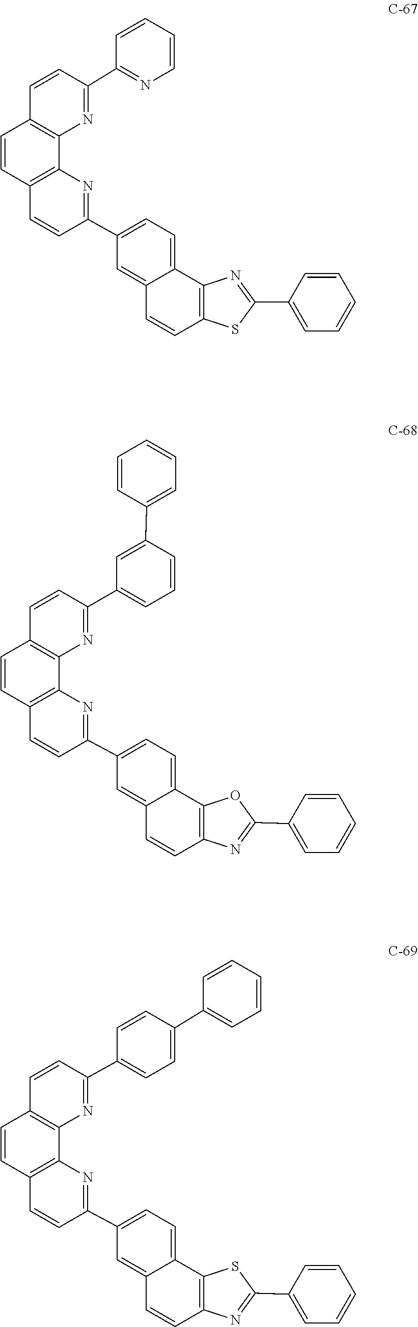 [SMILES: c1ccc(-c2nc3c(ccc4cc(-c5ccc6ccc7ccc(-c8ccccn8)nc7c6n5)ccc43)s2)cc1 ; c1ccc(-c2cccc(-c3ccc4ccc5ccc(-c6ccc7c(ccc8nc(-c9ccccc9)oc87)c6)nc5c4n3)c2)cc1 ; c1ccc(-c2ccc(-c3ccc4ccc5ccc(-c6ccc7c(ccc8nc(-c9ccccc9)sc87)c6)nc5c4n3)cc2)cc1]

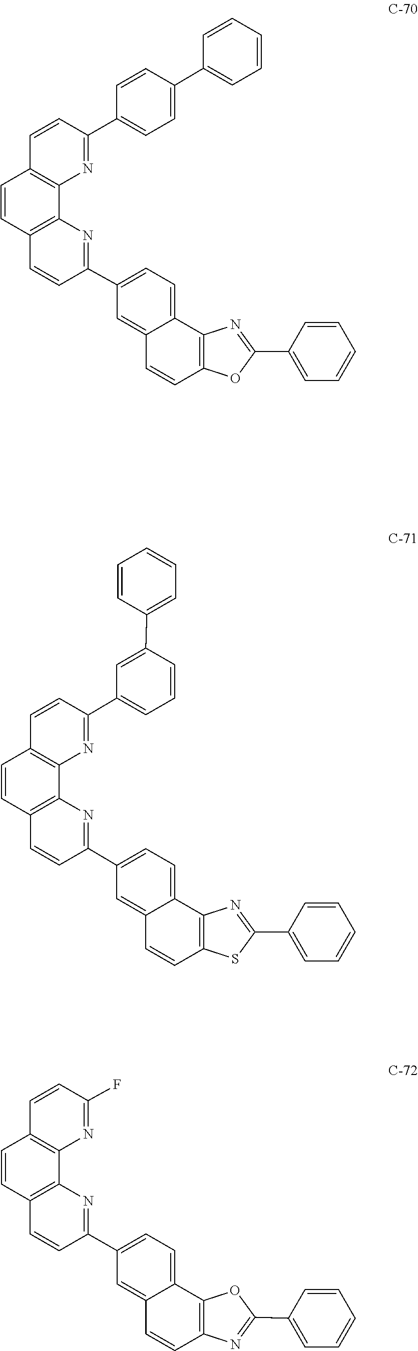 [SMILES: c1ccc(-c2ccc(-c3ccc4ccc5ccc(-c6ccc7c(ccc8oc(-c9ccccc9)nc87)c6)nc5c4n3)cc2)cc1 ; c1ccc(-c2cccc(-c3ccc4ccc5ccc(-c6ccc7c(ccc8sc(-c9ccccc9)nc87)c6)nc5c4n3)c2)cc1 ; Fc1ccc2ccc3ccc(-c4ccc5c(ccc6nc(-c7ccccc7)oc65)c4)nc3c2n1]

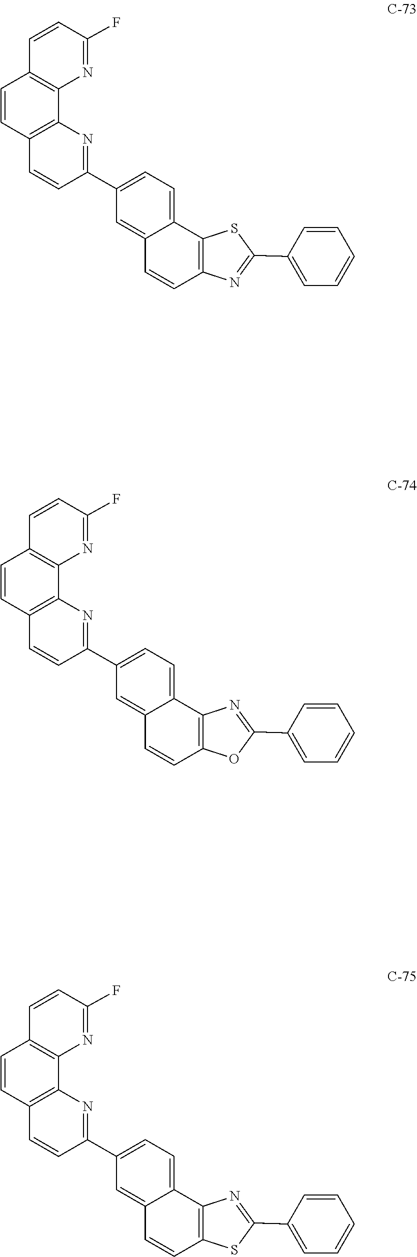 [SMILES: Fc1ccc2ccc3ccc(-c4ccc5c(ccc6nc(-c7ccccc7)sc65)c4)nc3c2n1 ; Fc1ccc2ccc3ccc(-c4ccc5c(ccc6oc(-c7ccccc7)nc65)c4)nc3c2n1 ; Fc1ccc2ccc3ccc(-c4ccc5c(ccc6sc(-c7ccccc7)nc65)c4)nc3c2n1]

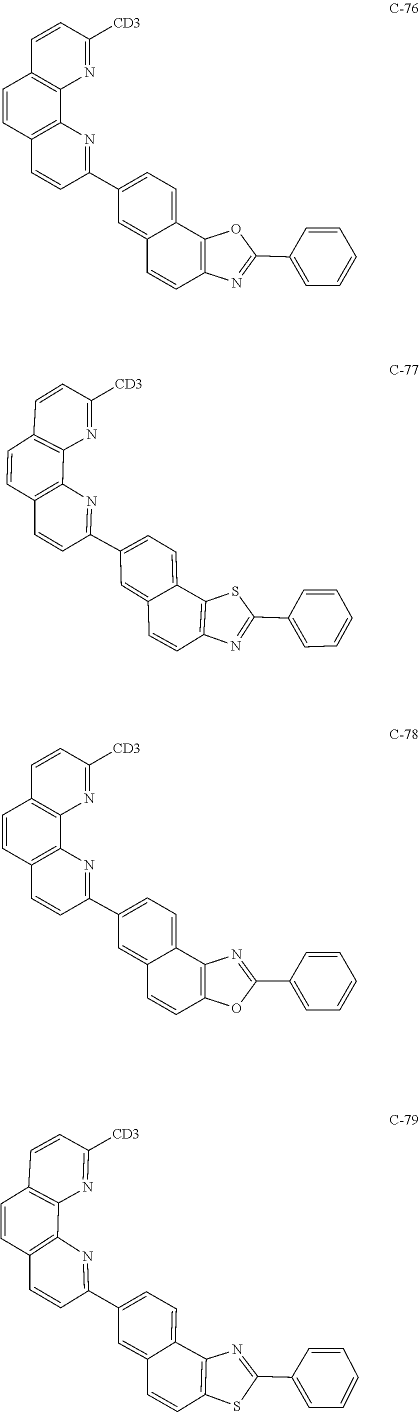 [SMILES: [2H]C([2H])([2H])c1ccc2ccc3ccc(-c4ccc5c(ccc6nc(-c7ccccc7)oc65)c4)nc3c2n1 ; [2H]C([2H])([2H])c1ccc2ccc3ccc(-c4ccc5c(ccc6nc(-c7ccccc7)sc65)c4)nc3c2n1 ; [2H]C([2H])([2H])c1ccc2ccc3ccc(-c4ccc5c(ccc6oc(-c7ccccc7)nc65)c4)nc3c2n1 ; [2H]C([2H])([2H])c1ccc2ccc3ccc(-c4ccc5c(ccc6sc(-c7ccccc7)nc65)c4)nc3c2n1]

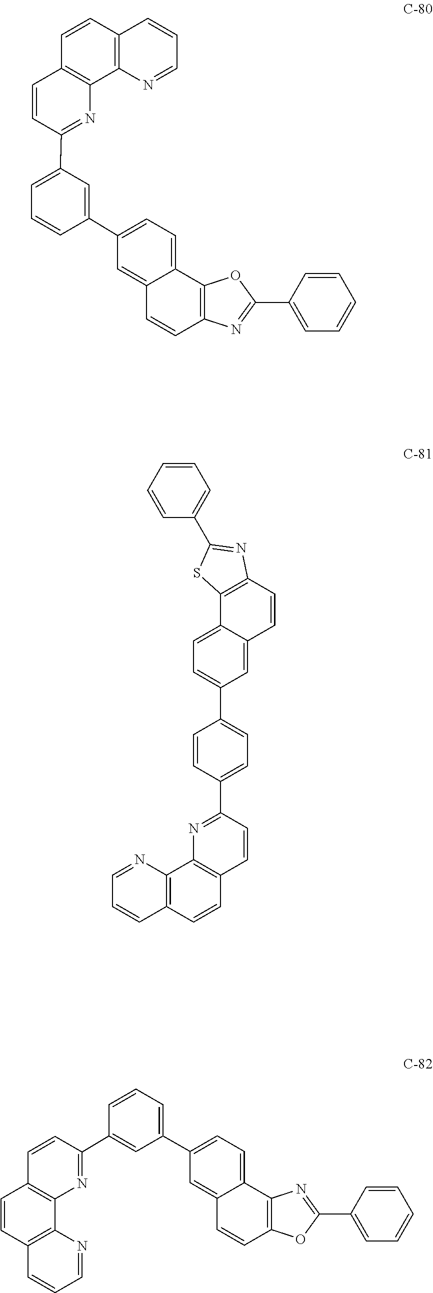 [SMILES: c1ccc(-c2nc3ccc4cc(-c5cccc(-c6ccc7ccc8cccnc8c7n6)c5)ccc4c3o2)cc1 ; c1ccc(-c2nc3ccc4cc(-c5ccc(-c6ccc7ccc8cccnc8c7n6)cc5)ccc4c3s2)cc1 ; c1ccc(-c2nc3c(ccc4cc(-c5cccc(-c6ccc7ccc8cccnc8c7n6)c5)ccc43)o2)cc1]

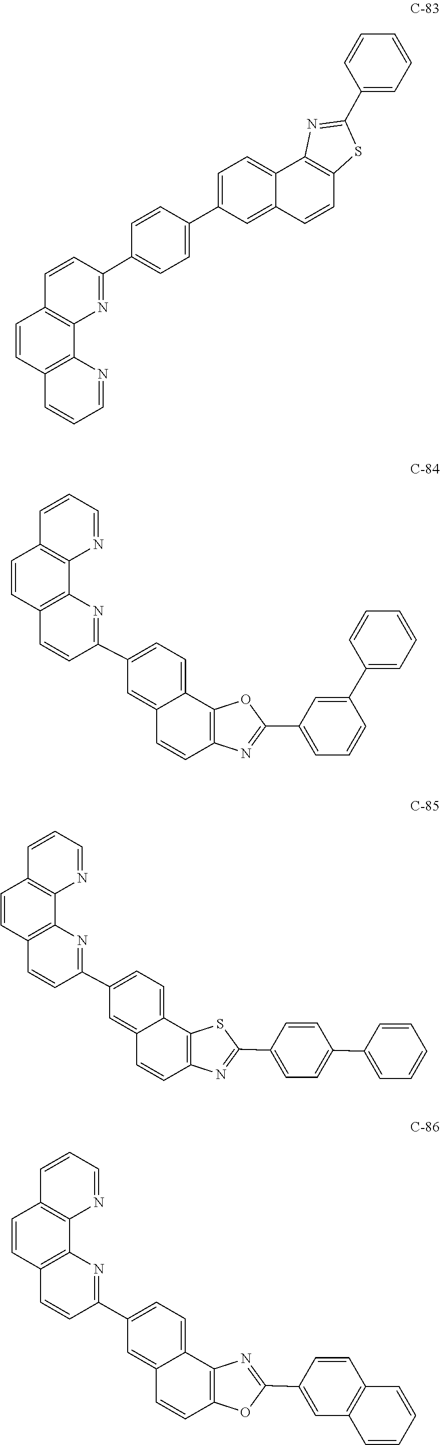 [SMILES: c1ccc(-c2nc3c(ccc4cc(-c5ccc(-c6ccc7ccc8cccnc8c7n6)cc5)ccc43)s2)cc1 ; c1ccc(-c2cccc(-c3nc4ccc5cc(-c6ccc7ccc8cccnc8c7n6)ccc5c4o3)c2)cc1 ; c1ccc(-c2ccc(-c3nc4ccc5cc(-c6ccc7ccc8cccnc8c7n6)ccc5c4s3)cc2)cc1 ; c1ccc2cc(-c3nc4c(ccc5cc(-c6ccc7ccc8cccnc8c7n6)ccc54)o3)ccc2c1]

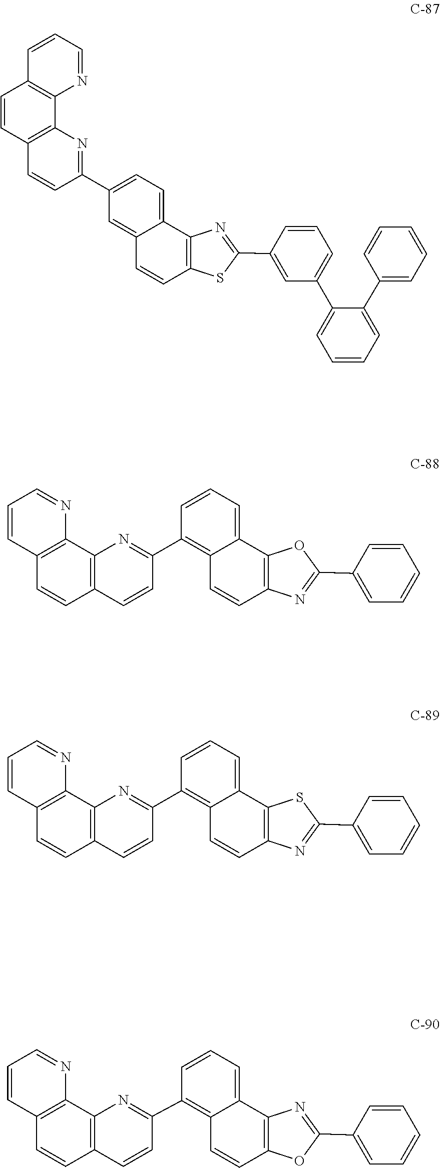 [SMILES: c1ccc(-c2ccccc2-c2cccc(-c3nc4c(ccc5cc(-c6ccc7ccc8cccnc8c7n6)ccc54)s3)c2)cc1 ; c1ccc(-c2nc3ccc4c(-c5ccc6ccc7cccnc7c6n5)cccc4c3o2)cc1 ; c1ccc(-c2nc3ccc4c(-c5ccc6ccc7cccnc7c6n5)cccc4c3s2)cc1 ; c1ccc(-c2nc3c(ccc4c(-c5ccc6ccc7cccnc7c6n5)cccc43)o2)cc1]

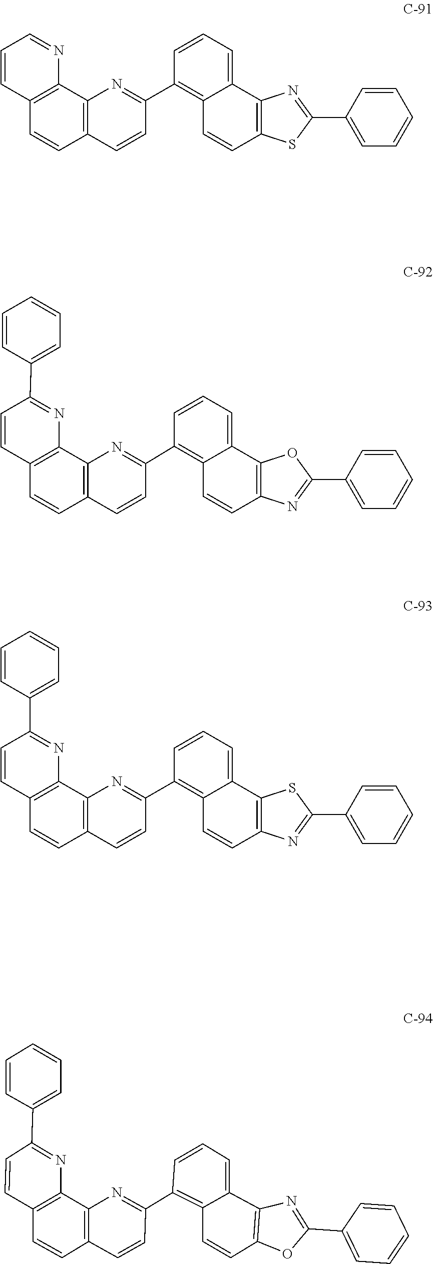 [SMILES: c1ccc(-c2nc3c(ccc4c(-c5ccc6ccc7cccnc7c6n5)cccc43)s2)cc1 ; c1ccc(-c2ccc3ccc4ccc(-c5cccc6c5ccc5nc(-c7ccccc7)oc56)nc4c3n2)cc1 ; c1ccc(-c2ccc3ccc4ccc(-c5cccc6c5ccc5nc(-c7ccccc7)sc56)nc4c3n2)cc1 ; c1ccc(-c2ccc3ccc4ccc(-c5cccc6c5ccc5oc(-c7ccccc7)nc56)nc4c3n2)cc1]

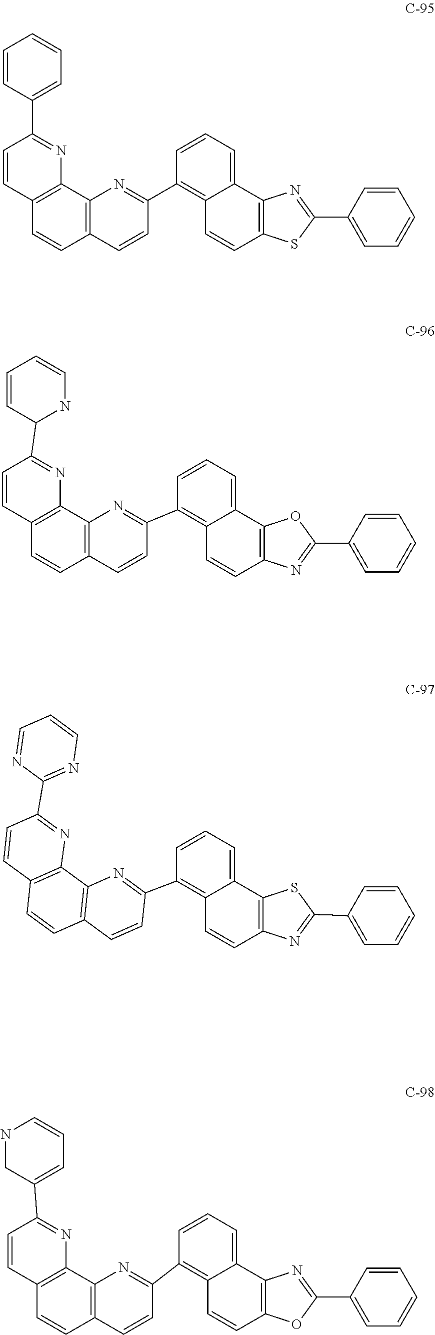

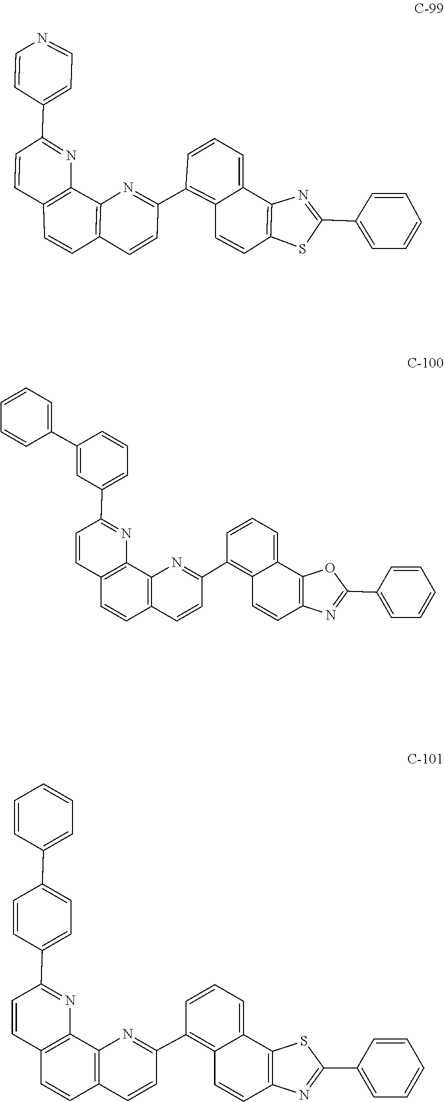 [SMILES: c1ccc(-c2nc3c(ccc4c(-c5ccc6ccc7ccc(-c8ccncc8)nc7c6n5)cccc43)s2)cc1 ; c1ccc(-c2cccc(-c3ccc4ccc5ccc(-c6cccc7c6ccc6nc(-c8ccccc8)oc67)nc5c4n3)c2)cc1 ; c1ccc(-c2ccc(-c3ccc4ccc5ccc(-c6cccc7c6ccc6nc(-c8ccccc8)sc67)nc5c4n3)cc2)cc1]

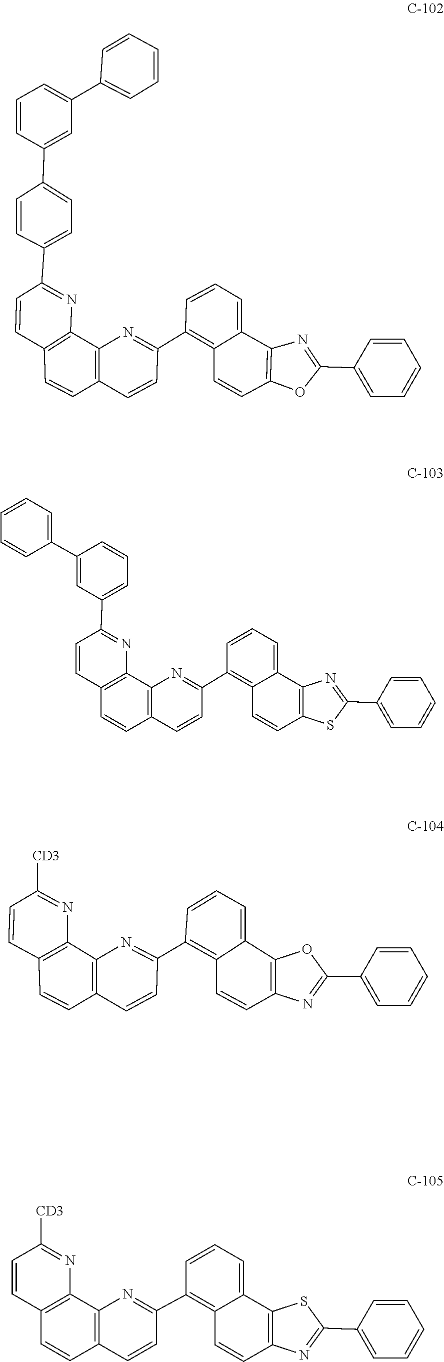 [SMILES: c1ccc(-c2cccc(-c3ccc(-c4ccc5ccc6ccc(-c7cccc8c7ccc7oc(-c9ccccc9)nc78)nc6c5n4)cc3)c2)cc1 ; c1ccc(-c2cccc(-c3ccc4ccc5ccc(-c6cccc7c6ccc6sc(-c8ccccc8)nc67)nc5c4n3)c2)cc1 ; [2H]C([2H])([2H])c1ccc2ccc3ccc(-c4cccc5c4ccc4nc(-c6ccccc6)oc45)nc3c2n1 ; [2H]C([2H])([2H])c1ccc2ccc3ccc(-c4cccc5c4ccc4nc(-c6ccccc6)sc45)nc3c2n1]

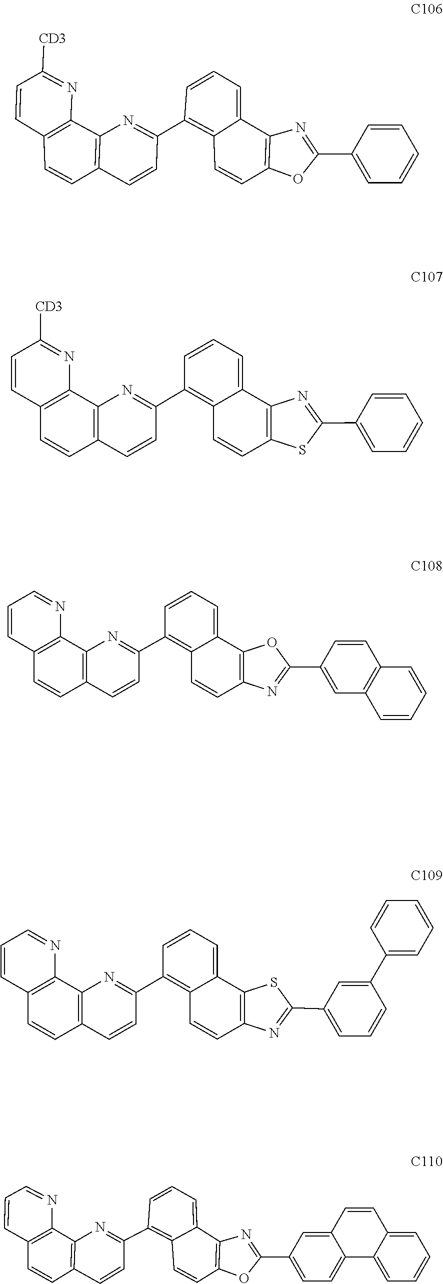 [SMILES: [2H]C([2H])([2H])c1ccc2ccc3ccc(-c4cccc5c4ccc4oc(-c6ccccc6)nc45)nc3c2n1 ; [2H]C([2H])([2H])c1ccc2ccc3ccc(-c4cccc5c4ccc4sc(-c6ccccc6)nc45)nc3c2n1 ; c1ccc2cc(-c3nc4ccc5c(-c6ccc7ccc8cccnc8c7n6)cccc5c4o3)ccc2c1 ; c1ccc(-c2cccc(-c3nc4ccc5c(-c6ccc7ccc8cccnc8c7n6)cccc5c4s3)c2)cc1 ; c1ccc2c(c1)ccc1cc(-c3nc4c(ccc5c(-c6ccc7ccc8cccnc8c7n6)cccc54)o3)ccc12]

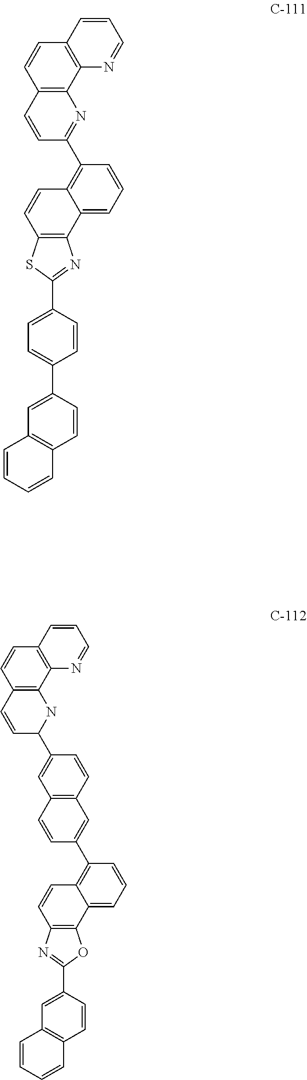

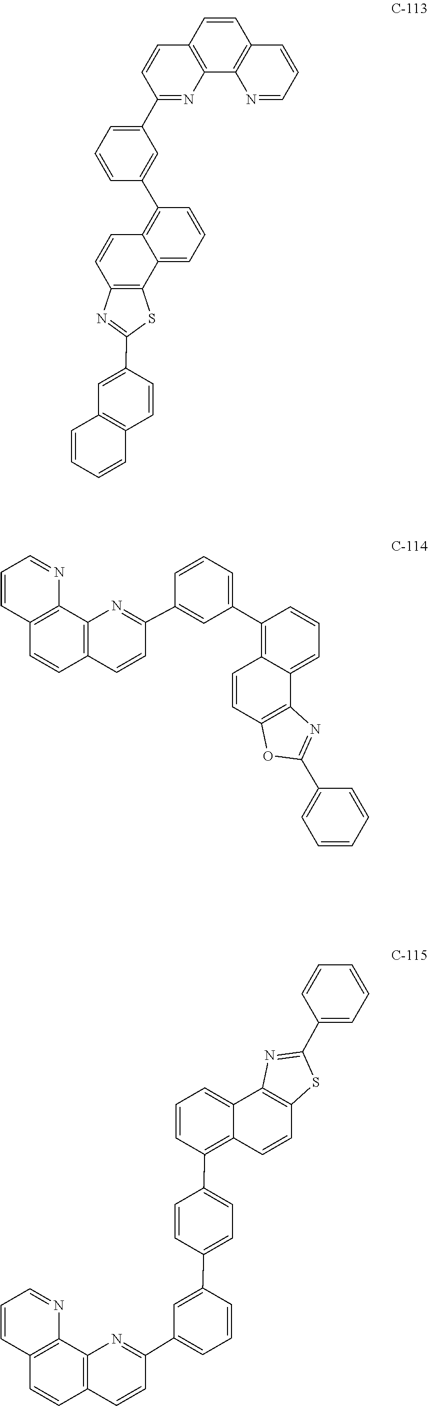 [SMILES: c1cc(-c2ccc3ccc4cccnc4c3n2)cc(-c2cccc3c2ccc2nc(-c4ccc5ccccc5c4)sc23)c1 ; c1ccc(-c2nc3c(ccc4c(-c5cccc(-c6ccc7ccc8cccnc8c7n6)c5)cccc43)o2)cc1 ; c1ccc(-c2nc3c(ccc4c(-c5ccc(-c6cccc(-c7ccc8ccc9cccnc9c8n7)c6)cc5)cccc43)s2)cc1]

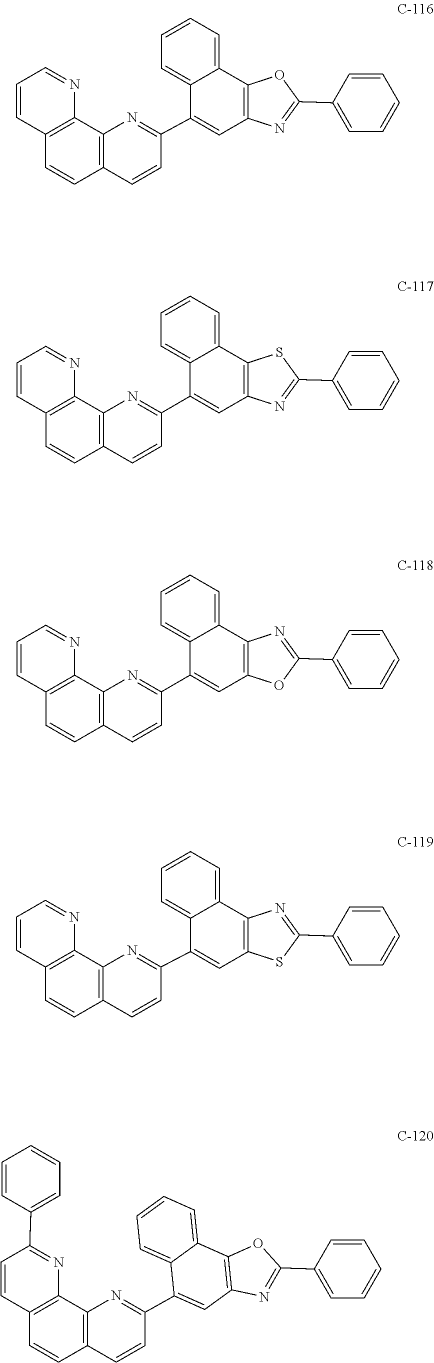 [SMILES: c1ccc(-c2nc3cc(-c4ccc5ccc6cccnc6c5n4)c4ccccc4c3o2)cc1 ; c1ccc(-c2nc3cc(-c4ccc5ccc6cccnc6c5n4)c4ccccc4c3s2)cc1 ; c1ccc(-c2nc3c(cc(-c4ccc5ccc6cccnc6c5n4)c4ccccc43)o2)cc1 ; c1ccc(-c2nc3c(cc(-c4ccc5ccc6cccnc6c5n4)c4ccccc43)s2)cc1 ; c1ccc(-c2ccc3ccc4ccc(-c5cc6nc(-c7ccccc7)oc6c6ccccc56)nc4c3n2)cc1]

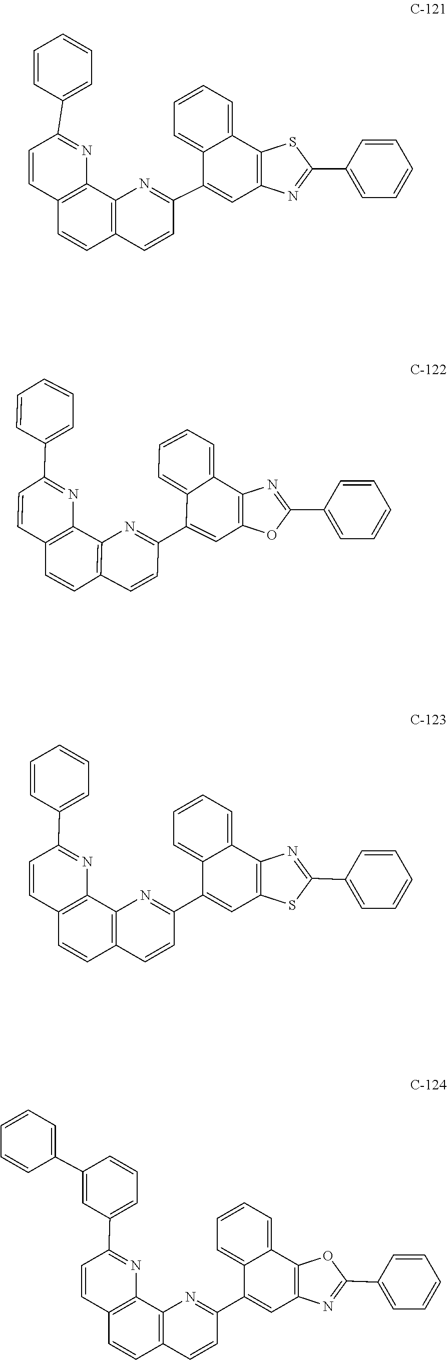 [SMILES: c1ccc(-c2ccc3ccc4ccc(-c5cc6nc(-c7ccccc7)sc6c6ccccc56)nc4c3n2)cc1 ; c1ccc(-c2ccc3ccc4ccc(-c5cc6oc(-c7ccccc7)nc6c6ccccc56)nc4c3n2)cc1 ; c1ccc(-c2ccc3ccc4ccc(-c5cc6sc(-c7ccccc7)nc6c6ccccc56)nc4c3n2)cc1 ; c1ccc(-c2cccc(-c3ccc4ccc5ccc(-c6cc7nc(-c8ccccc8)oc7c7ccccc67)nc5c4n3)c2)cc1]

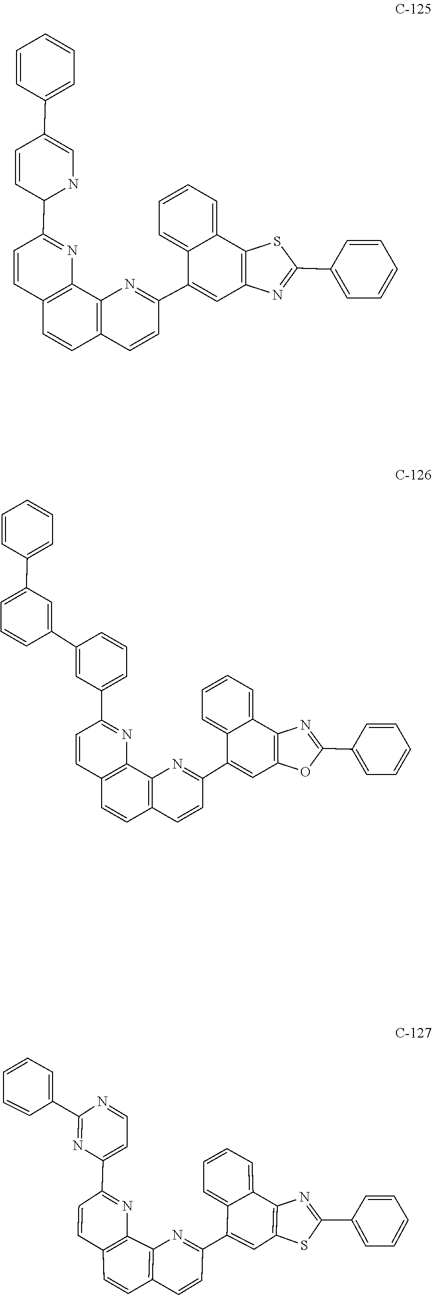

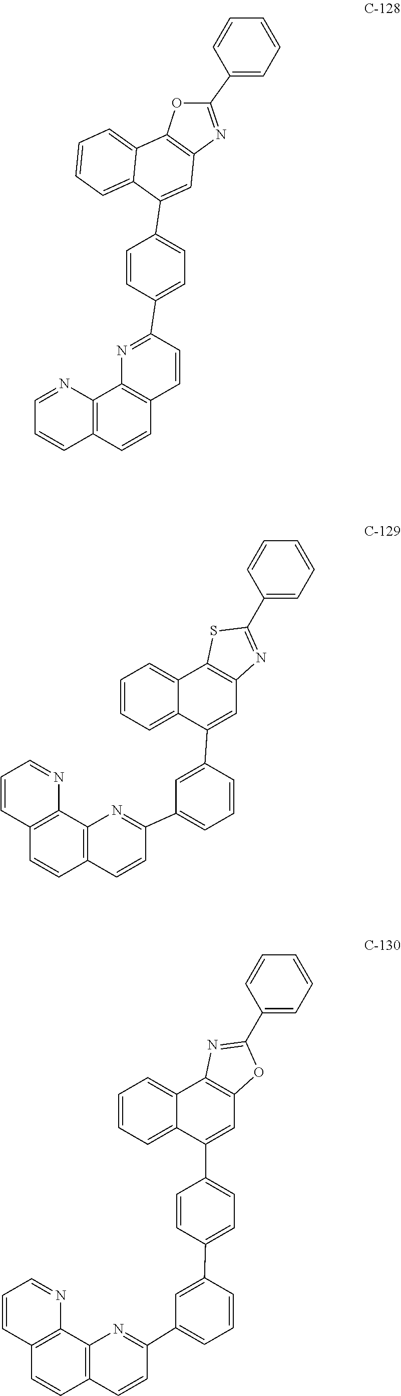 [SMILES: c1ccc(-c2nc3cc(-c4ccc(-c5ccc6ccc7cccnc7c6n5)cc4)c4ccccc4c3o2)cc1 ; c1ccc(-c2nc3cc(-c4cccc(-c5ccc6ccc7cccnc7c6n5)c4)c4ccccc4c3s2)cc1 ; c1ccc(-c2nc3c(cc(-c4ccc(-c5cccc(-c6ccc7ccc8cccnc8c7n6)c5)cc4)c4ccccc43)o2)cc1]

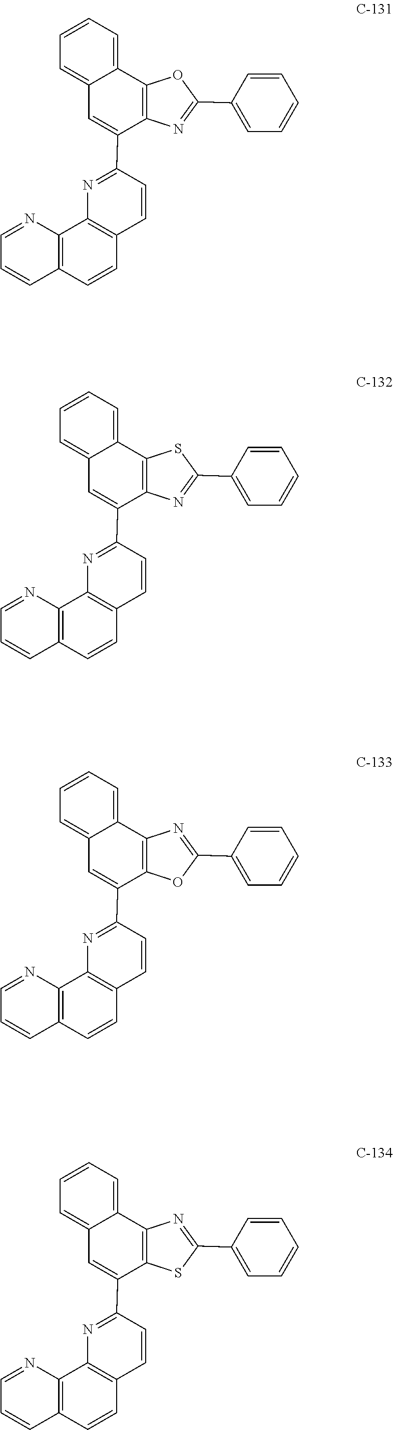 [SMILES: c1ccc(-c2nc3c(-c4ccc5ccc6cccnc6c5n4)cc4ccccc4c3o2)cc1 ; c1ccc(-c2nc3c(-c4ccc5ccc6cccnc6c5n4)cc4ccccc4c3s2)cc1 ; c1ccc(-c2nc3c(o2)c(-c2ccc4ccc5cccnc5c4n2)cc2ccccc23)cc1 ; c1ccc(-c2nc3c(s2)c(-c2ccc4ccc5cccnc5c4n2)cc2ccccc23)cc1]

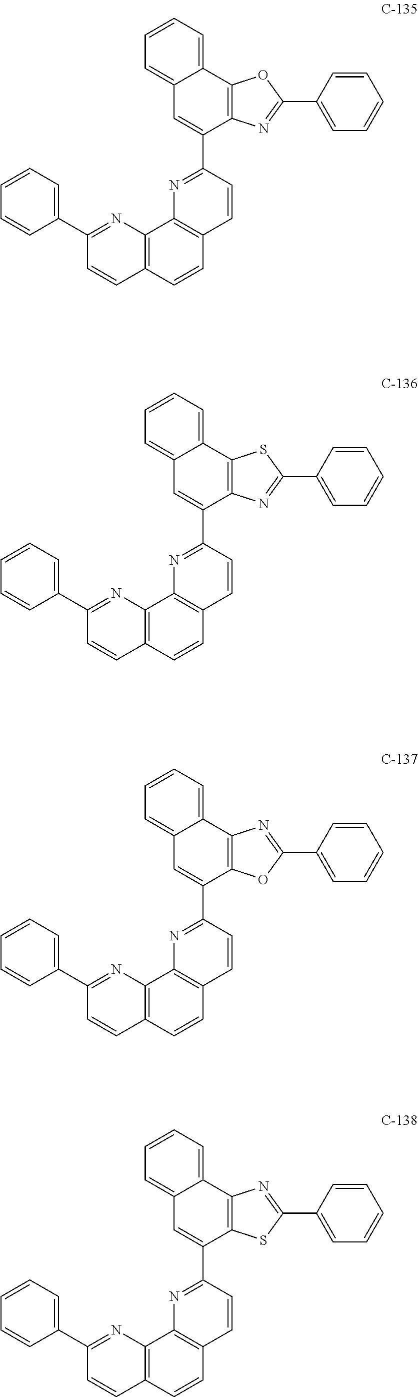 [SMILES: c1ccc(-c2ccc3ccc4ccc(-c5cc6ccccc6c6oc(-c7ccccc7)nc56)nc4c3n2)cc1 ; c1ccc(-c2ccc3ccc4ccc(-c5cc6ccccc6c6sc(-c7ccccc7)nc56)nc4c3n2)cc1 ; c1ccc(-c2ccc3ccc4ccc(-c5cc6ccccc6c6nc(-c7ccccc7)oc56)nc4c3n2)cc1 ; c1ccc(-c2ccc3ccc4ccc(-c5cc6ccccc6c6nc(-c7ccccc7)sc56)nc4c3n2)cc1]

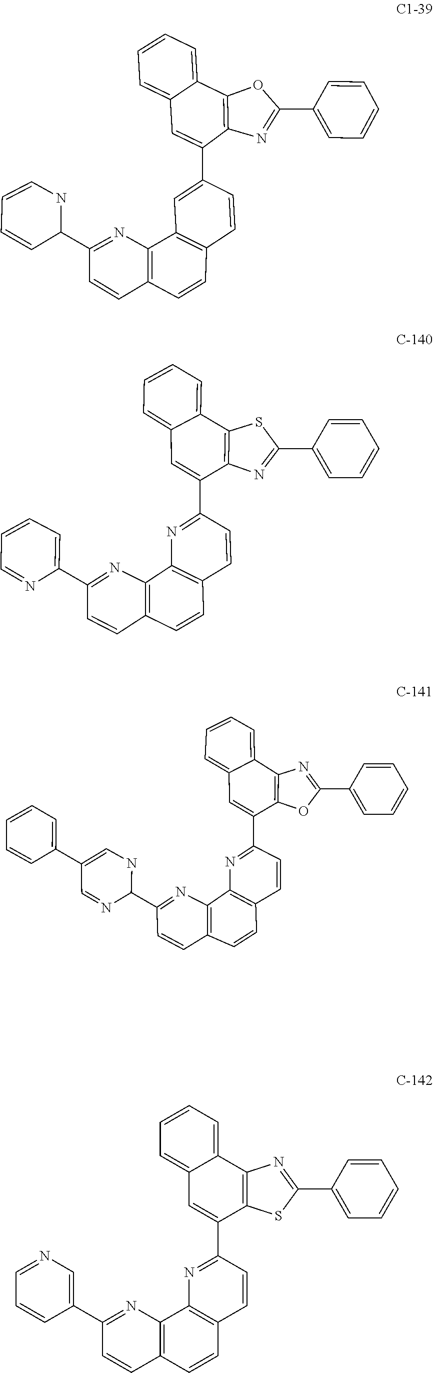

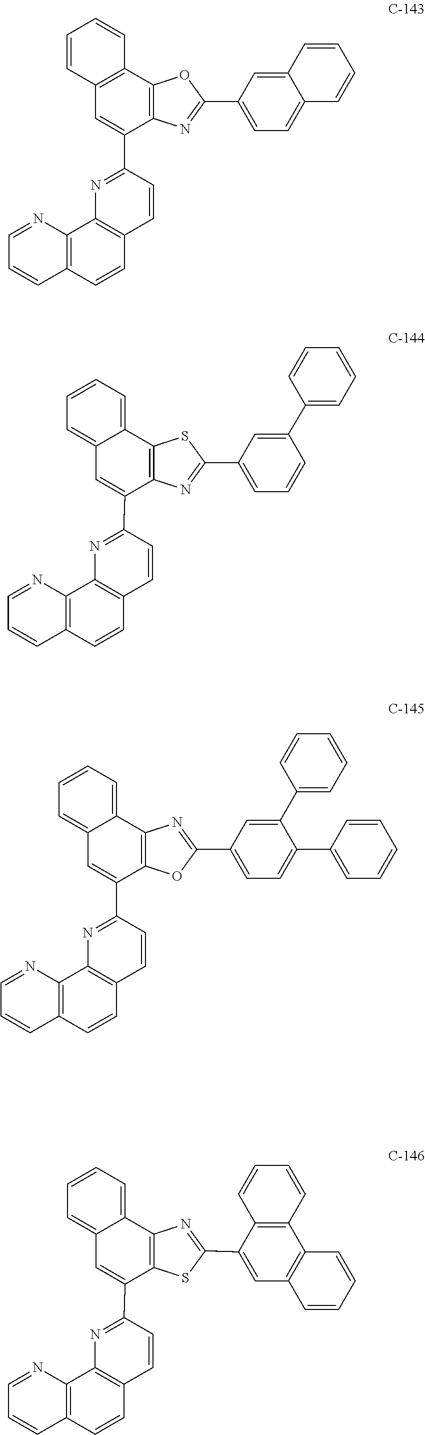 [SMILES: c1ccc2cc(-c3nc4c(-c5ccc6ccc7cccnc7c6n5)cc5ccccc5c4o3)ccc2c1 ; c1ccc(-c2cccc(-c3nc4c(-c5ccc6ccc7cccnc7c6n5)cc5ccccc5c4s3)c2)cc1 ; c1ccc(-c2ccc(-c3nc4c(o3)c(-c3ccc5ccc6cccnc6c5n3)cc3ccccc34)cc2-c2ccccc2)cc1 ; c1ccc2c(c1)cc(-c1nc3c(s1)c(-c1ccc4ccc5cccnc5c4n1)cc1ccccc13)c1ccccc12]

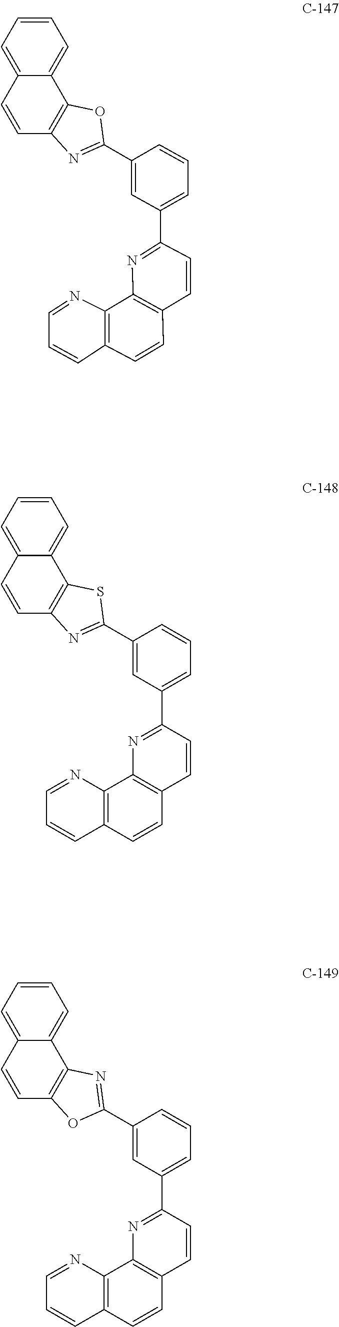 [SMILES: c1cc(-c2ccc3ccc4cccnc4c3n2)cc(-c2nc3ccc4ccccc4c3o2)c1 ; c1cc(-c2ccc3ccc4cccnc4c3n2)cc(-c2nc3ccc4ccccc4c3s2)c1 ; c1cc(-c2ccc3ccc4cccnc4c3n2)cc(-c2nc3c(ccc4ccccc43)o2)c1]

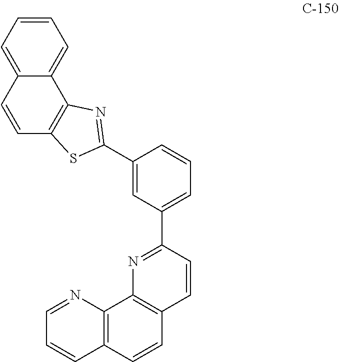 [SMILES: c1cc(-c2ccc3ccc4cccnc4c3n2)cc(-c2nc3c(ccc4ccccc43)s2)c1]

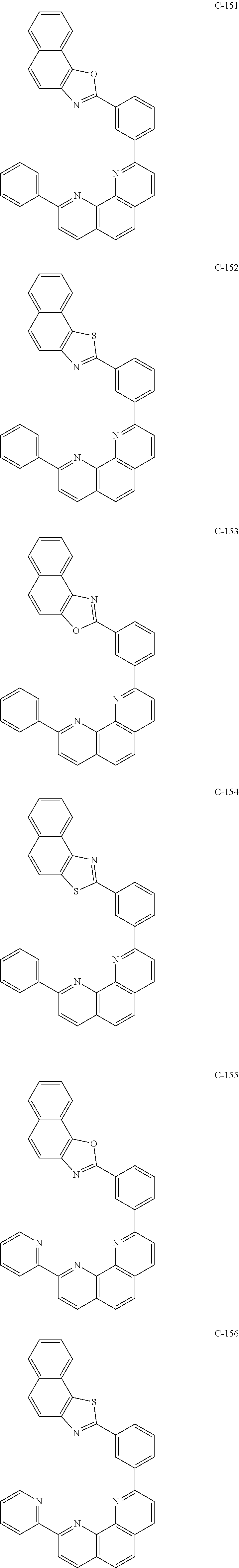 [SMILES: c1ccc(-c2ccc3ccc4ccc(-c5cccc(-c6nc7ccc8ccccc8c7o6)c5)nc4c3n2)cc1 ; c1ccc(-c2ccc3ccc4ccc(-c5cccc(-c6nc7ccc8ccccc8c7s6)c5)nc4c3n2)cc1 ; c1ccc(-c2ccc3ccc4ccc(-c5cccc(-c6nc7c(ccc8ccccc87)o6)c5)nc4c3n2)cc1 ; c1ccc(-c2ccc3ccc4ccc(-c5cccc(-c6nc7c(ccc8ccccc87)s6)c5)nc4c3n2)cc1 ; c1ccc(-c2ccc3ccc4ccc(-c5cccc(-c6nc7ccc8ccccc8c7o6)c5)nc4c3n2)nc1 ; c1ccc(-c2ccc3ccc4ccc(-c5cccc(-c6nc7ccc8ccccc8c7s6)c5)nc4c3n2)nc1]

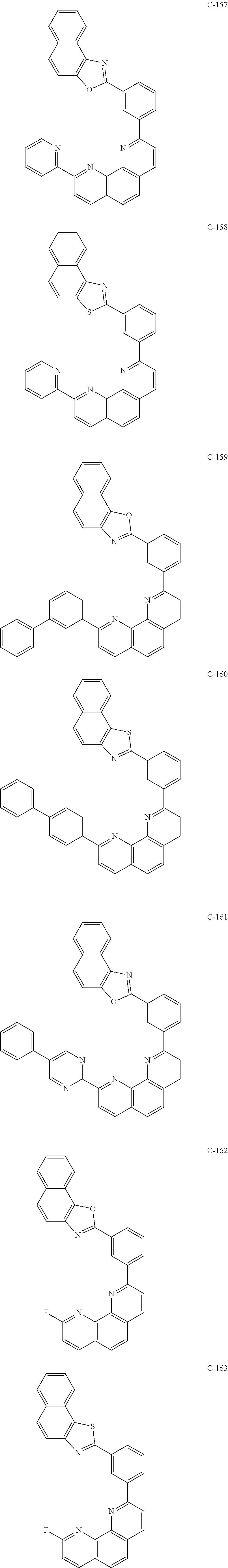

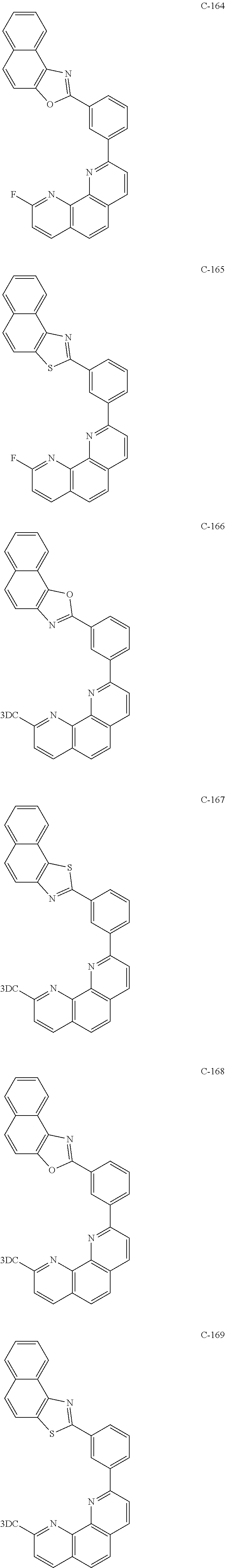

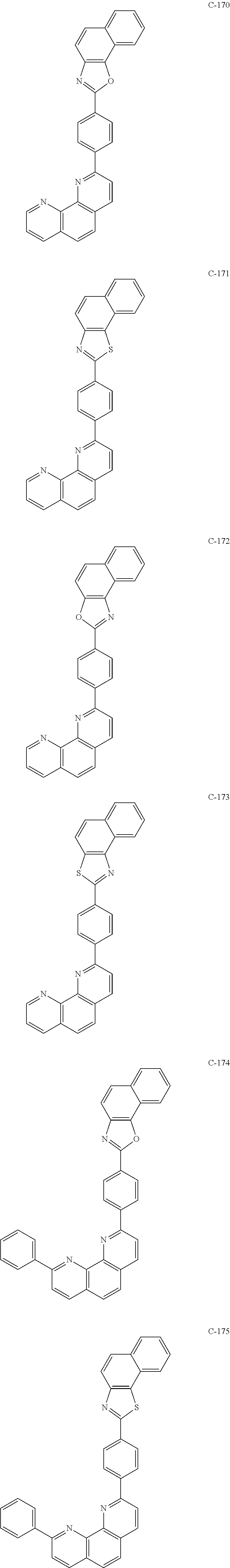 [SMILES: c1ccc2c(c1)ccc1nc(-c3ccc(-c4ccc5ccc6cccnc6c5n4)cc3)oc12 ; c1ccc2c(c1)ccc1nc(-c3ccc(-c4ccc5ccc6cccnc6c5n4)cc3)sc12 ; c1ccc2c(c1)ccc1oc(-c3ccc(-c4ccc5ccc6cccnc6c5n4)cc3)nc12 ; c1ccc2c(c1)ccc1sc(-c3ccc(-c4ccc5ccc6cccnc6c5n4)cc3)nc12 ; c1ccc(-c2ccc3ccc4ccc(-c5ccc(-c6nc7ccc8ccccc8c7o6)cc5)nc4c3n2)cc1 ; c1ccc(-c2ccc3ccc4ccc(-c5ccc(-c6nc7ccc8ccccc8c7s6)cc5)nc4c3n2)cc1]

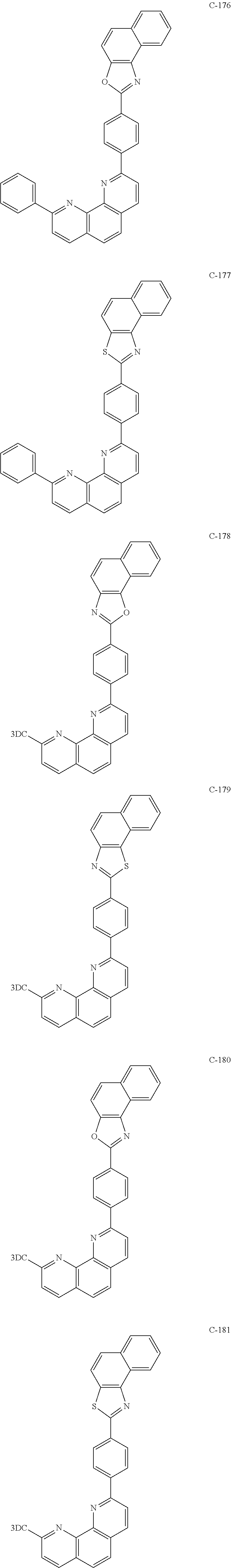

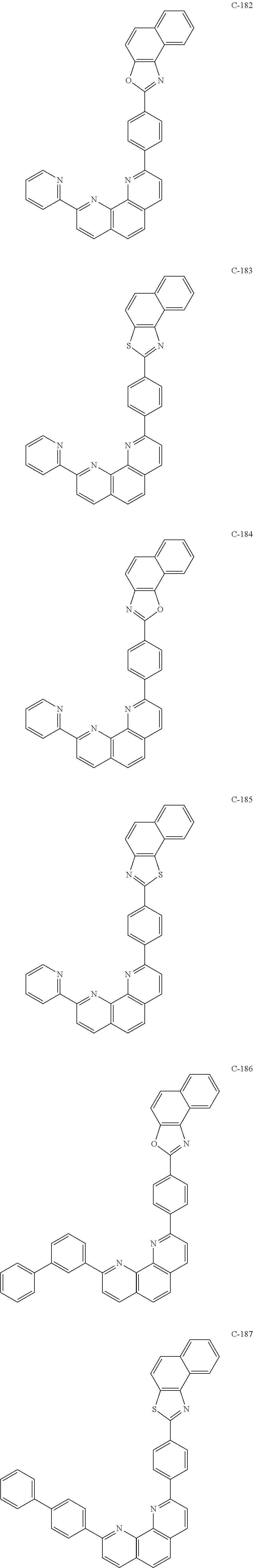 [SMILES: c1ccc(-c2ccc3ccc4ccc(-c5ccc(-c6nc7c(ccc8ccccc87)o6)cc5)nc4c3n2)nc1 ; c1ccc(-c2ccc3ccc4ccc(-c5ccc(-c6nc7c(ccc8ccccc87)s6)cc5)nc4c3n2)nc1 ; c1ccc(-c2ccc3ccc4ccc(-c5ccc(-c6nc7ccc8ccccc8c7o6)cc5)nc4c3n2)nc1 ; c1ccc(-c2ccc3ccc4ccc(-c5ccc(-c6nc7ccc8ccccc8c7s6)cc5)nc4c3n2)nc1 ; c1ccc(-c2cccc(-c3ccc4ccc5ccc(-c6ccc(-c7nc8c(ccc9ccccc98)o7)cc6)nc5c4n3)c2)cc1 ; c1ccc(-c2ccc(-c3ccc4ccc5ccc(-c6ccc(-c7nc8c(ccc9ccccc98)s7)cc6)nc5c4n3)cc2)cc1]

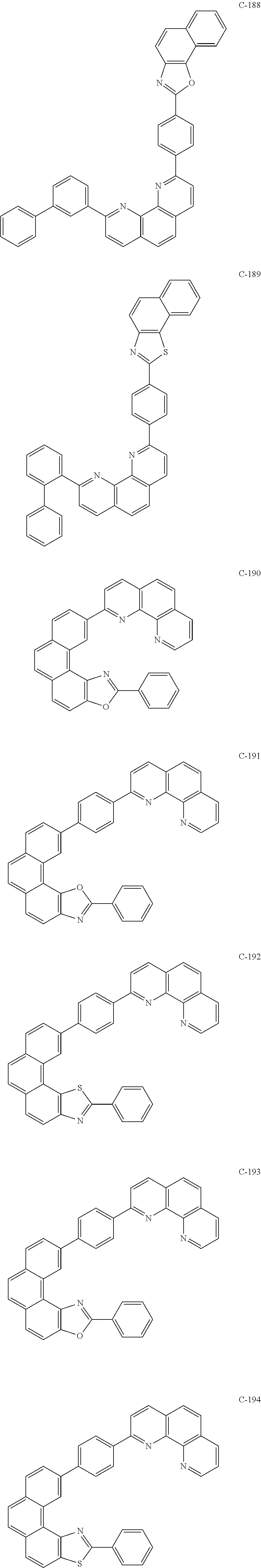

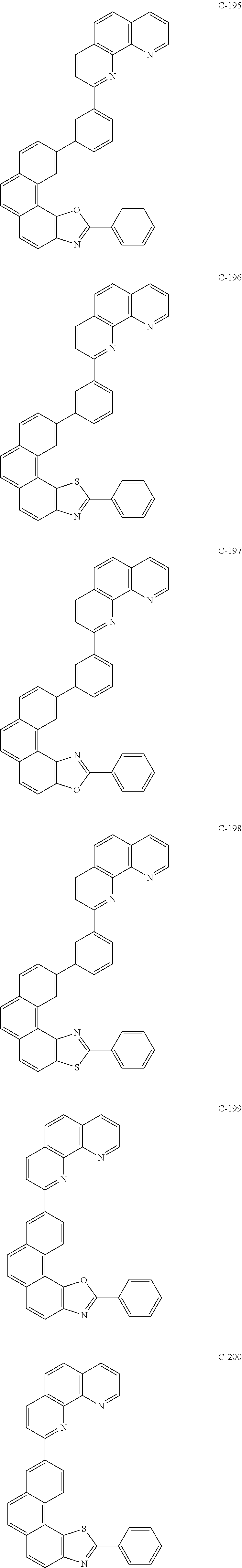 [SMILES: c1ccc(-c2nc3ccc4ccc5ccc(-c6cccc(-c7ccc8ccc9cccnc9c8n7)c6)cc5c4c3o2)cc1 ; c1ccc(-c2nc3ccc4ccc5ccc(-c6cccc(-c7ccc8ccc9cccnc9c8n7)c6)cc5c4c3s2)cc1 ; c1ccc(-c2nc3c(ccc4ccc5ccc(-c6cccc(-c7ccc8ccc9cccnc9c8n7)c6)cc5c43)o2)cc1 ; c1ccc(-c2nc3c(ccc4ccc5ccc(-c6cccc(-c7ccc8ccc9cccnc9c8n7)c6)cc5c43)s2)cc1 ; c1ccc(-c2nc3ccc4ccc5cc(-c6ccc7ccc8cccnc8c7n6)ccc5c4c3o2)cc1 ; c1ccc(-c2nc3ccc4ccc5cc(-c6ccc7ccc8cccnc8c7n6)ccc5c4c3s2)cc1]

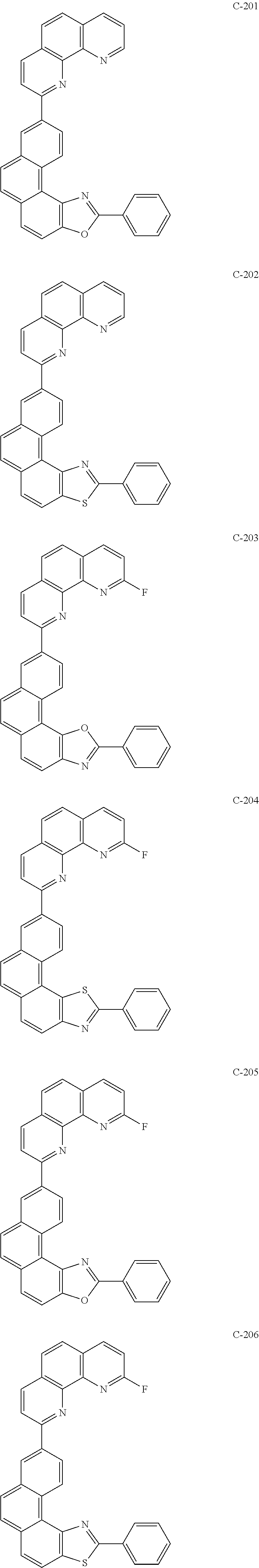 [SMILES: c1ccc(-c2nc3c(ccc4ccc5cc(-c6ccc7ccc8cccnc8c7n6)ccc5c43)o2)cc1 ; c1ccc(-c2nc3c(ccc4ccc5cc(-c6ccc7ccc8cccnc8c7n6)ccc5c43)s2)cc1 ; Fc1ccc2ccc3ccc(-c4ccc5c(ccc6ccc7nc(-c8ccccc8)oc7c65)c4)nc3c2n1 ; Fc1ccc2ccc3ccc(-c4ccc5c(ccc6ccc7nc(-c8ccccc8)sc7c65)c4)nc3c2n1 ; Fc1ccc2ccc3ccc(-c4ccc5c(ccc6ccc7oc(-c8ccccc8)nc7c65)c4)nc3c2n1 ; Fc1ccc2ccc3ccc(-c4ccc5c(ccc6ccc7sc(-c8ccccc8)nc7c65)c4)nc3c2n1]

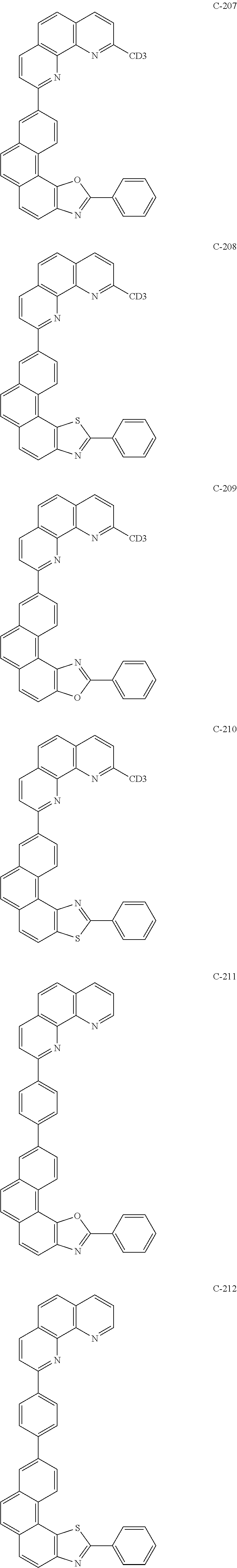 [SMILES: [2H]C([2H])([2H])c1ccc2ccc3ccc(-c4ccc5c(ccc6ccc7nc(-c8ccccc8)oc7c65)c4)nc3c2n1 ; [2H]C([2H])([2H])c1ccc2ccc3ccc(-c4ccc5c(ccc6ccc7nc(-c8ccccc8)sc7c65)c4)nc3c2n1 ; [2H]C([2H])([2H])c1ccc2ccc3ccc(-c4ccc5c(ccc6ccc7oc(-c8ccccc8)nc7c65)c4)nc3c2n1 ; [2H]C([2H])([2H])c1ccc2ccc3ccc(-c4ccc5c(ccc6ccc7sc(-c8ccccc8)nc7c65)c4)nc3c2n1 ; c1ccc(-c2nc3ccc4ccc5cc(-c6ccc(-c7ccc8ccc9cccnc9c8n7)cc6)ccc5c4c3o2)cc1 ; c1ccc(-c2nc3ccc4ccc5cc(-c6ccc(-c7ccc8ccc9cccnc9c8n7)cc6)ccc5c4c3s2)cc1]

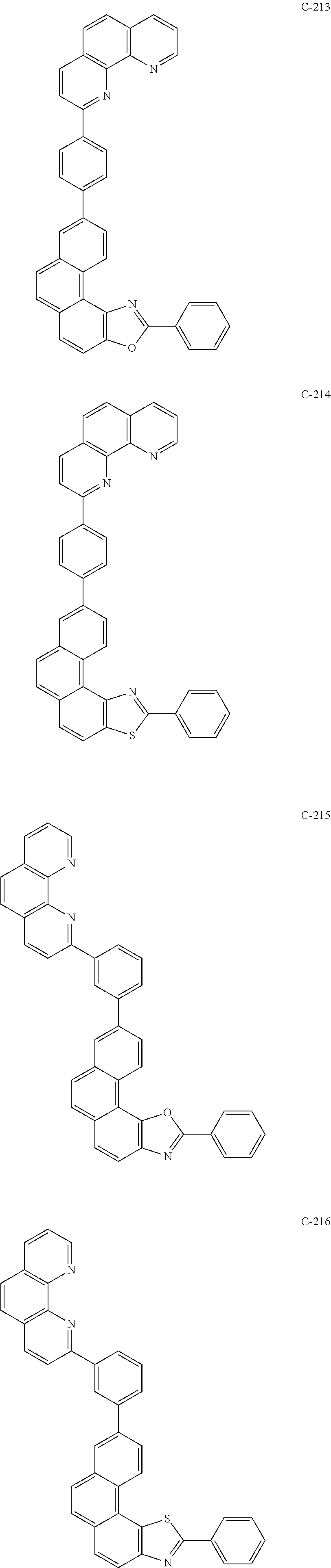 [SMILES: c1ccc(-c2nc3c(ccc4ccc5cc(-c6ccc(-c7ccc8ccc9cccnc9c8n7)cc6)ccc5c43)o2)cc1 ; c1ccc(-c2nc3c(ccc4ccc5cc(-c6ccc(-c7ccc8ccc9cccnc9c8n7)cc6)ccc5c43)s2)cc1 ; c1ccc(-c2nc3ccc4ccc5cc(-c6cccc(-c7ccc8ccc9cccnc9c8n7)c6)ccc5c4c3o2)cc1 ; c1ccc(-c2nc3ccc4ccc5cc(-c6cccc(-c7ccc8ccc9cccnc9c8n7)c6)ccc5c4c3s2)cc1]

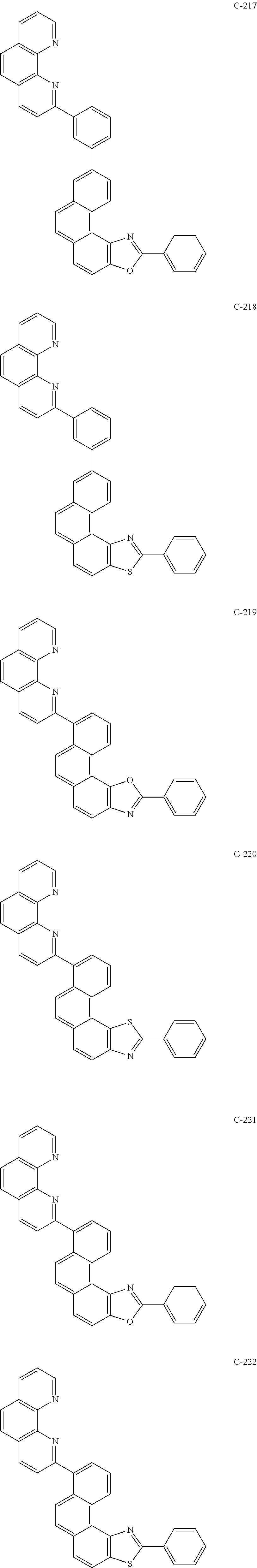 [SMILES: c1ccc(-c2nc3c(ccc4ccc5cc(-c6cccc(-c7ccc8ccc9cccnc9c8n7)c6)ccc5c43)o2)cc1 ; c1ccc(-c2nc3c(ccc4ccc5cc(-c6cccc(-c7ccc8ccc9cccnc9c8n7)c6)ccc5c43)s2)cc1 ; c1ccc(-c2nc3ccc4ccc5c(-c6ccc7ccc8cccnc8c7n6)cccc5c4c3o2)cc1 ; c1ccc(-c2nc3ccc4ccc5c(-c6ccc7ccc8cccnc8c7n6)cccc5c4c3s2)cc1 ; c1ccc(-c2nc3c(ccc4ccc5c(-c6ccc7ccc8cccnc8c7n6)cccc5c43)o2)cc1 ; c1ccc(-c2nc3c(ccc4ccc5c(-c6ccc7ccc8cccnc8c7n6)cccc5c43)s2)cc1]

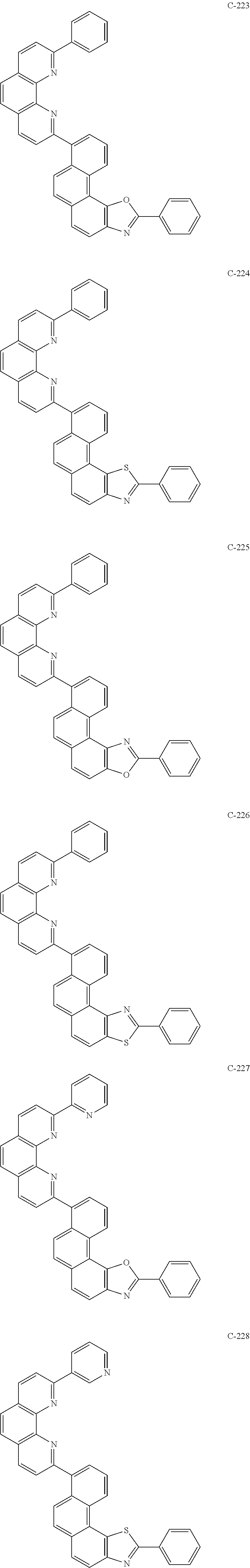

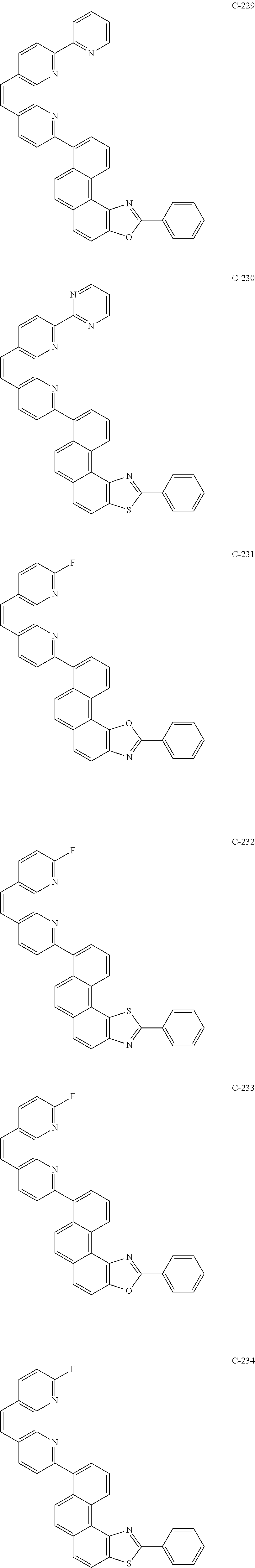 [SMILES: c1ccc(-c2nc3c(ccc4ccc5c(-c6ccc7ccc8ccc(-c9ccccn9)nc8c7n6)cccc5c43)o2)cc1 ; c1ccc(-c2nc3c(ccc4ccc5c(-c6ccc7ccc8ccc(-c9ncccn9)nc8c7n6)cccc5c43)s2)cc1 ; Fc1ccc2ccc3ccc(-c4cccc5c4ccc4ccc6nc(-c7ccccc7)oc6c45)nc3c2n1 ; Fc1ccc2ccc3ccc(-c4cccc5c4ccc4ccc6nc(-c7ccccc7)sc6c45)nc3c2n1 ; Fc1ccc2ccc3ccc(-c4cccc5c4ccc4ccc6oc(-c7ccccc7)nc6c45)nc3c2n1 ; Fc1ccc2ccc3ccc(-c4cccc5c4ccc4ccc6sc(-c7ccccc7)nc6c45)nc3c2n1]

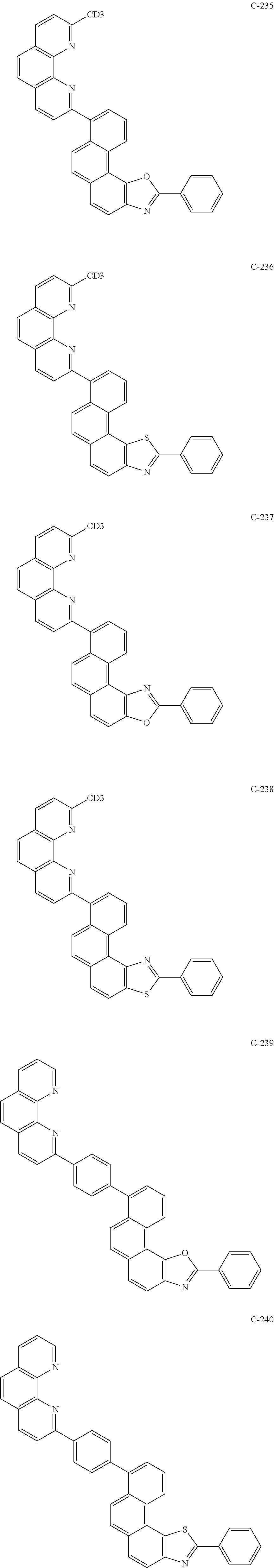 [SMILES: [2H]C([2H])([2H])c1ccc2ccc3ccc(-c4cccc5c4ccc4ccc6nc(-c7ccccc7)oc6c45)nc3c2n1 ; [2H]C([2H])([2H])c1ccc2ccc3ccc(-c4cccc5c4ccc4ccc6nc(-c7ccccc7)sc6c45)nc3c2n1 ; [2H]C([2H])([2H])c1ccc2ccc3ccc(-c4cccc5c4ccc4ccc6oc(-c7ccccc7)nc6c45)nc3c2n1 ; [2H]C([2H])([2H])c1ccc2ccc3ccc(-c4cccc5c4ccc4ccc6sc(-c7ccccc7)nc6c45)nc3c2n1 ; c1ccc(-c2nc3ccc4ccc5c(-c6ccc(-c7ccc8ccc9cccnc9c8n7)cc6)cccc5c4c3o2)cc1 ; c1ccc(-c2nc3ccc4ccc5c(-c6ccc(-c7ccc8ccc9cccnc9c8n7)cc6)cccc5c4c3s2)cc1]

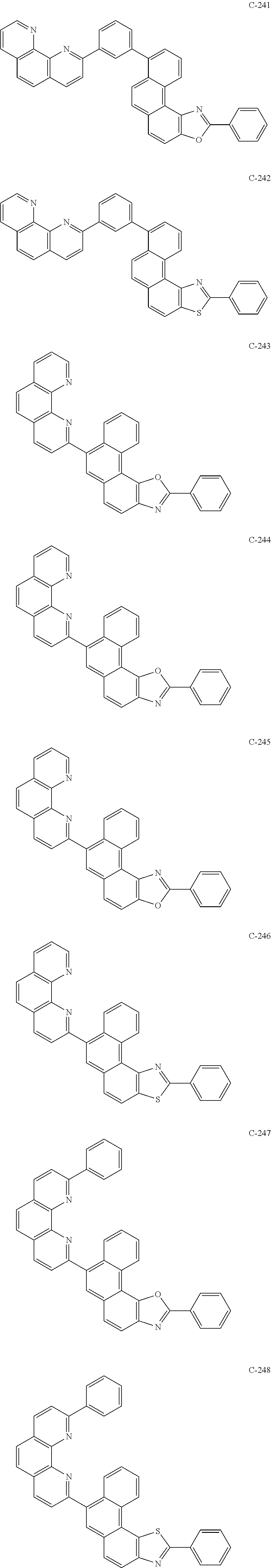

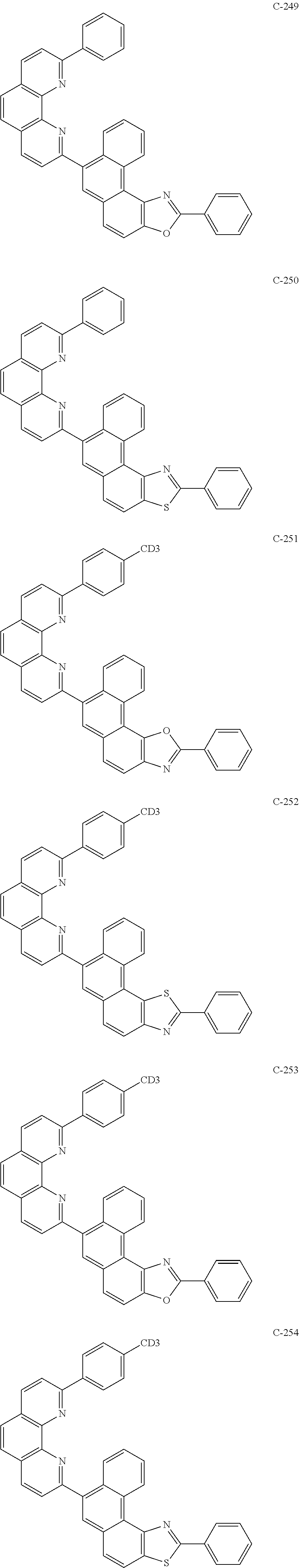

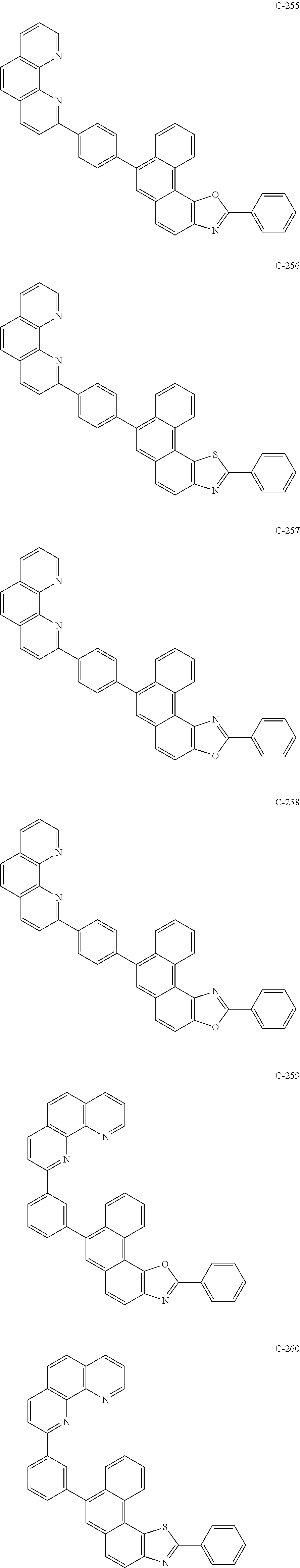 [SMILES: c1ccc(-c2nc3ccc4cc(-c5ccc(-c6ccc7ccc8cccnc8c7n6)cc5)c5ccccc5c4c3o2)cc1 ; c1ccc(-c2nc3ccc4cc(-c5ccc(-c6ccc7ccc8cccnc8c7n6)cc5)c5ccccc5c4c3s2)cc1 ; c1ccc(-c2nc3c(ccc4cc(-c5ccc(-c6ccc7ccc8cccnc8c7n6)cc5)c5ccccc5c43)o2)cc1 ; c1ccc(-c2nc3c(ccc4cc(-c5ccc(-c6ccc7ccc8cccnc8c7n6)cc5)c5ccccc5c43)o2)cc1 ; c1ccc(-c2nc3ccc4cc(-c5cccc(-c6ccc7ccc8cccnc8c7n6)c5)c5ccccc5c4c3o2)cc1 ; c1ccc(-c2nc3ccc4cc(-c5cccc(-c6ccc7ccc8cccnc8c7n6)c5)c5ccccc5c4c3s2)cc1]

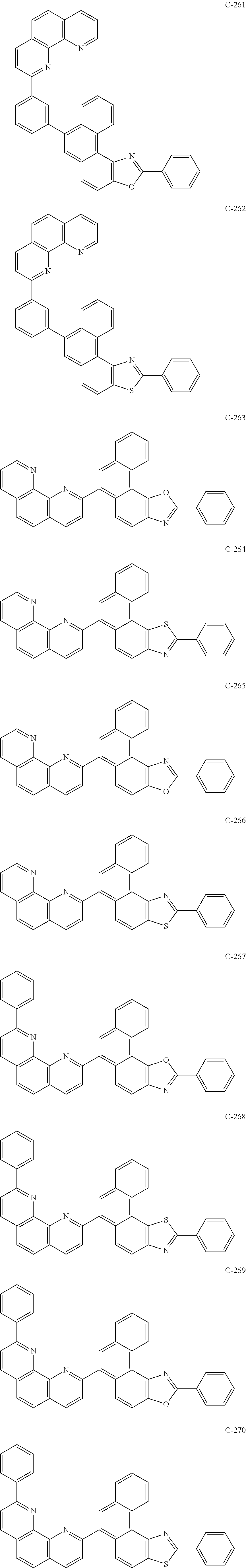

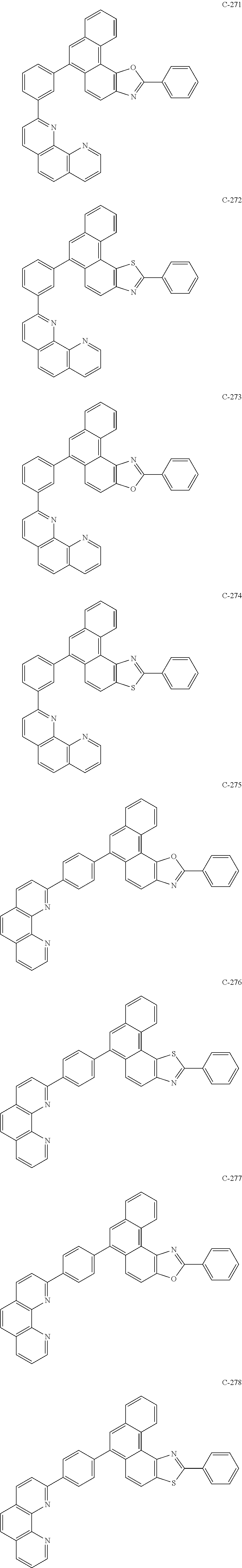

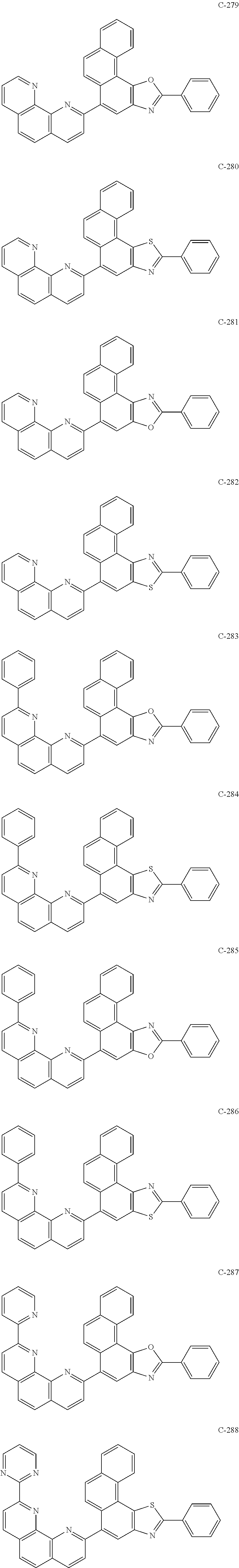

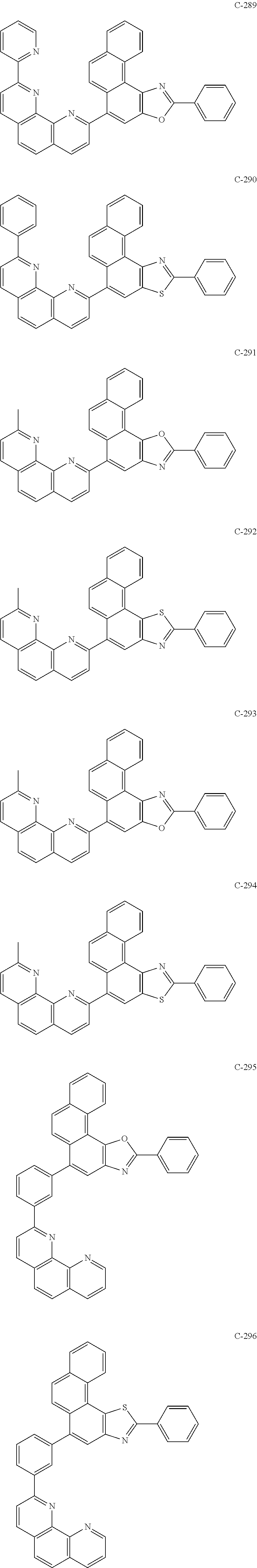

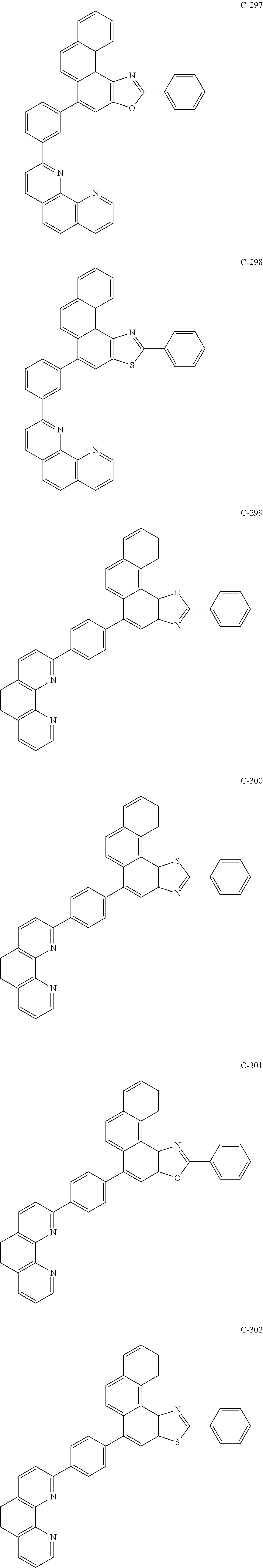 [SMILES: c1ccc(-c2nc3c(cc(-c4cccc(-c5ccc6ccc7cccnc7c6n5)c4)c4ccc5ccccc5c43)o2)cc1 ; c1ccc(-c2nc3c(cc(-c4cccc(-c5ccc6ccc7cccnc7c6n5)c4)c4ccc5ccccc5c43)s2)cc1 ; c1ccc(-c2nc3cc(-c4ccc(-c5ccc6ccc7cccnc7c6n5)cc4)c4ccc5ccccc5c4c3o2)cc1 ; c1ccc(-c2nc3cc(-c4ccc(-c5ccc6ccc7cccnc7c6n5)cc4)c4ccc5ccccc5c4c3s2)cc1 ; c1ccc(-c2nc3c(cc(-c4ccc(-c5ccc6ccc7cccnc7c6n5)cc4)c4ccc5ccccc5c43)o2)cc1 ; c1ccc(-c2nc3c(cc(-c4ccc(-c5ccc6ccc7cccnc7c6n5)cc4)c4ccc5ccccc5c43)s2)cc1]

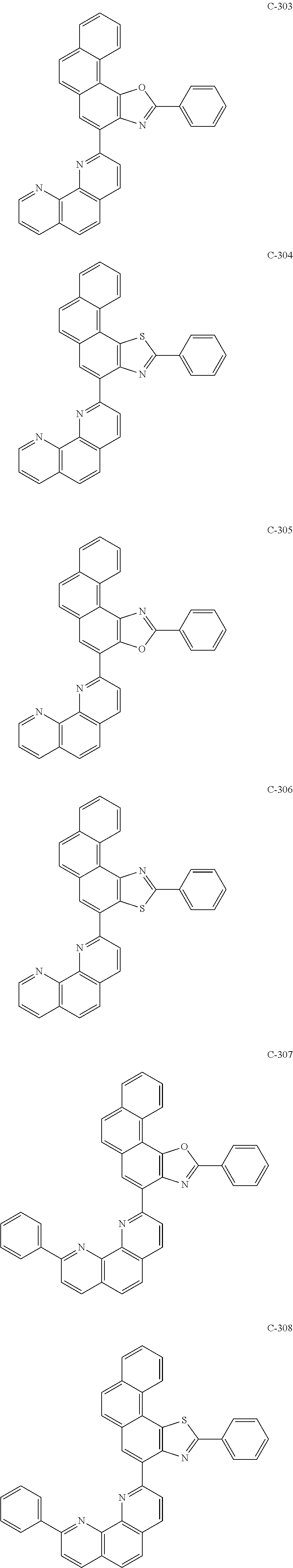 [SMILES: c1ccc(-c2nc3c(-c4ccc5ccc6cccnc6c5n4)cc4ccc5ccccc5c4c3o2)cc1 ; c1ccc(-c2nc3c(-c4ccc5ccc6cccnc6c5n4)cc4ccc5ccccc5c4c3s2)cc1 ; c1ccc(-c2nc3c(o2)c(-c2ccc4ccc5cccnc5c4n2)cc2ccc4ccccc4c23)cc1 ; c1ccc(-c2nc3c(s2)c(-c2ccc4ccc5cccnc5c4n2)cc2ccc4ccccc4c23)cc1 ; c1ccc(-c2ccc3ccc4ccc(-c5cc6ccc7ccccc7c6c6oc(-c7ccccc7)nc56)nc4c3n2)cc1 ; c1ccc(-c2ccc3ccc4ccc(-c5cc6ccc7ccccc7c6c6sc(-c7ccccc7)nc56)nc4c3n2)cc1]

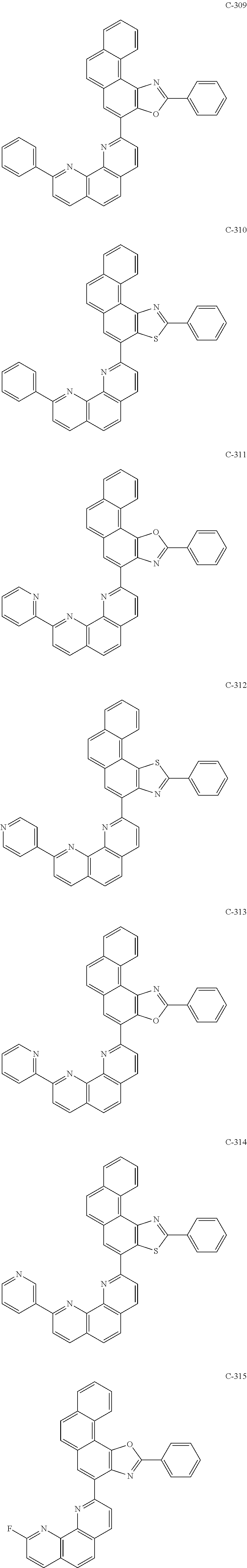

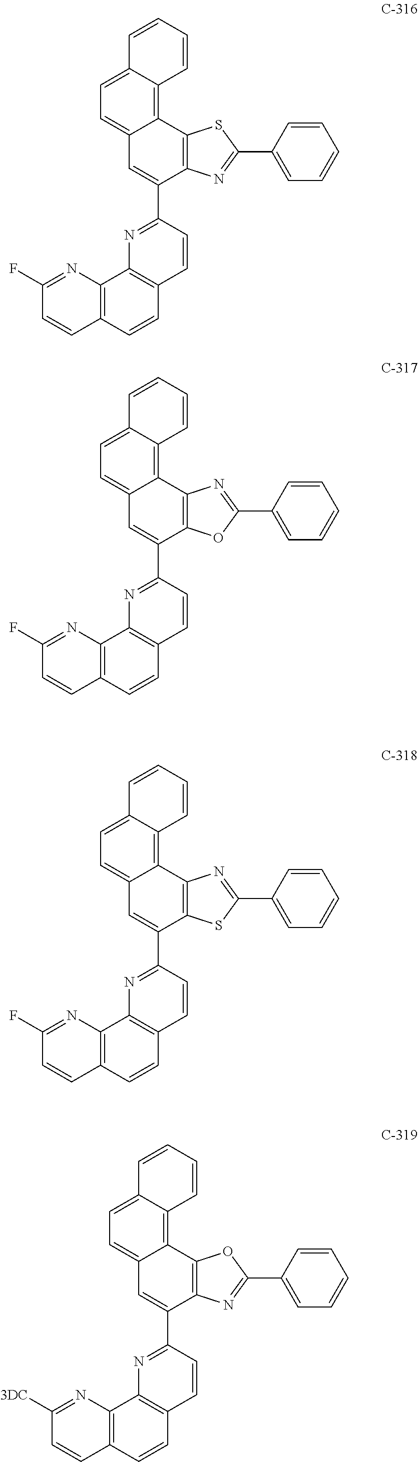

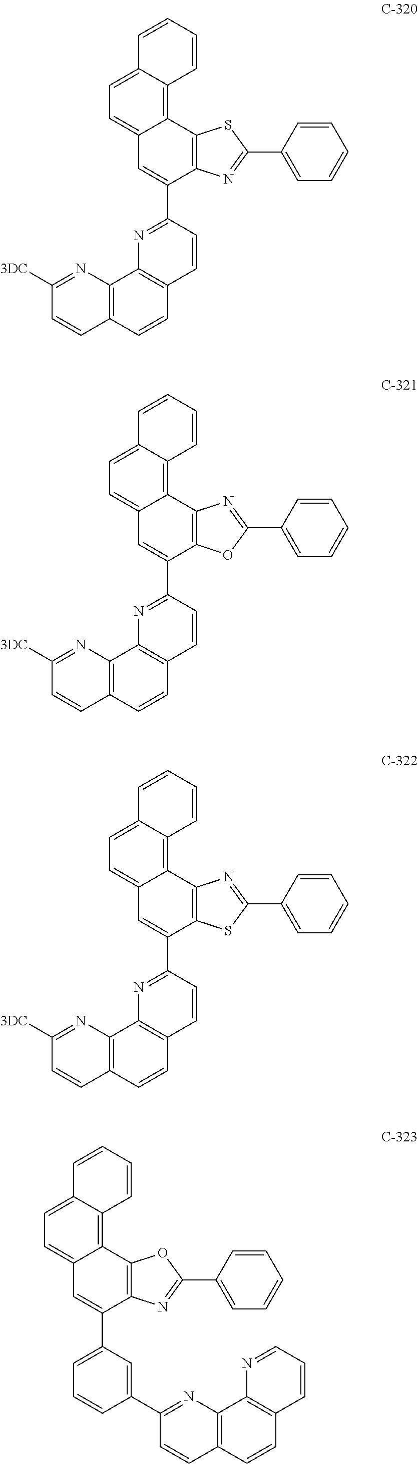

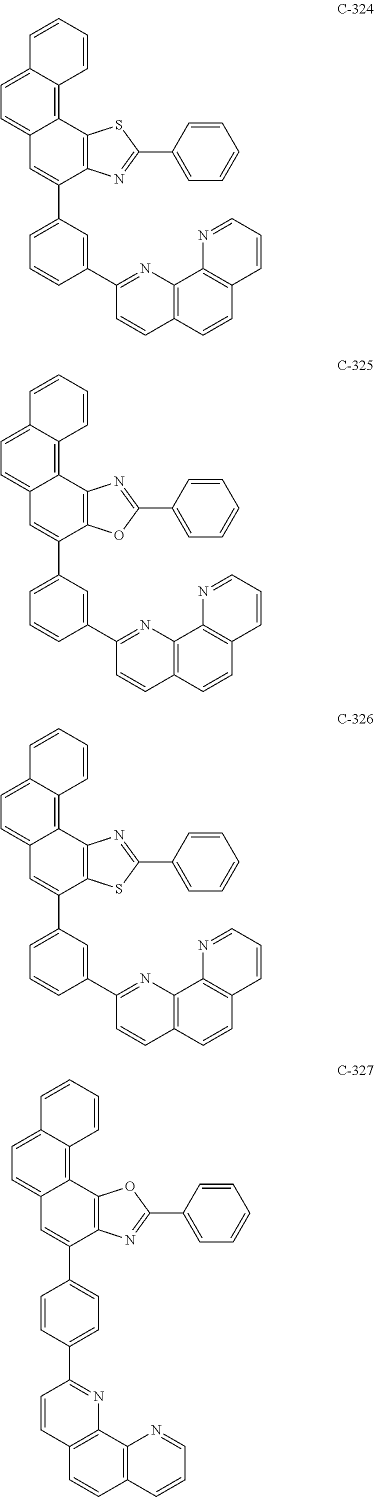 [SMILES: c1ccc(-c2nc3c(-c4cccc(-c5ccc6ccc7cccnc7c6n5)c4)cc4ccc5ccccc5c4c3s2)cc1 ; c1ccc(-c2nc3c(o2)c(-c2cccc(-c4ccc5ccc6cccnc6c5n4)c2)cc2ccc4ccccc4c23)cc1 ; c1ccc(-c2nc3c(s2)c(-c2cccc(-c4ccc5ccc6cccnc6c5n4)c2)cc2ccc4ccccc4c23)cc1 ; c1ccc(-c2nc3c(-c4ccc(-c5ccc6ccc7cccnc7c6n5)cc4)cc4ccc5ccccc5c4c3o2)cc1]

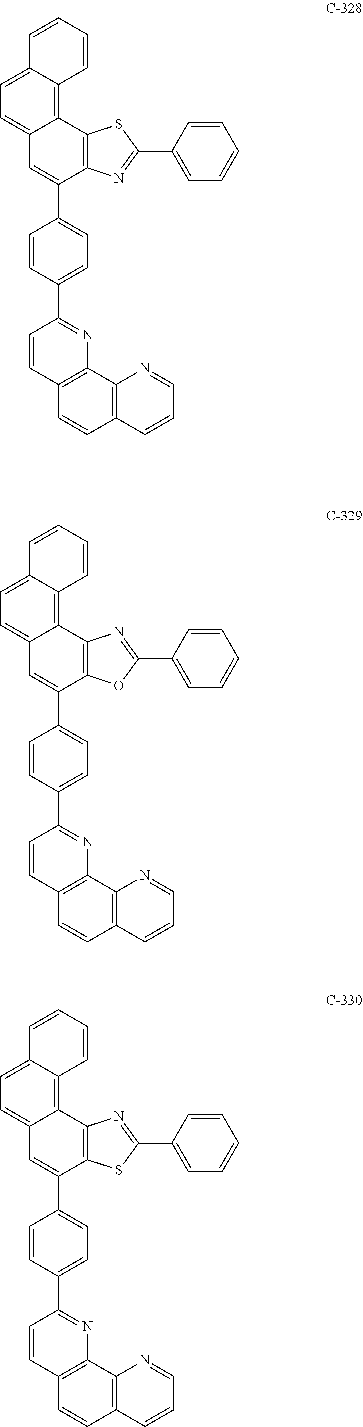 [SMILES: c1ccc(-c2nc3c(-c4ccc(-c5ccc6ccc7cccnc7c6n5)cc4)cc4ccc5ccccc5c4c3s2)cc1 ; c1ccc(-c2nc3c(o2)c(-c2ccc(-c4ccc5ccc6cccnc6c5n4)cc2)cc2ccc4ccccc4c23)cc1 ; c1ccc(-c2nc3c(s2)c(-c2ccc(-c4ccc5ccc6cccnc6c5n4)cc2)cc2ccc4ccccc4c23)cc1]

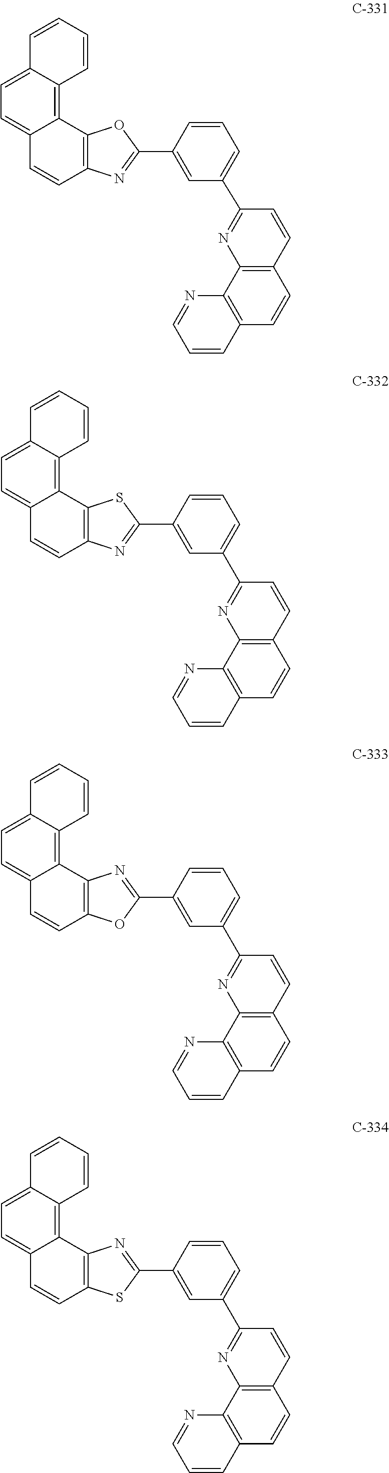 [SMILES: c1cc(-c2ccc3ccc4cccnc4c3n2)cc(-c2nc3ccc4ccc5ccccc5c4c3o2)c1 ; c1cc(-c2ccc3ccc4cccnc4c3n2)cc(-c2nc3ccc4ccc5ccccc5c4c3s2)c1 ; c1cc(-c2ccc3ccc4cccnc4c3n2)cc(-c2nc3c(ccc4ccc5ccccc5c43)o2)c1 ; c1cc(-c2ccc3ccc4cccnc4c3n2)cc(-c2nc3c(ccc4ccc5ccccc5c43)s2)c1]

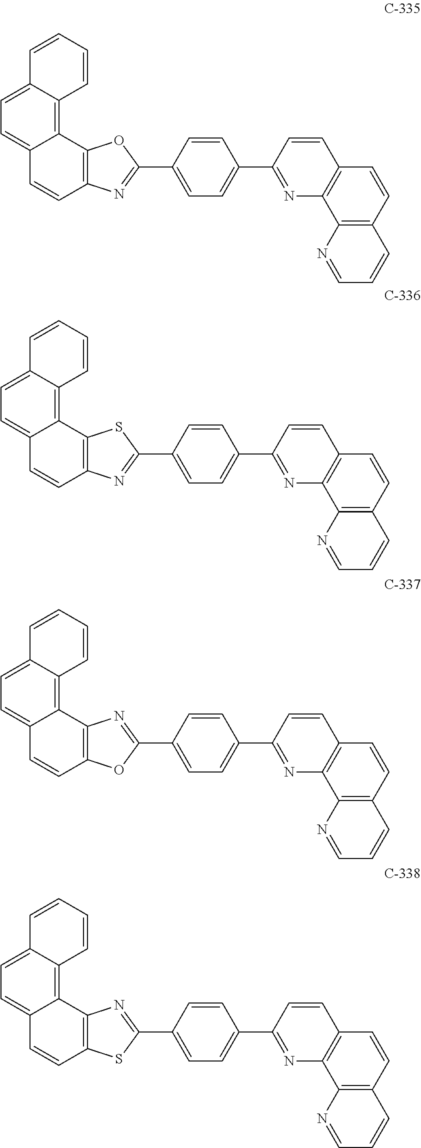 [SMILES: c1ccc2c(c1)ccc1ccc3nc(-c4ccc(-c5ccc6ccc7cccnc7c6n5)cc4)oc3c12 ; c1ccc2c(c1)ccc1ccc3nc(-c4ccc(-c5ccc6ccc7cccnc7c6n5)cc4)sc3c12 ; c1ccc2c(c1)ccc1ccc3oc(-c4ccc(-c5ccc6ccc7cccnc7c6n5)cc4)nc3c12 ; c1ccc2c(c1)ccc1ccc3sc(-c4ccc(-c5ccc6ccc7cccnc7c6n5)cc4)nc3c12]

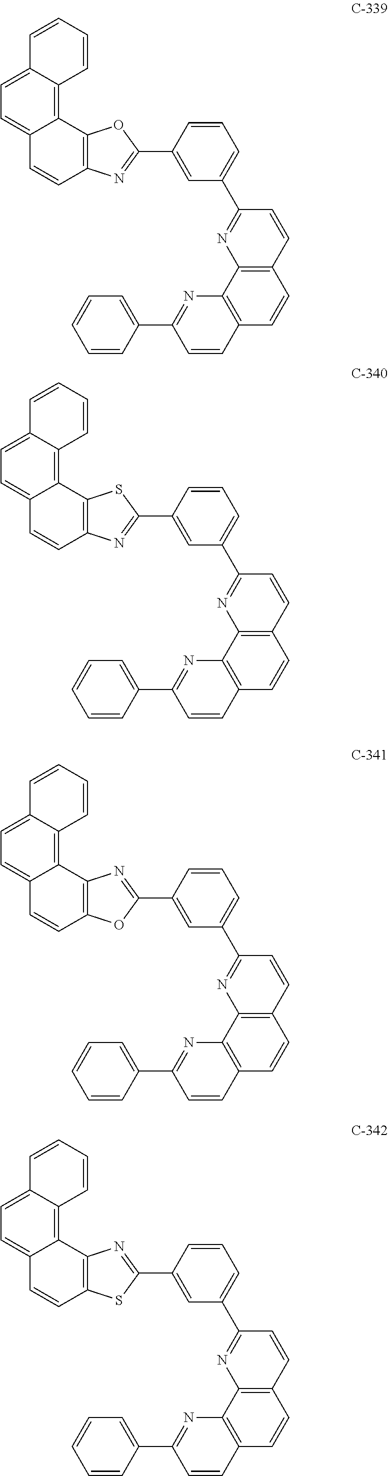

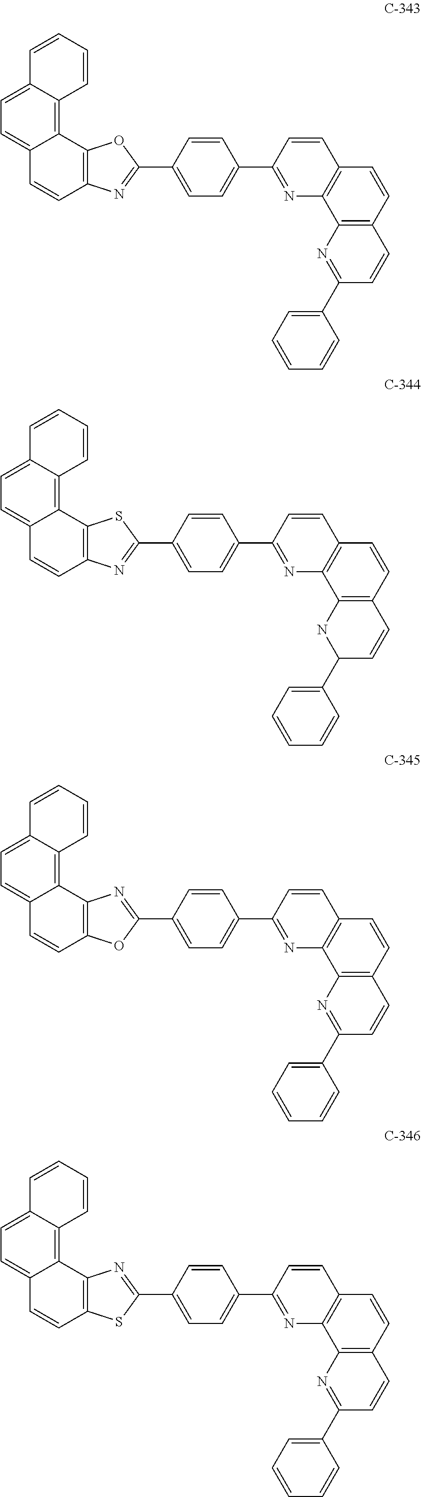

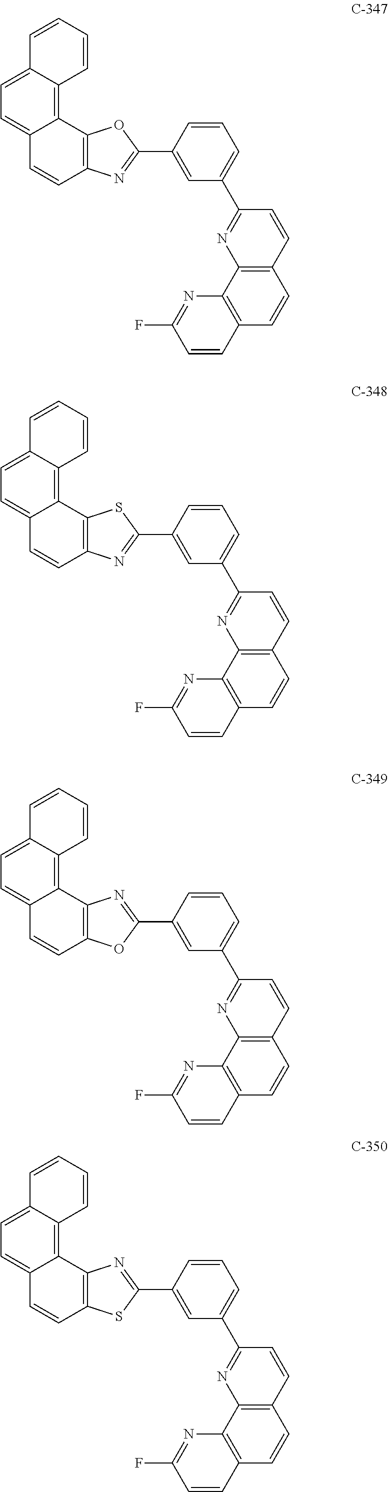 [SMILES: Fc1ccc2ccc3ccc(-c4cccc(-c5nc6ccc7ccc8ccccc8c7c6o5)c4)nc3c2n1 ; Fc1ccc2ccc3ccc(-c4cccc(-c5nc6ccc7ccc8ccccc8c7c6s5)c4)nc3c2n1 ; Fc1ccc2ccc3ccc(-c4cccc(-c5nc6c(ccc7ccc8ccccc8c76)o5)c4)nc3c2n1 ; Fc1ccc2ccc3ccc(-c4cccc(-c5nc6c(ccc7ccc8ccccc8c76)s5)c4)nc3c2n1]

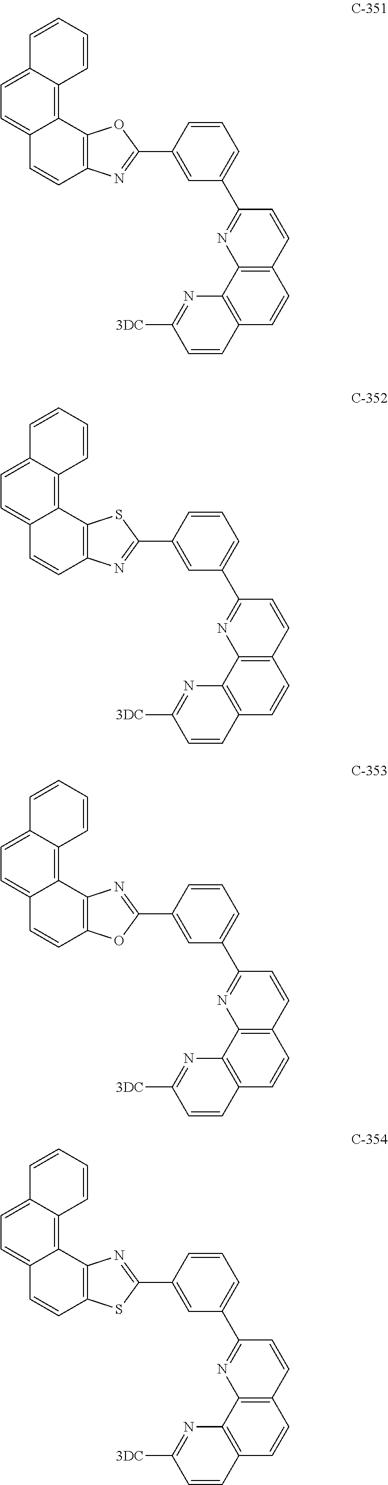

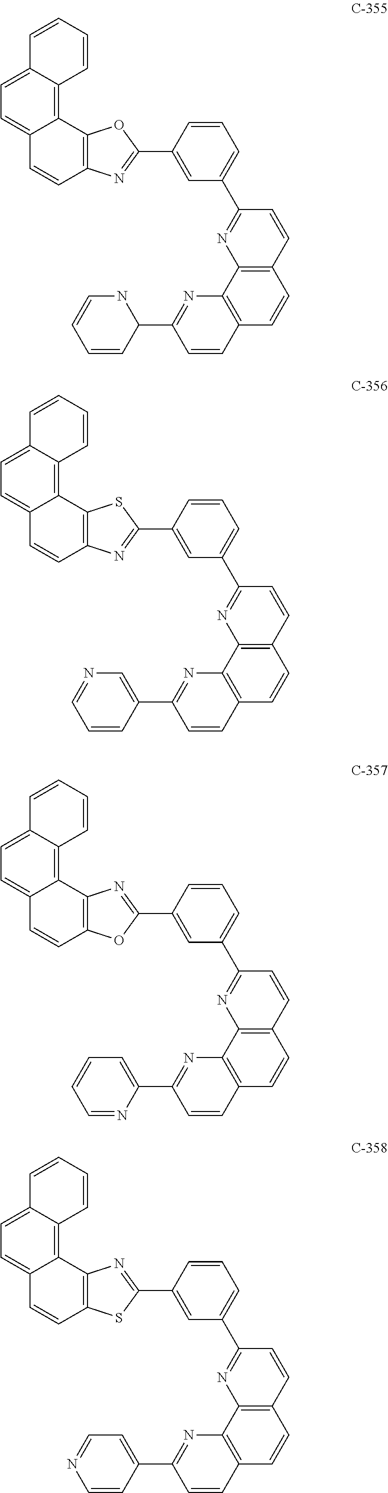 [SMILES: C1=CNC(c2ccc3ccc4ccc(-c5cccc(-c6nc7ccc8ccc9ccccc9c8c7o6)c5)nc4c3n2)C=C1 ; c1cncc(-c2ccc3ccc4ccc(-c5cccc(-c6nc7ccc8ccc9ccccc9c8c7s6)c5)nc4c3n2)c1 ; c1ccc(-c2ccc3ccc4ccc(-c5cccc(-c6nc7c(ccc8ccc9ccccc9c87)o6)c5)nc4c3n2)nc1 ; c1cc(-c2ccc3ccc4ccc(-c5ccncc5)nc4c3n2)cc(-c2nc3c(ccc4ccc5ccccc5c43)s2)c1]

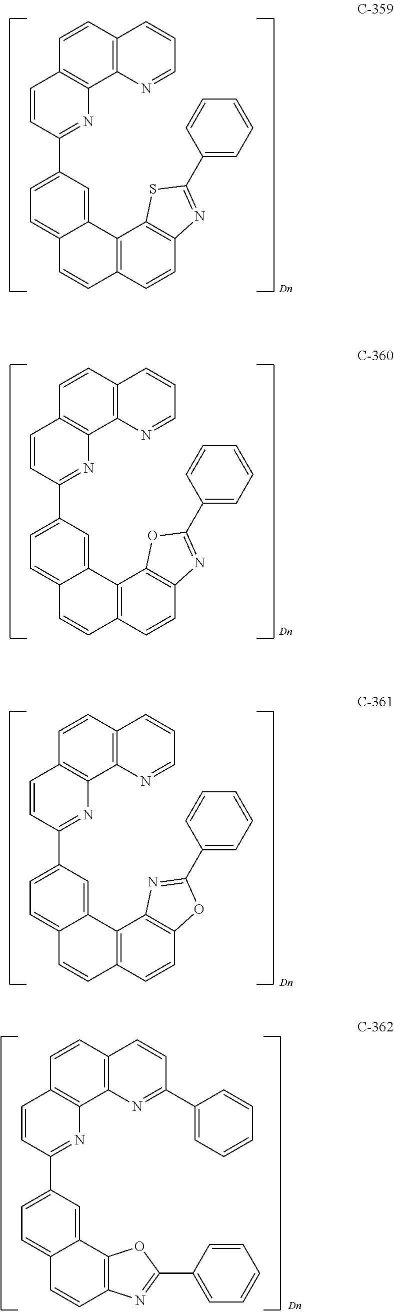

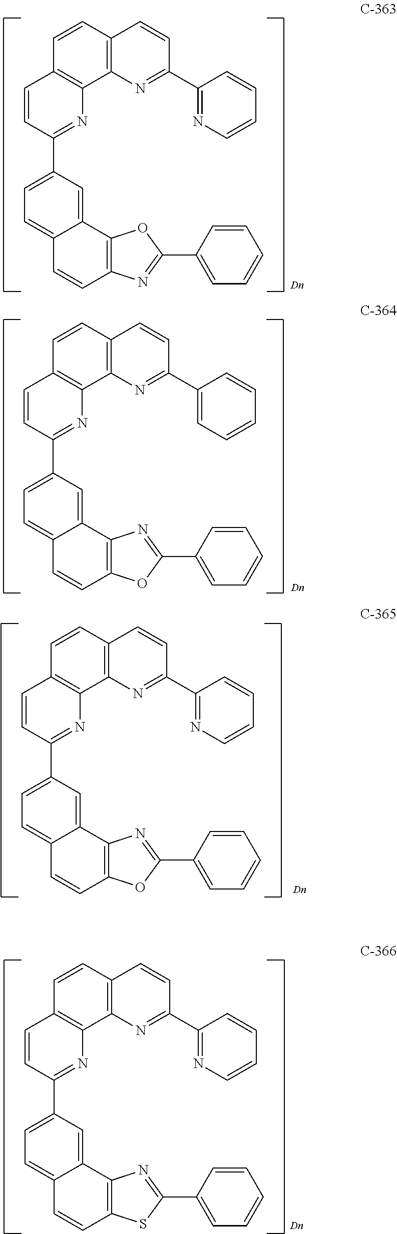

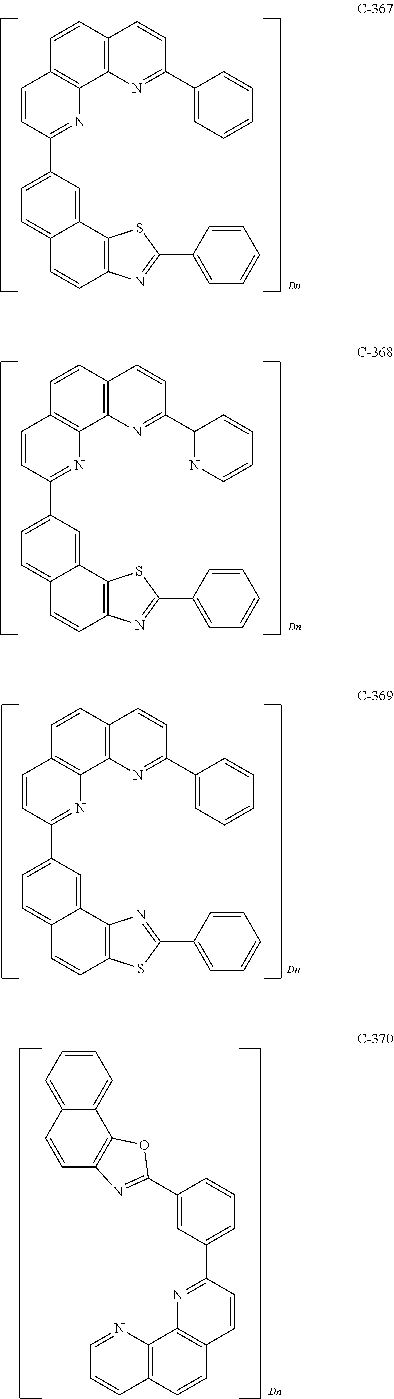

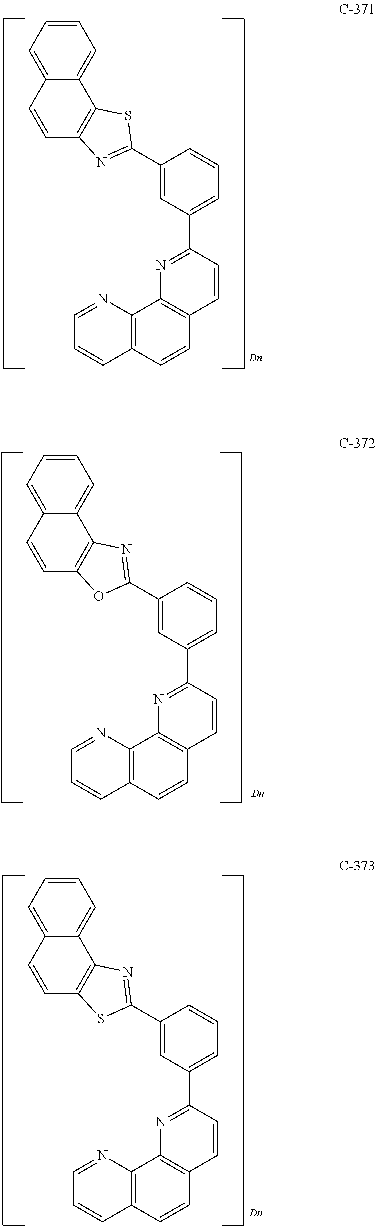

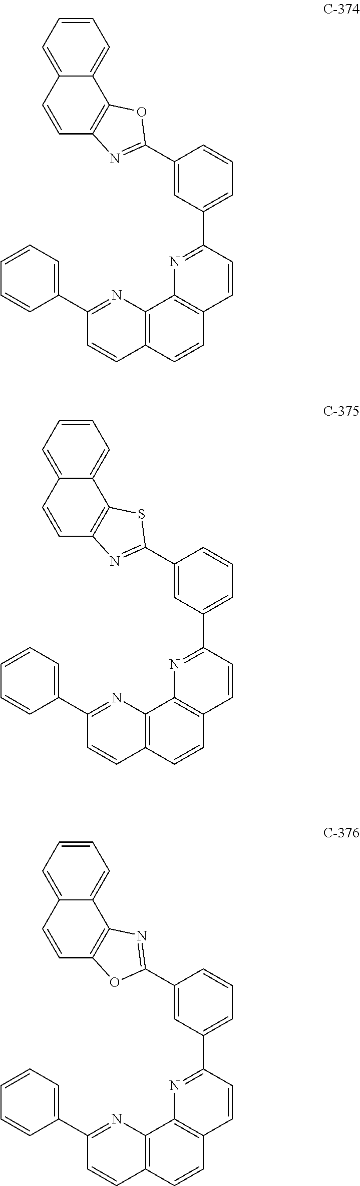 [SMILES: c1ccc(-c2ccc3ccc4ccc(-c5cccc(-c6nc7ccc8ccccc8c7o6)c5)nc4c3n2)cc1 ; c1ccc(-c2ccc3ccc4ccc(-c5cccc(-c6nc7ccc8ccccc8c7s6)c5)nc4c3n2)cc1 ; c1ccc(-c2ccc3ccc4ccc(-c5cccc(-c6nc7c(ccc8ccccc87)o6)c5)nc4c3n2)cc1]

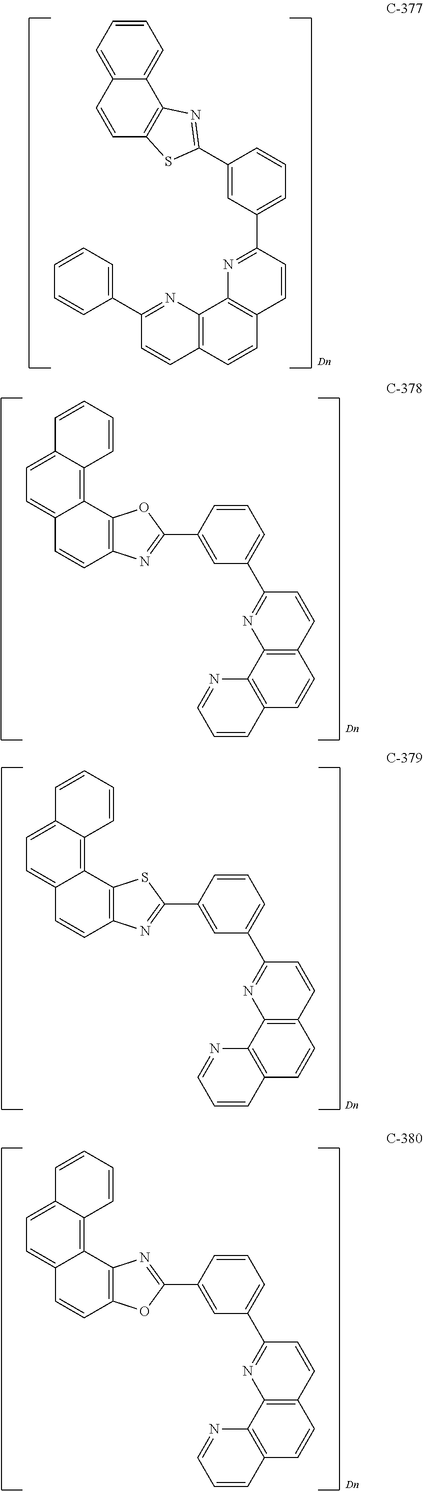

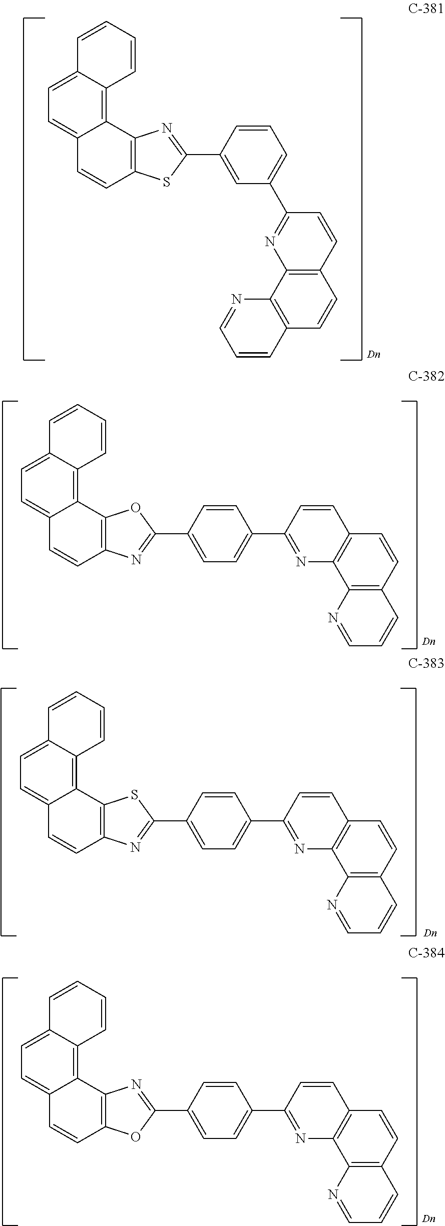

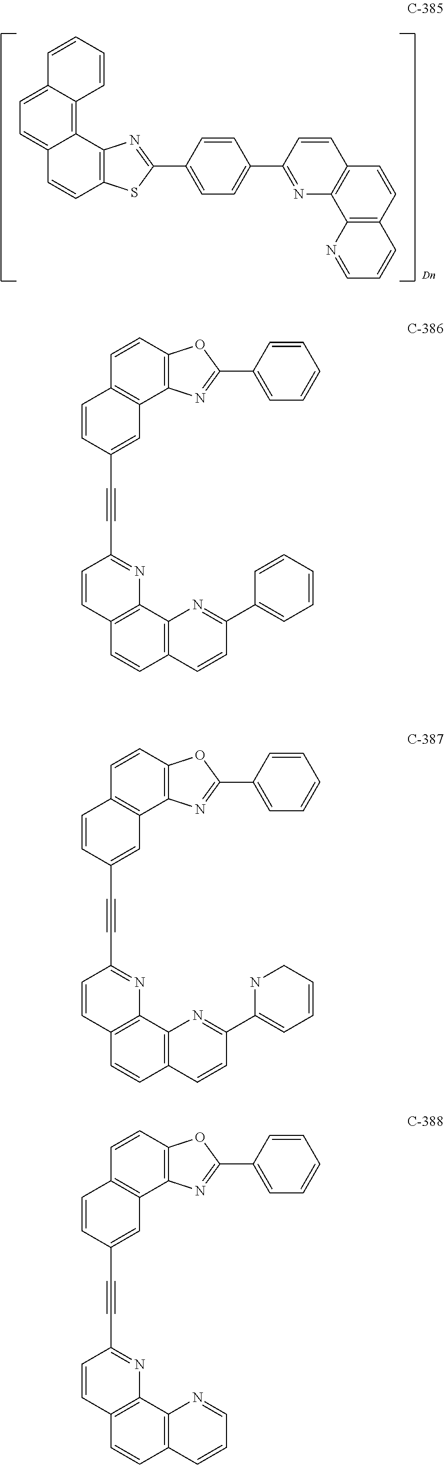

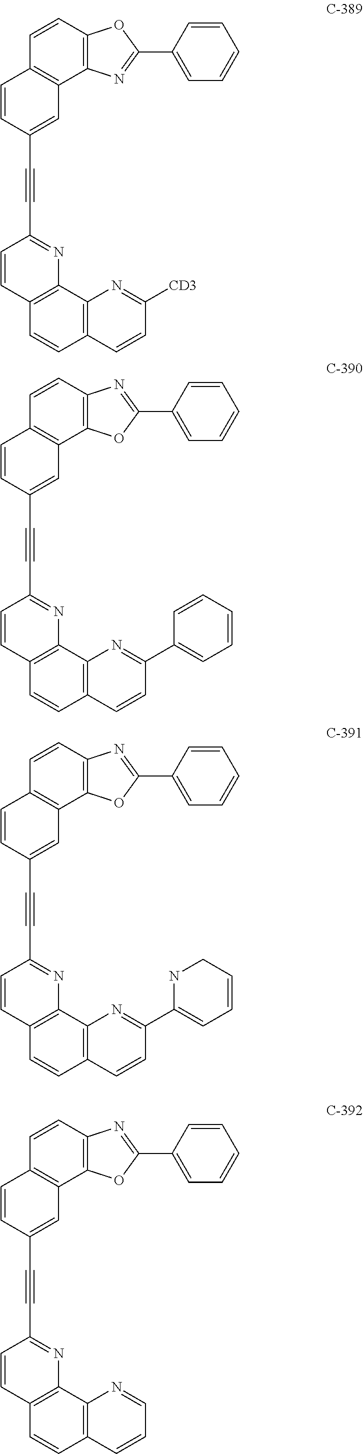

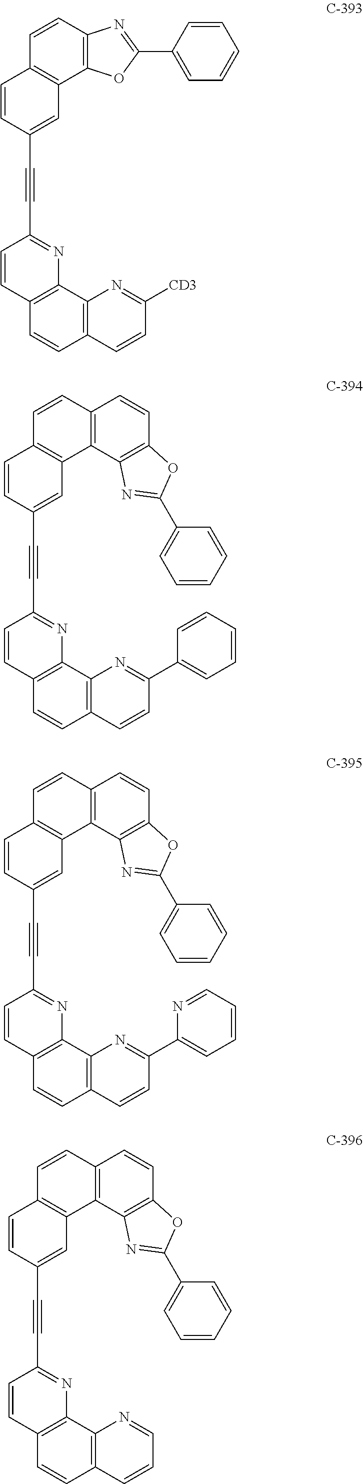

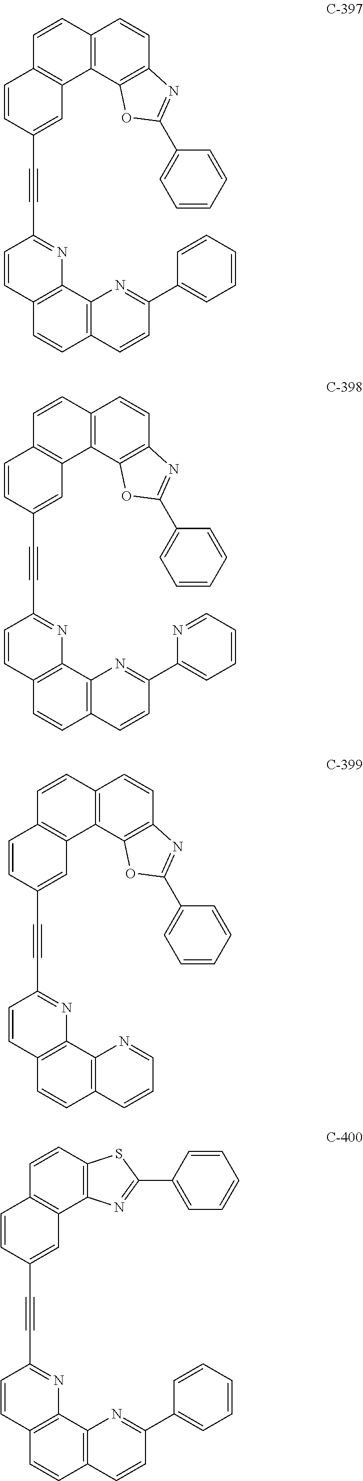

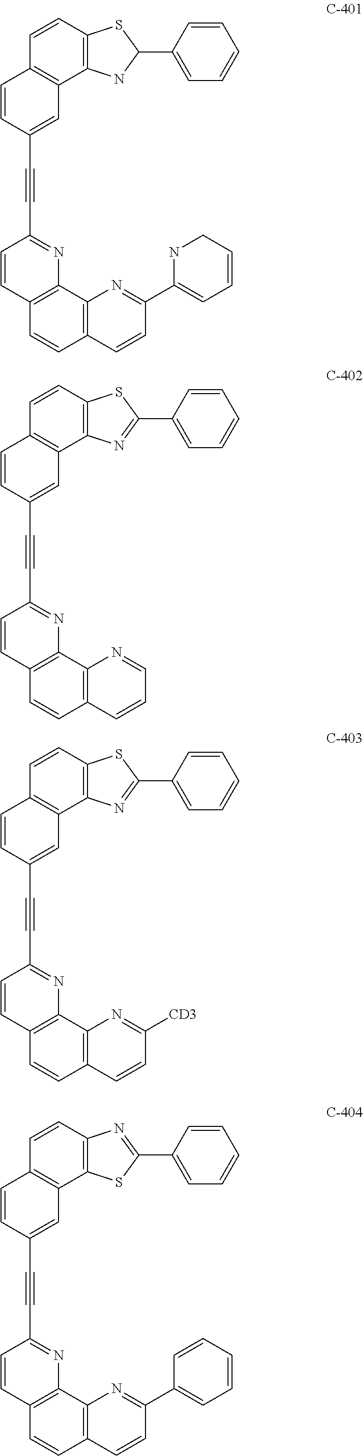

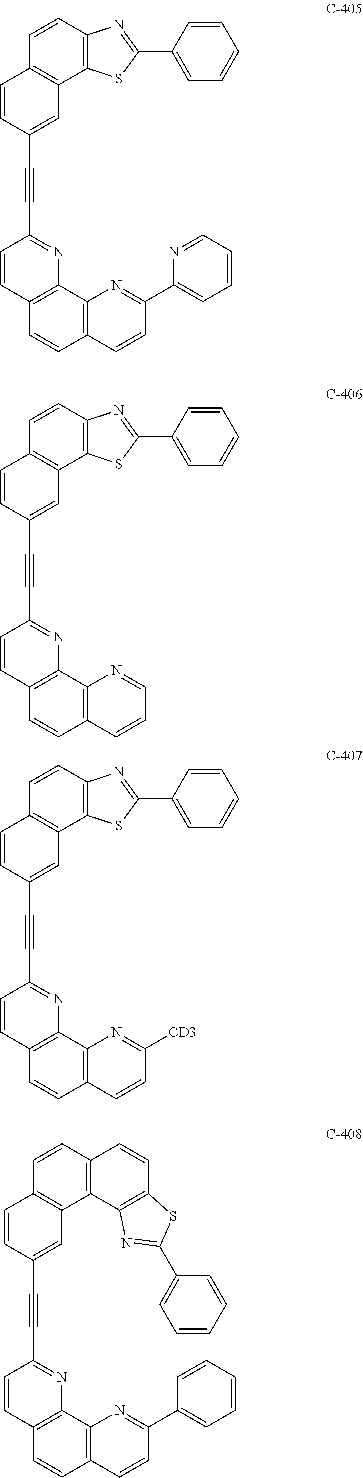

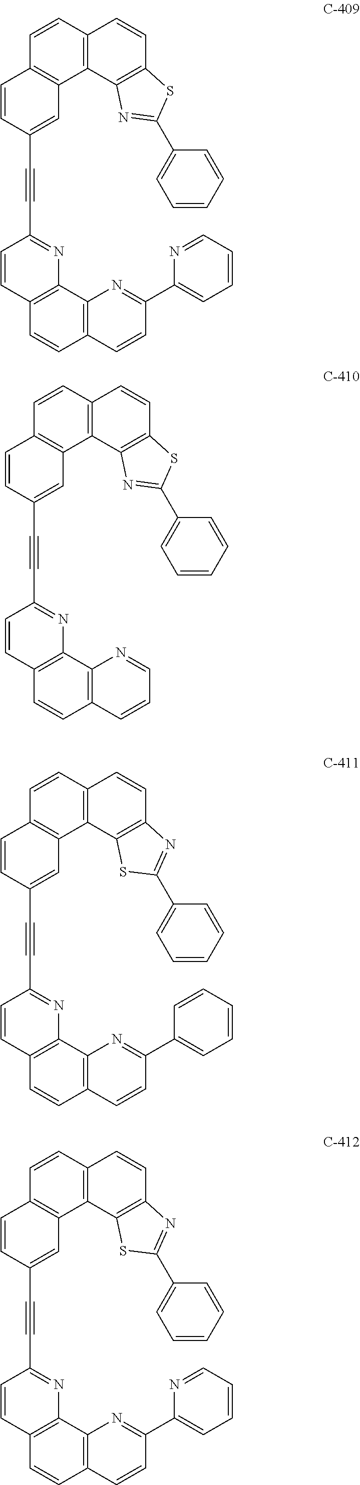

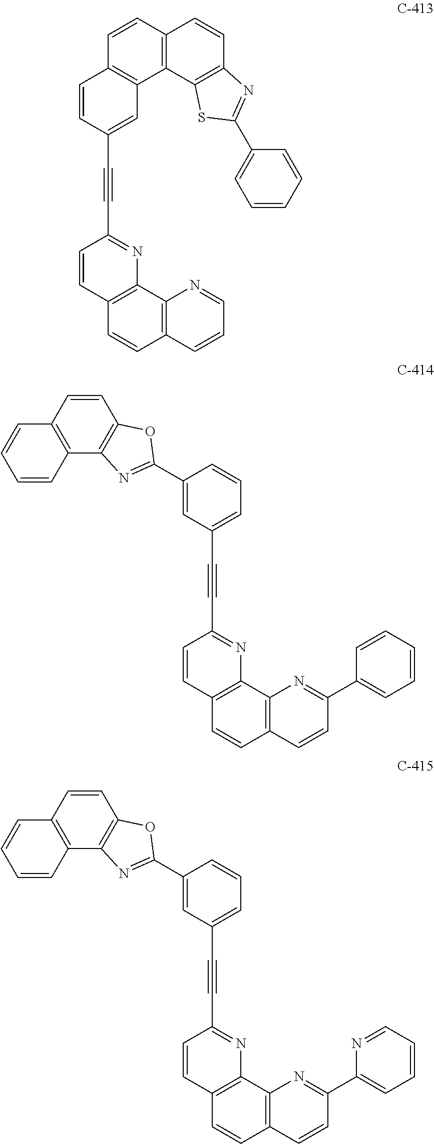 [SMILES: C(#Cc1ccc2ccc3cccnc3c2n1)c1ccc2ccc3ccc4nc(-c5ccccc5)sc4c3c2c1 ; C(#Cc1ccc2ccc3ccc(-c4ccccc4)nc3c2n1)c1cccc(-c2nc3c(ccc4ccccc43)o2)c1 ; C(#Cc1ccc2ccc3ccc(-c4ccccn4)nc3c2n1)c1cccc(-c2nc3c(ccc4ccccc43)o2)c1]

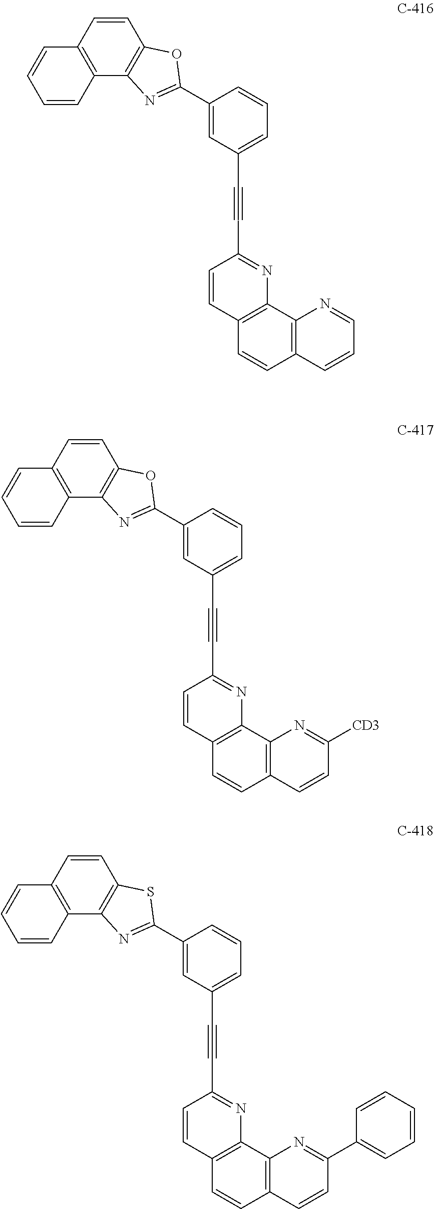 [SMILES: C(#Cc1ccc2ccc3cccnc3c2n1)c1cccc(-c2nc3c(ccc4ccccc43)o2)c1 ; [2H]C([2H])([2H])c1ccc2ccc3ccc(C#Cc4cccc(-c5nc6c(ccc7ccccc76)o5)c4)nc3c2n1 ; C(#Cc1ccc2ccc3ccc(-c4ccccc4)nc3c2n1)c1cccc(-c2nc3c(ccc4ccccc43)s2)c1]

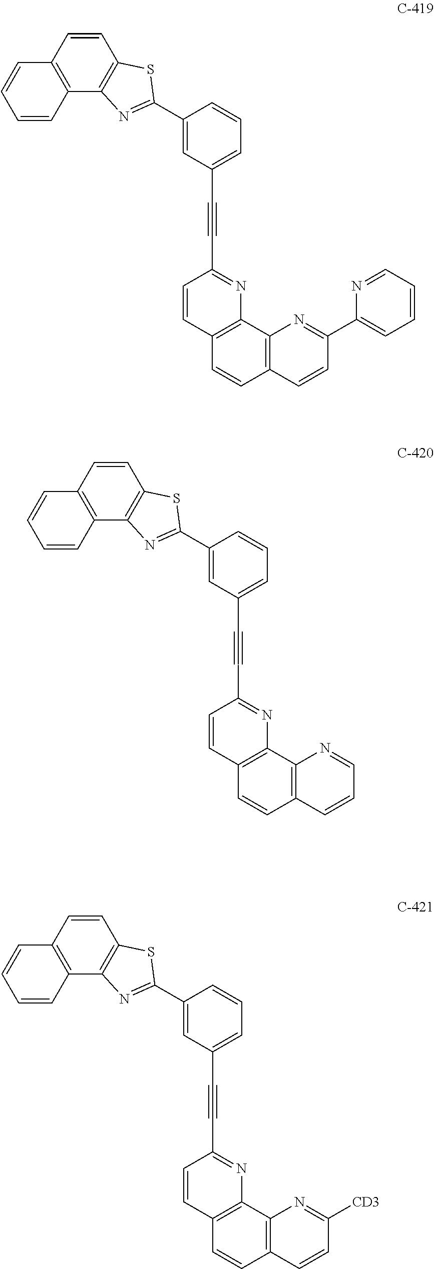 [SMILES: C(#Cc1ccc2ccc3ccc(-c4ccccn4)nc3c2n1)c1cccc(-c2nc3c(ccc4ccccc43)s2)c1 ; C(#Cc1ccc2ccc3cccnc3c2n1)c1cccc(-c2nc3c(ccc4ccccc43)s2)c1 ; [2H]C([2H])([2H])c1ccc2ccc3ccc(C#Cc4cccc(-c5nc6c(ccc7ccccc76)s5)c4)nc3c2n1]

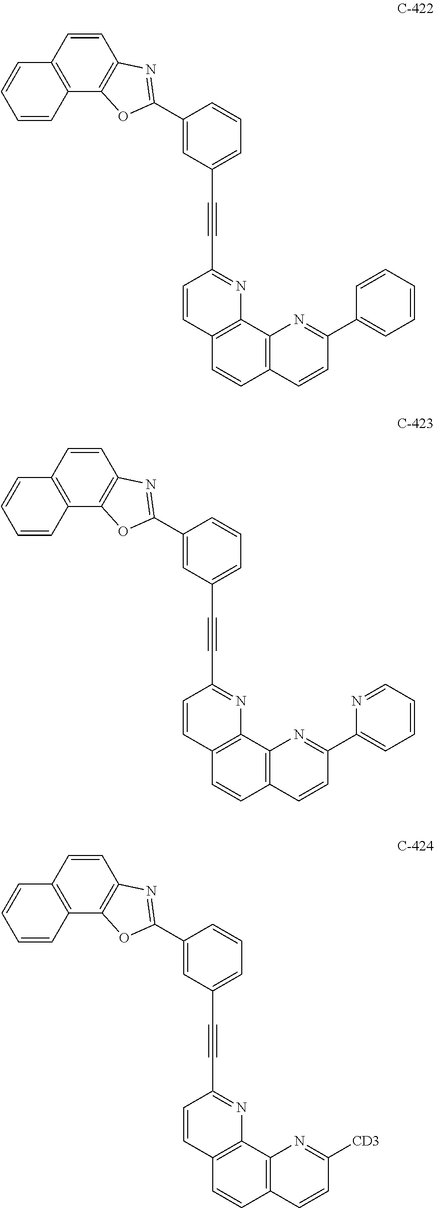 [SMILES: C(#Cc1ccc2ccc3ccc(-c4ccccc4)nc3c2n1)c1cccc(-c2nc3ccc4ccccc4c3o2)c1 ; C(#Cc1ccc2ccc3ccc(-c4ccccn4)nc3c2n1)c1cccc(-c2nc3ccc4ccccc4c3o2)c1 ; [2H]C([2H])([2H])c1ccc2ccc3ccc(C#Cc4cccc(-c5nc6ccc7ccccc7c6o5)c4)nc3c2n1]

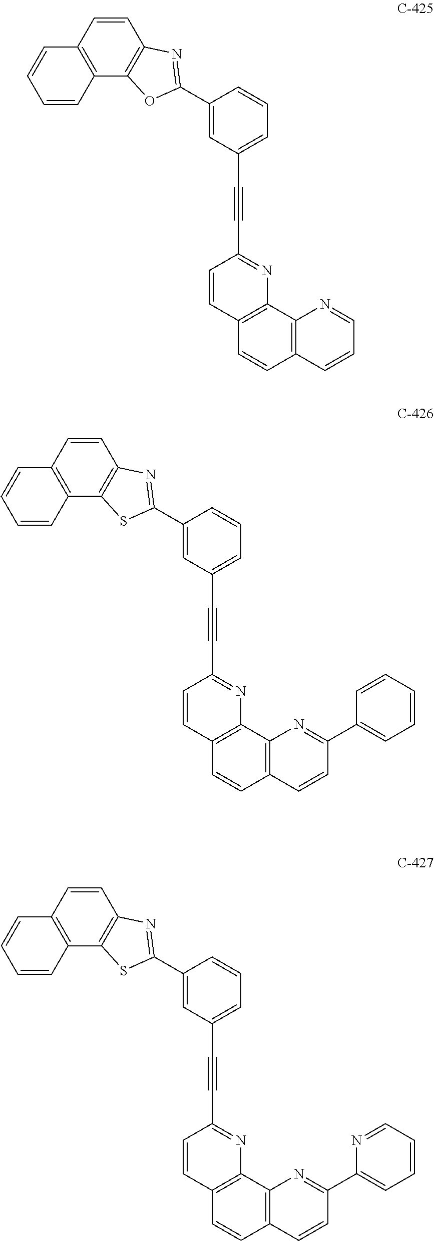 [SMILES: C(#Cc1ccc2ccc3cccnc3c2n1)c1cccc(-c2nc3ccc4ccccc4c3o2)c1 ; C(#Cc1ccc2ccc3ccc(-c4ccccc4)nc3c2n1)c1cccc(-c2nc3ccc4ccccc4c3s2)c1 ; C(#Cc1ccc2ccc3ccc(-c4ccccn4)nc3c2n1)c1cccc(-c2nc3ccc4ccccc4c3s2)c1]

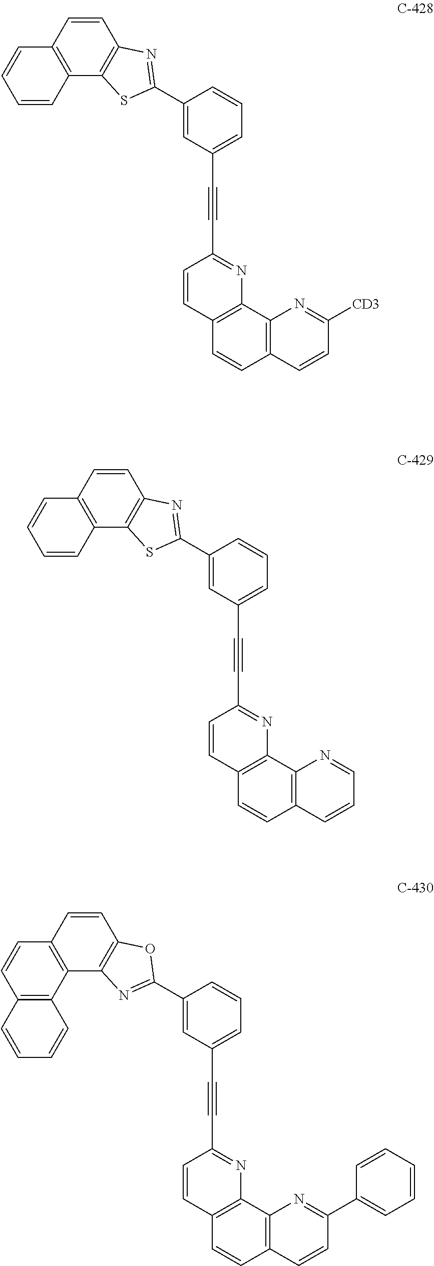 [SMILES: [2H]C([2H])([2H])c1ccc2ccc3ccc(C#Cc4cccc(-c5nc6ccc7ccccc7c6s5)c4)nc3c2n1 ; C(#Cc1ccc2ccc3cccnc3c2n1)c1cccc(-c2nc3ccc4ccccc4c3s2)c1 ; C(#Cc1ccc2ccc3ccc(-c4ccccc4)nc3c2n1)c1cccc(-c2nc3c(ccc4ccc5ccccc5c43)o2)c1]

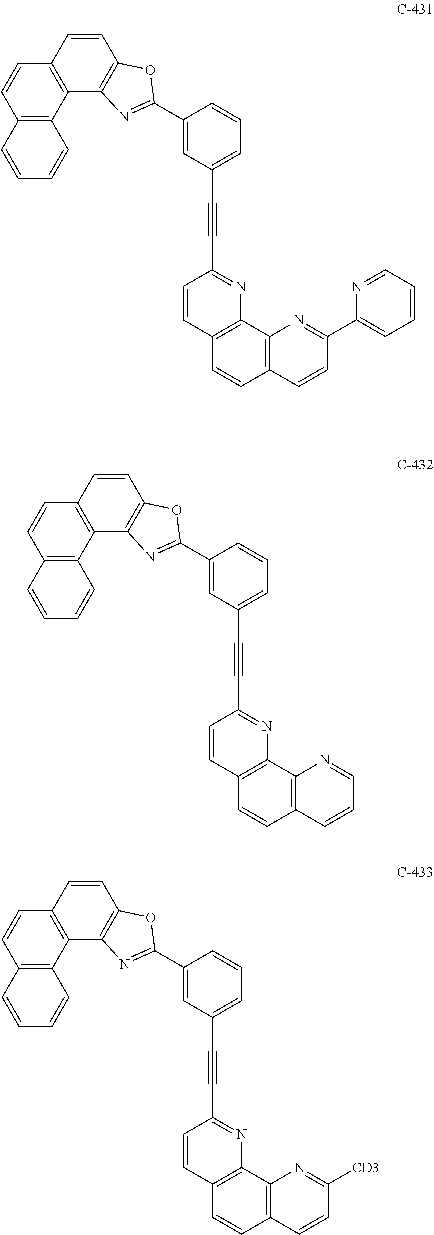

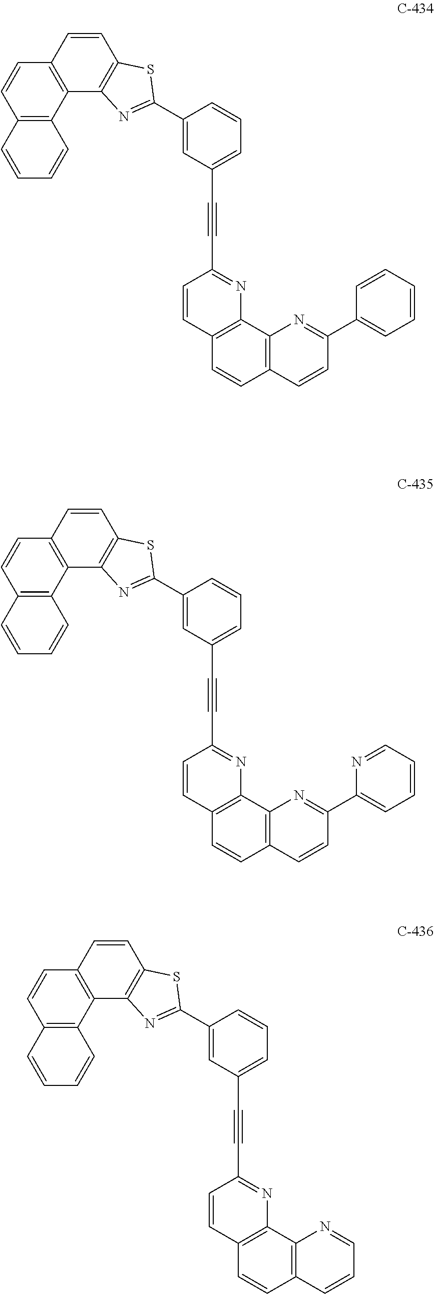 [SMILES: C(#Cc1ccc2ccc3ccc(-c4ccccc4)nc3c2n1)c1cccc(-c2nc3c(ccc4ccc5ccccc5c43)s2)c1 ; C(#Cc1ccc2ccc3ccc(-c4ccccn4)nc3c2n1)c1cccc(-c2nc3c(ccc4ccc5ccccc5c43)s2)c1 ; C(#Cc1ccc2ccc3cccnc3c2n1)c1cccc(-c2nc3c(ccc4ccc5ccccc5c43)s2)c1]

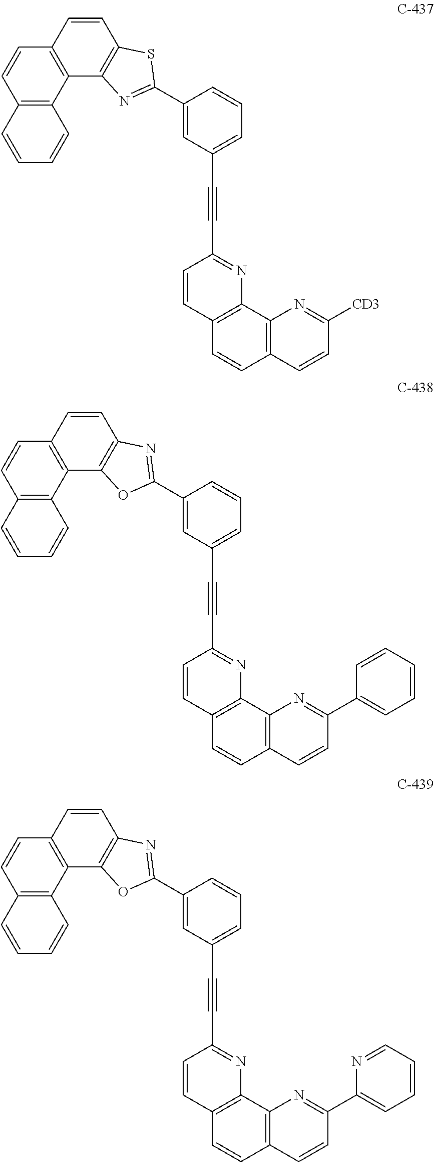 [SMILES: [2H]C([2H])([2H])c1ccc2ccc3ccc(C#Cc4cccc(-c5nc6c(ccc7ccc8ccccc8c76)s5)c4)nc3c2n1 ; C(#Cc1ccc2ccc3ccc(-c4ccccc4)nc3c2n1)c1cccc(-c2nc3ccc4ccc5ccccc5c4c3o2)c1 ; C(#Cc1ccc2ccc3ccc(-c4ccccn4)nc3c2n1)c1cccc(-c2nc3ccc4ccc5ccccc5c4c3o2)c1]

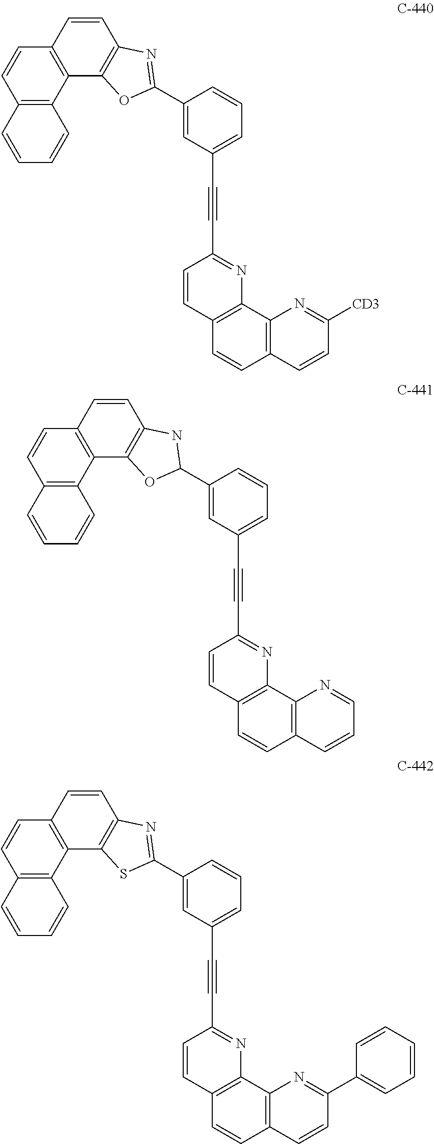

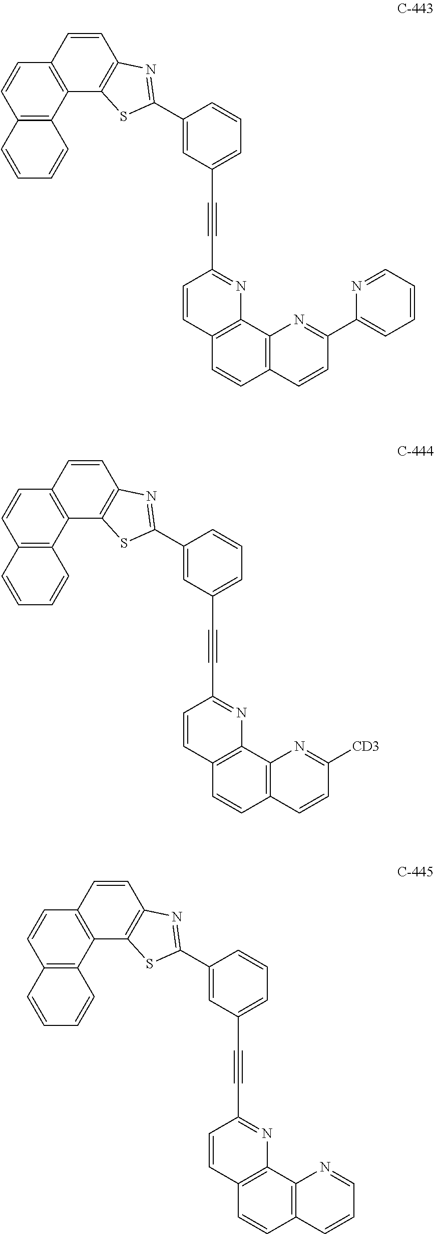 [SMILES: C(#Cc1ccc2ccc3ccc(-c4ccccn4)nc3c2n1)c1cccc(-c2nc3ccc4ccc5ccccc5c4c3s2)c1 ; [2H]C([2H])([2H])c1ccc2ccc3ccc(C#Cc4cccc(-c5nc6ccc7ccc8ccccc8c7c6s5)c4)nc3c2n1 ; C(#Cc1ccc2ccc3cccnc3c2n1)c1cccc(-c2nc3ccc4ccc5ccccc5c4c3s2)c1]

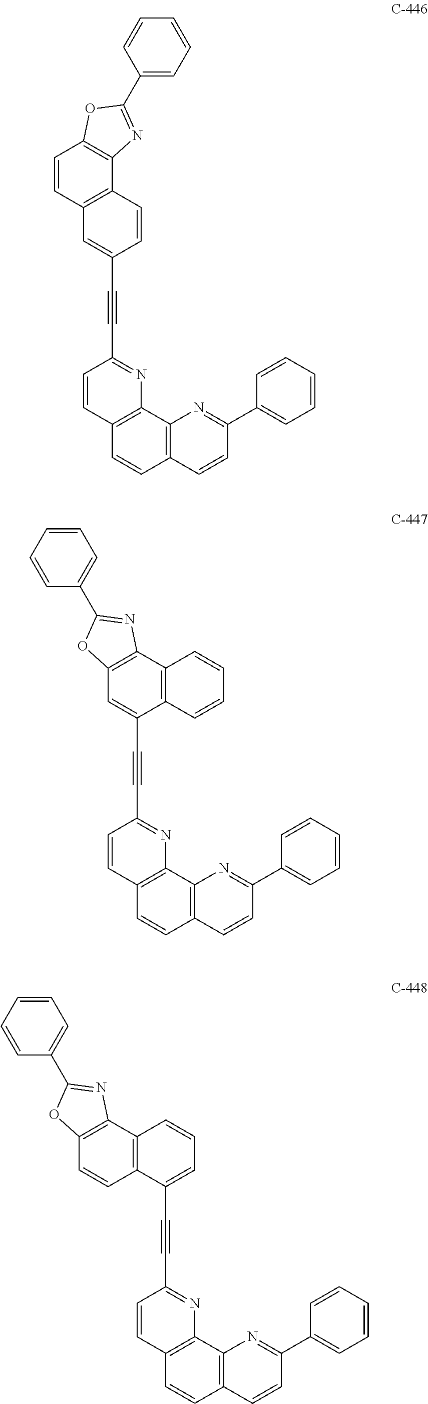 [SMILES: C(#Cc1ccc2ccc3ccc(-c4ccccc4)nc3c2n1)c1ccc2c(ccc3oc(-c4ccccc4)nc32)c1 ; C(#Cc1cc2oc(-c3ccccc3)nc2c2ccccc12)c1ccc2ccc3ccc(-c4ccccc4)nc3c2n1 ; C(#Cc1cccc2c1ccc1oc(-c3ccccc3)nc12)c1ccc2ccc3ccc(-c4ccccc4)nc3c2n1]

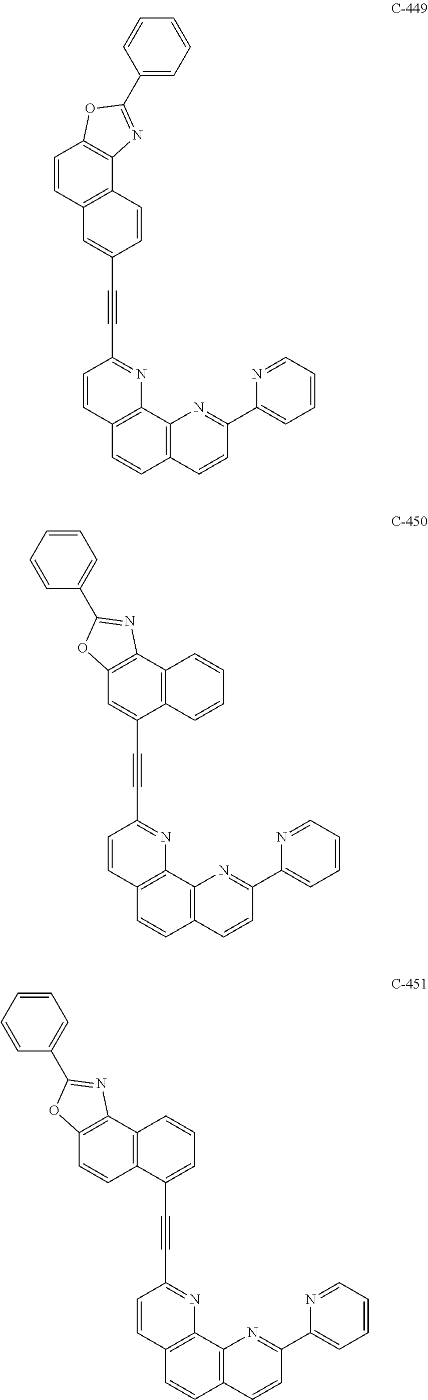 [SMILES: C(#Cc1ccc2ccc3ccc(-c4ccccn4)nc3c2n1)c1ccc2c(ccc3oc(-c4ccccc4)nc32)c1 ; C(#Cc1cc2oc(-c3ccccc3)nc2c2ccccc12)c1ccc2ccc3ccc(-c4ccccn4)nc3c2n1 ; C(#Cc1cccc2c1ccc1oc(-c3ccccc3)nc12)c1ccc2ccc3ccc(-c4ccccn4)nc3c2n1]

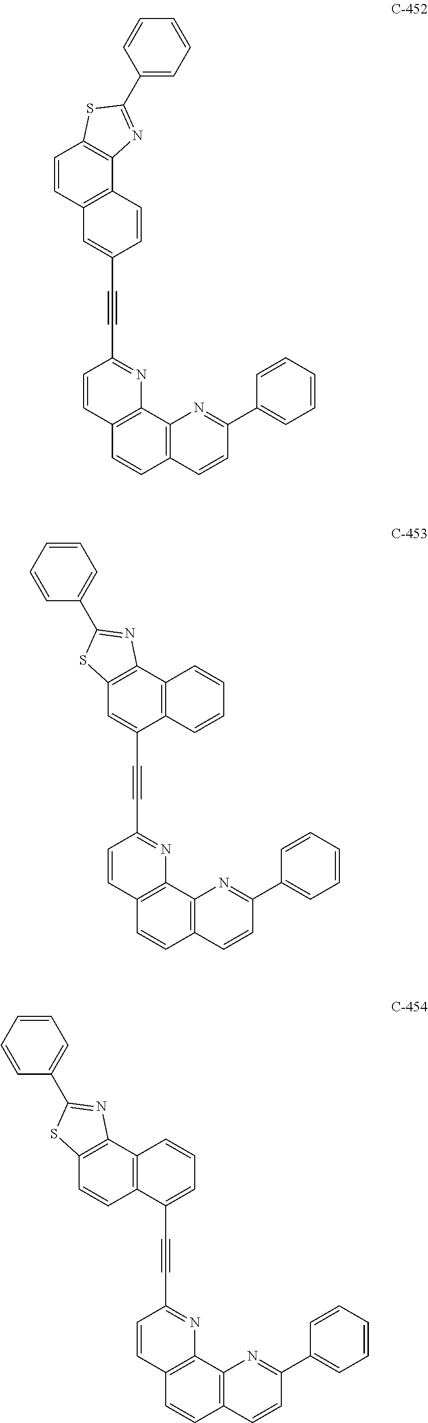 [SMILES: C(#Cc1ccc2ccc3ccc(-c4ccccc4)nc3c2n1)c1ccc2c(ccc3sc(-c4ccccc4)nc32)c1 ; C(#Cc1cc2sc(-c3ccccc3)nc2c2ccccc12)c1ccc2ccc3ccc(-c4ccccc4)nc3c2n1 ; C(#Cc1cccc2c1ccc1sc(-c3ccccc3)nc12)c1ccc2ccc3ccc(-c4ccccc4)nc3c2n1]

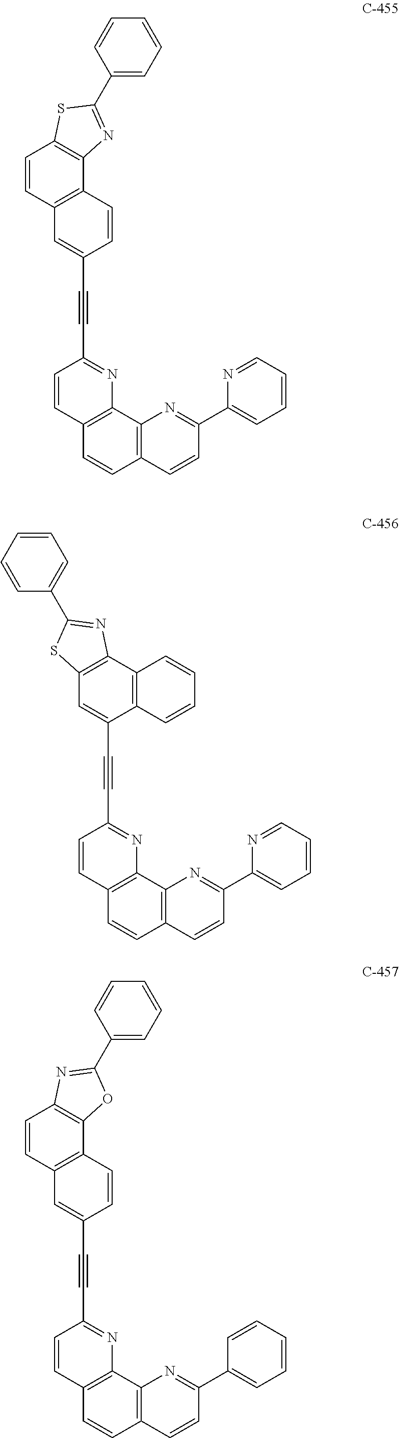

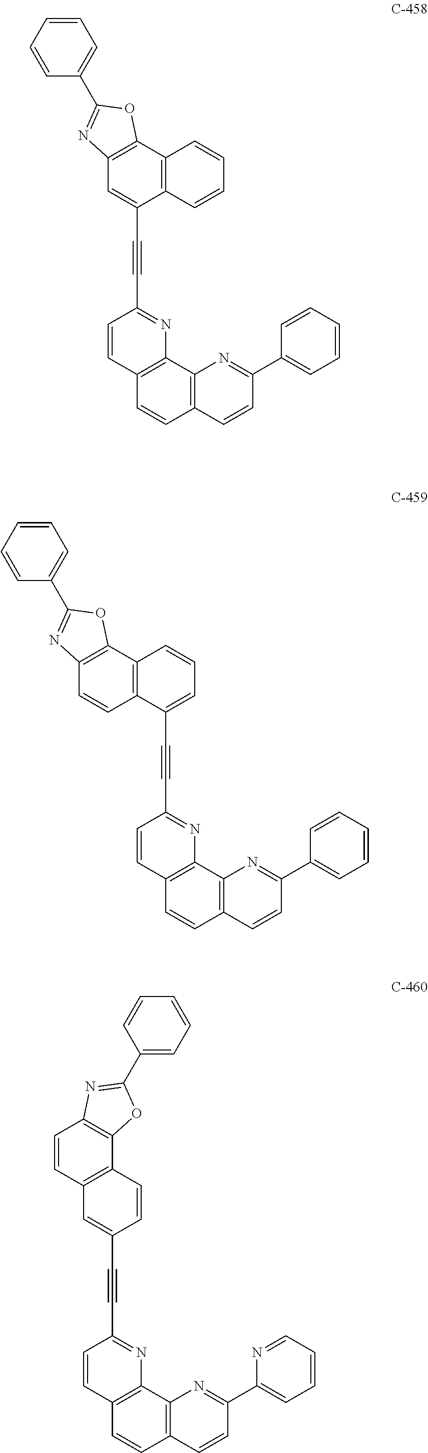 [SMILES: C(#Cc1cc2nc(-c3ccccc3)oc2c2ccccc12)c1ccc2ccc3ccc(-c4ccccc4)nc3c2n1 ; C(#Cc1cccc2c1ccc1nc(-c3ccccc3)oc12)c1ccc2ccc3ccc(-c4ccccc4)nc3c2n1 ; C(#Cc1ccc2ccc3ccc(-c4ccccn4)nc3c2n1)c1ccc2c(ccc3nc(-c4ccccc4)oc32)c1]

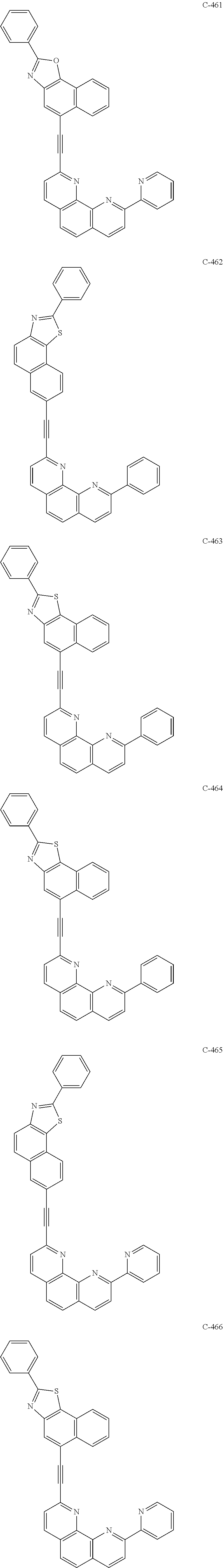 [SMILES: C(#Cc1cc2nc(-c3ccccc3)oc2c2ccccc12)c1ccc2ccc3ccc(-c4ccccn4)nc3c2n1 ; C(#Cc1ccc2ccc3ccc(-c4ccccc4)nc3c2n1)c1ccc2c(ccc3nc(-c4ccccc4)sc32)c1 ; C(#Cc1cc2nc(-c3ccccc3)sc2c2ccccc12)c1ccc2ccc3ccc(-c4ccccc4)nc3c2n1 ; C(#Cc1cc2nc(-c3ccccc3)sc2c2ccccc12)c1ccc2ccc3ccc(-c4ccccc4)nc3c2n1 ; C(#Cc1ccc2ccc3ccc(-c4ccccn4)nc3c2n1)c1ccc2c(ccc3nc(-c4ccccc4)sc32)c1 ; C(#Cc1cc2nc(-c3ccccc3)sc2c2ccccc12)c1ccc2ccc3ccc(-c4ccccn4)nc3c2n1]

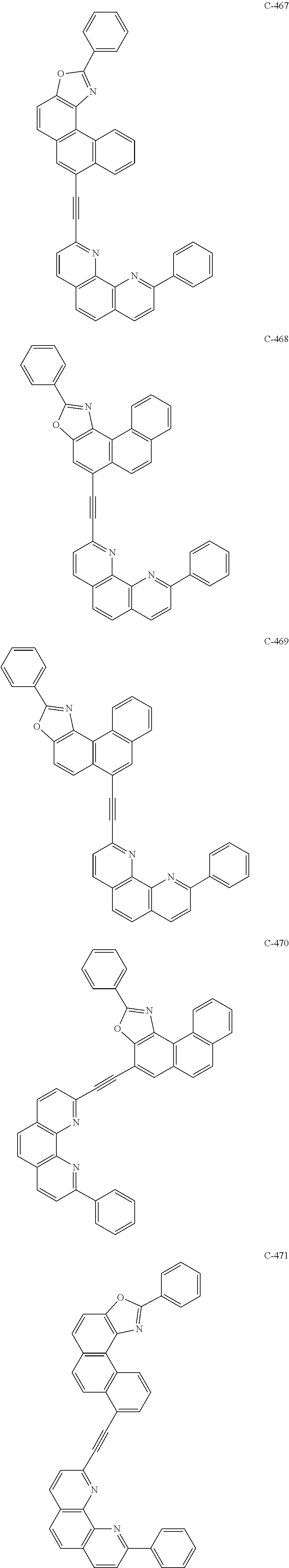 [SMILES: C(#Cc1cc2ccc3oc(-c4ccccc4)nc3c2c2ccccc12)c1ccc2ccc3ccc(-c4ccccc4)nc3c2n1 ; C(#Cc1cc2oc(-c3ccccc3)nc2c2c1ccc1ccccc12)c1ccc2ccc3ccc(-c4ccccc4)nc3c2n1 ; C(#Cc1cc2ccccc2c2c1ccc1oc(-c3ccccc3)nc12)c1ccc2ccc3ccc(-c4ccccc4)nc3c2n1 ; C(#Cc1cc2ccc3ccccc3c2c2nc(-c3ccccc3)oc12)c1ccc2ccc3ccc(-c4ccccc4)nc3c2n1 ; C(#Cc1cccc2c1ccc1ccc3oc(-c4ccccc4)nc3c12)c1ccc2ccc3ccc(-c4ccccc4)nc3c2n1]

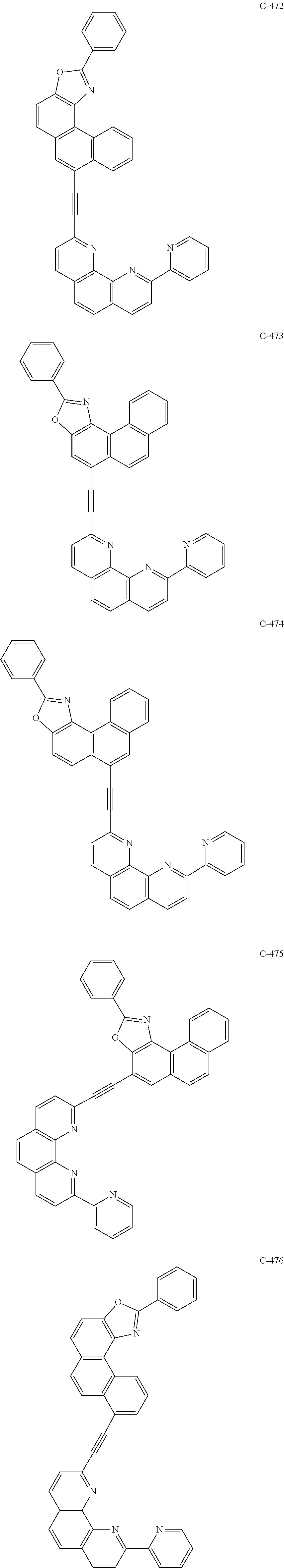 [SMILES: C(#Cc1cc2ccc3oc(-c4ccccc4)nc3c2c2ccccc12)c1ccc2ccc3ccc(-c4ccccn4)nc3c2n1 ; C(#Cc1cc2oc(-c3ccccc3)nc2c2c1ccc1ccccc12)c1ccc2ccc3ccc(-c4ccccn4)nc3c2n1 ; C(#Cc1cc2ccccc2c2c1ccc1oc(-c3ccccc3)nc12)c1ccc2ccc3ccc(-c4ccccn4)nc3c2n1 ; C(#Cc1cc2ccc3ccccc3c2c2nc(-c3ccccc3)oc12)c1ccc2ccc3ccc(-c4ccccn4)nc3c2n1 ; C(#Cc1cccc2c1ccc1ccc3oc(-c4ccccc4)nc3c12)c1ccc2ccc3ccc(-c4ccccn4)nc3c2n1]

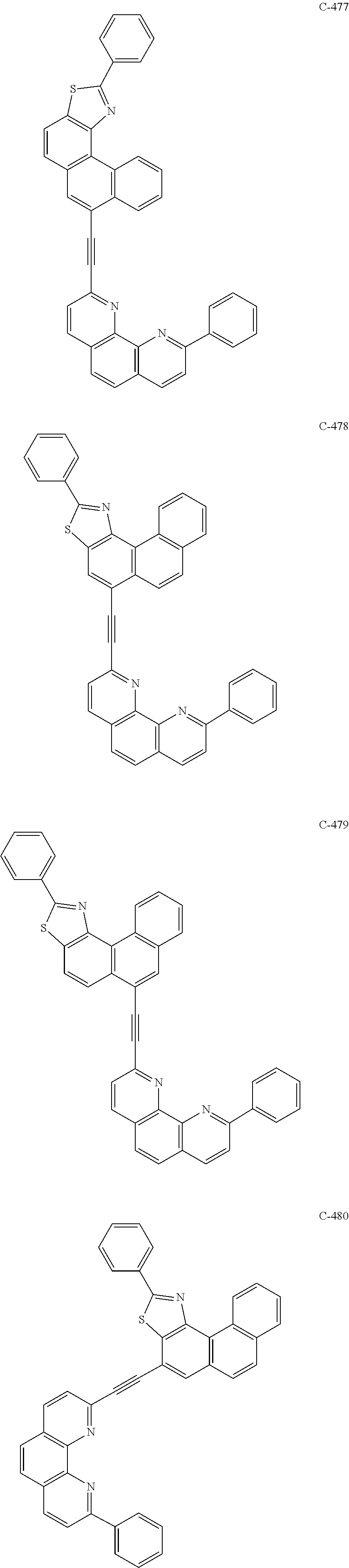 [SMILES: C(#Cc1cc2ccc3sc(-c4ccccc4)nc3c2c2ccccc12)c1ccc2ccc3ccc(-c4ccccc4)nc3c2n1 ; C(#Cc1cc2sc(-c3ccccc3)nc2c2c1ccc1ccccc12)c1ccc2ccc3ccc(-c4ccccc4)nc3c2n1 ; C(#Cc1cc2ccccc2c2c1ccc1sc(-c3ccccc3)nc12)c1ccc2ccc3ccc(-c4ccccc4)nc3c2n1 ; C(#Cc1cc2ccc3ccccc3c2c2nc(-c3ccccc3)sc12)c1ccc2ccc3ccc(-c4ccccc4)nc3c2n1]

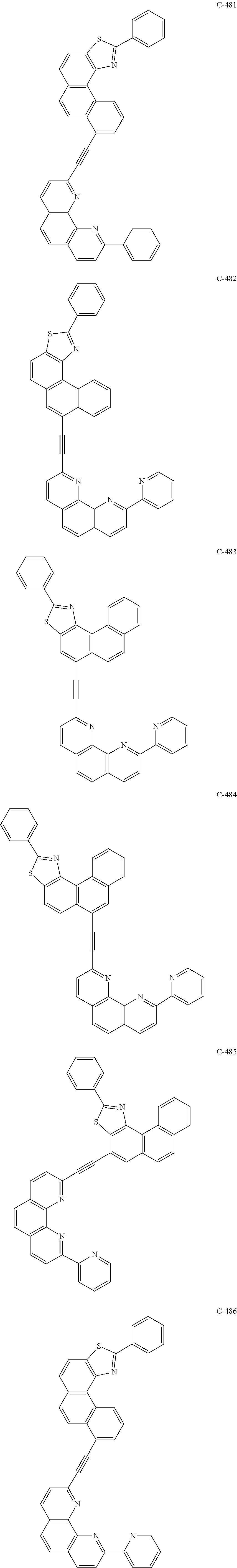

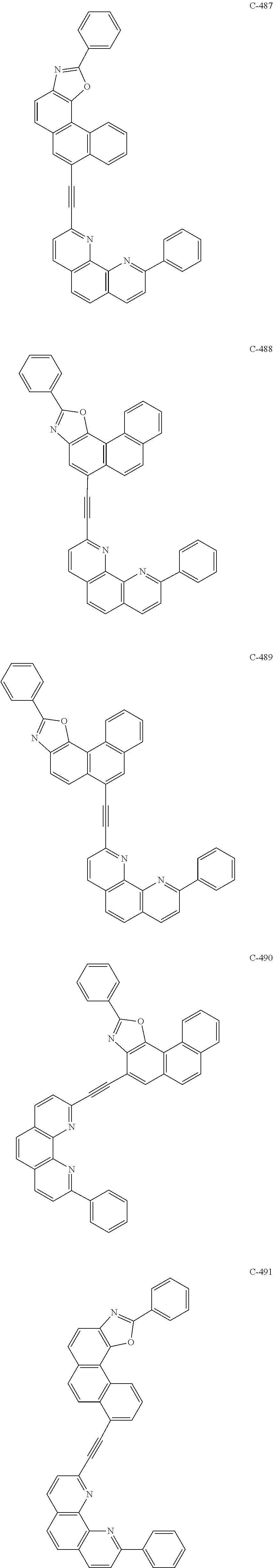 [SMILES: C(#Cc1cc2ccc3nc(-c4ccccc4)oc3c2c2ccccc12)c1ccc2ccc3ccc(-c4ccccc4)nc3c2n1 ; C(#Cc1cc2nc(-c3ccccc3)oc2c2c1ccc1ccccc12)c1ccc2ccc3ccc(-c4ccccc4)nc3c2n1 ; C(#Cc1cc2ccccc2c2c1ccc1nc(-c3ccccc3)oc12)c1ccc2ccc3ccc(-c4ccccc4)nc3c2n1 ; C(#Cc1cc2ccc3ccccc3c2c2oc(-c3ccccc3)nc12)c1ccc2ccc3ccc(-c4ccccc4)nc3c2n1 ; C(#Cc1cccc2c1ccc1ccc3nc(-c4ccccc4)oc3c12)c1ccc2ccc3ccc(-c4ccccc4)nc3c2n1]

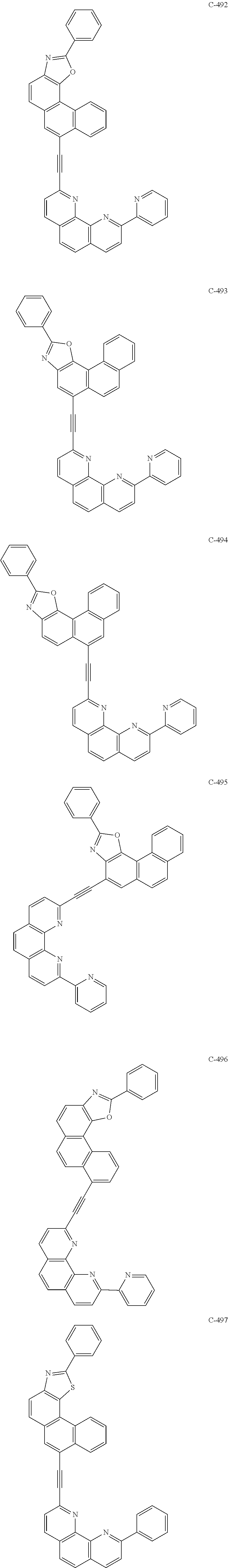

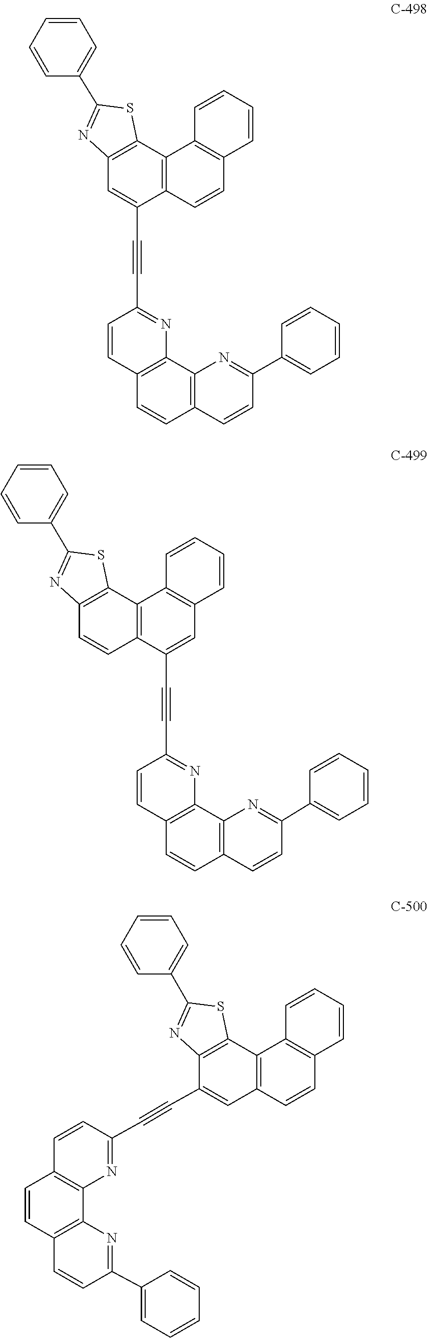 [SMILES: C(#Cc1cc2nc(-c3ccccc3)sc2c2c1ccc1ccccc12)c1ccc2ccc3ccc(-c4ccccc4)nc3c2n1 ; C(#Cc1cc2ccccc2c2c1ccc1nc(-c3ccccc3)sc12)c1ccc2ccc3ccc(-c4ccccc4)nc3c2n1 ; C(#Cc1cc2ccc3ccccc3c2c2sc(-c3ccccc3)nc12)c1ccc2ccc3ccc(-c4ccccc4)nc3c2n1]

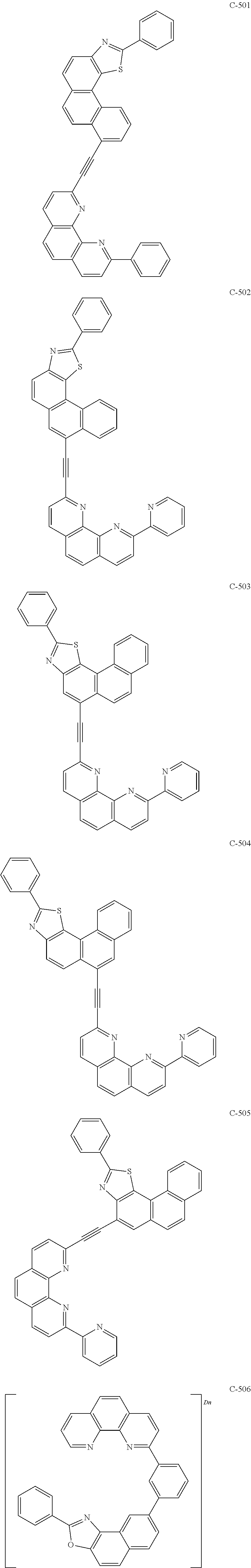

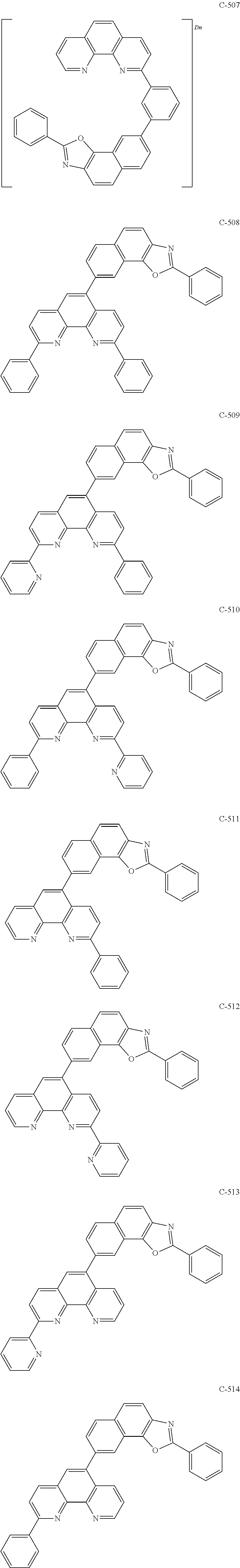

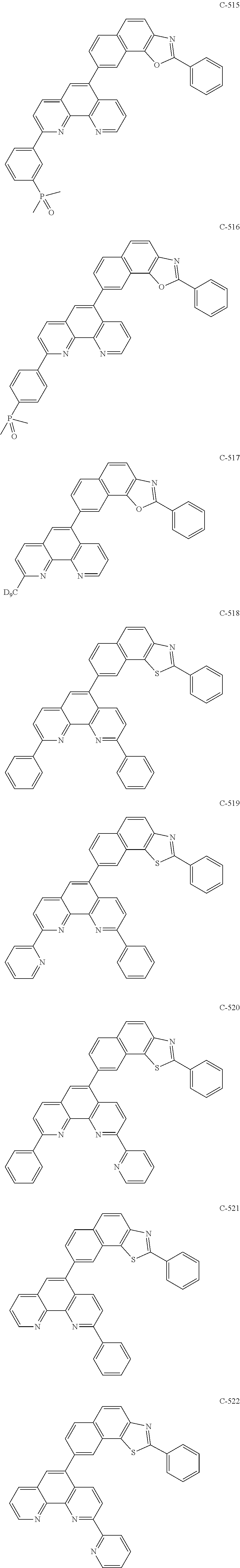

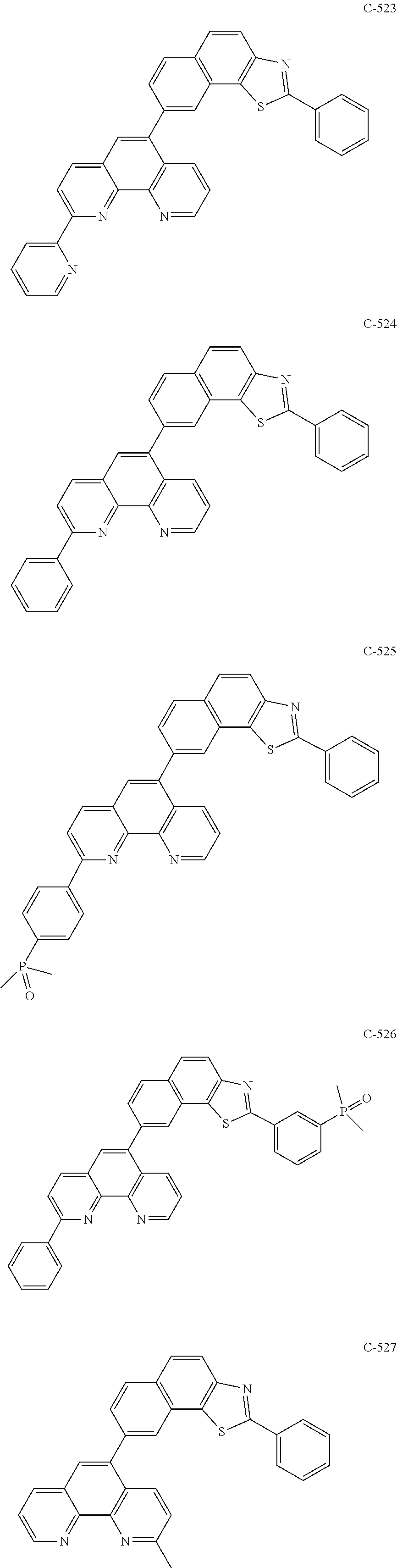 [SMILES: c1ccc(-c2nc3ccc4ccc(-c5cc6ccc(-c7ccccn7)nc6c6ncccc56)cc4c3s2)cc1 ; c1ccc(-c2ccc3cc(-c4ccc5ccc6nc(-c7ccccc7)sc6c5c4)c4cccnc4c3n2)cc1 ; CP(C)(=O)c1ccc(-c2ccc3cc(-c4ccc5ccc6nc(-c7ccccc7)sc6c5c4)c4cccnc4c3n2)cc1 ; CP(C)(=O)c1cccc(-c2nc3ccc4ccc(-c5cc6ccc(-c7ccccc7)nc6c6ncccc56)cc4c3s2)c1 ; Cc1ccc2c(-c3ccc4ccc5nc(-c6ccccc6)sc5c4c3)cc3cccnc3c2n1]

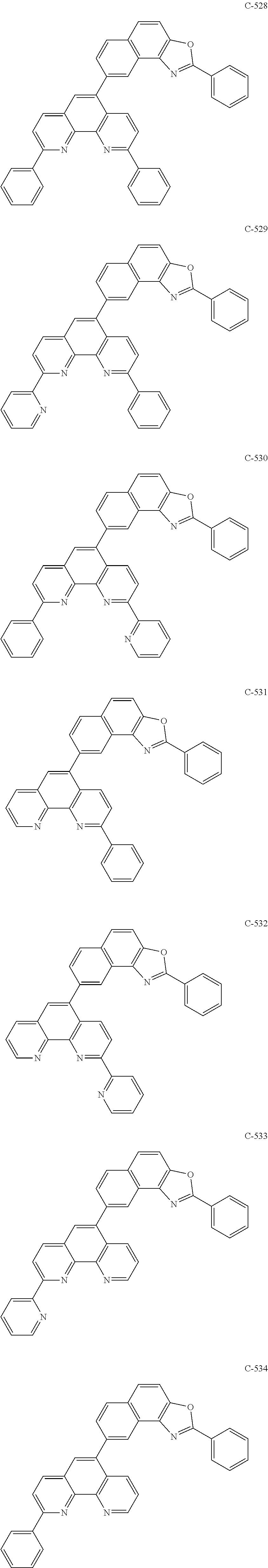

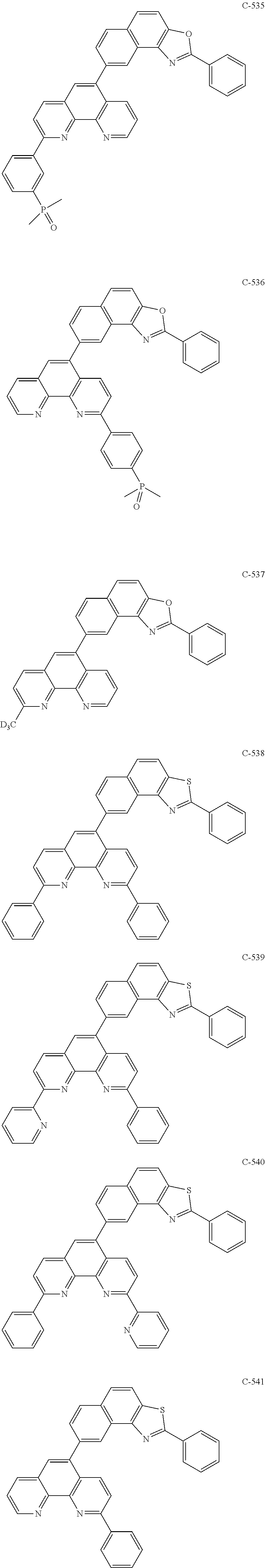

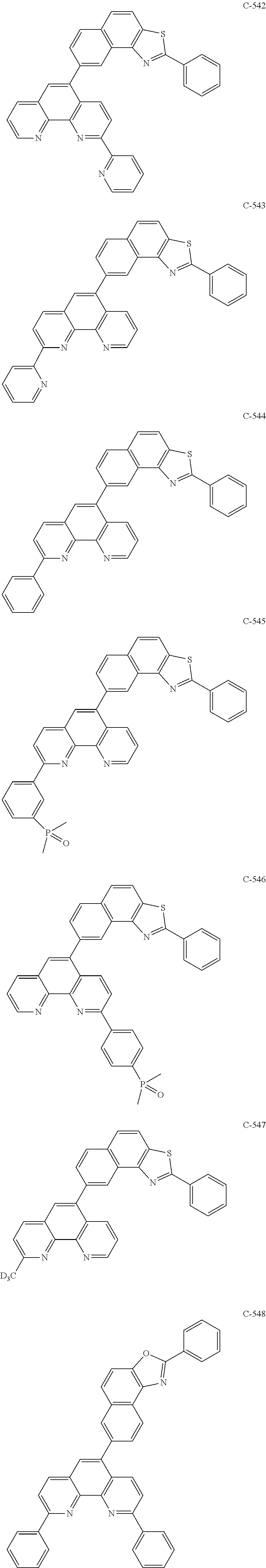

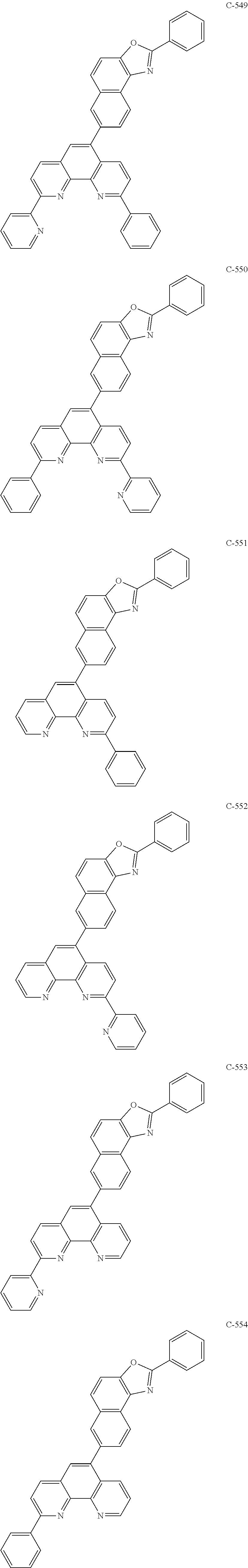

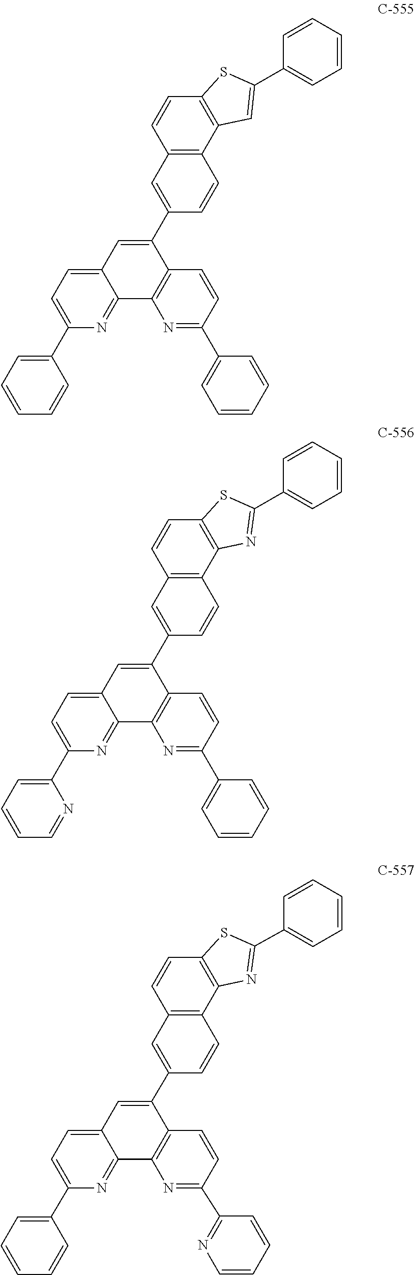

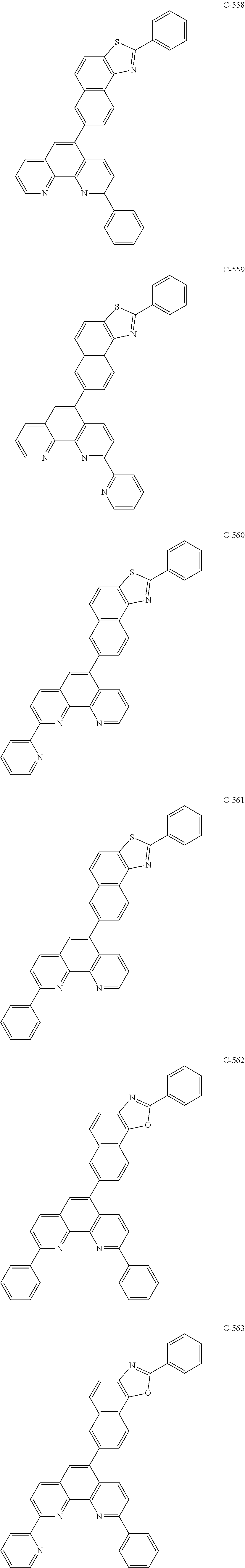

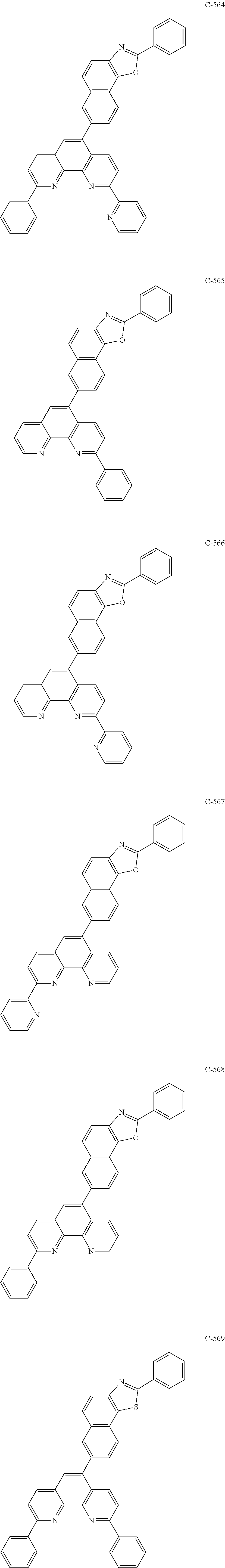

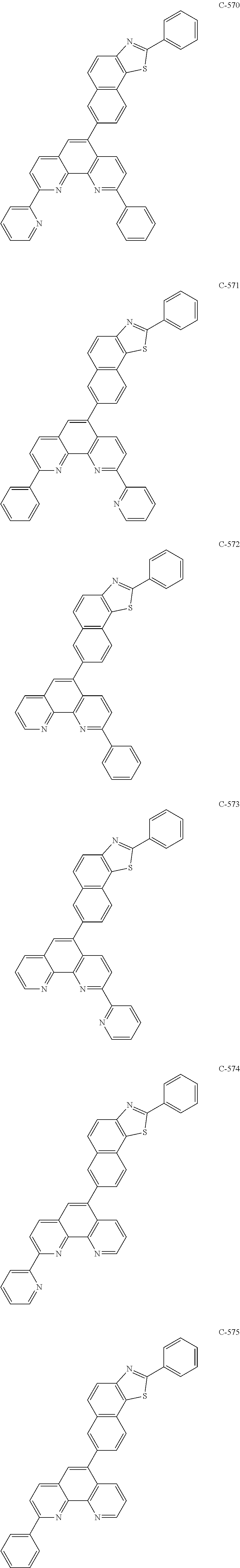

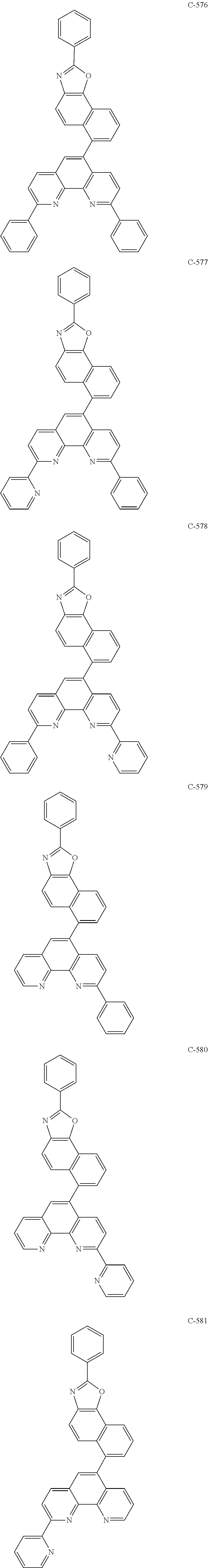 [SMILES: c1ccc(-c2ccc3cc(-c4cccc5c4ccc4nc(-c6ccccc6)oc45)c4ccc(-c5ccccc5)nc4c3n2)cc1 ; c1ccc(-c2ccc3c(-c4cccc5c4ccc4nc(-c6ccccc6)oc45)cc4ccc(-c5ccccn5)nc4c3n2)cc1 ; c1ccc(-c2ccc3cc(-c4cccc5c4ccc4nc(-c6ccccc6)oc45)c4ccc(-c5ccccn5)nc4c3n2)cc1 ; c1ccc(-c2ccc3c(-c4cccc5c4ccc4nc(-c6ccccc6)oc45)cc4cccnc4c3n2)cc1 ; c1ccc(-c2nc3ccc4c(-c5cc6cccnc6c6nc(-c7ccccn7)ccc56)cccc4c3o2)cc1 ; c1ccc(-c2nc3ccc4c(-c5cc6ccc(-c7ccccn7)nc6c6ncccc56)cccc4c3o2)cc1]

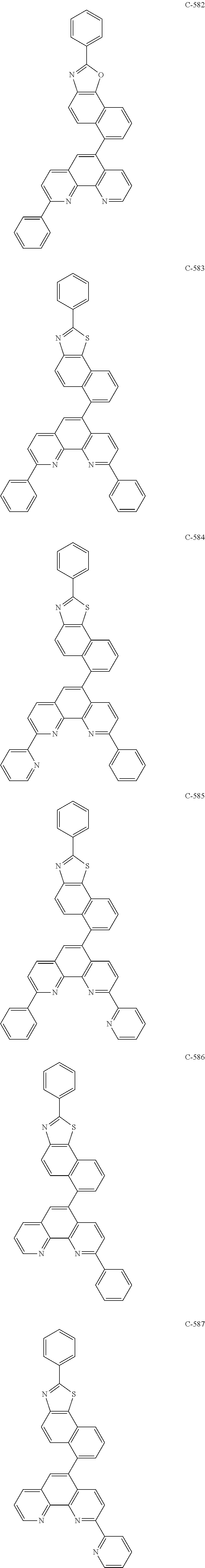 [SMILES: c1ccc(-c2ccc3cc(-c4cccc5c4ccc4nc(-c6ccccc6)oc45)c4cccnc4c3n2)cc1 ; c1ccc(-c2ccc3cc(-c4cccc5c4ccc4nc(-c6ccccc6)sc45)c4ccc(-c5ccccc5)nc4c3n2)cc1 ; c1ccc(-c2ccc3c(-c4cccc5c4ccc4nc(-c6ccccc6)sc45)cc4ccc(-c5ccccn5)nc4c3n2)cc1 ; c1ccc(-c2ccc3cc(-c4cccc5c4ccc4nc(-c6ccccc6)sc45)c4ccc(-c5ccccn5)nc4c3n2)cc1 ; c1ccc(-c2ccc3c(-c4cccc5c4ccc4nc(-c6ccccc6)sc45)cc4cccnc4c3n2)cc1 ; c1ccc(-c2nc3ccc4c(-c5cc6cccnc6c6nc(-c7ccccn7)ccc56)cccc4c3s2)cc1]

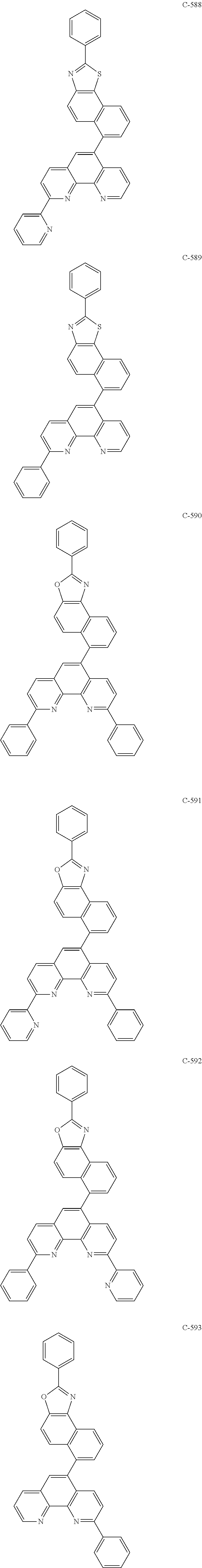 [SMILES: c1ccc(-c2nc3ccc4c(-c5cc6ccc(-c7ccccn7)nc6c6ncccc56)cccc4c3s2)cc1 ; c1ccc(-c2ccc3cc(-c4cccc5c4ccc4nc(-c6ccccc6)sc45)c4cccnc4c3n2)cc1 ; c1ccc(-c2ccc3cc(-c4cccc5c4ccc4oc(-c6ccccc6)nc45)c4ccc(-c5ccccc5)nc4c3n2)cc1 ; c1ccc(-c2ccc3c(-c4cccc5c4ccc4oc(-c6ccccc6)nc45)cc4ccc(-c5ccccn5)nc4c3n2)cc1 ; c1ccc(-c2ccc3cc(-c4cccc5c4ccc4oc(-c6ccccc6)nc45)c4ccc(-c5ccccn5)nc4c3n2)cc1 ; c1ccc(-c2ccc3c(-c4cccc5c4ccc4oc(-c6ccccc6)nc45)cc4cccnc4c3n2)cc1]

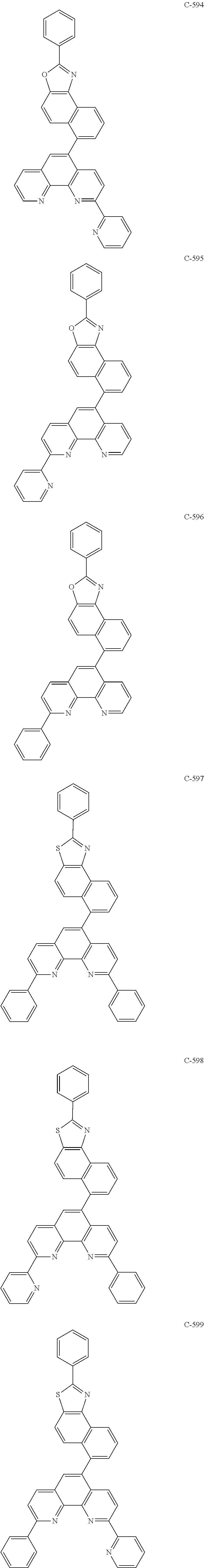

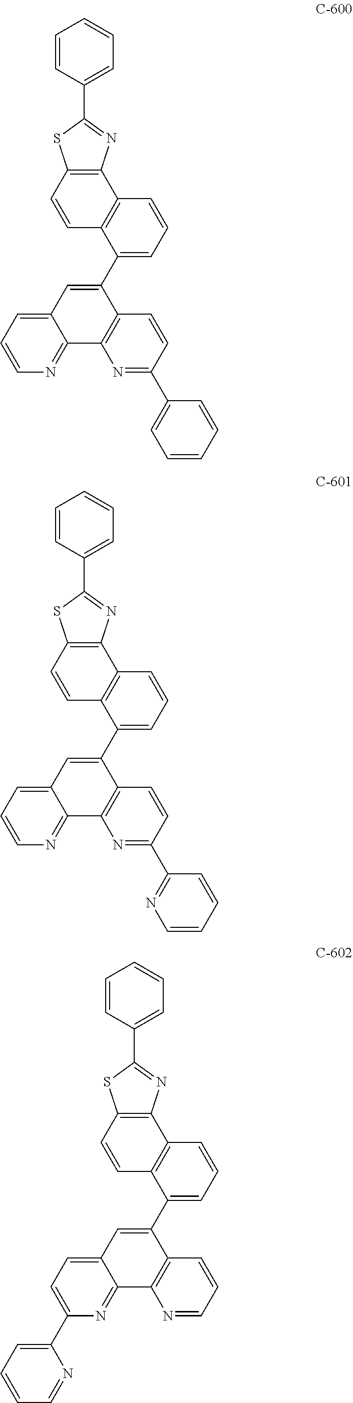

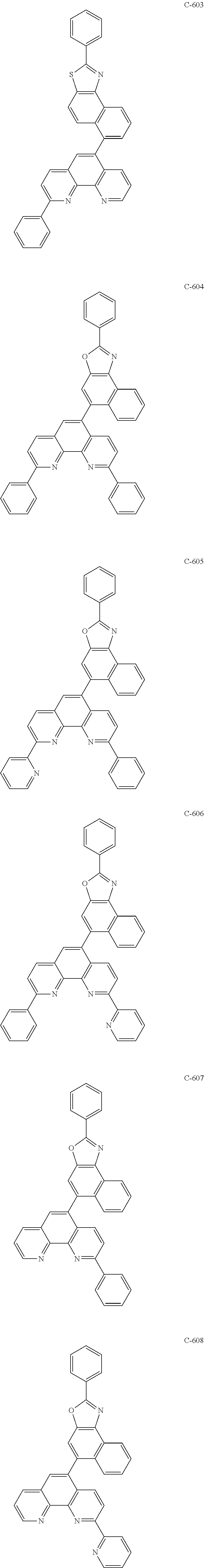 [SMILES: c1ccc(-c2ccc3cc(-c4cccc5c4ccc4sc(-c6ccccc6)nc45)c4cccnc4c3n2)cc1 ; c1ccc(-c2ccc3cc(-c4cc5oc(-c6ccccc6)nc5c5ccccc45)c4ccc(-c5ccccc5)nc4c3n2)cc1 ; c1ccc(-c2ccc3c(-c4cc5oc(-c6ccccc6)nc5c5ccccc45)cc4ccc(-c5ccccn5)nc4c3n2)cc1 ; c1ccc(-c2ccc3cc(-c4cc5oc(-c6ccccc6)nc5c5ccccc45)c4ccc(-c5ccccn5)nc4c3n2)cc1 ; c1ccc(-c2ccc3c(-c4cc5oc(-c6ccccc6)nc5c5ccccc45)cc4cccnc4c3n2)cc1 ; c1ccc(-c2nc3c(cc(-c4cc5cccnc5c5nc(-c6ccccn6)ccc45)c4ccccc43)o2)cc1]

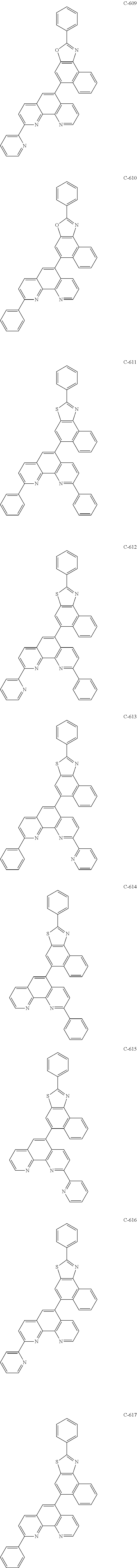

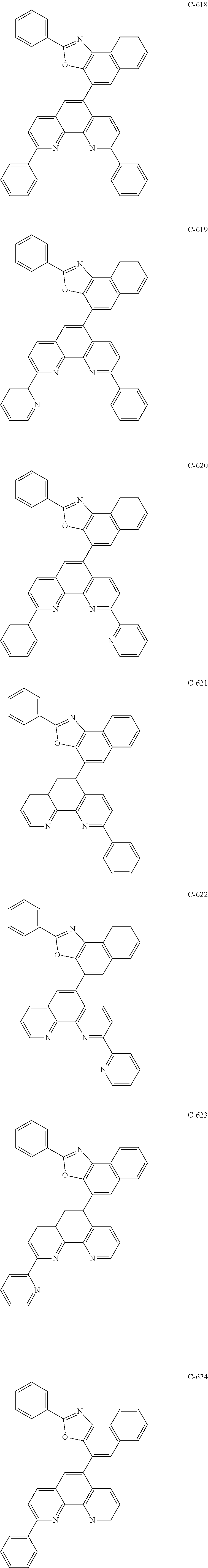

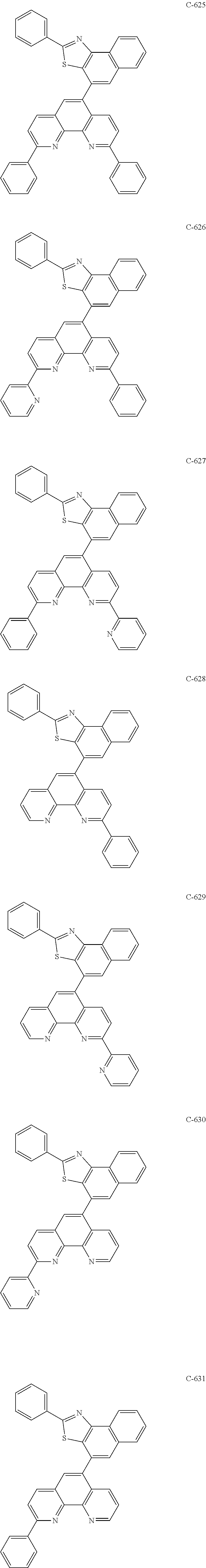

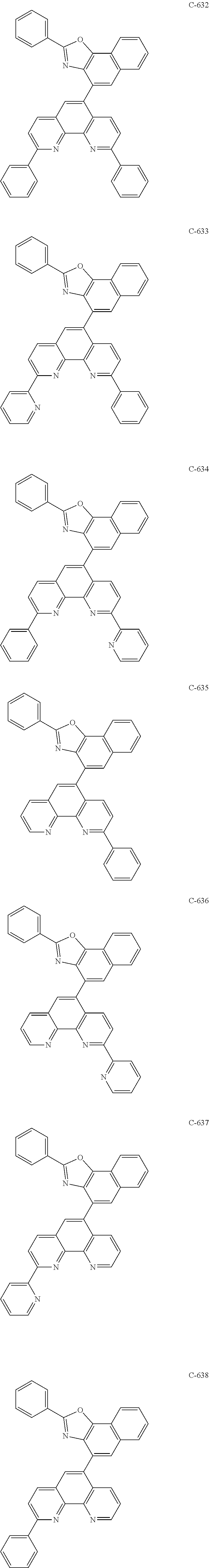

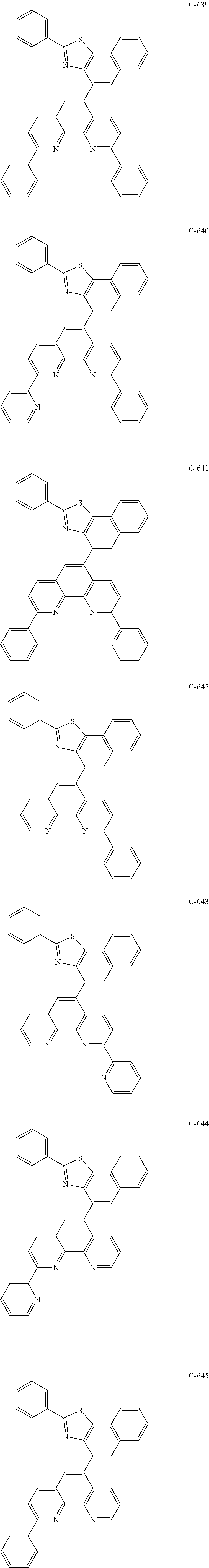

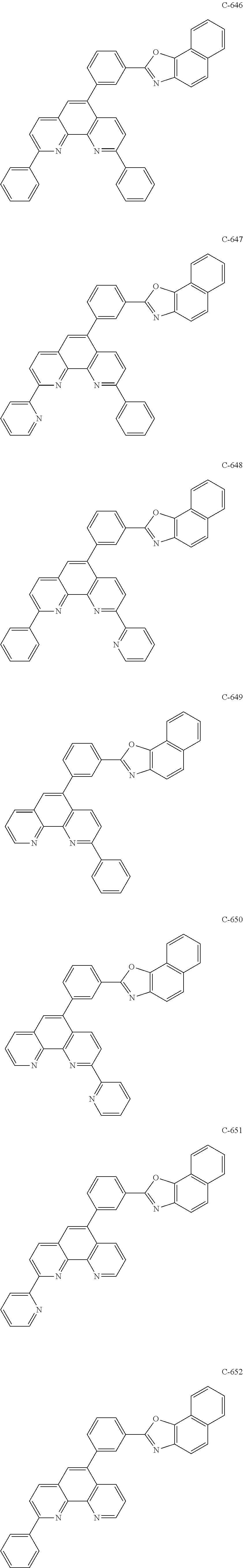

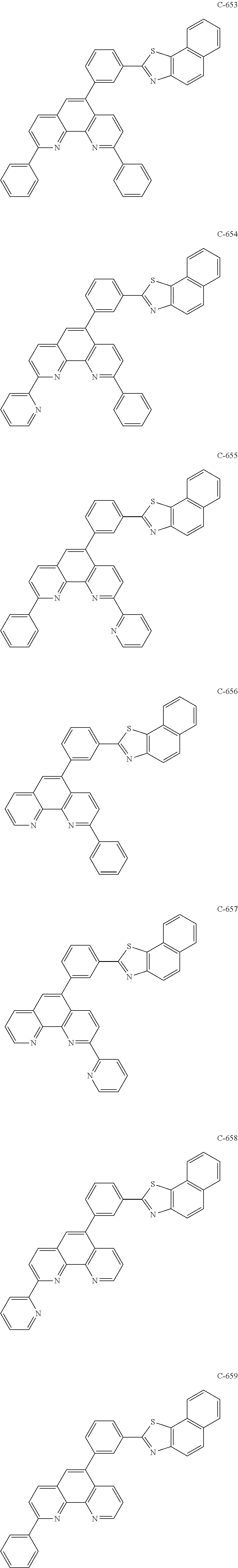

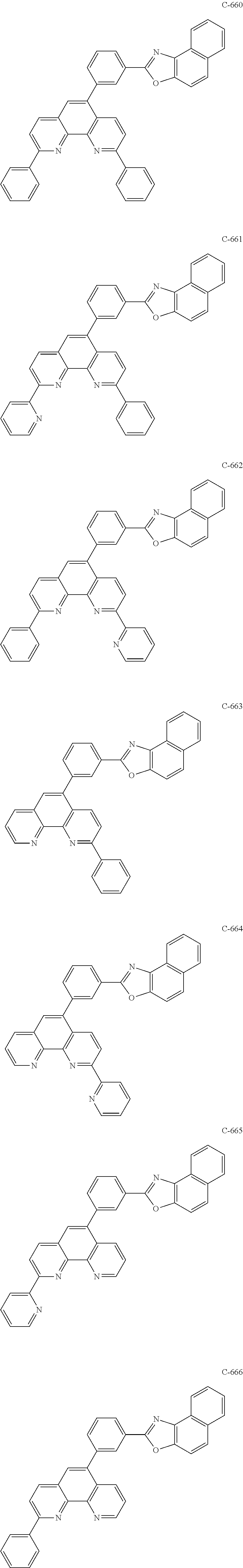

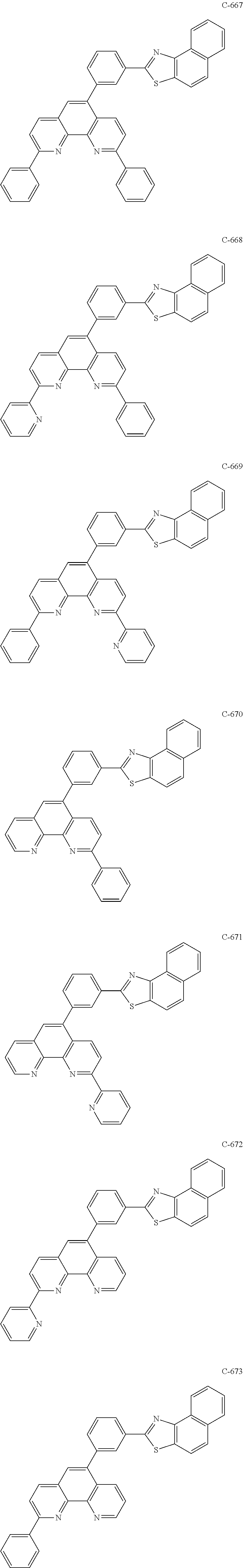

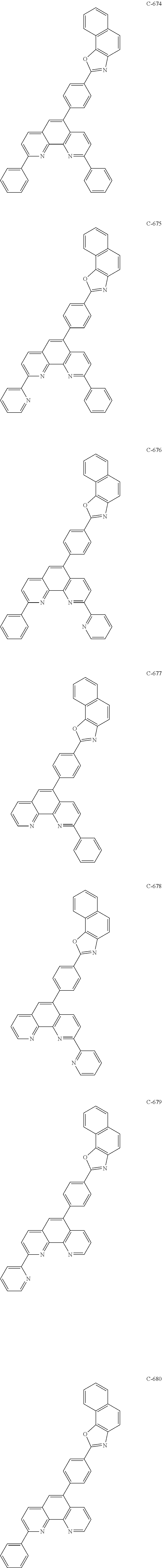

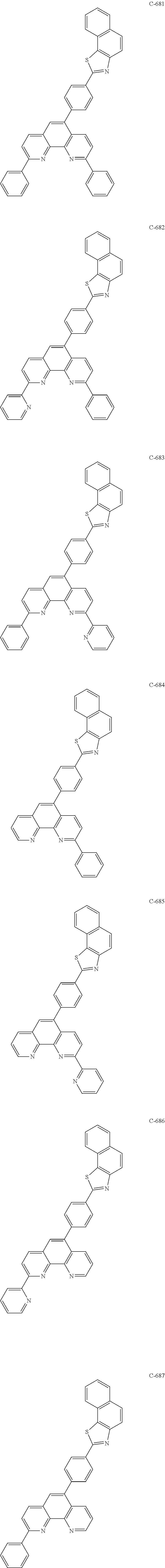

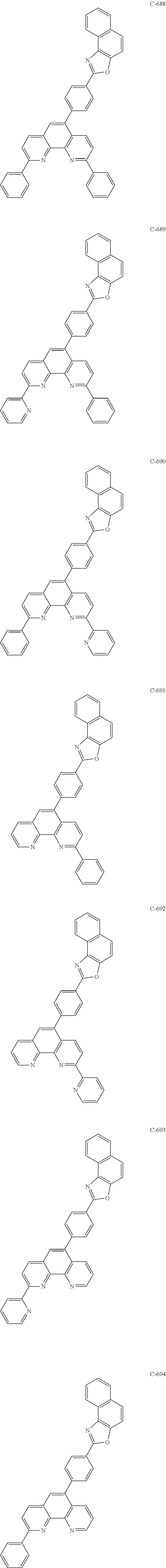

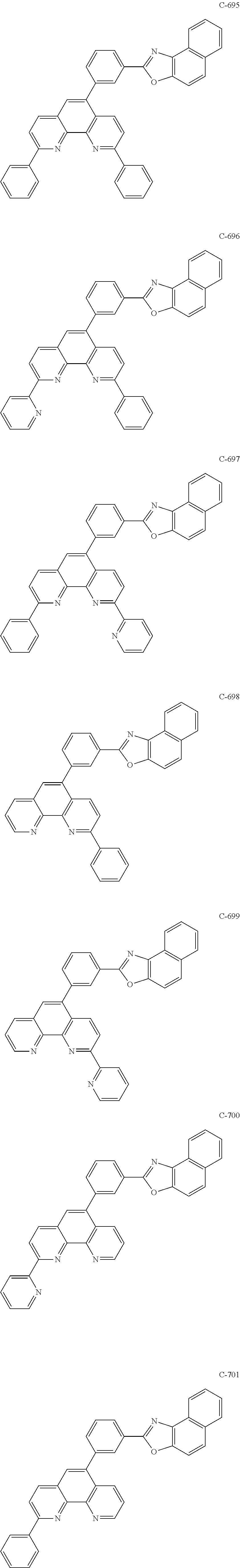

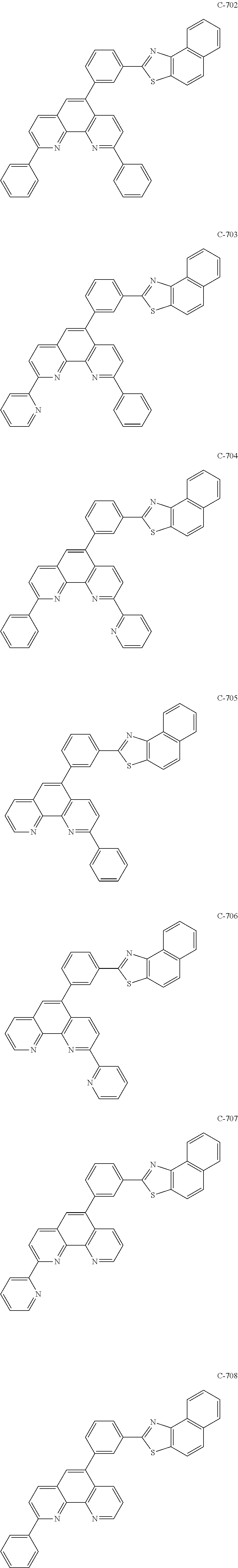

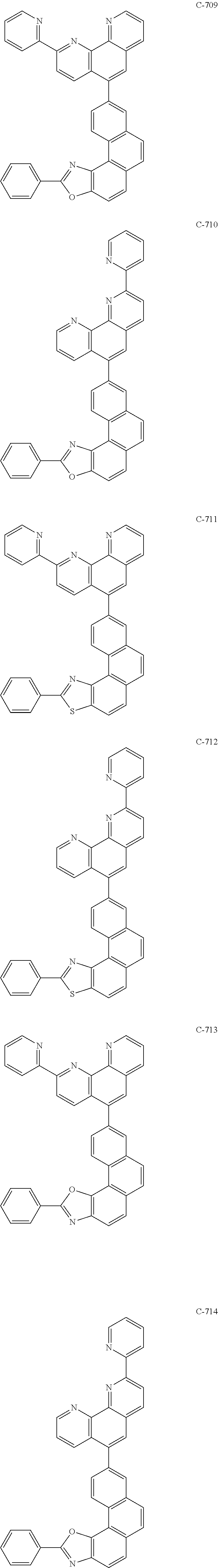 [SMILES: c1ccc(-c2nc3c(ccc4ccc5cc(-c6cc7cccnc7c7nc(-c8ccccn8)ccc67)ccc5c43)o2)cc1 ; c1ccc(-c2nc3c(ccc4ccc5cc(-c6cc7ccc(-c8ccccn8)nc7c7ncccc67)ccc5c43)o2)cc1 ; c1ccc(-c2nc3c(ccc4ccc5cc(-c6cc7cccnc7c7nc(-c8ccccn8)ccc67)ccc5c43)s2)cc1 ; c1ccc(-c2nc3c(ccc4ccc5cc(-c6cc7ccc(-c8ccccn8)nc7c7ncccc67)ccc5c43)s2)cc1 ; c1ccc(-c2nc3ccc4ccc5cc(-c6cc7cccnc7c7nc(-c8ccccn8)ccc67)ccc5c4c3o2)cc1 ; c1ccc(-c2nc3ccc4ccc5cc(-c6cc7ccc(-c8ccccn8)nc7c7ncccc67)ccc5c4c3o2)cc1]

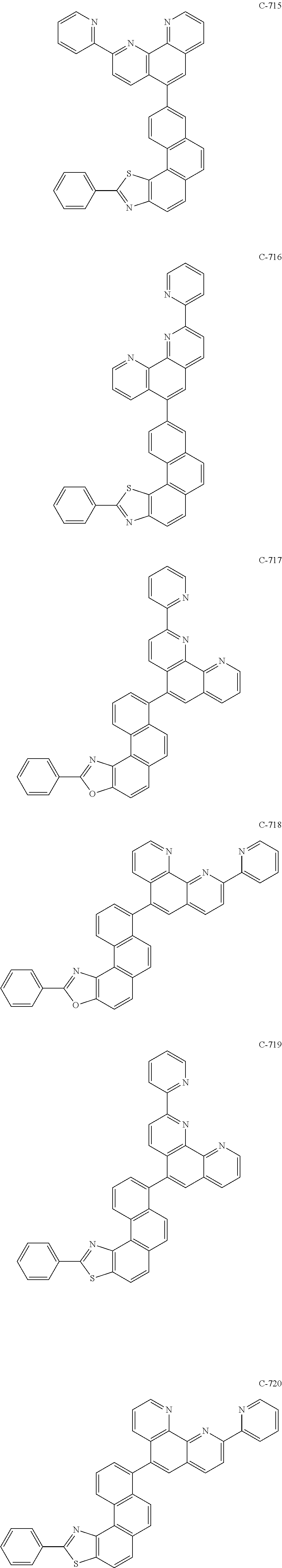

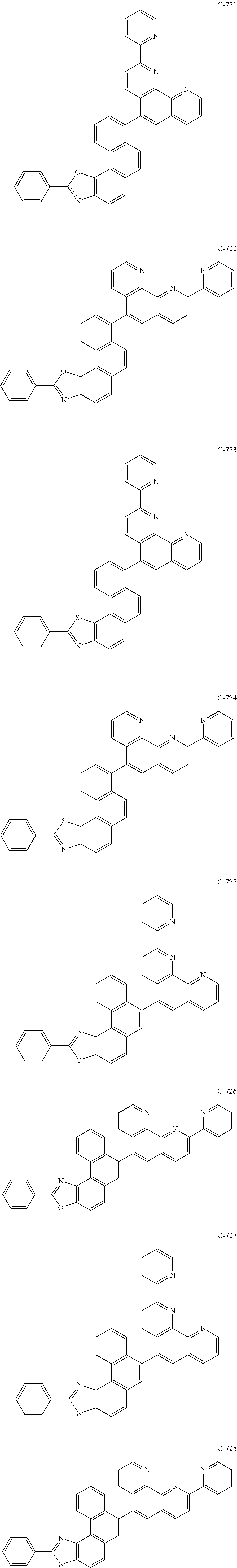

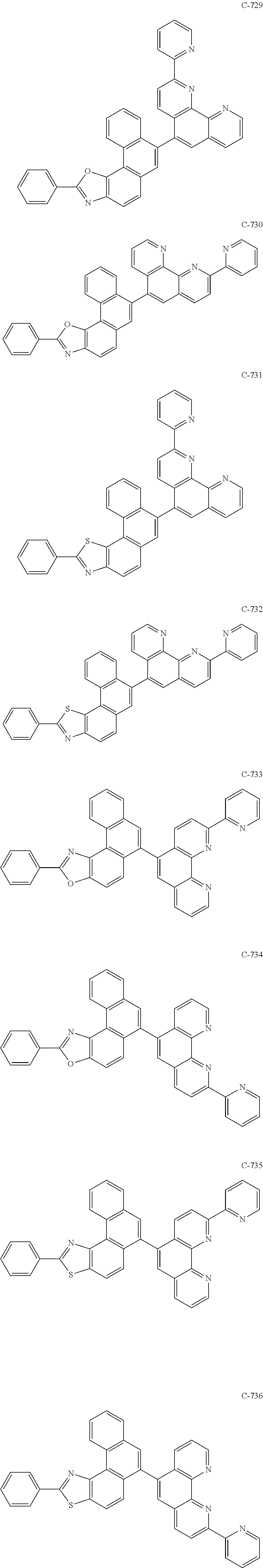

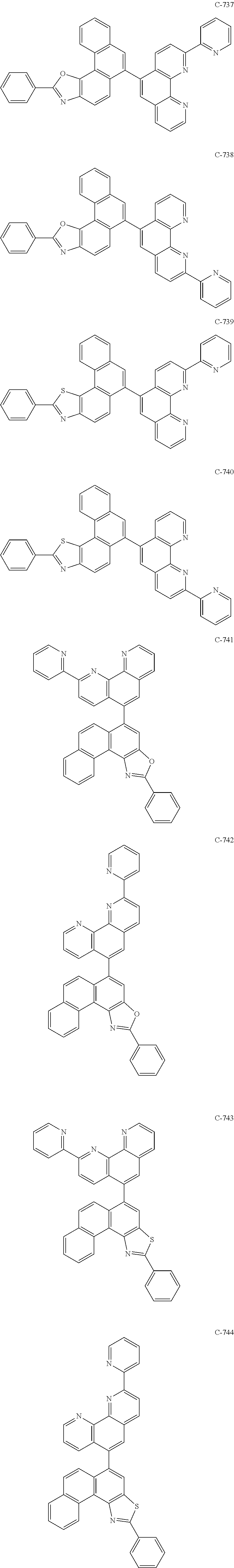

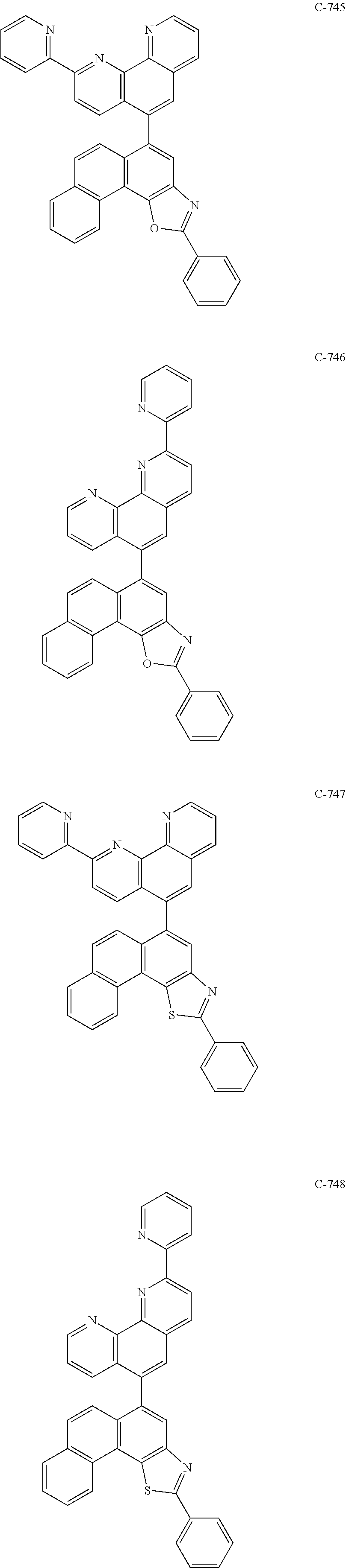 [SMILES: c1ccc(-c2nc3cc(-c4cc5cccnc5c5nc(-c6ccccn6)ccc45)c4ccc5ccccc5c4c3o2)cc1 ; c1ccc(-c2nc3cc(-c4cc5ccc(-c6ccccn6)nc5c5ncccc45)c4ccc5ccccc5c4c3o2)cc1 ; c1ccc(-c2nc3cc(-c4cc5cccnc5c5nc(-c6ccccn6)ccc45)c4ccc5ccccc5c4c3s2)cc1 ; c1ccc(-c2nc3cc(-c4cc5ccc(-c6ccccn6)nc5c5ncccc45)c4ccc5ccccc5c4c3s2)cc1]

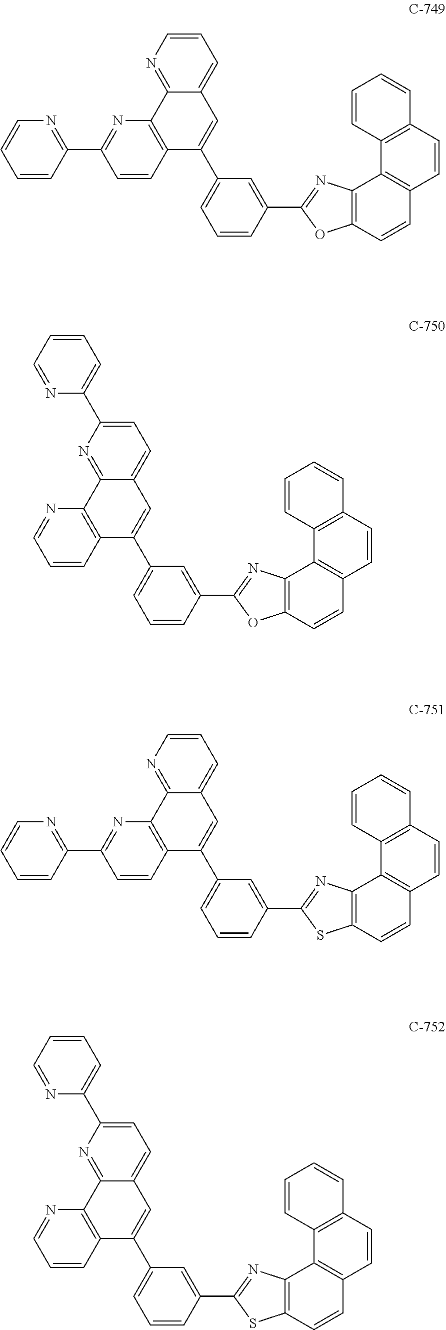 [SMILES: c1ccc(-c2ccc3c(-c4cccc(-c5nc6c(ccc7ccc8ccccc8c76)o5)c4)cc4cccnc4c3n2)nc1 ; c1ccc(-c2ccc3cc(-c4cccc(-c5nc6c(ccc7ccc8ccccc8c76)o5)c4)c4cccnc4c3n2)nc1 ; c1ccc(-c2ccc3c(-c4cccc(-c5nc6c(ccc7ccc8ccccc8c76)s5)c4)cc4cccnc4c3n2)nc1 ; c1ccc(-c2ccc3cc(-c4cccc(-c5nc6c(ccc7ccc8ccccc8c76)s5)c4)c4cccnc4c3n2)nc1]

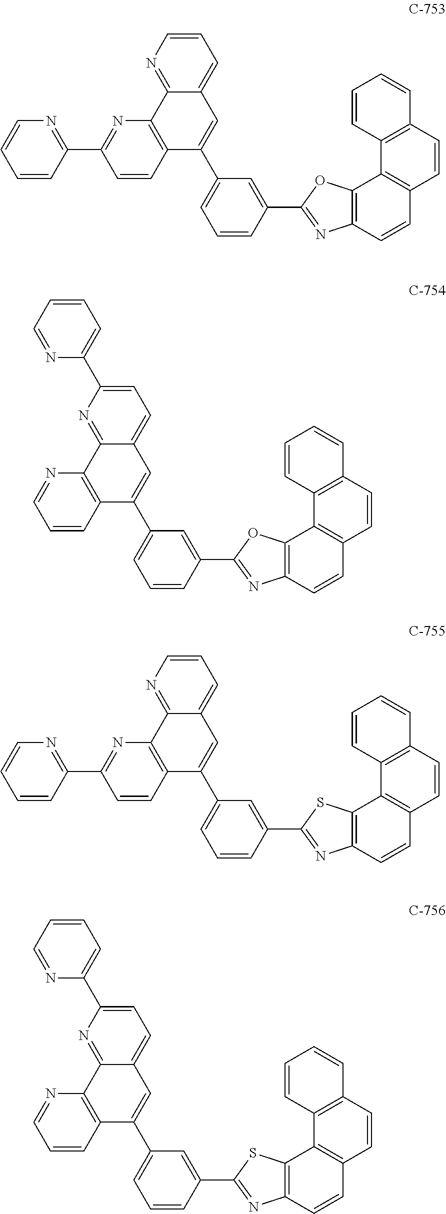 [SMILES: c1ccc(-c2ccc3c(-c4cccc(-c5nc6ccc7ccc8ccccc8c7c6o5)c4)cc4cccnc4c3n2)nc1 ; c1ccc(-c2ccc3cc(-c4cccc(-c5nc6ccc7ccc8ccccc8c7c6o5)c4)c4cccnc4c3n2)nc1 ; c1ccc(-c2ccc3c(-c4cccc(-c5nc6ccc7ccc8ccccc8c7c6s5)c4)cc4cccnc4c3n2)nc1 ; c1ccc(-c2ccc3cc(-c4cccc(-c5nc6ccc7ccc8ccccc8c7c6s5)c4)c4cccnc4c3n2)nc1]

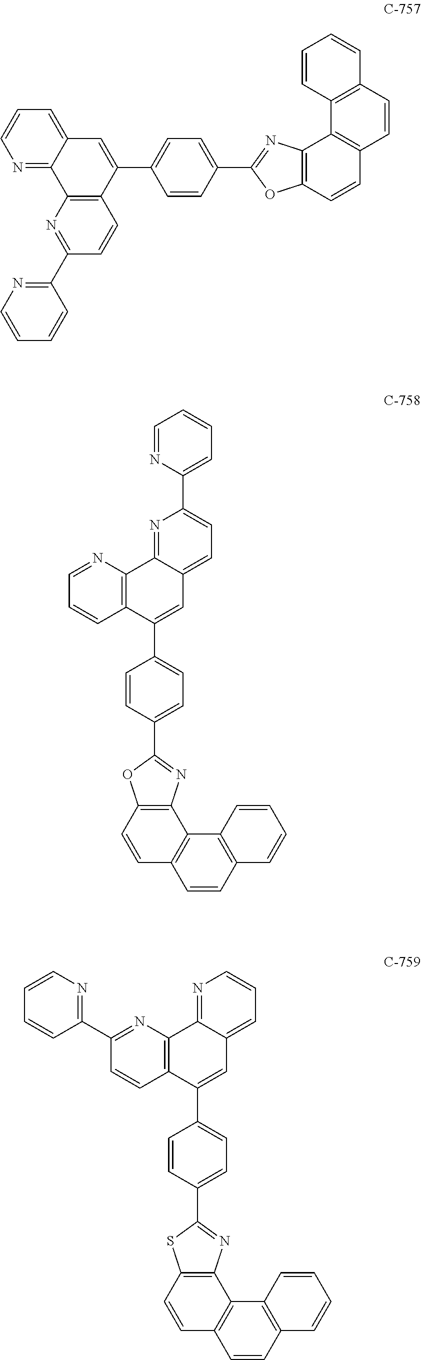 [SMILES: c1ccc(-c2ccc3c(-c4ccc(-c5nc6c(ccc7ccc8ccccc8c76)o5)cc4)cc4cccnc4c3n2)nc1 ; c1ccc(-c2ccc3cc(-c4ccc(-c5nc6c(ccc7ccc8ccccc8c76)o5)cc4)c4cccnc4c3n2)nc1 ; c1ccc(-c2ccc3c(-c4ccc(-c5nc6c(ccc7ccc8ccccc8c76)s5)cc4)cc4cccnc4c3n2)nc1]

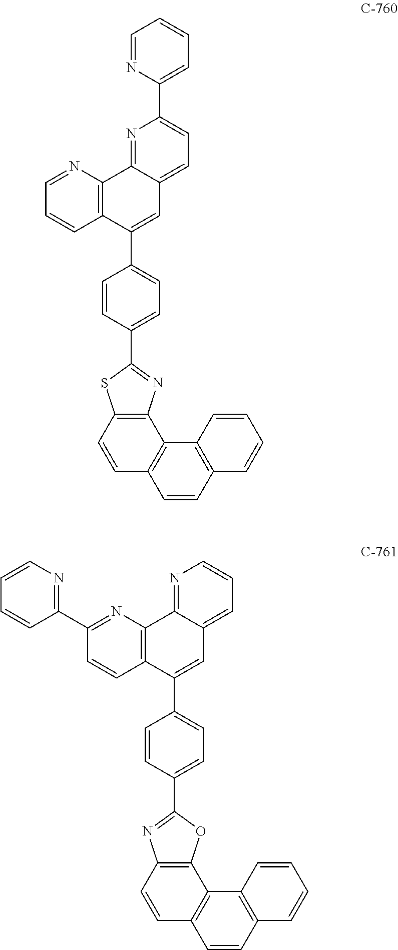 [SMILES: c1ccc(-c2ccc3cc(-c4ccc(-c5nc6c(ccc7ccc8ccccc8c76)s5)cc4)c4cccnc4c3n2)nc1 ; c1ccc(-c2ccc3c(-c4ccc(-c5nc6ccc7ccc8ccccc8c7c6o5)cc4)cc4cccnc4c3n2)nc1]

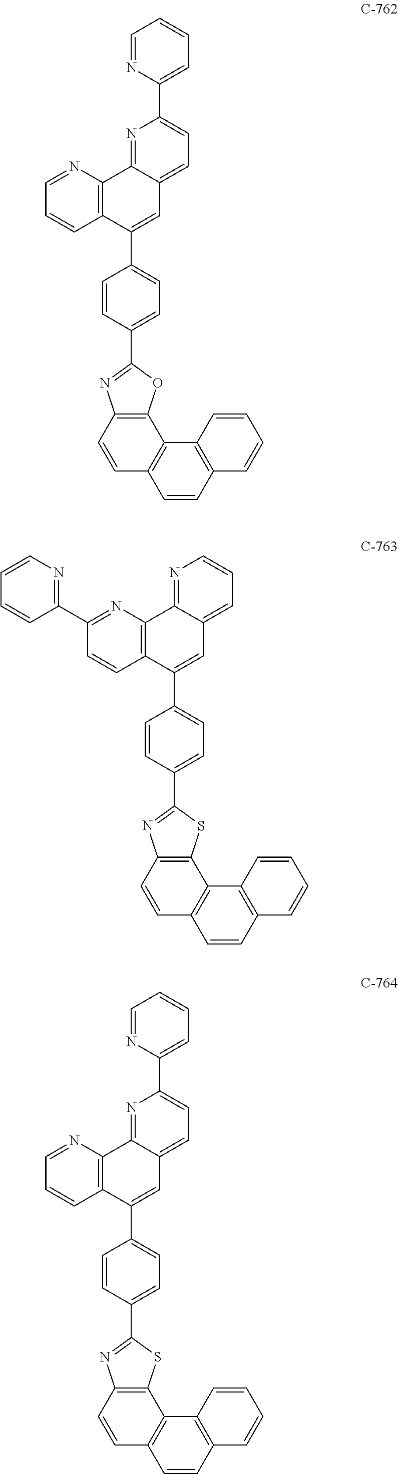 [SMILES: c1ccc(-c2ccc3cc(-c4ccc(-c5nc6ccc7ccc8ccccc8c7c6o5)cc4)c4cccnc4c3n2)nc1 ; c1ccc(-c2ccc3c(-c4ccc(-c5nc6ccc7ccc8ccccc8c7c6s5)cc4)cc4cccnc4c3n2)nc1 ; c1ccc(-c2ccc3cc(-c4ccc(-c5nc6ccc7ccc8ccccc8c7c6s5)cc4)c4cccnc4c3n2)nc1]

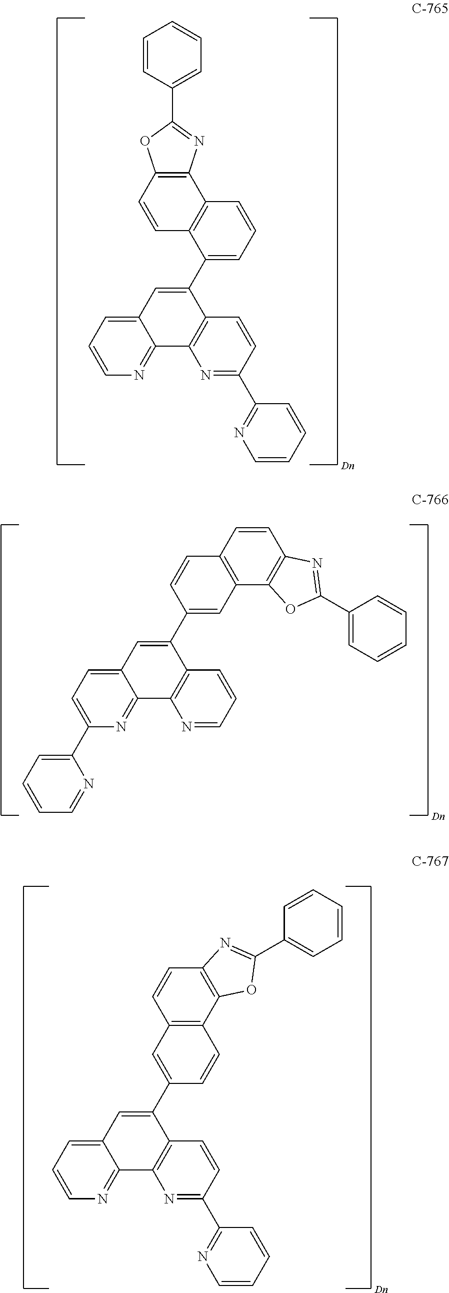

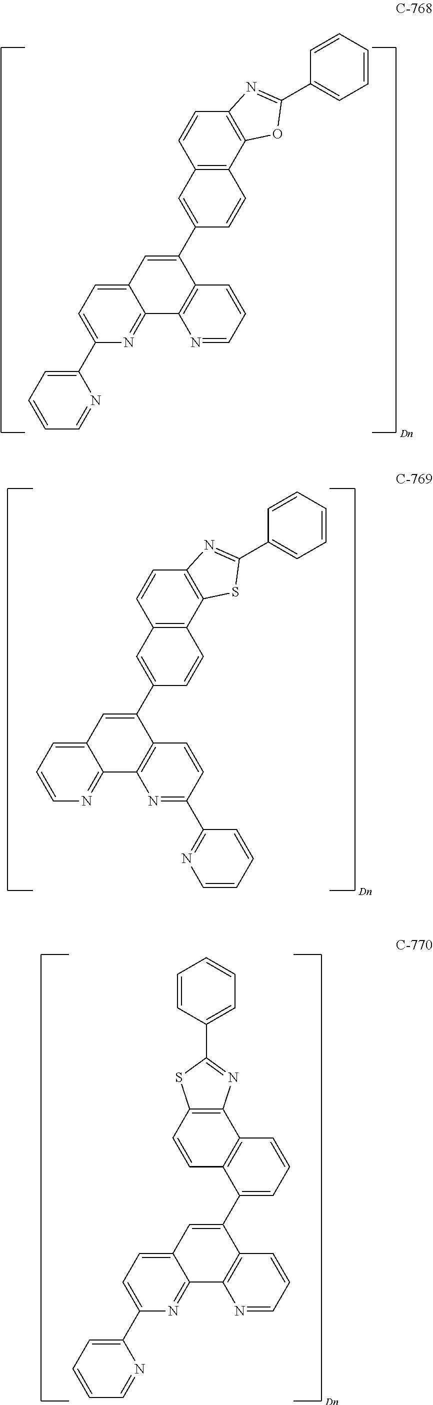

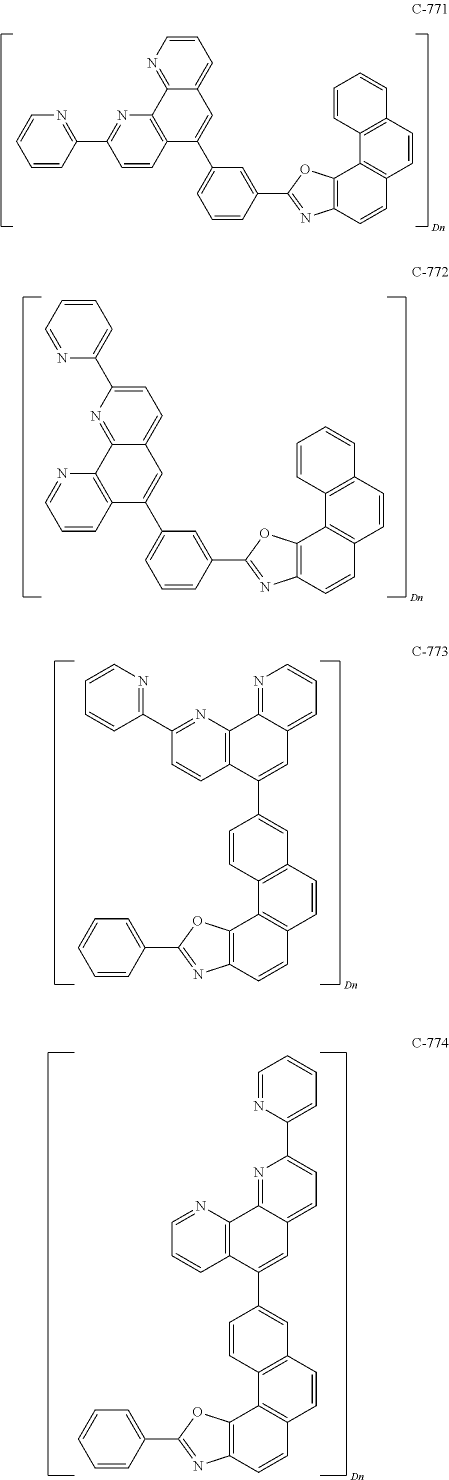

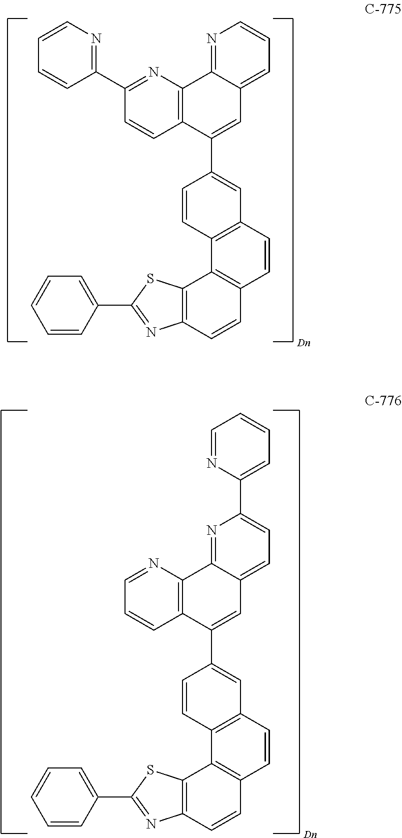

wherein “Dₙ” in the above compounds means that n number of hydrogens is replaced by deuterium, wherein n represents an integer of 1 or more, up to the total number of hydrogens of the compound. Specifically, n is an integer of at least 1 and at most the number of hydrogens present in the compound. In one example, in a compound represented by Formula 1 that includes deuterium, the deuterium substitution ratio is preferably up to about 100% of the total number of hydrogens, more preferably up to about 95%, even more preferably up to about 90%, and still more preferably up to about 85%. A compound of Formula 1 substituted at the above deuterium substitution ratio can have increased bond dissociation energy due to deuteration, thereby increasing the stability of the compound, and an organic electroluminescent device comprising the compound can exhibit improved emission characteristics.

The compound of Formula 1 may be coordinated to an alkali metal or an alkaline earth metal. In addition, the compound of Formula 1 may be a complex compound comprising an alkali metal, an alkaline earth metal, a lanthanoid, an actinoid, or a transition metal. The alkali metal may be one or more selected from the group consisting of Li, Na, K, Rb, Cs, Fr, and Yb. The alkaline earth metal may be one or more selected from the group consisting of Be, Mg, Ca, Sr, Ba, and Ra. The lanthanoid may be one or more selected from the group consisting of La, Ce, Pr, Nd, Pm, Sm, Eu, Gd, Tb, Dy, Ho, Er, Tm, Yb, and Lu. The actinoid may be one or more selected from the group consisting of Ac, Th, Pa, U, Np, Pu, Am, Cm, Bk, Cf, Es, Fm, Md, No, and Lr. The transition metal may be one or more selected from the group consisting of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Ds, Rg, and Cn.

The compound represented by Formula 1 according to the present disclosure can be prepared by synthesis methods known to those skilled in the art. For example, the compound of the present disclosure can be prepared with reference to Reaction Scheme 1 and Reaction Scheme 2 below, but is not limited thereto.

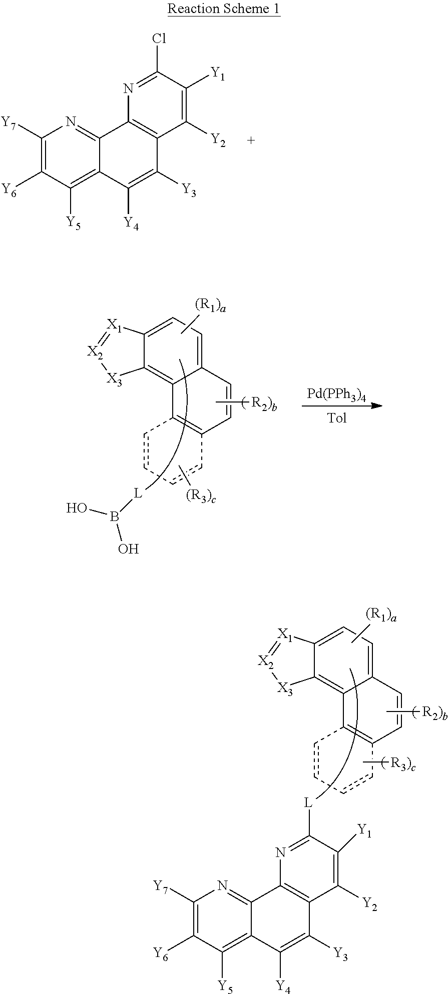

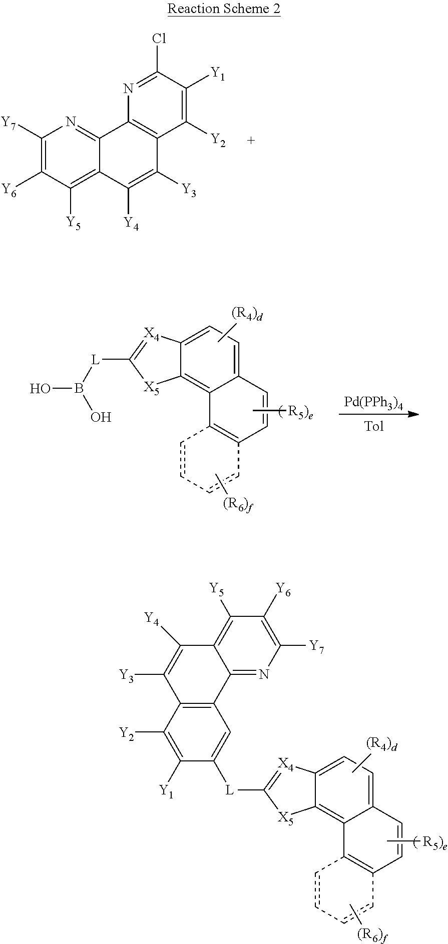

In Reaction Schemes 1 and 2, Y₁ to Y₇, R₁ to R₆, L, X₁ to X₅, and a to f are as defined in Formula 1.

The present disclosure provides an organic electroluminescent material, an organic electroluminescent device, or an n-type charge generation material comprising the compound.

In one embodiment, an organic electroluminescent device comprises: a first electrode; a second electrode; a plurality of light-emitting units disposed between the first electrode and the second electrode; and at least one charge generation layer disposed between adjacent light-emitting units among the plurality of light-emitting units; wherein the charge generation layer may comprise the compound represented by Formula 1.

In one embodiment, the organic electroluminescent device may be an organic electroluminescent device having a tandem structure. In an example of a tandem organic electroluminescent device, a single light-emitting unit (emission section) may be constituted by two or more light-emitting units connected by a charge generation layer. The organic electroluminescent device may include, on a substrate, a first electrode and a second electrode facing each other, and, laminated between the first and second electrodes, two or more light-emitting units each having a light-emitting layer that emits light in a specific wavelength range, for example, three or more light-emitting units. A plurality of emission sections may be included, and each emission section may include a hole transport region, a light-emitting layer, and an electron transport region; the hole transport region may include a hole injection layer and a hole transport layer; the electron transport region may include an electron transport layer and an electron injection layer; and, in one example, the number of light-emitting layers included in the emission section may be three or more. The plurality of light-emitting units may be configured to emit the same color or different colors. In addition, a single light-emitting unit may include one or more light-emitting layers, and the plurality of light-emitting layers may be light-emitting layers of the same color or of different colors. This may include one or more charge generation layers located between each light-emitting unit.

The charge generation layer means a layer in which holes and electrons are generated when a voltage is applied. When the number of light-emitting units is three or more, a charge generation layer may be located between each light-emitting unit. In this case, a plurality of charge generation layers may be the same as or different from one another. By disposing the charge generation layer between the light-emitting units, the current efficiency in each light-emitting unit is increased, and charges can be smoothly distributed. Specifically, the charge generation layer may be provided between two adjacent stacks so that a tandem organic electroluminescent device can be driven with only one pair of an anode and a cathode, without a separate internal electrode positioned between the stacks. The charge generation layer may be constituted by an n-type charge generation layer and a p-type charge generation layer. The n-type charge generation layer may comprise the compound of Formula 1 of the present disclosure. The p-type charge generation layer may be formed of a metal or an organic material doped with a p-type dopant. For example, the metal may be one or more alloys selected from the group consisting of Al, Cu, Fe, Pb, Zn, Au, Pt, W, In, Mo, Ni, and Ti. In addition, for the p-type doped organic material, the p-type dopant and the host may be materials that are conventionally used.

According to another embodiment, the organic electroluminescent device comprises: a first electrode; a second electrode; and one or more organic layers interposed between the first electrode and the second electrode, wherein the organic layer(s) include a hole transport layer, a light-emitting layer, a hole auxiliary layer, an electron blocking layer, a charge generation layer, and a light-emitting auxiliary layer. In one example, at least one layer among the hole transport layer, the light-emitting layer, the hole auxiliary layer, the electron blocking layer, the charge generation layer, and the light-emitting auxiliary layer may comprise the compound represented by Formula 1. For example, the charge generation layer may comprise a compound represented by Formula 1. In one example, the organic electroluminescent material of the present disclosure comprises at least one compound selected from Compounds C-1 to C-776, and the organic electroluminescent material may be included in the same organic layer, for example, in the charge generation layer.

The organic layer, in addition to a hole transport layer, a light-emitting layer, a hole auxiliary layer, an electron-blocking layer, a charge generation layer, and a light-emitting auxiliary layer, may further comprise at least one layer selected from a hole injection layer, an electron transport layer, an electron injection layer, an interlayer, a hole-blocking layer, and an electron buffer layer. The organic layer may further comprise an amine-based compound and/or an azine-based compound in addition to the compound(s) of the present disclosure. Specifically, the hole injection layer, the hole transport layer, the hole auxiliary layer, the light-emitting layer, the light-emitting auxiliary layer, or the electron-blocking layer may contain an amine-based compound, for example, an arylamine-based compound or a styrylarylamine-based compound, as a hole injection material, a hole transport material, a hole auxiliary material, a light-emitting material, a light-emitting auxiliary material, or an electron-blocking material. Further, the electron transport layer, the electron injection layer, the electron buffer layer, and the hole-blocking layer may contain an azine-based compound as an electron transport material, an electron injection material, an electron buffer material, and a hole-blocking material. Further, the organic layer may additionally comprise one or more metals selected from the group consisting of Group 1 metals, Group 2 metals, 4th-period transition metals, 5th-period transition metals, lanthanide metals, and organometallic compounds of d-transition elements, or one or more complex compounds comprising such a metal.

The compound of the present disclosure according to one embodiment may be used as a light-emitting material for a white organic light-emitting device. Various structures have been suggested for the white organic light-emitting device, such as a parallel side-by-side arrangement method, a stacking arrangement method, a CCM (color conversion material) method, etc., according to the arrangement of R (red), G (green), YG (yellowish green), or B (blue) light-emitting units. In addition, the organic electroluminescent compound according to one embodiment may also be applied to the organic electroluminescent device comprising a QD (quantum dot).

One of the first electrode and the second electrode may be an anode and the other may be a cathode, wherein the first electrode and the second electrode may each be formed as a transmissive conductive material, a transflective conductive material, or a reflective conductive material. The organic electroluminescent device may be a top emission type, a bottom emission type, or a dual-side emission type according to the kinds of the material forming the first electrode and the second electrode.

The light-emitting layer comprises one or more hosts and one or more dopants. As the dopant included in the organic electroluminescent device of the present disclosure, one or more phosphorescent or fluorescent dopants may be used, and a fluorescent dopant is preferred.

A hole injection layer, a hole transport layer, an electron-blocking layer, or a combination thereof can be used between the anode and the light-emitting layer. The hole injection layer may be multi-layered in order to lower the hole injection barrier (or hole injection voltage) from the anode to the hole transport layer or the electron-blocking layer, wherein each of the multi-layers may use two compounds simultaneously. Also, the hole injection layer may be doped as a p-dopant. Also, the electron-blocking layer may be placed between the hole transport layer (or hole injection layer) and the light-emitting layer, and can confine the excitons within the light-emitting layer by blocking the overflow of electrons from the light-emitting layer to prevent light emission leakage. The hole transport layer or the electron-blocking layer may be multi-layered, wherein each layer may use a plurality of compounds.

An electron buffer layer, a hole-blocking layer, an electron transport layer, an electron injection layer, or a combination thereof can be used between the light-emitting layer and the cathode. The electron buffer layer may be multi-layered in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layered, wherein each layer may use a plurality of compounds. Further, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron-blocking layer. The light-emitting auxiliary layer, the hole auxiliary layer, or the electron-blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ (1≤x≤2), AlOₓ (1≤x≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare earth metals, and mixtures thereof. Also, a reductive dopant layer may be employed as a charge generating layer to prepare an organic electroluminescent device having two or more light-emitting layers and emitting white light.

According to one embodiment, the present disclosure can provide a display device comprising a compound represented by Formula 1. In addition, by using the organic electroluminescent device of the present disclosure, it is possible to manufacture a display device, for example a smartphone, a tablet, a notebook, a PC, a TV, or a vehicle display device, or a lighting device, for example an outdoor or indoor lighting device.

Hereinafter, a method for preparing the organic electroluminescent compound according to the present disclosure and the physical properties thereof, and the driving voltage, current efficiency, and lifetime characteristics of the OLED according to the present disclosure will be described. However, the following Examples are provided to describe the characteristics of the compound and the OLED according to the present disclosure for a detailed understanding, and the present disclosure is not limited to the following Examples.

### [Example 1] Synthesis of Compound C-2

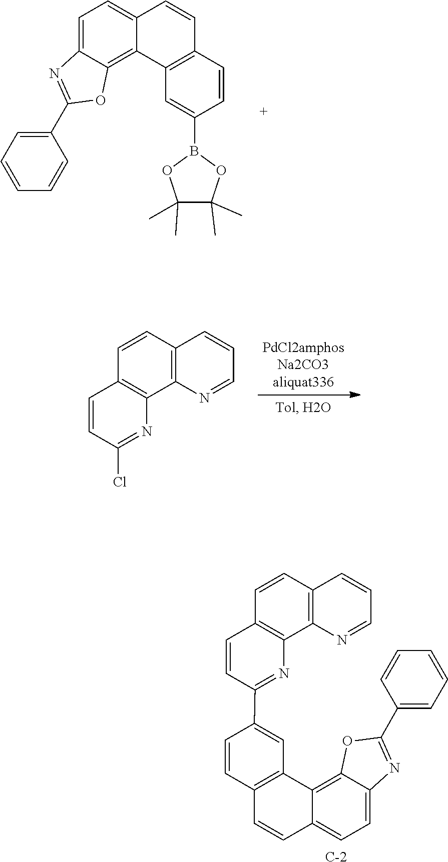

2-Phenyl-10-(4,4,5,5-tetramethyl-1,3,2-dioxaborolan-2-yl)phenanthro[3,4-d]oxazole (15.0 g, 35.60 mmol), 2-chloro-1,10-phenanthroline (9.2 g, 42.72 mmol), PdCl₂amphos (1.8 g, 2.49 mmol), Aliquat 336 (1.4 g, 3.56 mmol), and Na₂CO₃ (7.5 g, 71.20 mmol) were added to 180 mL of toluene and 60 mL of distilled water, and then stirred under reflux at 130° C. After 17 hours, the reaction mixture was cooled to room temperature, MeOH was added to the precipitated solid compound, the reaction mixture was then stirred for 30 minutes, and filtration was performed. Thereafter, silica filtration was performed, and recrystallization was carried out to obtain Compound C-2 (6.5 g, yield: 38.7%).

| | MW | M.P. |
|---|---|---|
| C-2 | 473.54 | 268° C. |

### [Example 2] Synthesis of Compound C-1

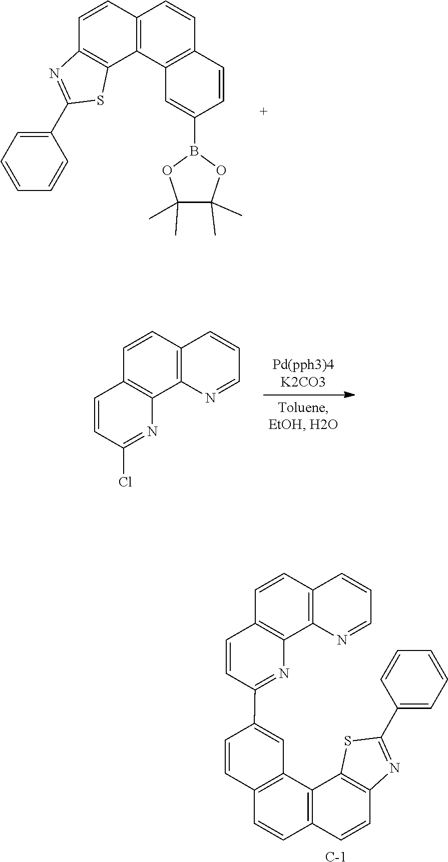

2-Phenyl-10-(4,4,5,5-tetramethyl-1,3,2-dioxaborolan-2-yl)phenanthro[3,4-d]thiazole (10.0 g, 22.86 mmol), 2-chloro-1,10-phenanthroline (5.9 g, 27.43 mmol), Pd(pph₃)₄ (1.3 g, 1.14 mmol), and K₂CO₃ (6.3 g, 45.72 mmol) were added to 115 mL of toluene, 40 mL of EtOH, and 40 mL of water, and then stirred under reflux at 130° C. After 16 hours, the reaction mixture was cooled to room temperature; water was added to the precipitated solid compound, the reaction mixture was then stirred for 30 minutes, and filtration was performed. Thereafter, silica filtration was performed, and recrystallization was carried out to obtain Compound C-1 (6.0 g, yield: 53.6%).

| | MW | M.P. |
|---|---|---|
| C-1 | 489.60 | 225° C. |

### [Example 3] Synthesis of Compound C-4

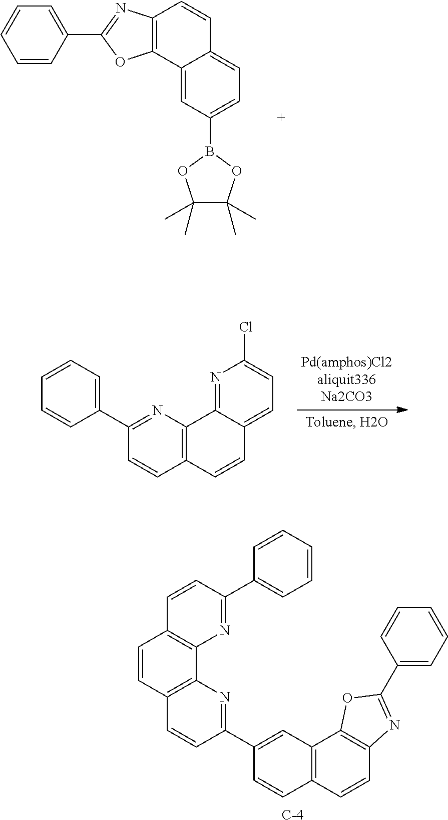

2-Phenyl-8-(4,4,5,5-tetramethyl-1,3,2-dioxaborolan-2-yl)naphtho[2,1-d]oxazole (10.0 g, 26.94 mmol), 2-chloro-9-phenyl-1,10-phenanthroline (9.4 g, 32.32 mmol), PdCl₂amphos (1.3 g, 1.88 mmol), Aliquat 336 (1.1 g, 2.69 mmol), and Na₂CO₃ (5.7 g, 53.88 mmol) were added to 135 mL of toluene and 45 mL of water, and then stirred under reflux at 130° C. After 3 hours, the reaction mixture was cooled to room temperature, the resulting solid was filtered off, and the resultant was separated by column chromatography and recrystallized to obtain Compound C-4 (6.9 g, yield: 51.49%).

| | MW | M.P. |
|---|---|---|
| C-4 | 499.57 | 243° C. |

### [Example 4] Synthesis of Compound C-6

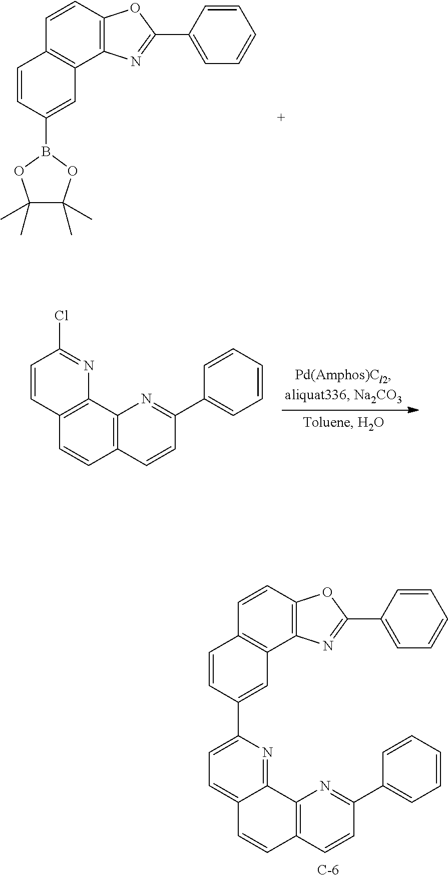

(2-Phenyl-8-(4,4,5,5-tetramethyl-1,3,2-dioxaborolan-2-yl)naphtho[1,2-d]oxazole (10.0 g, 26.93 mmol), 2-chloro-9-phenyl-1,10-phenanthroline (8.6 g, 29.63 mmol), Pd(Amphos)Cl₂ (1.3 g, 1.88 mmol), Aliquat 336 (2.2 g, 5.38 mmol), and Na₂CO₃ (5.7 g, 53.86 mmol) were dissolved in 135 mL of toluene and 45 mL of H₂O. The reaction mixture was stirred under reflux at 130° C. for 2 hours. The mixture was cooled to room temperature, and the layers were separated. The material was then subjected to silica filtration and recrystallized to obtain Compound C-6 (10.7 g, yield: 79.85%).

| | MW | M.P. |
|---|---|---|
| C-6 | 499.57 | 245° C. |

### [Example 5] Synthesis of Compound C-7

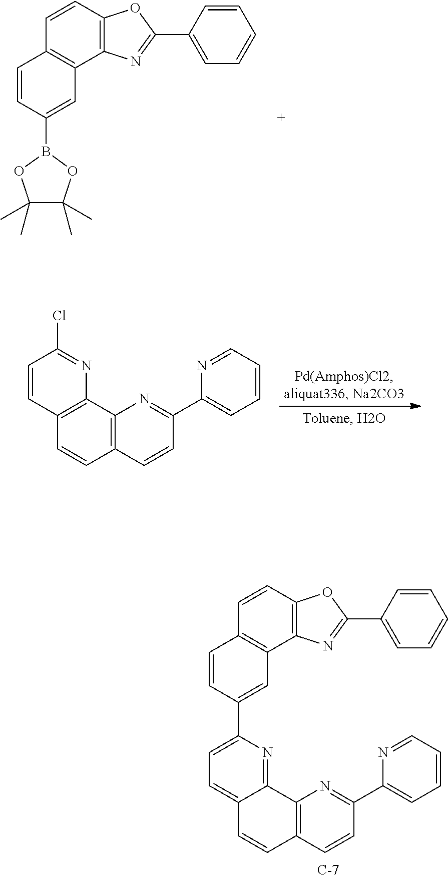

2-Phenyl-8-(4,4,5,5-tetramethyl-1,3,2-dioxaborolan-2-yl)naphtho[1,2-d]oxazole (10.0 g, 26.94 mmol), 2-chloro-9-(pyridin-2-yl)-1,10-phenanthroline (8.6 g, 29.63 mmol), Pd(Amphos)Cl₂ (1.3 g, 1.88 mmol), Aliquat 336 (2.2 g, 5.38 mmol), and Na₂CO₃ (5.7 g, 53.86 mmol) were dissolved in 135 mL of toluene and 45 mL of H₂O. The reaction mixture was stirred under reflux at 130° C. for 3 hours. Thereafter, the mixture was cooled to room temperature, the resulting solid was filtered, and the resultant was separated by column chromatography and recrystallized to obtain Compound C-7 (5.4 g, yield: 40.29%).

| | MW | M.P. |
|---|---|---|
| C-7 | 500.56 | 308° C. |

### [Example 6] Synthesis of Compound C-36

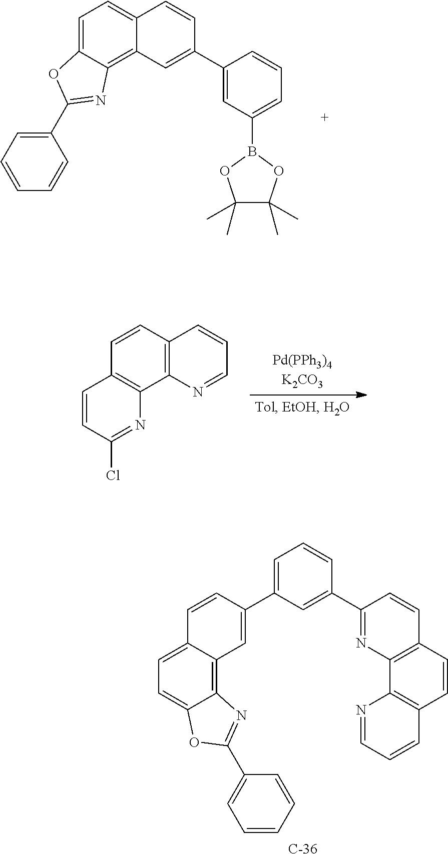

2-Phenyl-8-(3-(4,4,5,5-tetramethyl-1,3,2-dioxaborolan-2-yl)phenyl)naphtho[1,2-d]oxazole (9.6 g, 21 mmol), 2-chloro-1,10-phenanthroline (5.5 g, 26 mmol), Pd(PPh₃)₄ (1.2 g, 1.1 mmol), and K₂CO₃ (5.9 g, 43 mmol) were added to 110 mL of toluene, 35 mL of ethanol, and 35 mL of distilled water and then stirred under reflux at 135° C. After 4 hours, the mixture was cooled to room temperature, and the precipitated solid was collected by filtration. The solid was then filtered through silica gel and recrystallized to obtain Compound C-36 (8.2 g, yield: 78%).

| | MW | M.P. |
|---|---|---|
| C-36 | 499.57 | 286° C. |

### [Example 7] Synthesis of Compound C-38

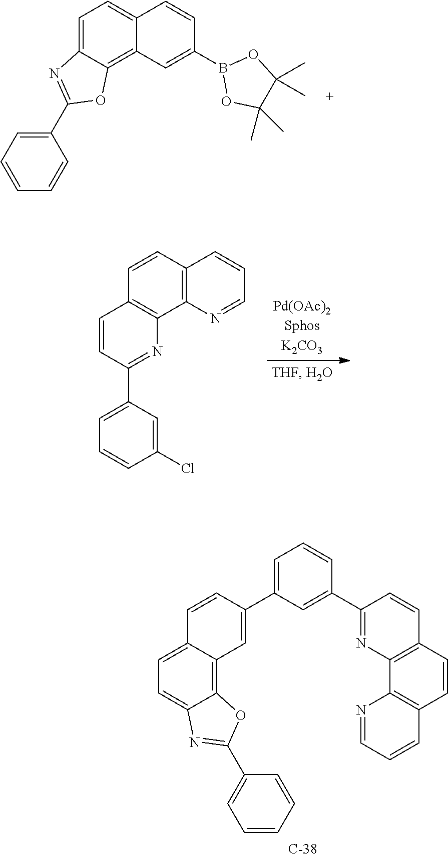

2-Phenyl-8-(4,4,5,5-tetramethyl-1,3,2-dioxaborolan-2-yl)naphtho[2,1-d]oxazole (8.7 g, 24 mmol), 2-(3-chlorophenyl)-1,10-phenanthroline (6.7 g, 23 mmol), Pd(OAc)₂ (0.26 g, 1.0 mmol), SPhos (0.95 g, 2.3 mmol), and K₂CO₃ (9.6 g, 69 mmol) were added to 150 mL of THF and 15 mL of distilled water and then stirred under reflux at 70° C. After 6 hours, the mixture was cooled to room temperature, and the layers were separated. The material was then filtered through silica gel and recrystallized to obtain Compound C-38 (6.5 g, yield: 57%).

| | MW | M.P. |
|---|---|---|
| C-38 | 499.57 | 285° C. |

### [Example 8] Synthesis of Compound C-3

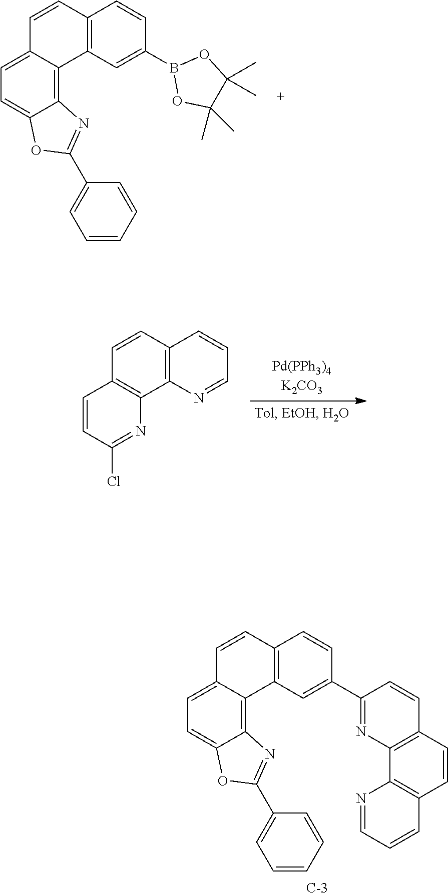

(2-Phenyl-10-(4,4,5,5-tetramethyl-1,3,2-dioxaborolan-2-yl)phenanthro[4,3-d]oxazole (11 g, 25 mmol), 2-chloro-1,10-phenanthroline (5.3 g, 24 mmol), Pd(PPh₃)₄ (1.4 g, 1.2 mmol), and K₂CO₃ (10 g, 73 mmol) were added to 135 mL of toluene, 42 mL of ethanol, and 42 mL of distilled water and then stirred under reflux at 100° C. After 18 hours, the mixture was cooled to room temperature, and the precipitated solid was collected by filtration and then filtered through silica gel. The material was subsequently recrystallized to obtain Compound C-3 (9.0 g, yield: 78%).

| | MW | M.P. |
|---|---|---|
| C-3 | 473.54 | 280° C. |

### [Example 9] Synthesis of Compound C-5

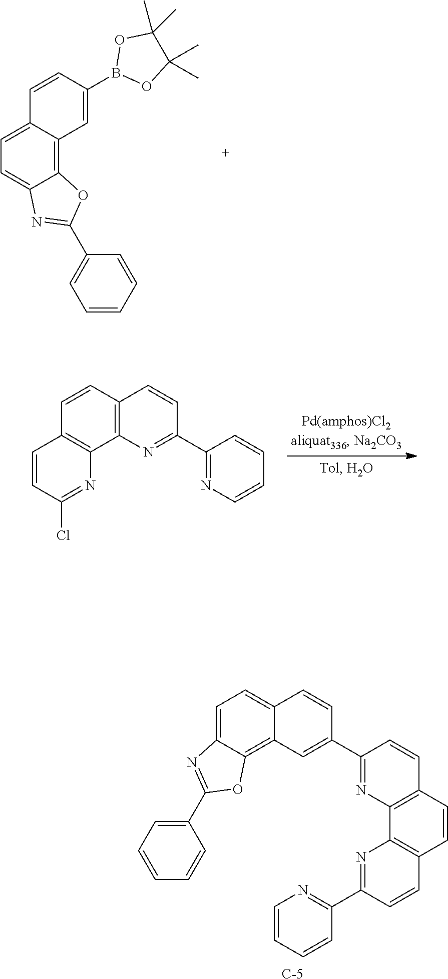

2-Phenyl-8-(4,4,5,5-tetramethyl-1,3,2-dioxaborolan-2-yl)naphtho[2,1-d]oxazole (13 g, 34 mmol), 2-chloro-9-(pyridin-2-yl)-1,10-phenanthroline (10 g, 34 mmol), Pd(amphos)Cl₂ (1.7 g, 2.4 mmol), Aliquat 336 (1.9 g, 4.8 mmol), and Na₂CO₃ (9.1 g, 86 mmol) were added to 140 mL of toluene and 35 mL of distilled water, and the mixture was refluxed with stirring at 130° C. After 18 hours, the reaction mixture was cooled to room temperature, and the precipitated solid was filtered. The solid was then purified by silica filtration and subsequently recrystallized to afford Compound C-5 (7.1 g, yield: 42%).

| | MW | M.P. |
|---|---|---|
| C-5 | 500.56 | 278° C. |

### [Device Examples 1 to 5] Preparation of OLEDs being Deposited the Compound According to the Present Disclosure as an n-Type Charge Generation Layer Material

OLEDs according to the present disclosure were prepared. First, the transparent ITO thin-film electrode on an OLED glass substrate (manufactured by Geomatec) was ultrasonically cleaned sequentially using acetone and isopropyl alcohol, then stored in isopropyl alcohol until use. Next, the ITO substrate was mounted on the substrate holder of a vacuum deposition apparatus. A cell in the vacuum vapor deposition apparatus was charged with Compound HI-1 and another cell with Compound HT-3; the two materials were evaporated at different rates so that Compound HI-1 was doped in an amount of 3 wt % based on the total amount of Compounds HI-1 and HT-3, thereby depositing a hole injection layer having 5 nm thickness. Next, Compound HT-3 was deposited as a first hole transport layer having a thickness of 30 nm on the hole injection layer. Compound HT-4 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a first light-emitting layer was deposited as follows: a cell in the vacuum deposition apparatus was charged with Compound H-1 as a host and another cell with Compound D-1 as a dopant; the two materials were evaporated at different rates so that the dopant was doped in an amount of 2 wt % based on the total amount of the host and the dopant, thereby depositing a first light-emitting layer having a thickness of 20 nm on the second hole transport layer. Next, Compound ET-1 was deposited on the first light-emitting layer to form a first hole blocking layer with a thickness of 5 nm. Subsequently, Compound ET-2 was deposited as an electron transport layer material to form a first electron transport layer with a thickness of 12 nm. Thereafter, an n-type charge generation layer was formed by depositing Yb so that it was 2 wt % with respect to the compound in Table 1 below, to form a n-type charge generation layer with a thickness of 9 nm. Thereafter, a p-type charge generation layer with a thickness of 6 nm was deposited by doping Compound HI-1 in an amount of 6 wt % based on the total amount of Compounds HI-1 and HT-3. Subsequently, Compound HT-3 was deposited to a thickness of 30 nm to form a third hole transport layer, and Compound HT-4 was deposited to a thickness of 5 nm to form a fourth hole transport layer. Thereafter, a second light-emitting layer was deposited as follows: a cell in the vacuum deposition apparatus was charged with Compound H-1 as a host and Compound D-1 as a dopant; the two materials were evaporated at different rates so that the dopant was doped in an amount of 2 wt % based on the total amount of the host and the dopant, thereby forming a second light-emitting layer with a thickness of 20 nm on the fourth hole transport layer. On the second light-emitting layer, Compound ET-1 was deposited as a second hole-blocking layer to a thickness of 5 nm, and, as materials for the second electron transport layer, two cells in the vacuum deposition apparatus were respectively charged with Compound ET-2 and Compound EI-1, after which the two materials were deposited at a weight ratio of 2:1 to a thickness of 25 nm. Next, Compound Yb was added to two another cells as an electron injection layer, evaporated to form an electron injection layer having a thickness of 1 nm on the second electron transport layer. Next, an Al cathode having a thickness of 80 nm was deposited on the electron injection layer by another vacuum vapor deposition apparatus. Thus, OLEDs were produced. Each compound used for all of the materials was purified by vacuum sublimation at 10⁻⁶ Torr.

### [Device Comparative Example 1] Preparation of an OLED being Deposited a Conventional Compound as an N-Type Charge Generation Layer Material

An OLED was manufactured in the same manner as in Device Example 1, except that the compounds in Table 1 below were used as N-type charge generation layer materials.

The driving voltage and the luminous efficiency at a luminance of 1,000 nit, and for the time (lifespan; T₉₅) required for the light intensity to decrease from 100% to 95% when the lifetime is evaluated under 2× acceleration of the OLEDs of Device Examples 1 to 5 and Device Comparative Example 1 produced as described above were measured, and the results thereof are shown in Table 1 below.

TABLE 1

| | N-Type | | | |
|---|---|---|---|---|
| | Charge | Driving | Luminous | |
| | Generation | Voltage | Efficiency | Lifespan |
| | Layer | [V] | [cd/A] | T₉₅ [hr] |
| Device | C-5 | 6.6 | 7.7 | 176.9 |
| Example 1 | | | | |
| Device | C-3 | 6.5 | 7.7 | 156.8 |
| Example 2 | | | | |
| Device | C-1 | 6.5 | 7.7 | 158.4 |
| Example 3 | | | | |
| Device | C-36 | 6.6 | 7.7 | 220.0 |
| Example 4 | | | | |
| Device | C-38 | 6.6 | 7.7 | 214.8 |
| Example 5 | | | | |
| Device | N-1 | 6.6 | 7.7 | 148.9 |
| Comparative | | | | |
| Example 1 | | | | |

From Table 1 above, it can be confirmed that the organic electroluminescent device including the organic electroluminescent compound according to the present disclosure in the N-type charge generation layer exhibits improved lifespan characteristics compared to the organic electroluminescent device using the conventional compound as the N-type charge generation layer material.

### [Device Example 6] Preparation of an OLED being Deposited the Compound According to the Present Disclosure as an n-Type Charge Generation Layer Material

An OLED was prepared in the same manner as in Device Example 1, except that the compound shown in Table 2 below was used as the N-type charge generation layer material and the Compound H-2-D17 was used as the host material for the light-emitting layer.

### [Device Comparative Example 2] Preparation of an OLED being Deposited a Conventional Compound as an N-Type Charge Generation Layer Material

An OLED was fabricated in the same manner as in Device Example 6, except that the compound shown in Table 2 below was used as the N-type charge generation layer material.

The OLEDs prepared described above according to Device Example 6 and Device Comparative Example 2 were evaluated with respect to the driving voltage at a luminance of 1,000 nit and the device lifetime under a two-times accelerated condition. The change in driving voltage until the luminance decreased to 95% (T₉₅) was measured, and the results are shown in Table 2 below. The progressive driving voltage (ΔV) refers to the difference between the initial driving voltage and the driving voltage at T₉₅.

TABLE 2

| | | Driving | Progressive Driving |
|---|---|---|---|
| | N-Type Charge | Voltage | Voltage (ΔV) |
| | Generation Layer | [V] | [%] |
| Device | C-7 | 6.4 | 100.5 |
| Example 6 | | | |
| Device | N-1 | 6.6 | 103 |
| Comparative | | | |
| Example 2 | | | |

As can be seen from Table 2, the organic electroluminescent device according to the present disclosure, by including the organic electroluminescent compound in the N-type charge generation layer, exhibits improved driving-voltage stability, thereby suppressing an increase in power consumption and deterioration of device lifetime.

The compounds used in Device Examples 1 to 6 and Device Comparative Examples 1 and 2 are specifically shown in Table 3 below.

TABLE 3

| | |
|---|---|
| Hole Injection Layer/ Hole Transport Layer | 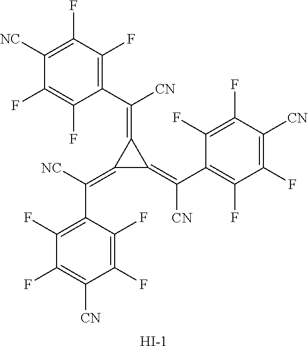 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 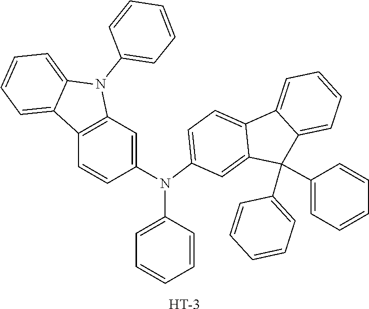 |
| | 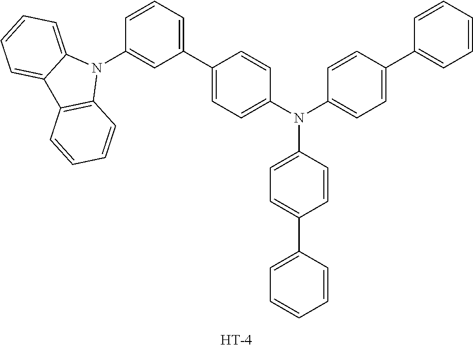 |
| Light-Emitting Layer | 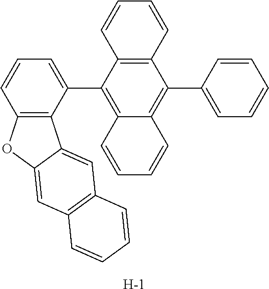 |
| | 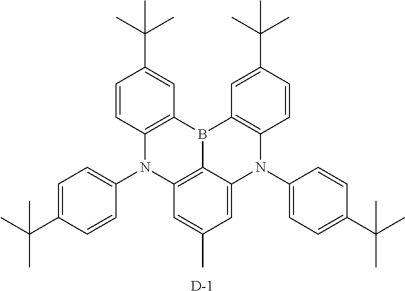 |
| | 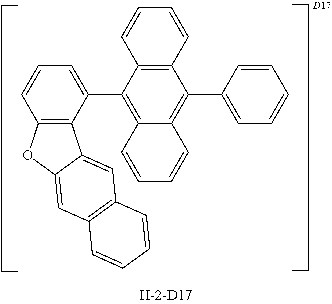 |
| Hole-Blocking Layer/ Electron Transport Layer/ Electron Injection Layer | 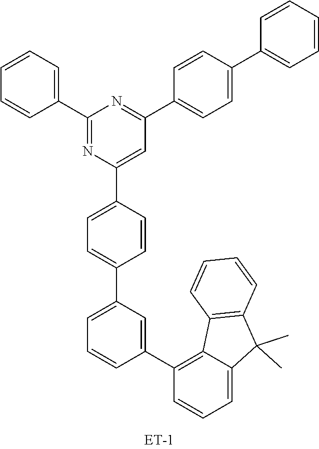 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | 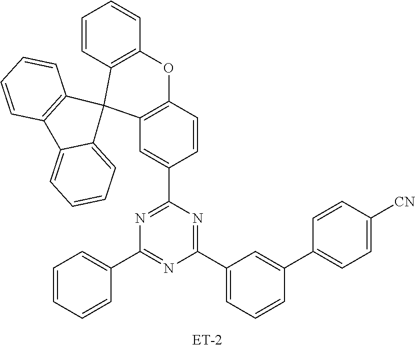 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | 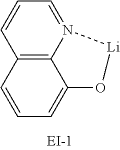 |
| N-Type Charge Generation Layer | 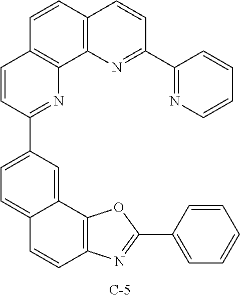 |
| | 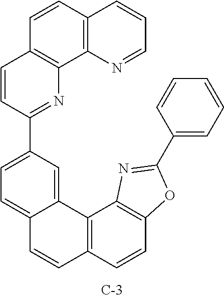 |
| | 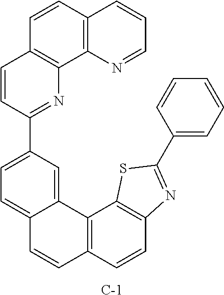 |
| | 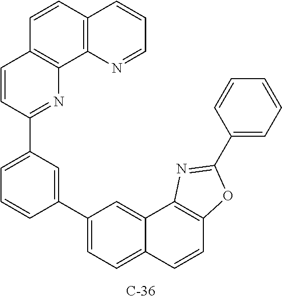 |
| | 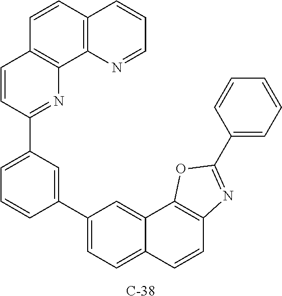 |
| | 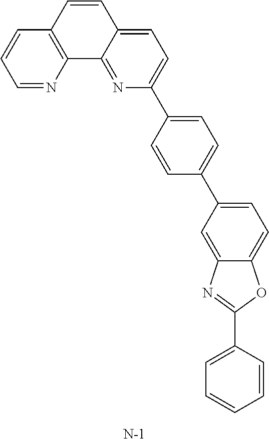 |
| | 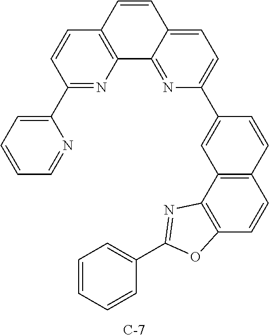 |

1. An organic electroluminescent compound represented by the following Formula 1: 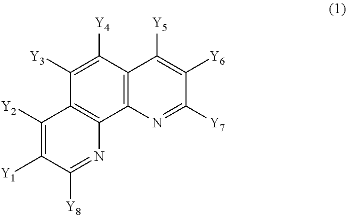
wherein,
Y₁ to Y₆ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring of a (C₃-C₃₀)aliphatic ring and a (C₆-C₃₀)aromatic ring; or may be linked to an adjacent substituent to form a ring; and
Y₇ and Y₈ each independently represent hydrogen, deuterium, halogen, cyano, a substituted or unsubstituted phenyl, a substituted or unsubstituted biphenyl, a substituted or unsubstituted terphenyl, a substituted or unsubstituted (C₁-C₃₀)alkyl, a substituted or unsubstituted (C₂-C₃₀)alkynyl, or a substituted or unsubstituted (3- to 30-membered)heteroaryl;
provided that at least one of Y₁ to Y₈ is represented by the following Formula 1-a or 1-b, or is HAr, which is a substituted or unsubstituted (3- to 30-membered)heteroaryl; 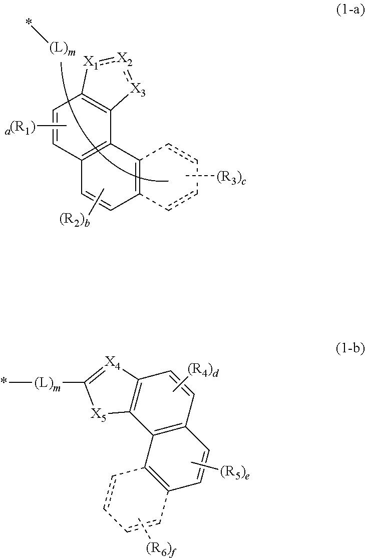
wherein,
the dotted line indicates the presence or absence of each ring;
L represents a single bond, a substituted or unsubstituted (C₆-C₃₀)arylene, a substituted or unsubstituted (3- to 30-membered)heteroarylene, or a substituted or unsubstituted (C₂-C₃₀)alkynylene;
X₁ to X₃ each independently represent —N═, —O—, —S—, or —CRₐ—; provided that one of X₁ to X₃ is —N═, another one of X₁ to X₃ is —O— or —S—, and the remaining one of X₁ to X₃ is —O—, —S—, or —CRₐ—;
X₄ and X₅ each independently represent —N═, —O—, or —S—; provided that one of X₄ and X₅ is —N═ and the other is —O— or —S—;
R₁ to R₃ each independently represent a position for bonding to L, hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, or a substituted or unsubstituted (C₁-C₃₀)alkoxy; or may be linked to an adjacent substituent to form a ring;
Rₐ and R₄ to R₆ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, or a substituted or unsubstituted (C₁-C₃₀)alkoxy; or may be linked to an adjacent substituent to form a ring;
m is an integer of 1 or 2, and when m is 2, each L may be the same or different;
a and d are each independently an integer of 1 or 2, and b, c, e, and f are each independently an integer of 1 to 4; and
when a to f are integers of 2 or more, each of R₁ to each of R₆ may be the same or different.

2. The organic electroluminescent compound according to claim 1, wherein the substituted alkyl, the substituted alkenyl, the substituted alkynyl(ene), the substituted aryl(ene), the substituted heteroaryl(ene), the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, and the substituted fused-ring group of an aliphatic ring and an aromatic ring are each independently substituted with one or more selected from the group consisting of: deuterium, halogen, cyano, carboxyl, nitro, hydroxy, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, a (3- to 30-membered)heteroaryl unsubstituted or a substituted with (C₆-C₃₀)aryl, a (C₆-C₃₀)aryl unsubstituted or a substituted with (3- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused-ring group of a (C₃-C₃₀)aliphatic ring and a (C₆-C₃₀)aromatic ring, amino, mono- or di(C₁-C₃₀)alkylamino, a substituted or unsubstituted mono- or di(C₆-C₃₀)arylamino, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, (C₁-C₃₀)alkyl(C₆-C₃₀)aryl, and phosphine oxide.

3. The organic electroluminescent compound according to claim 1, wherein Formula 1 is represented by any one of the following Formulas 1-1 to 1-6: 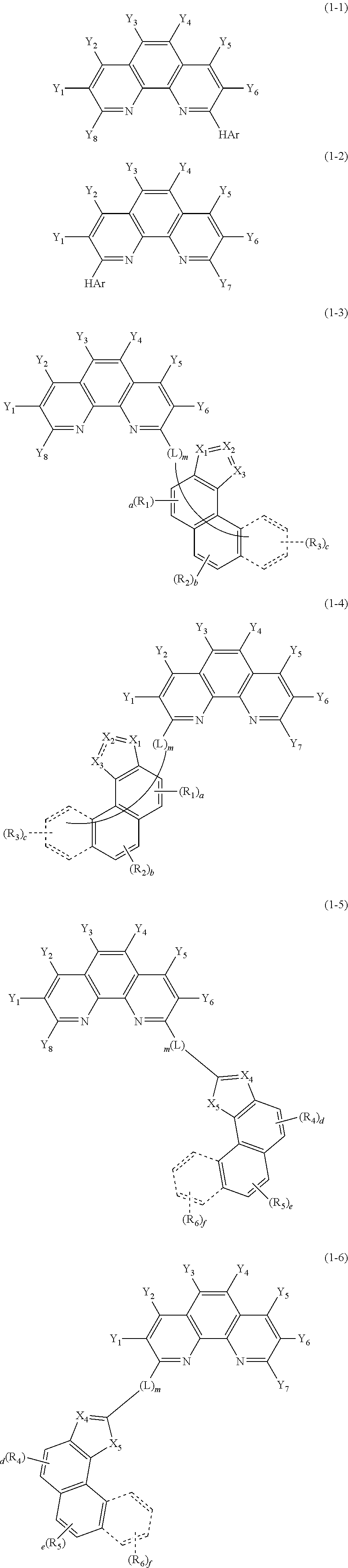
wherein,
Y₁ to Y₈, HAr, L, X₁ to X₅, R₁ to R₆, m, and a to f are as defined in claim 1.

4. The organic electroluminescent compound according to claim 1, wherein Formula 1 is represented by any one of the following Formulas 1-7 to 1-12: 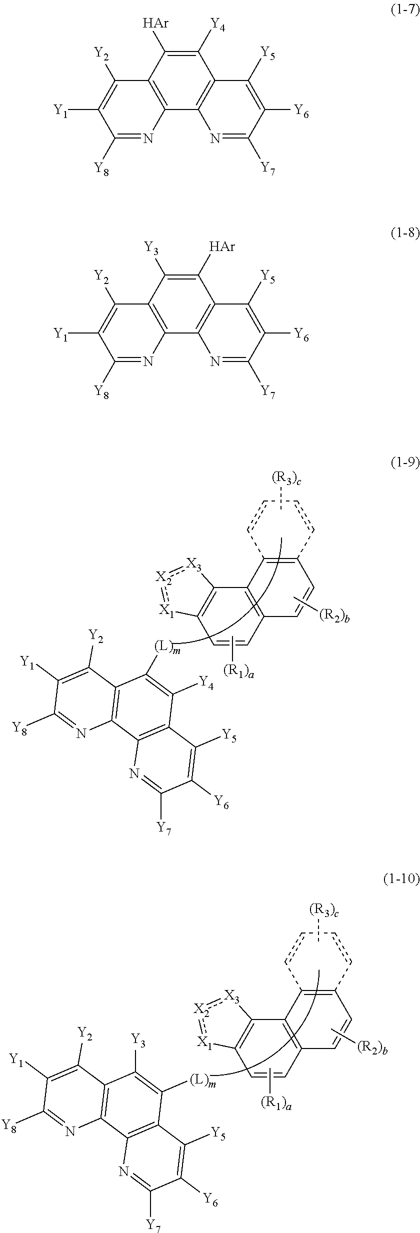 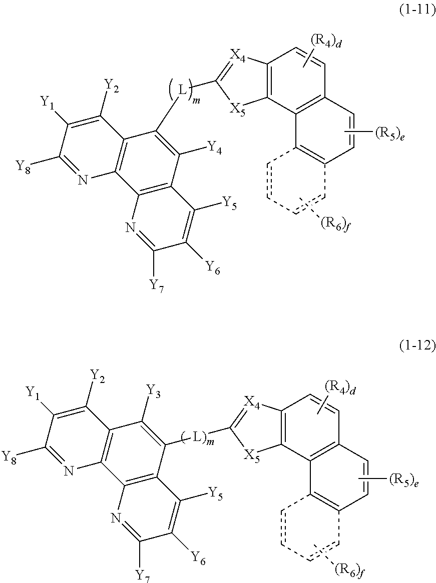
wherein,
Y₁ to Y₈, HAr, L, X₁ to X₅, R₁ to R₆, m, and a to f are as defined in claim 1.

5. The organic electroluminescent compound according to claim 1, wherein Formula 1-a is represented by any one of the following Formulas 1-a-1 to 1-a-10, and Formula 1-b is represented by any one of the following Formulas 1-b-1 and 1-b-2: 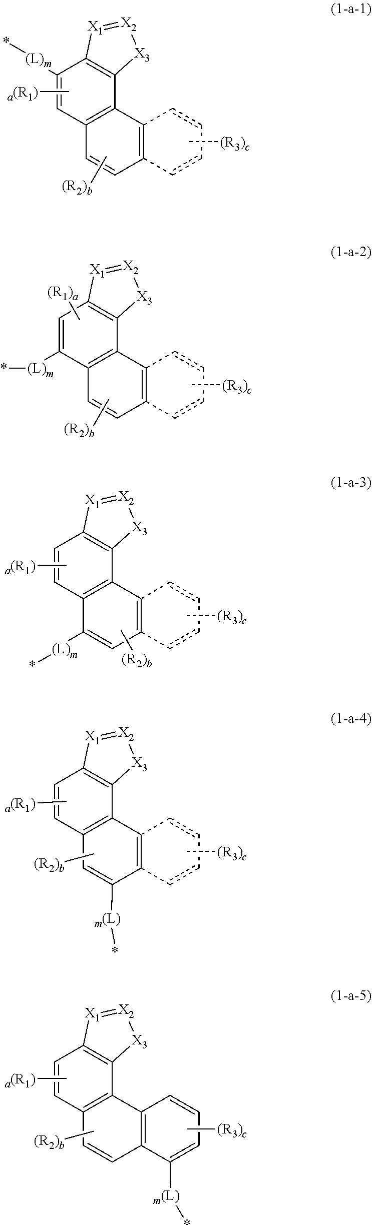 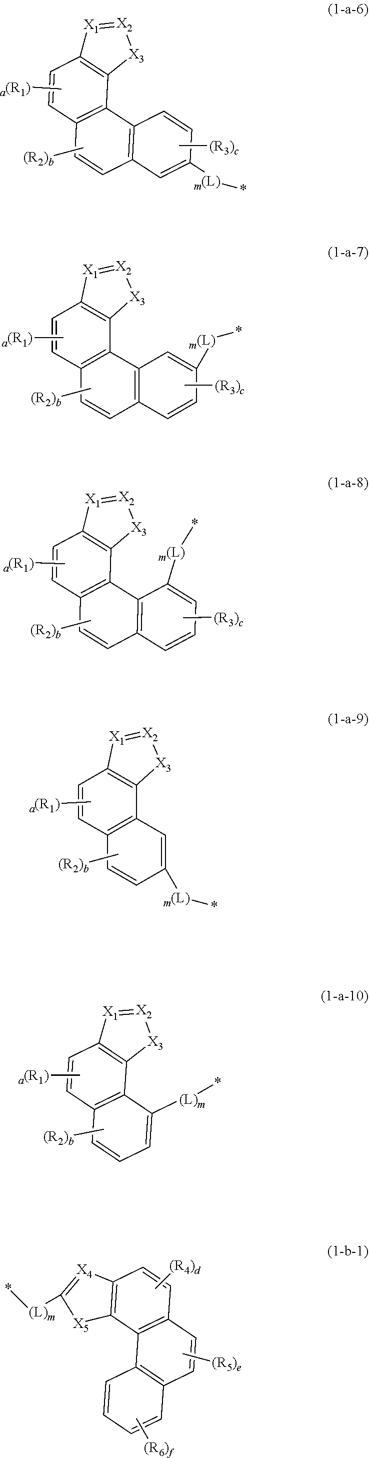 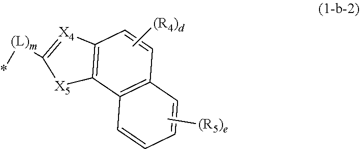
wherein,
R₁ to R₆, X₁ to X₅, L, a to f, and m are as defined in claim 1.

6. The organic electroluminescent compound according to claim 1, wherein the compound represented by Formula 1 is selected from the following compounds: 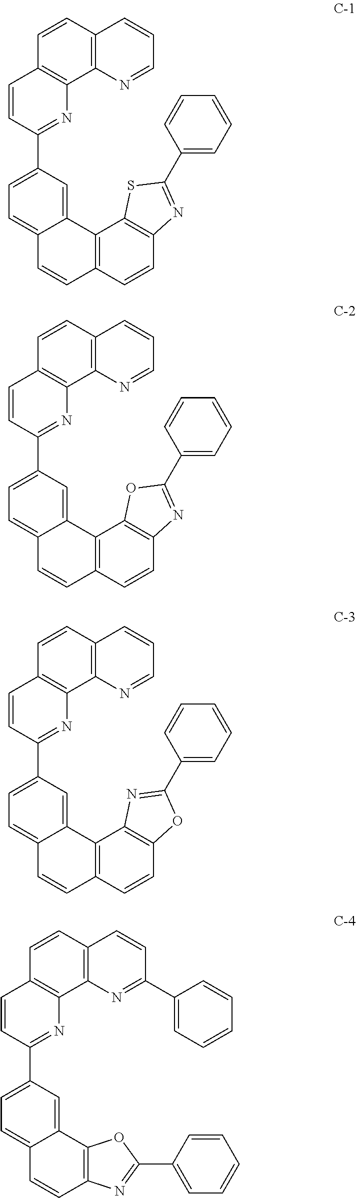 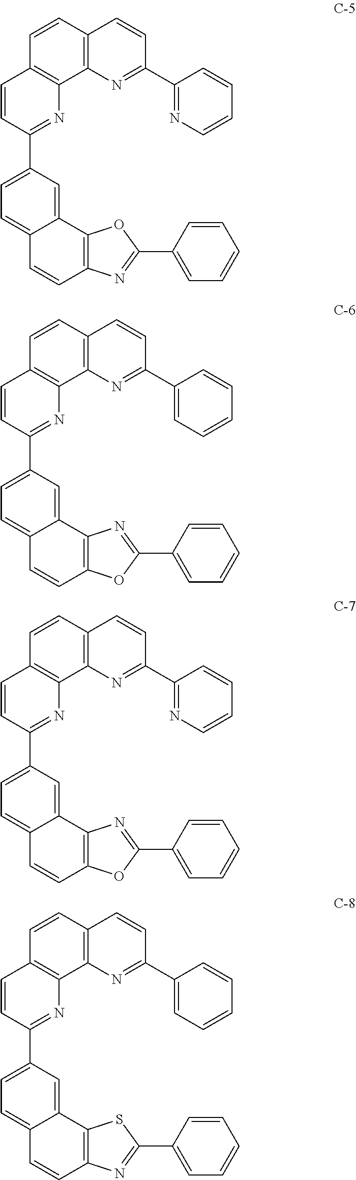 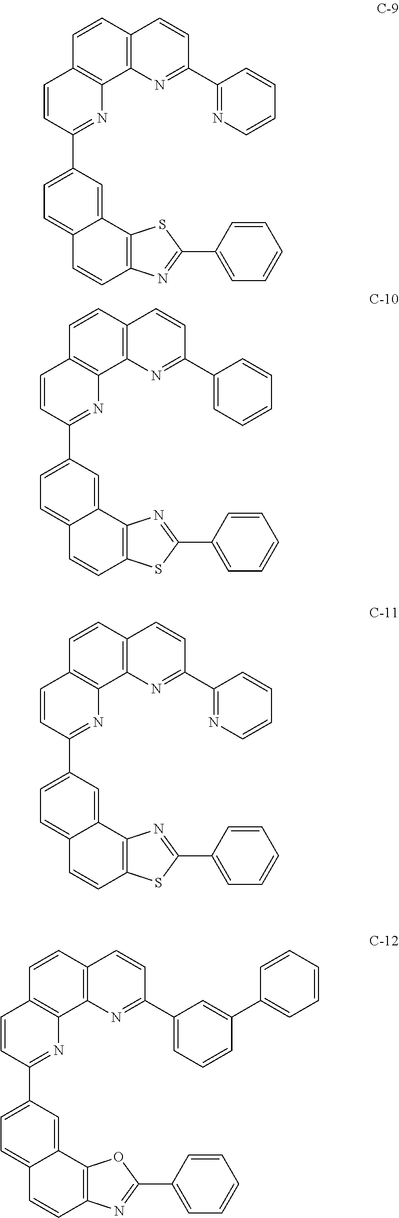 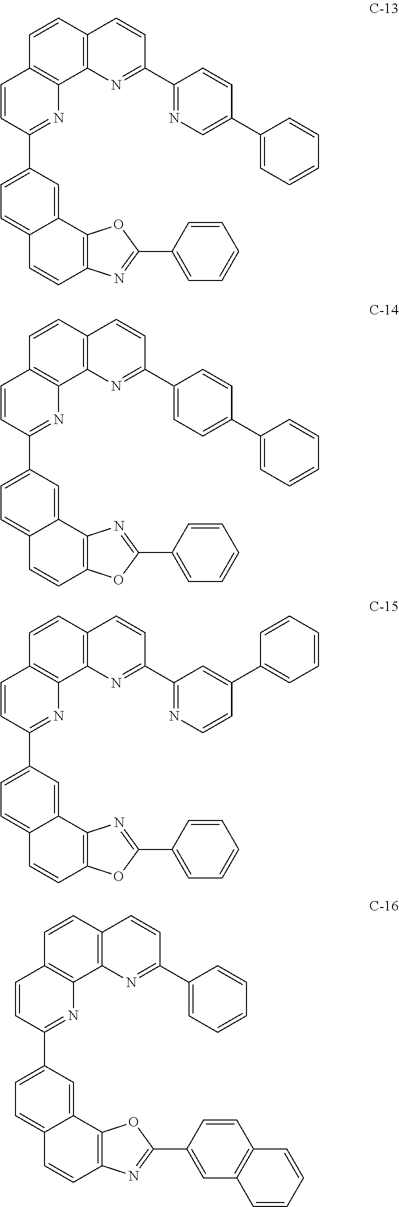 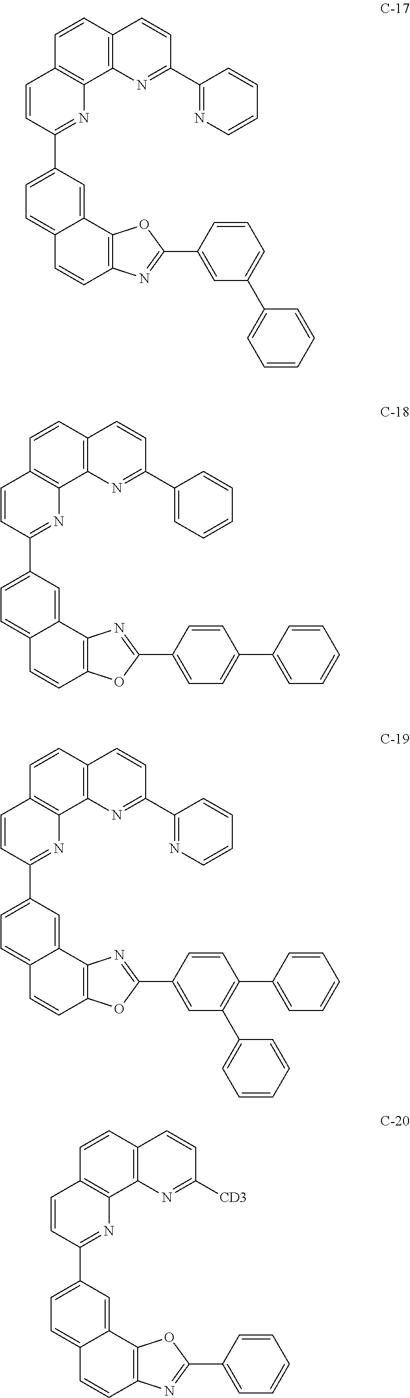 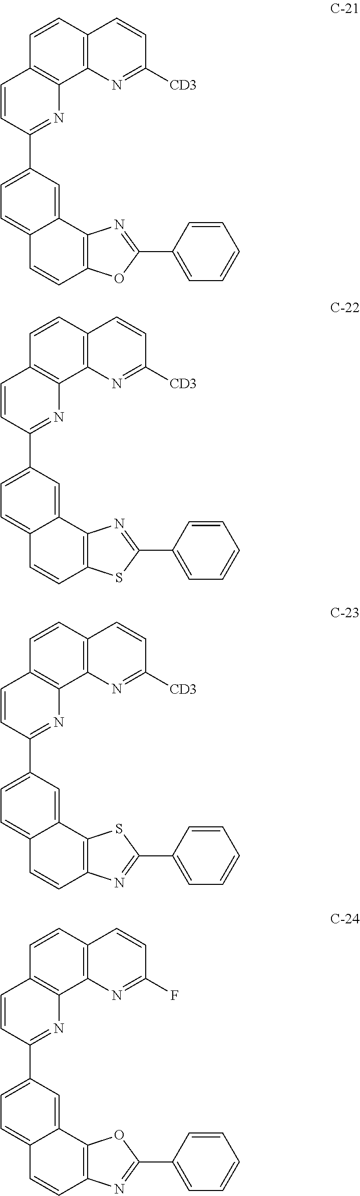 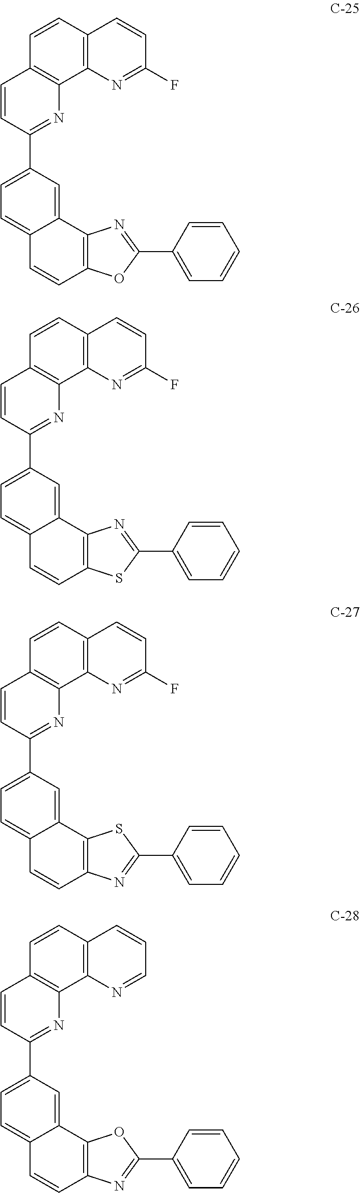 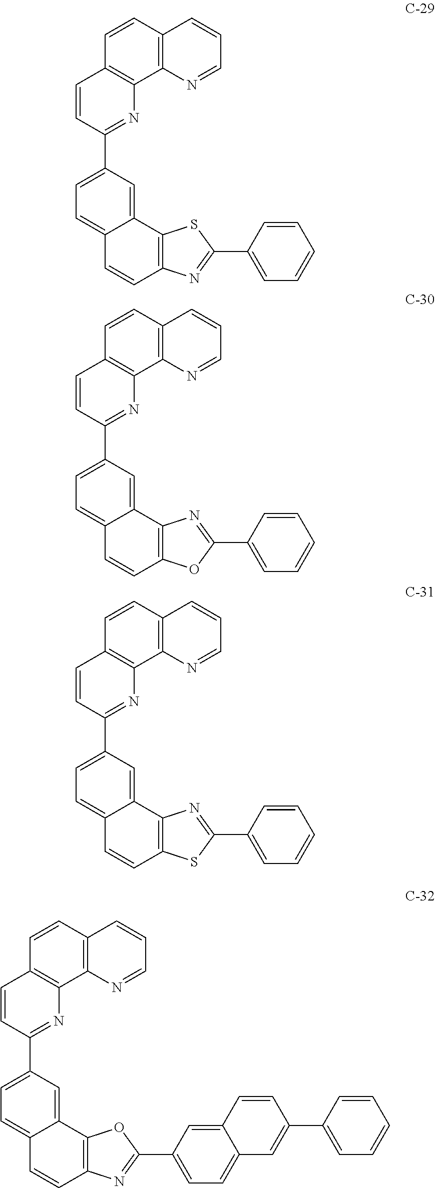 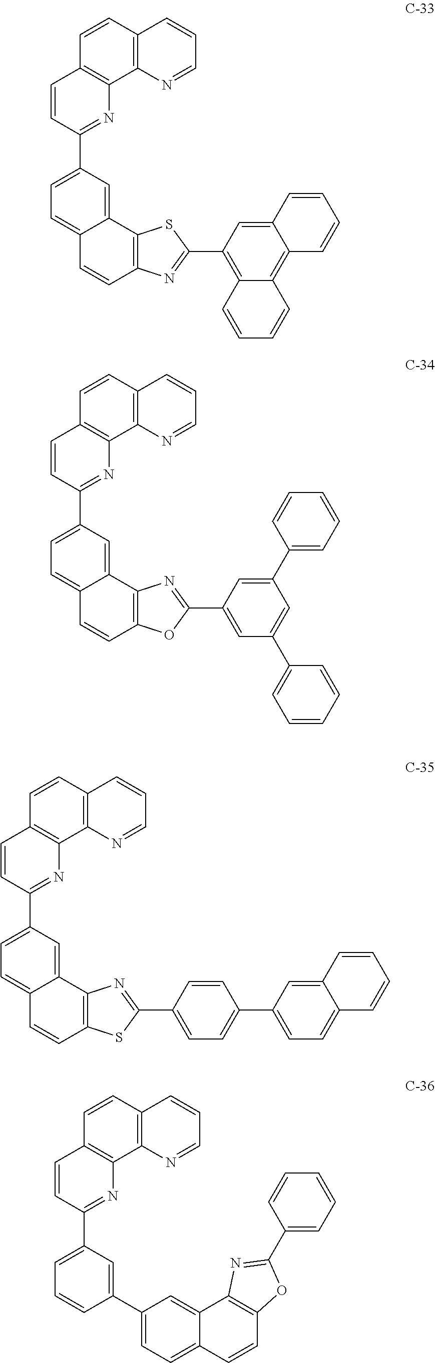 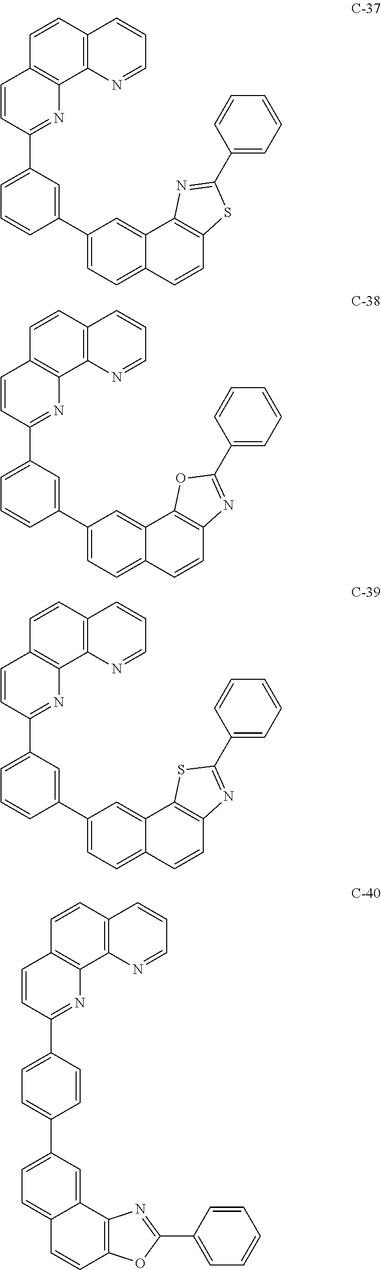 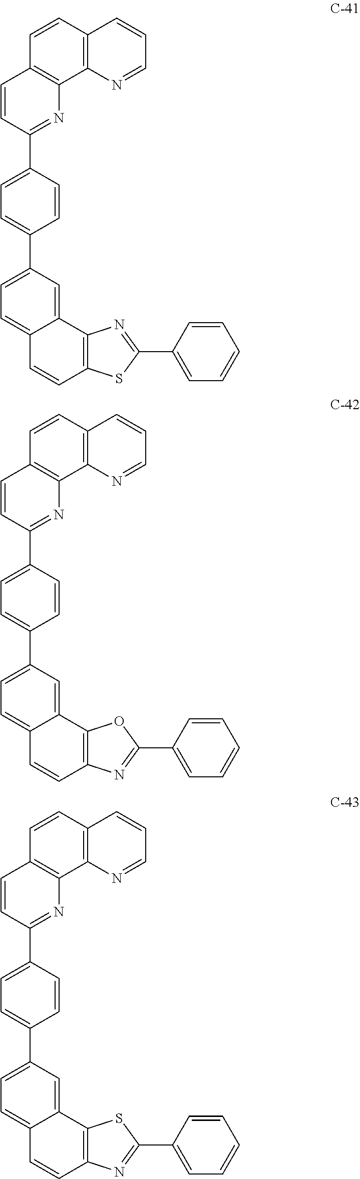 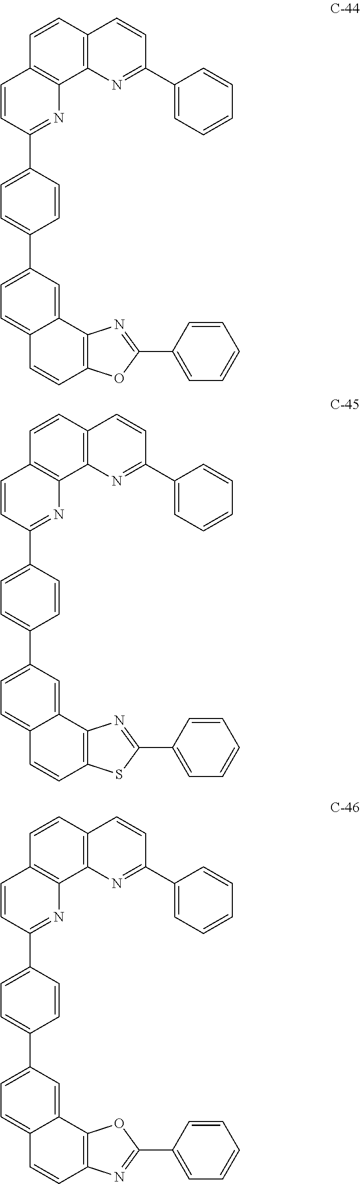 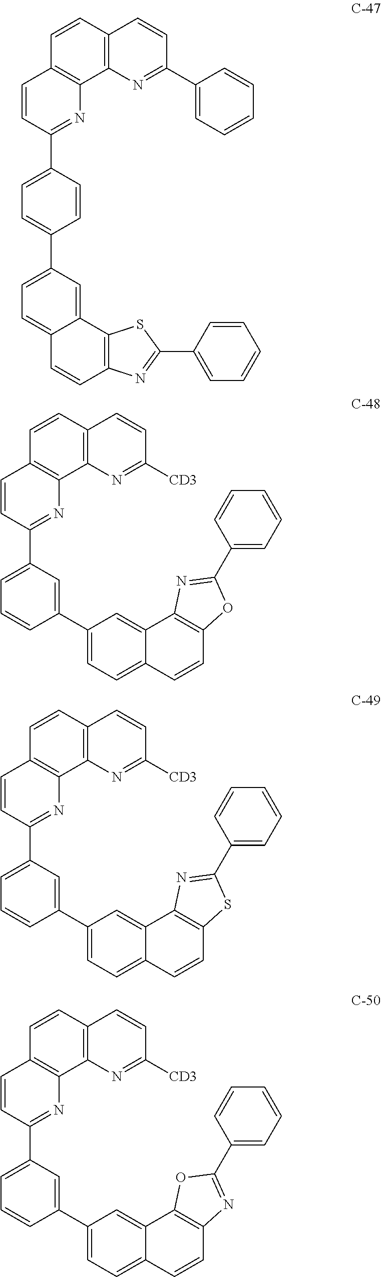 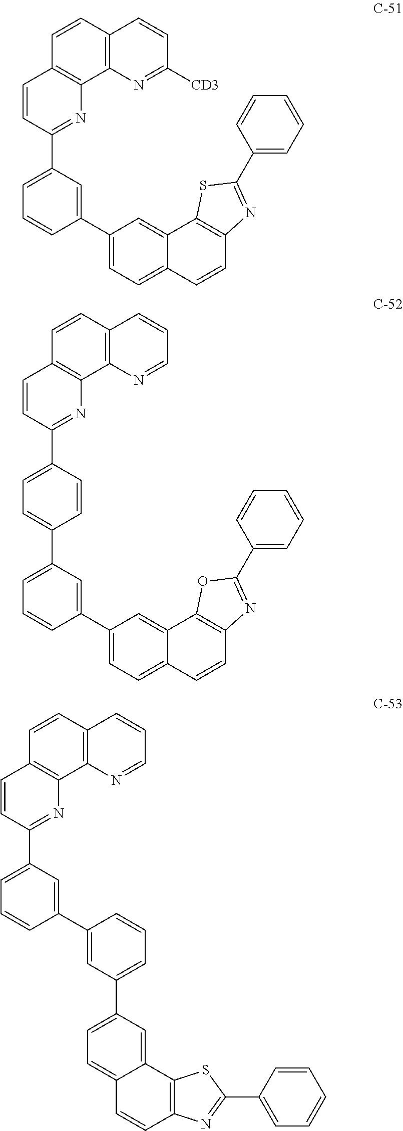 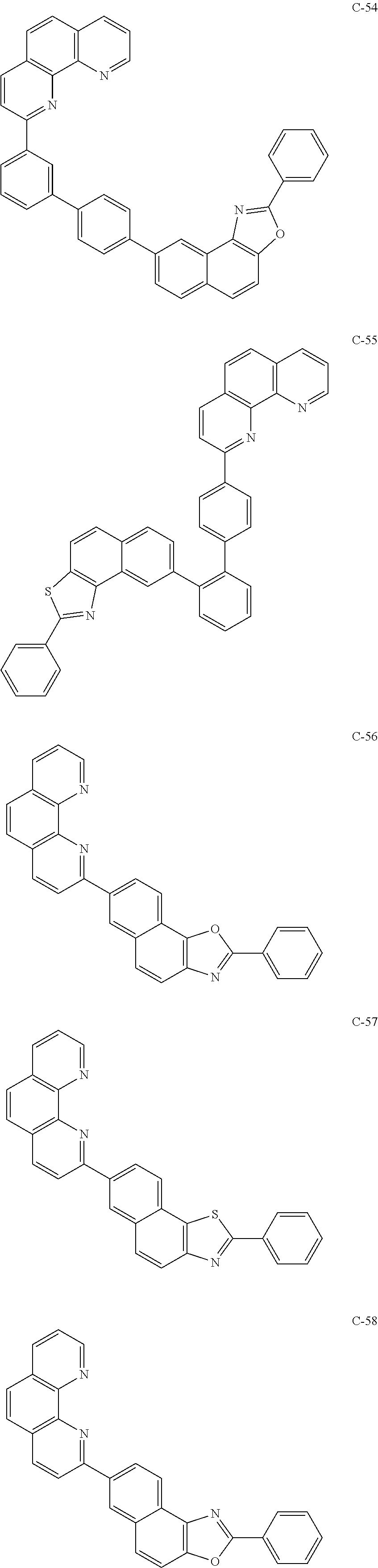 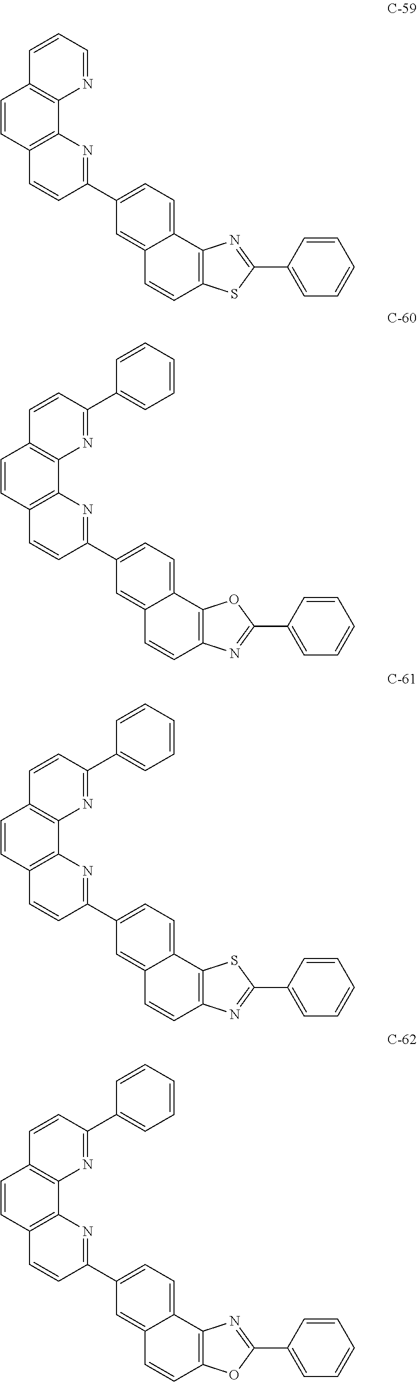 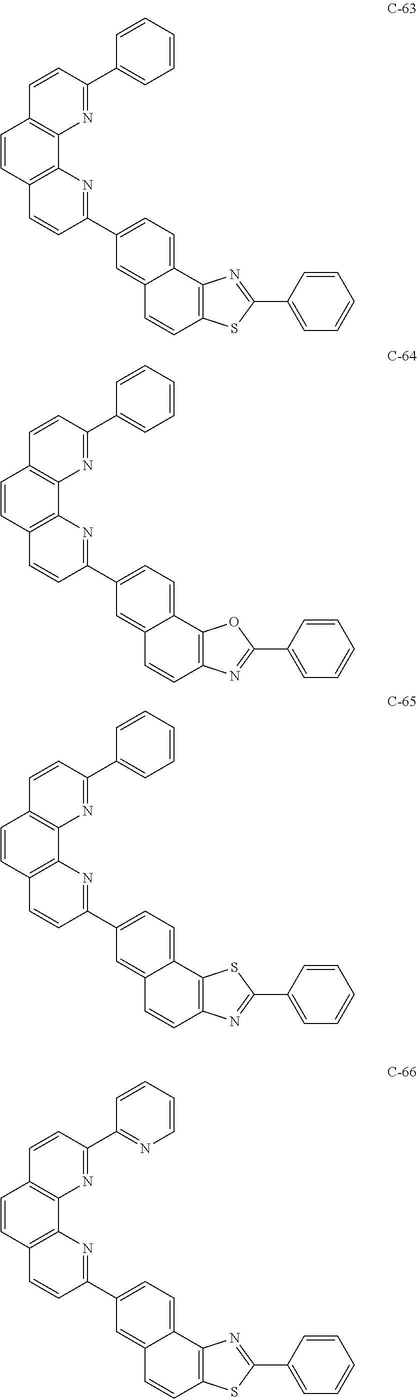 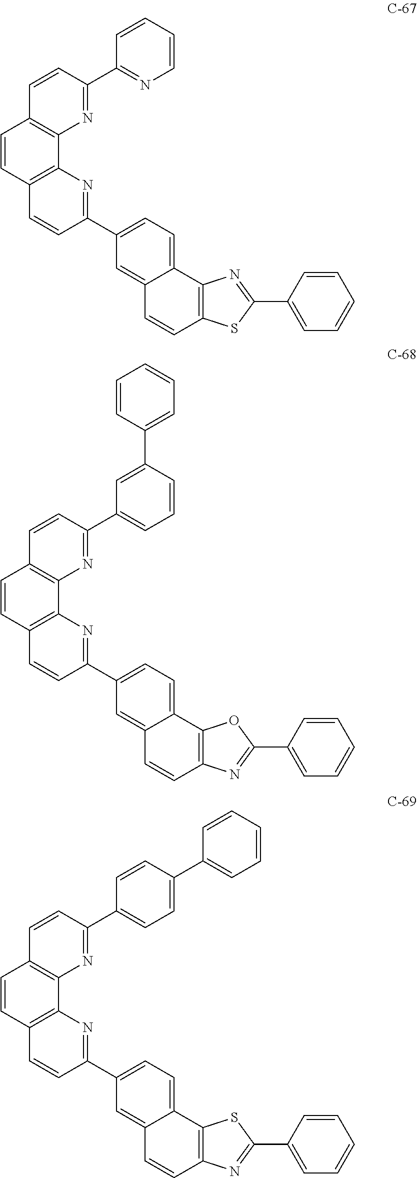 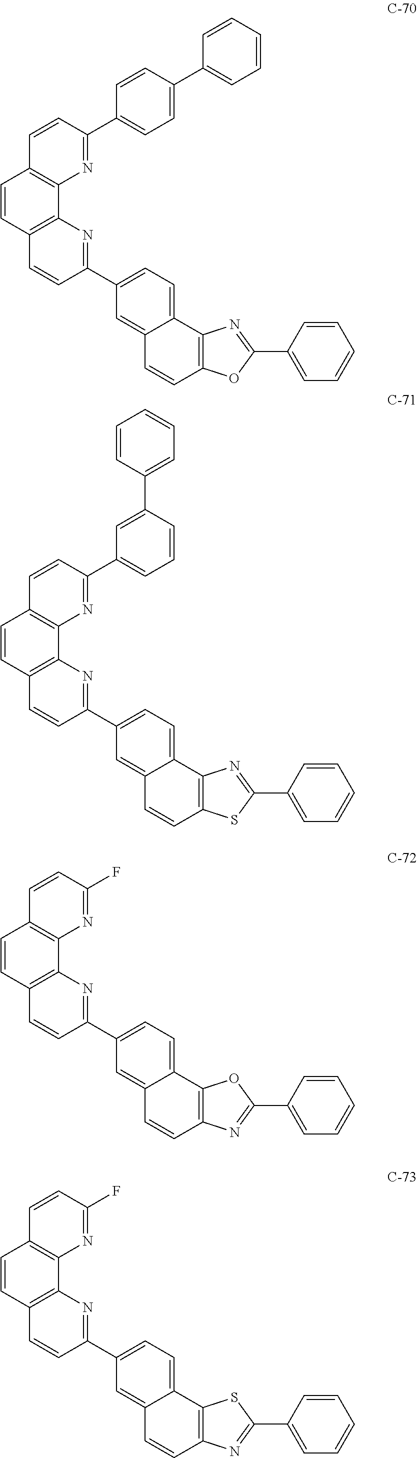 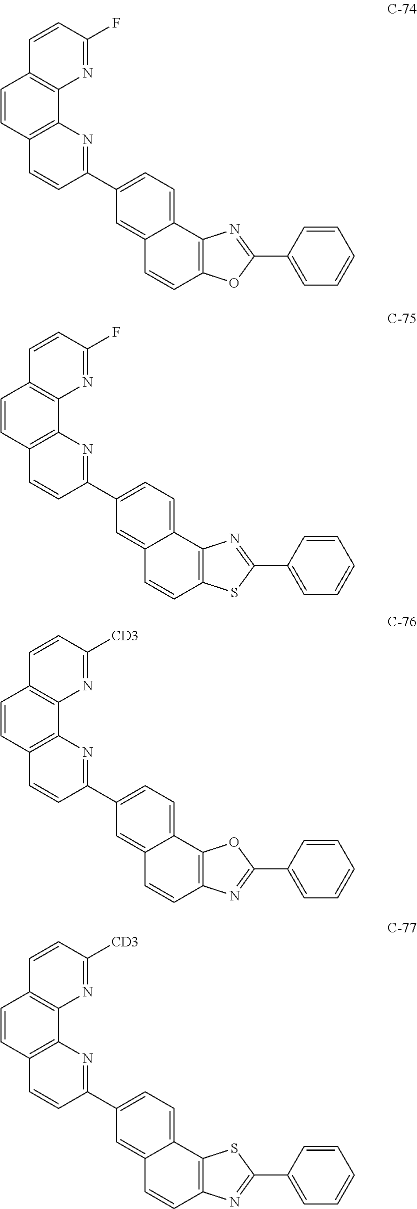 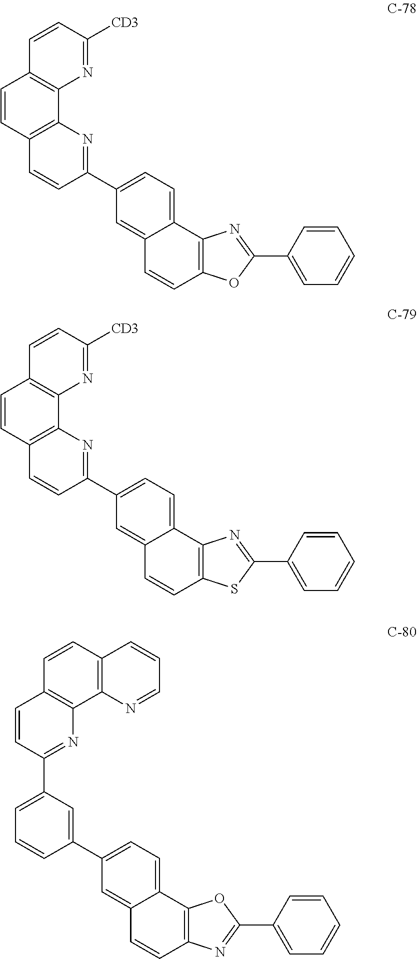 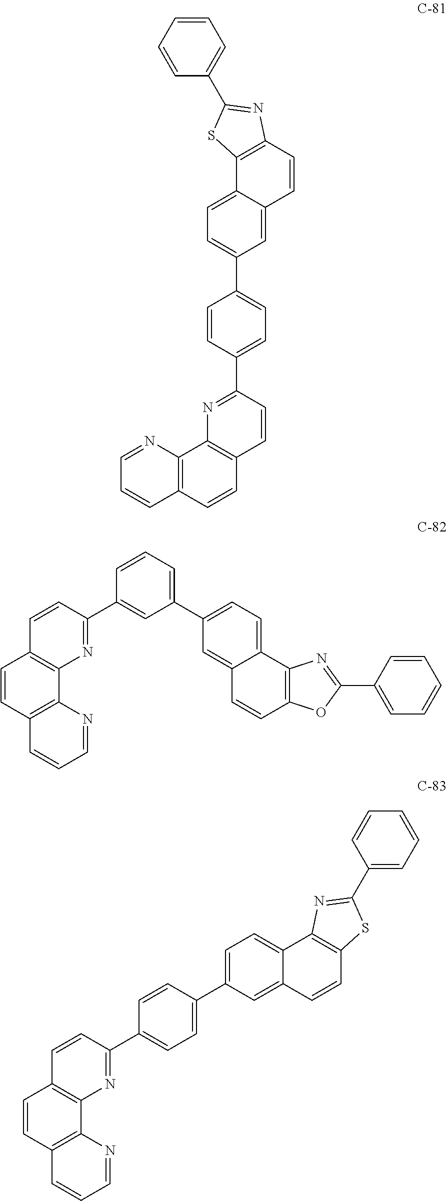 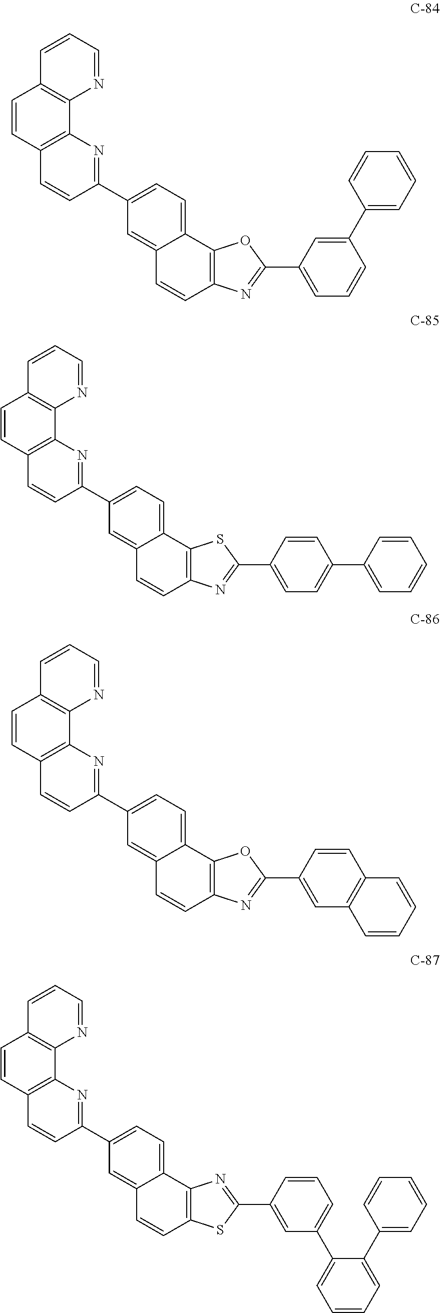 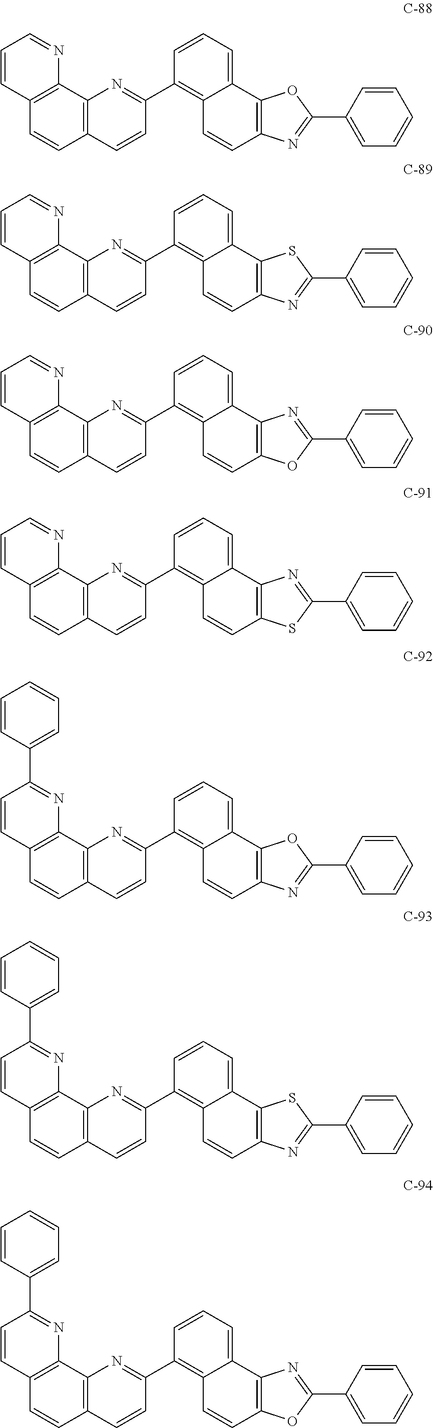 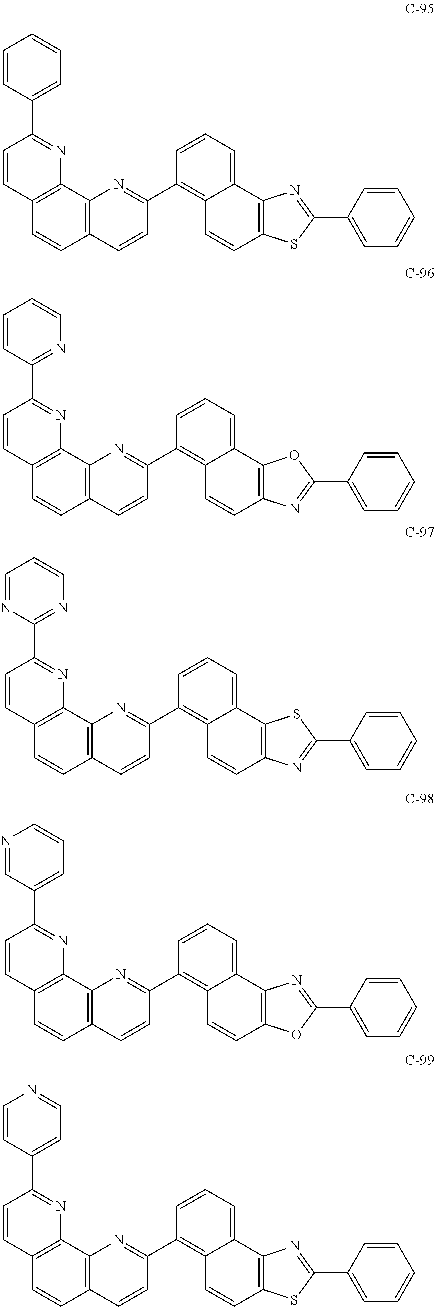 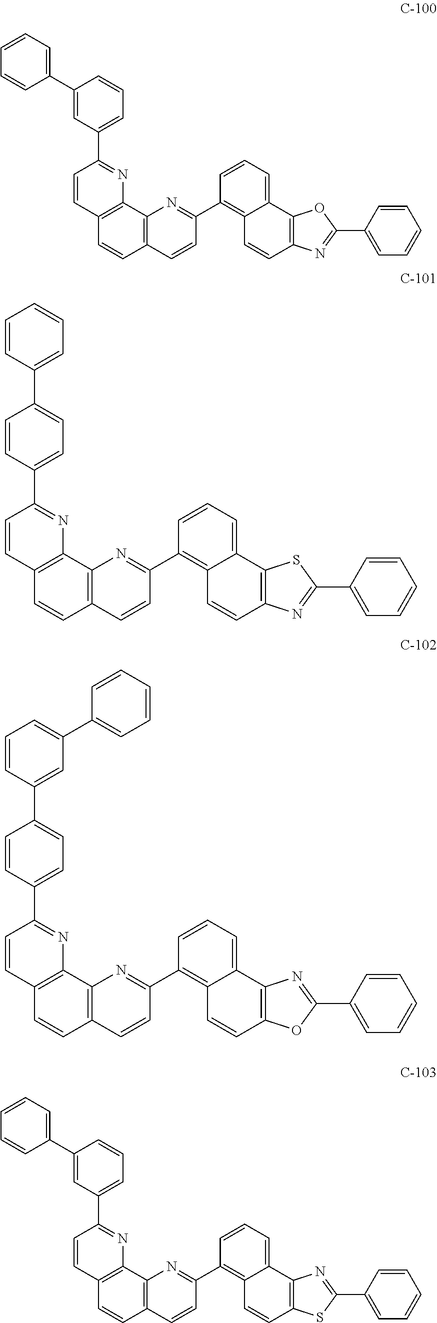 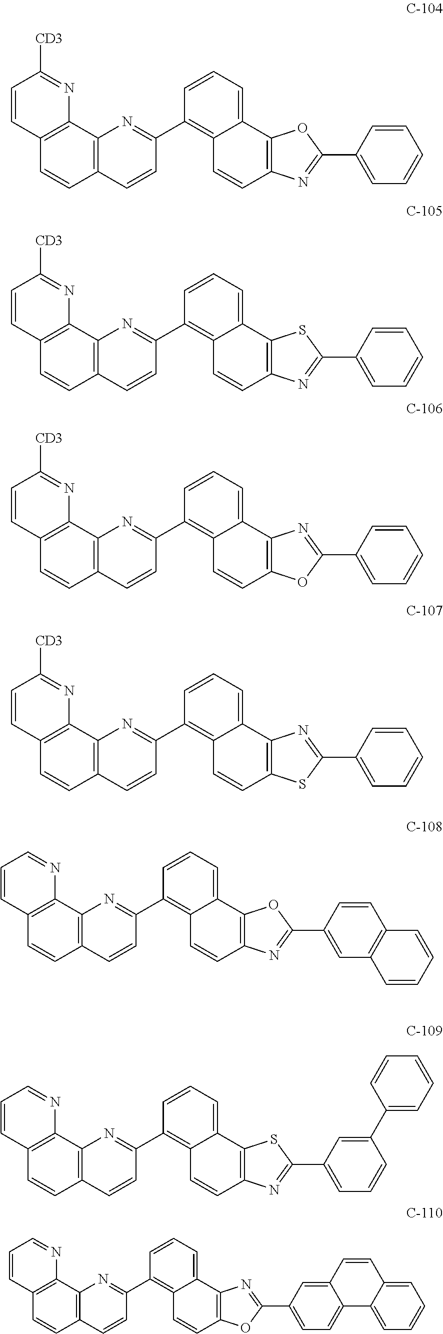 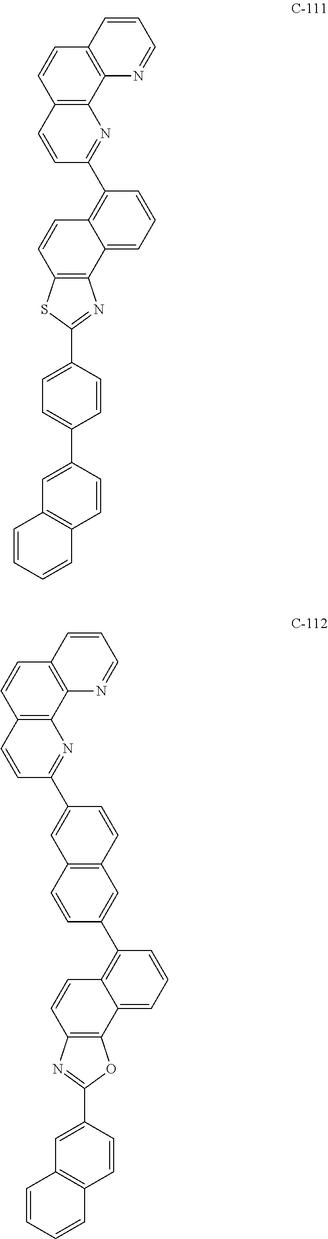 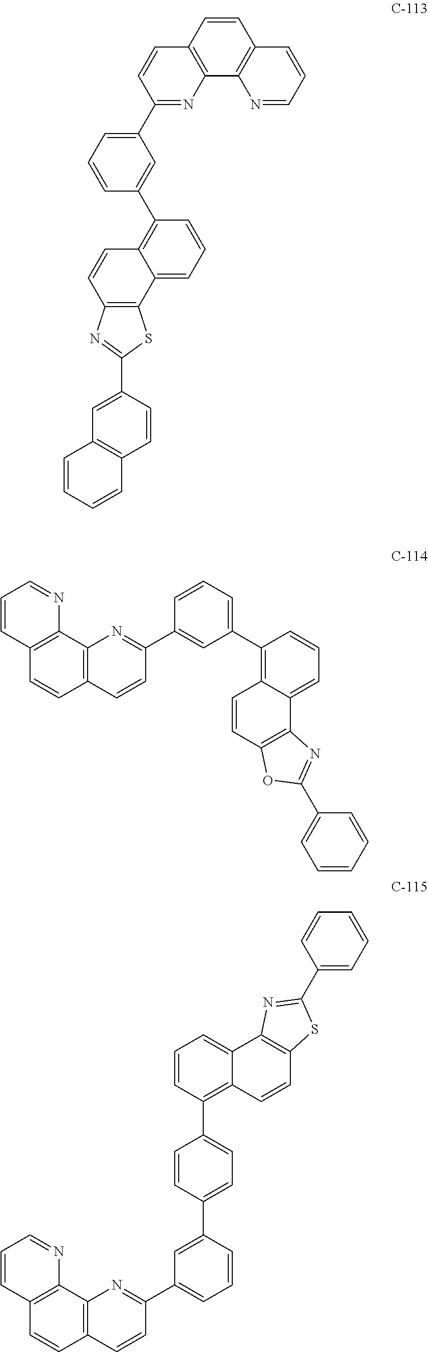 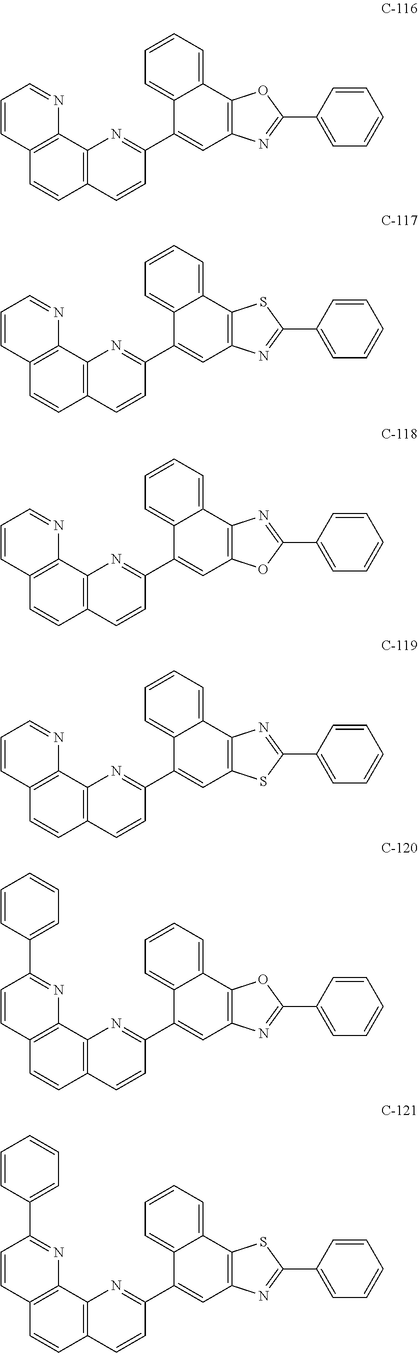 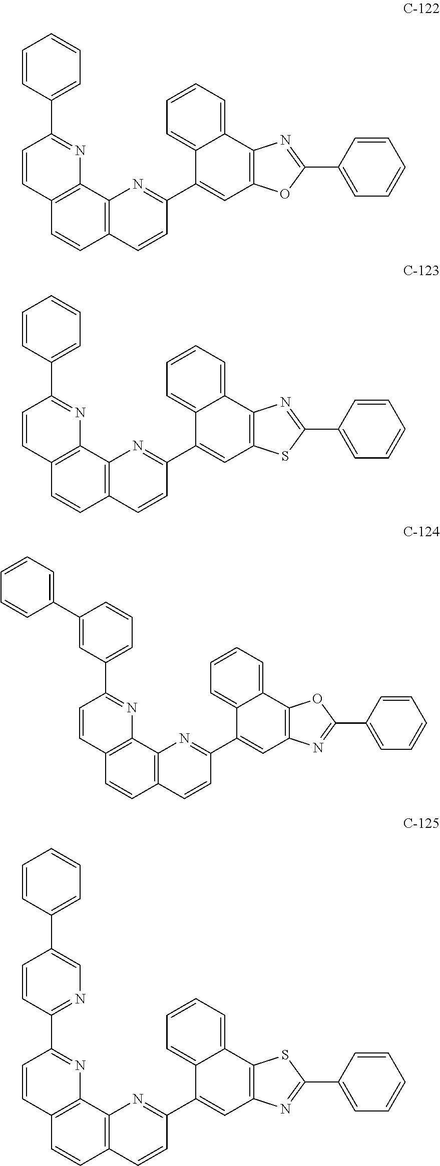 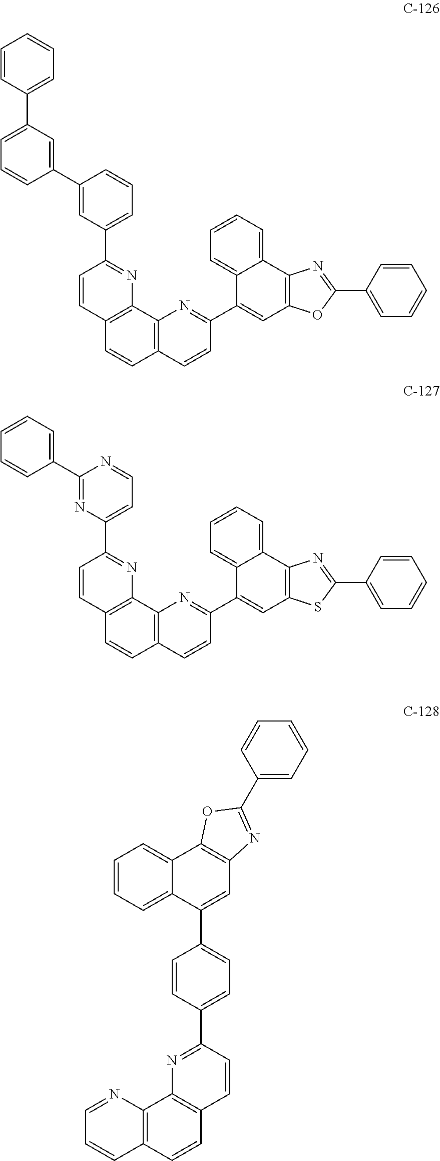 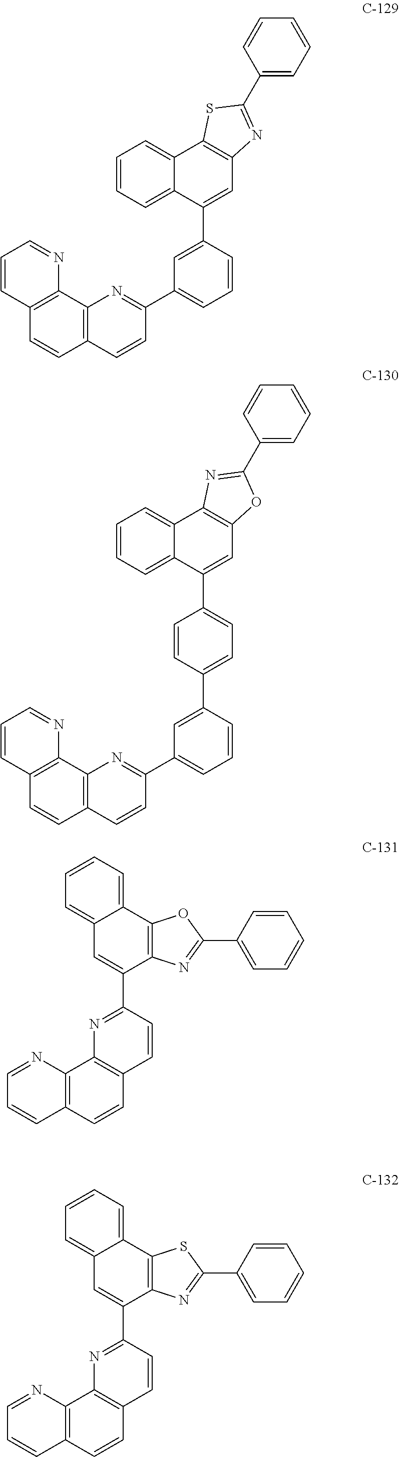 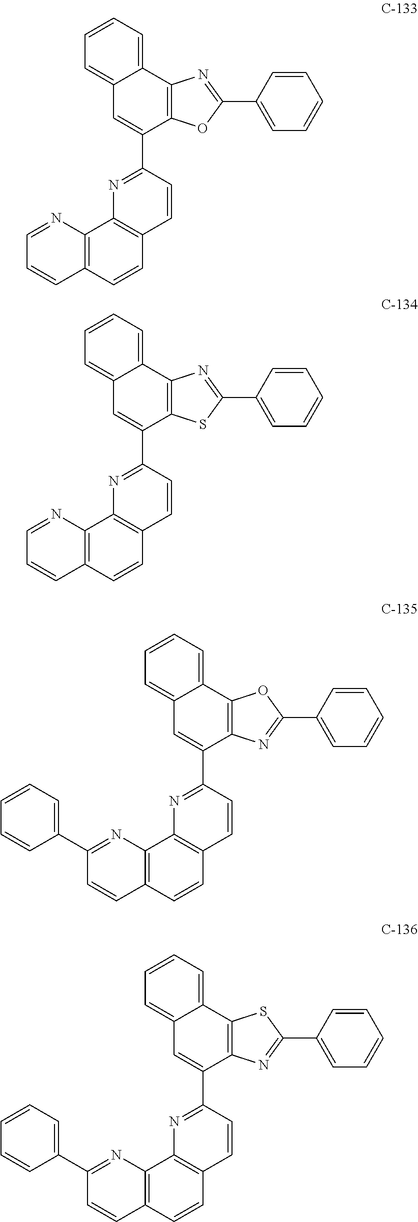 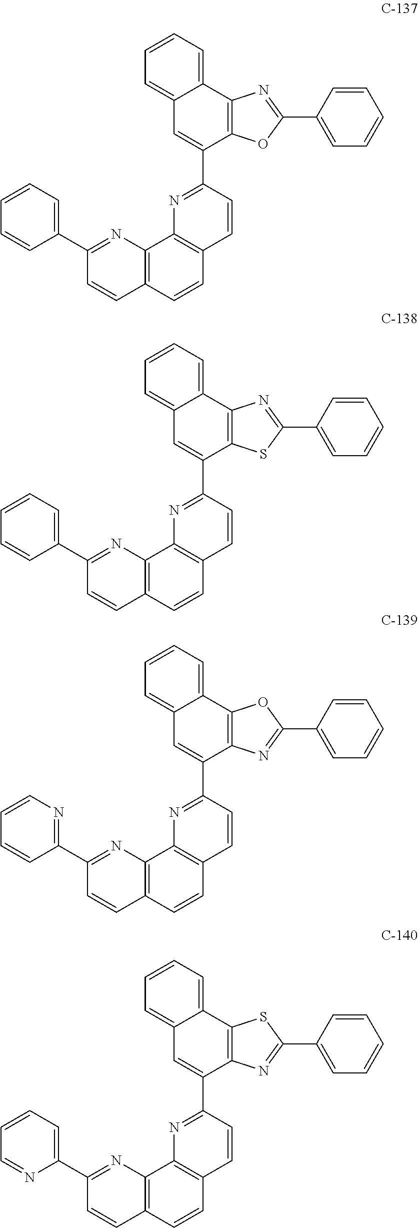 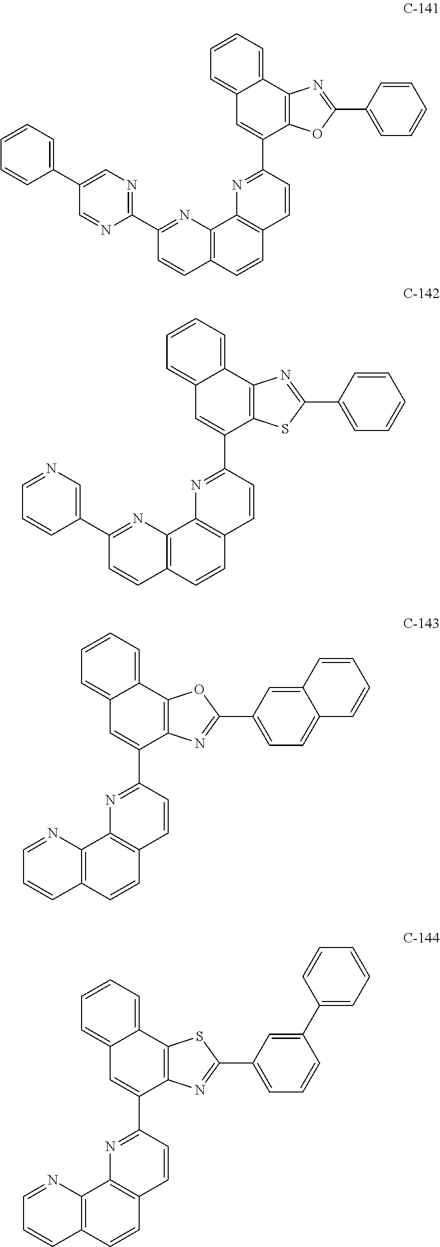 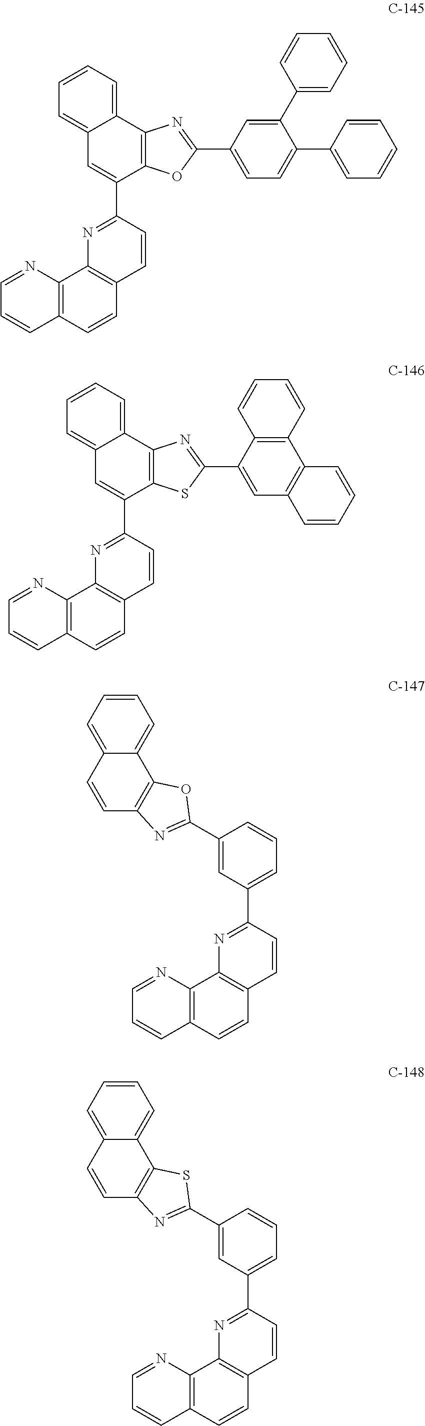 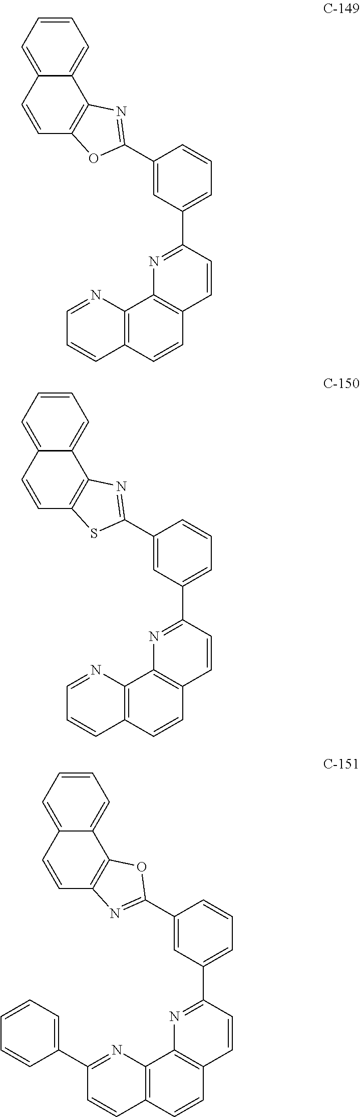 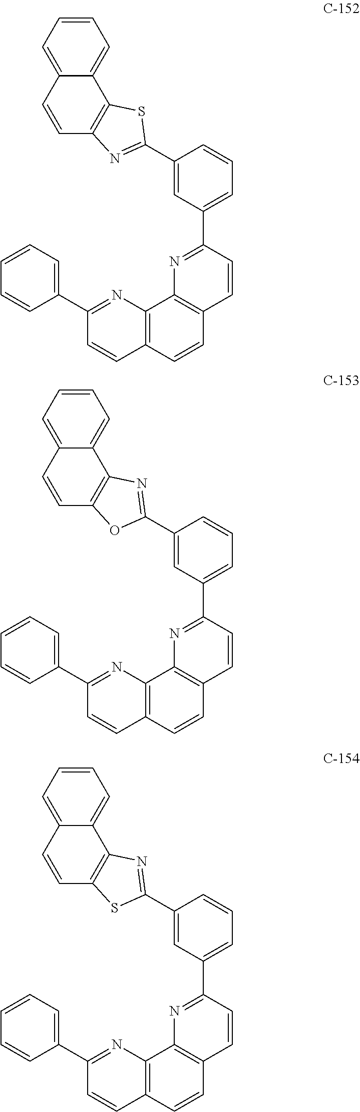 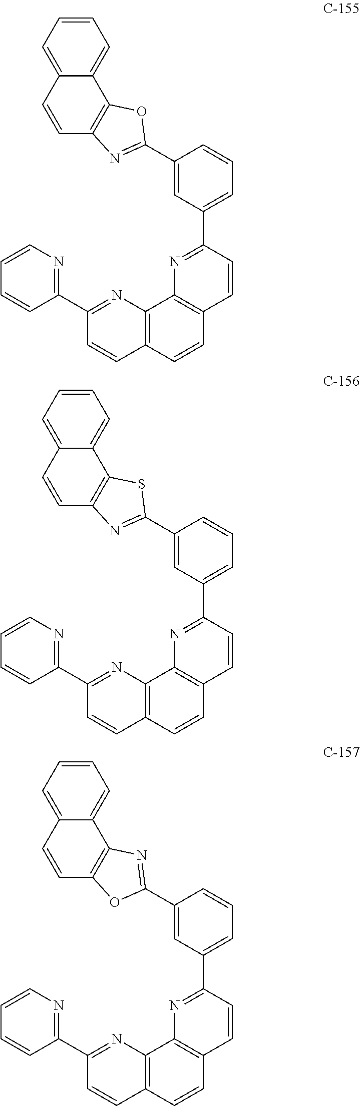 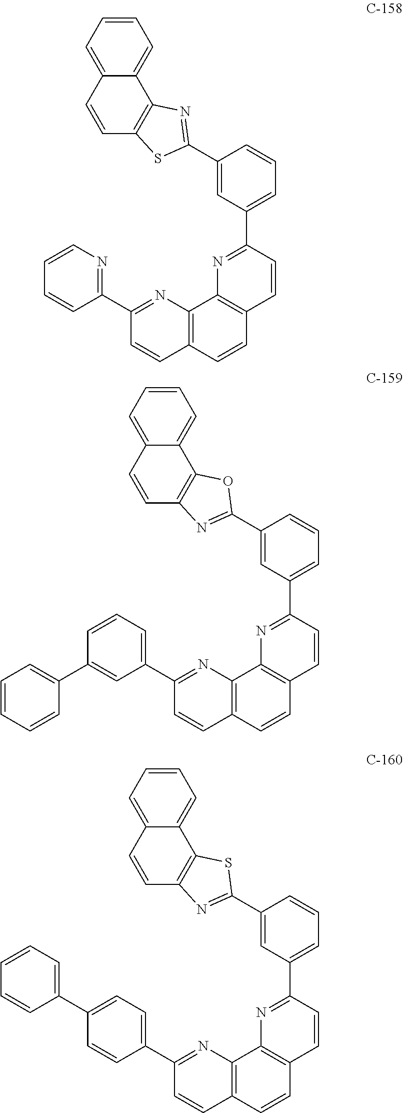 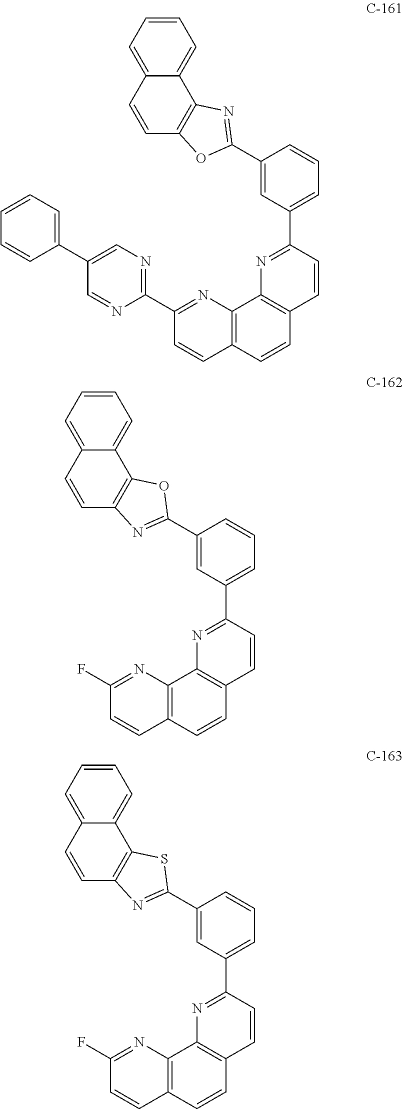 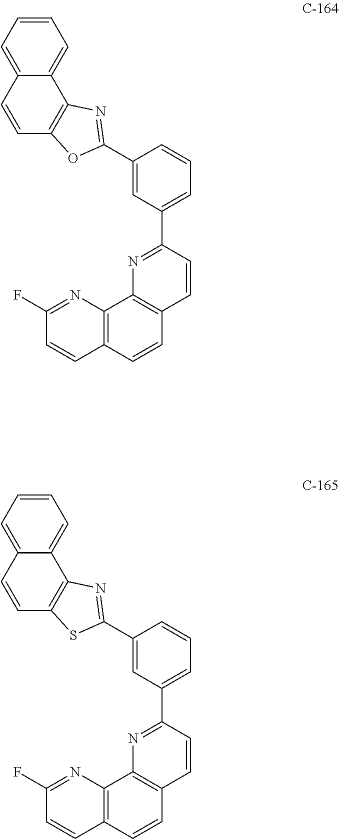 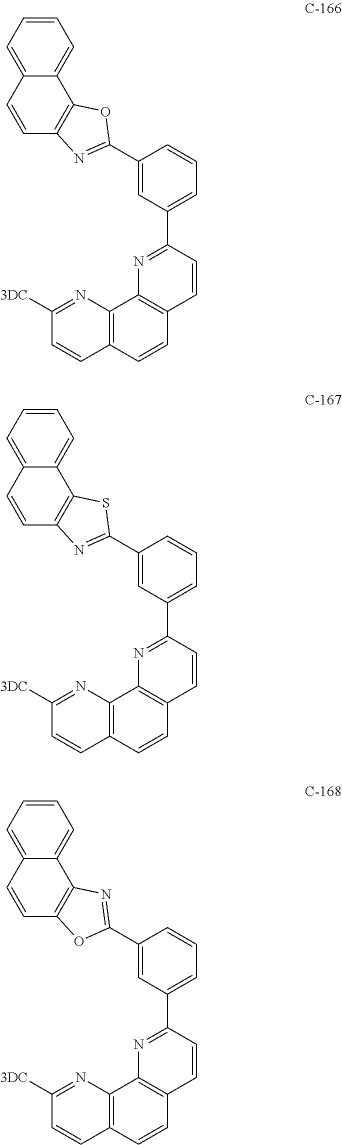 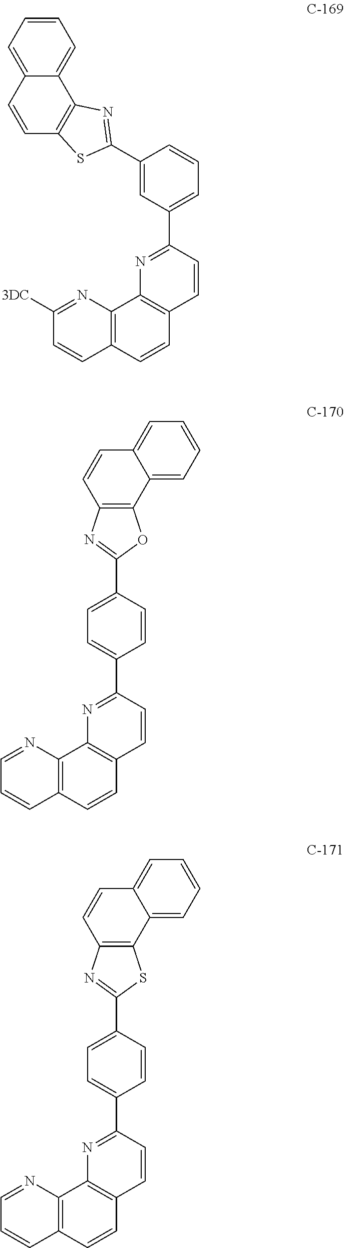 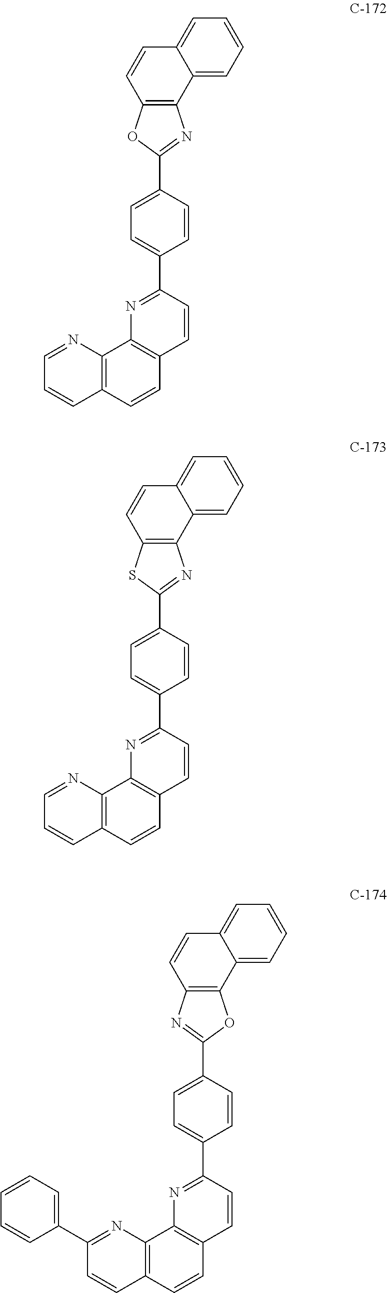 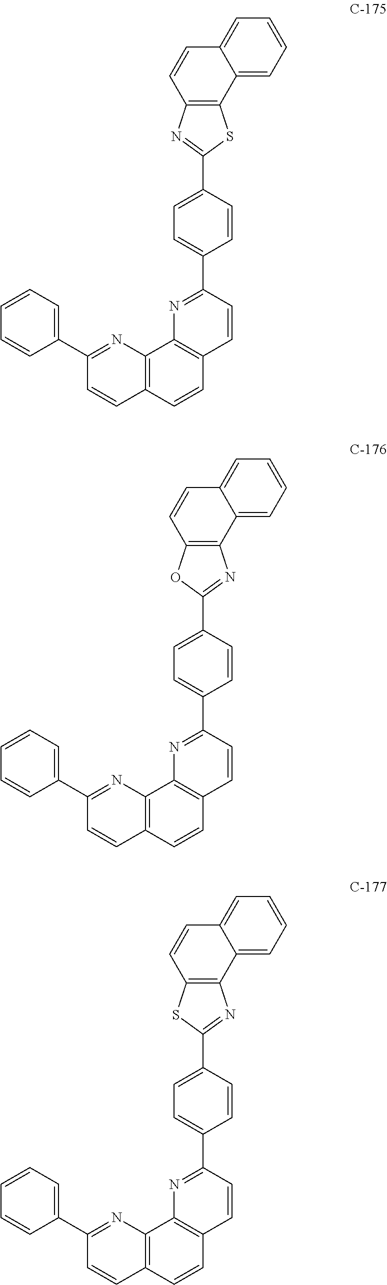 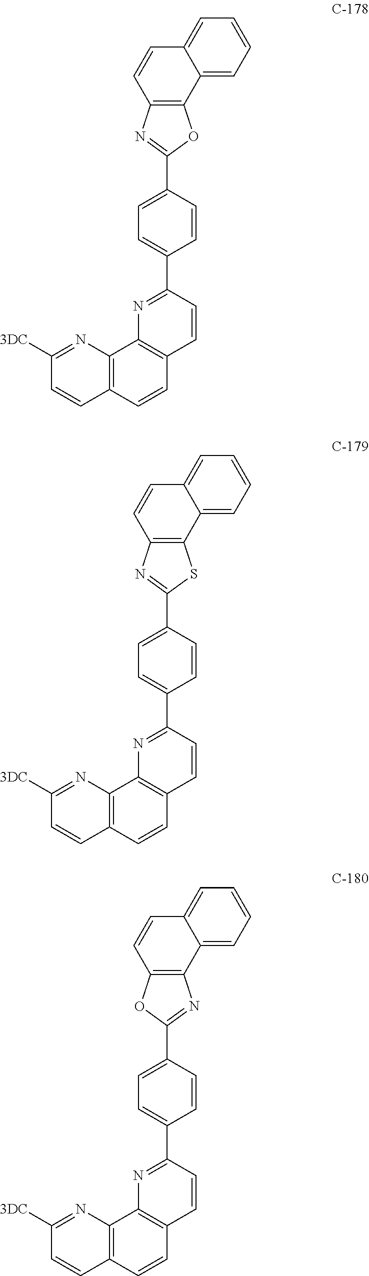 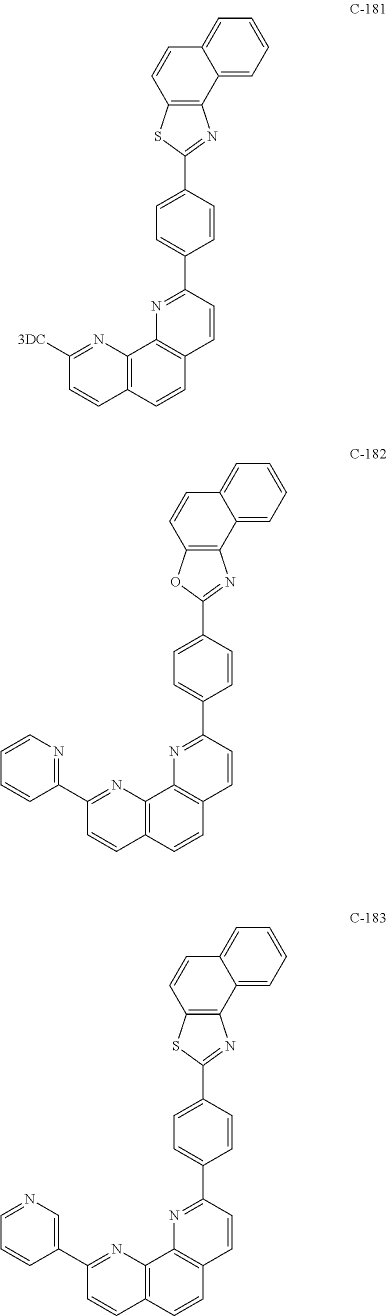 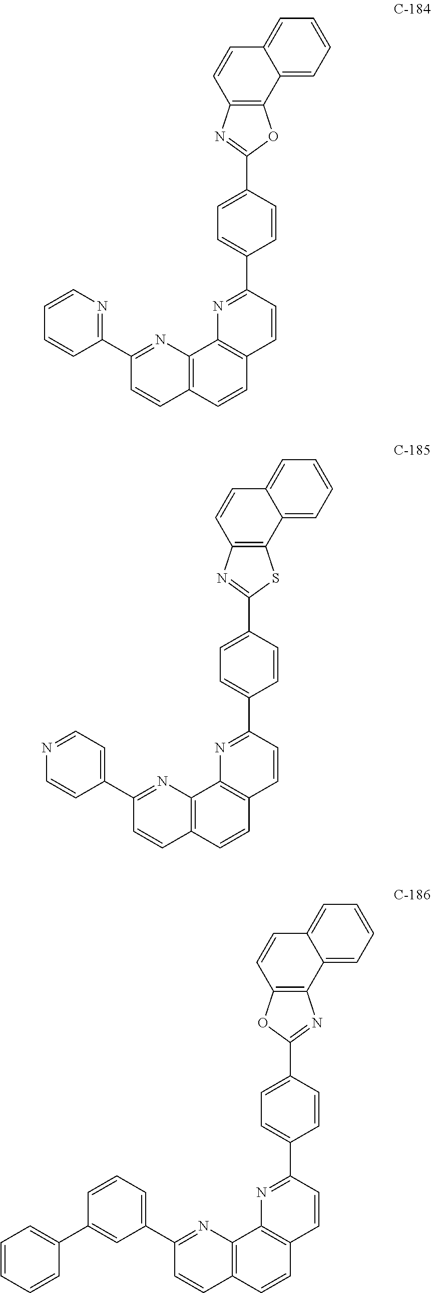 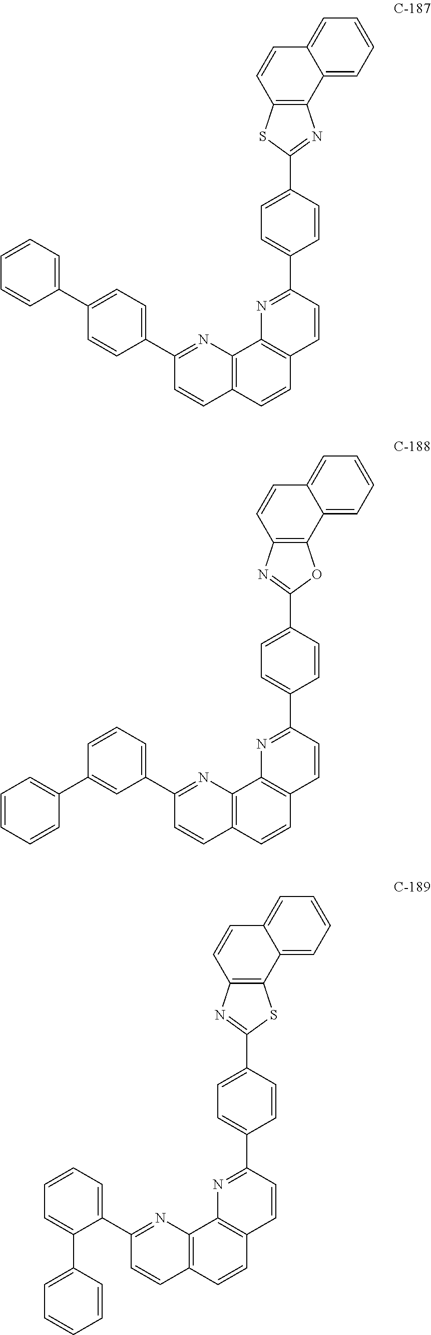 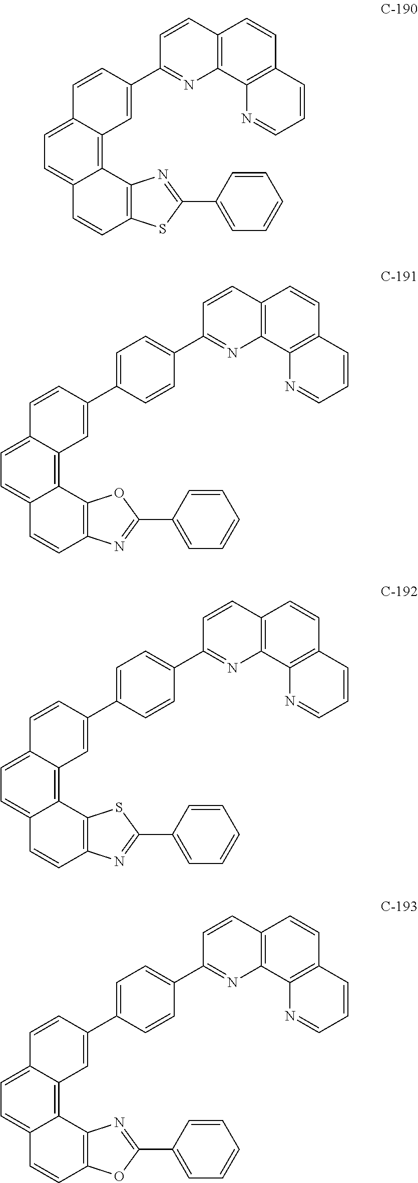 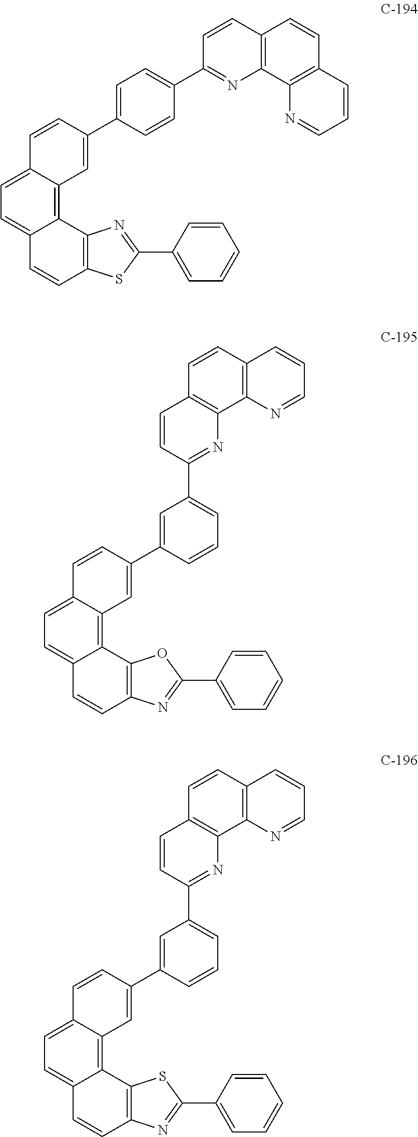 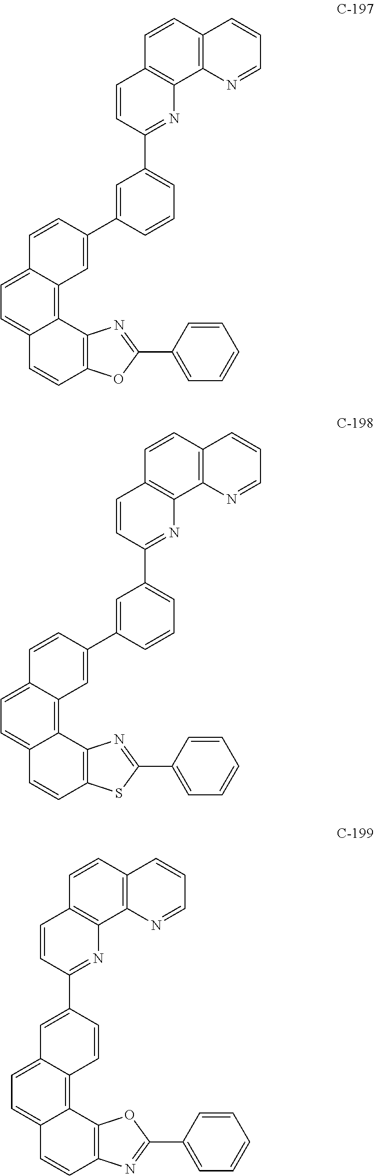 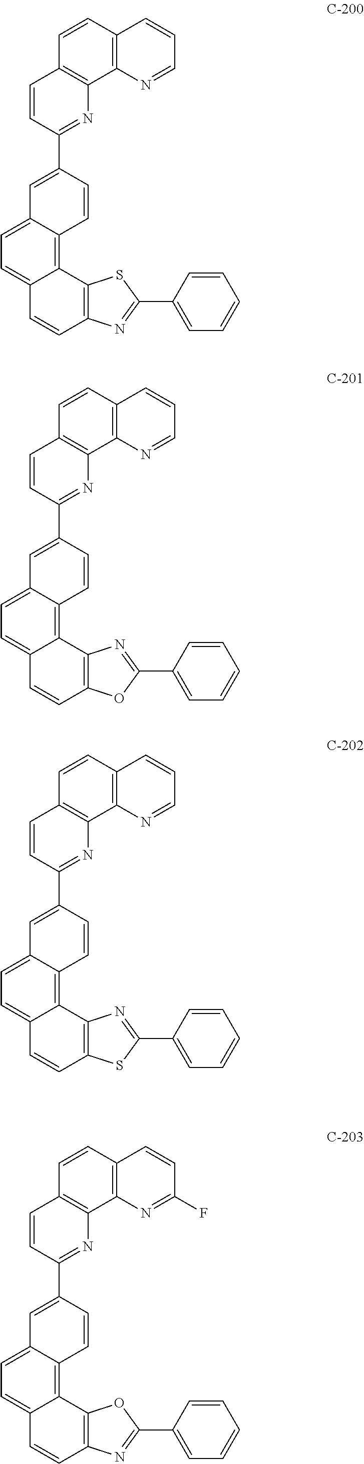 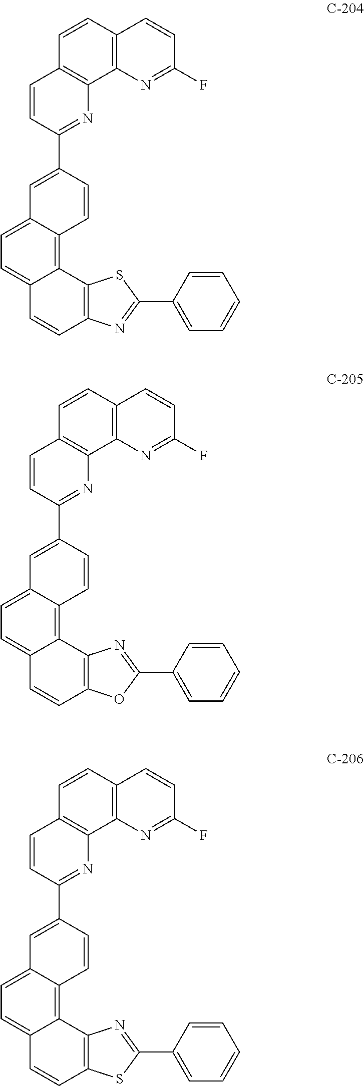 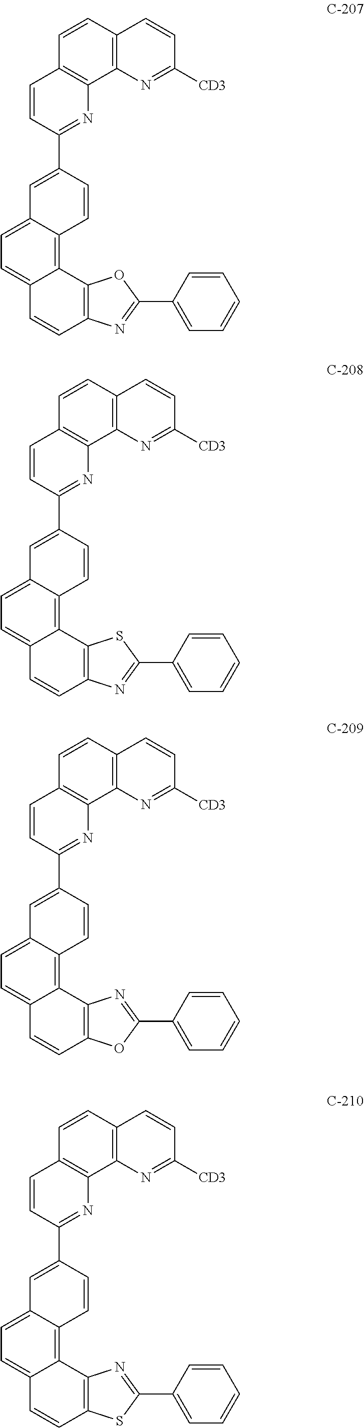 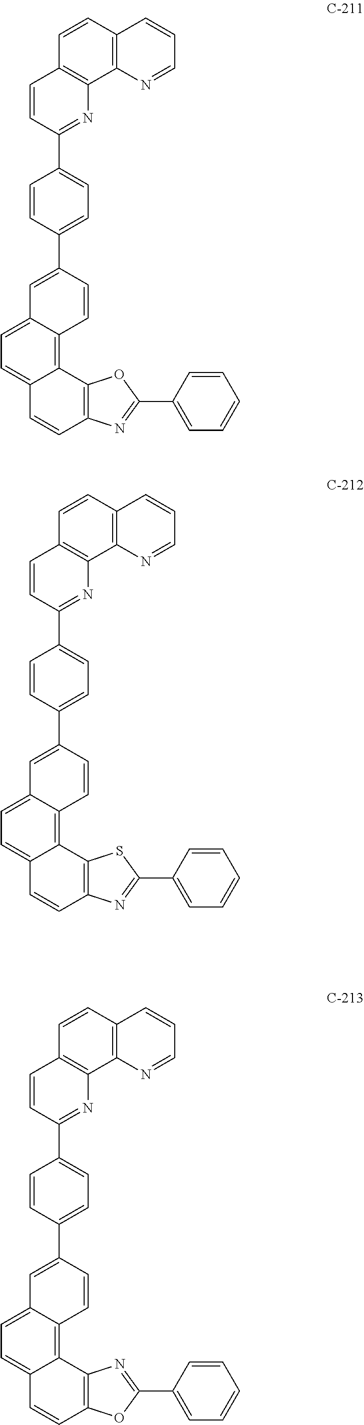 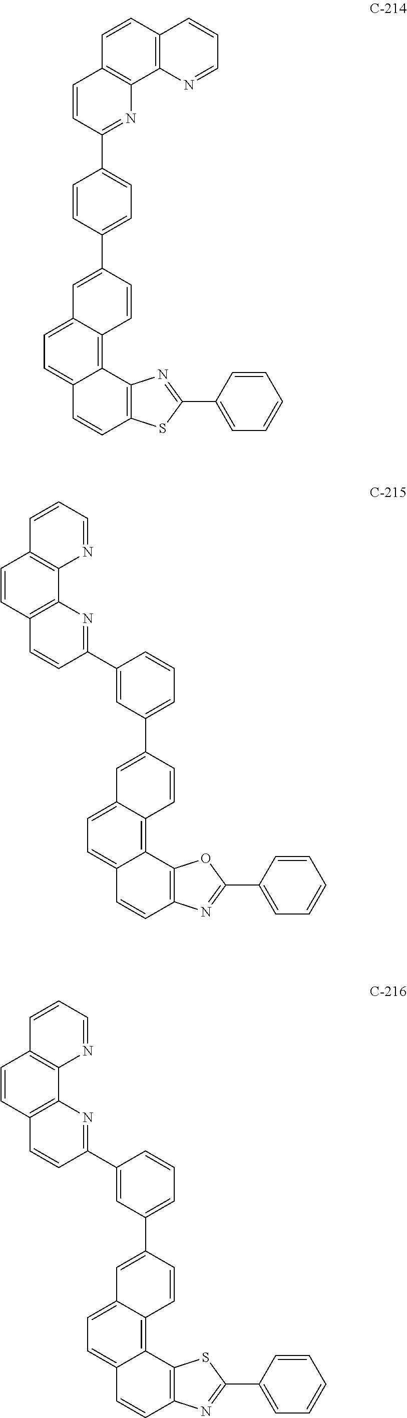 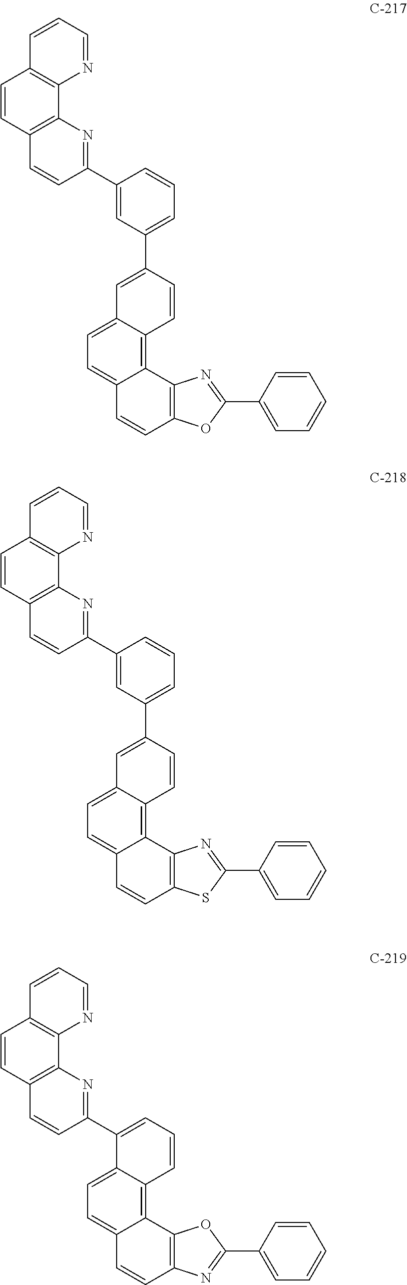 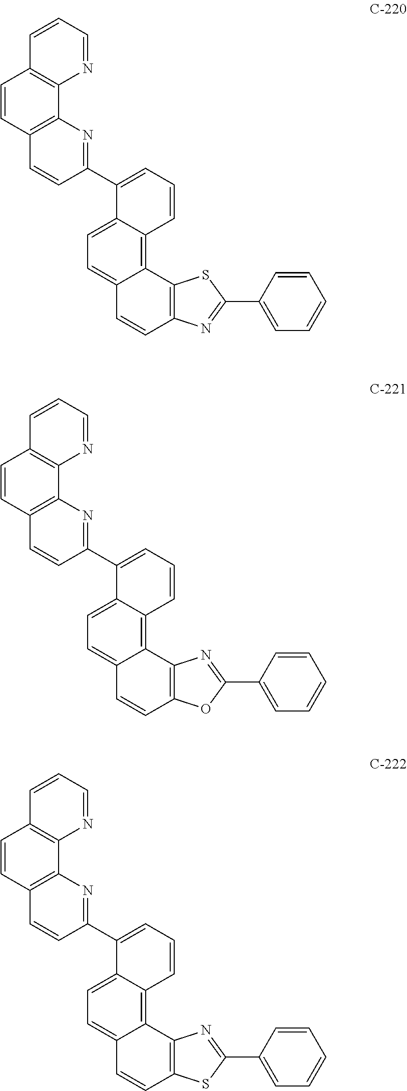 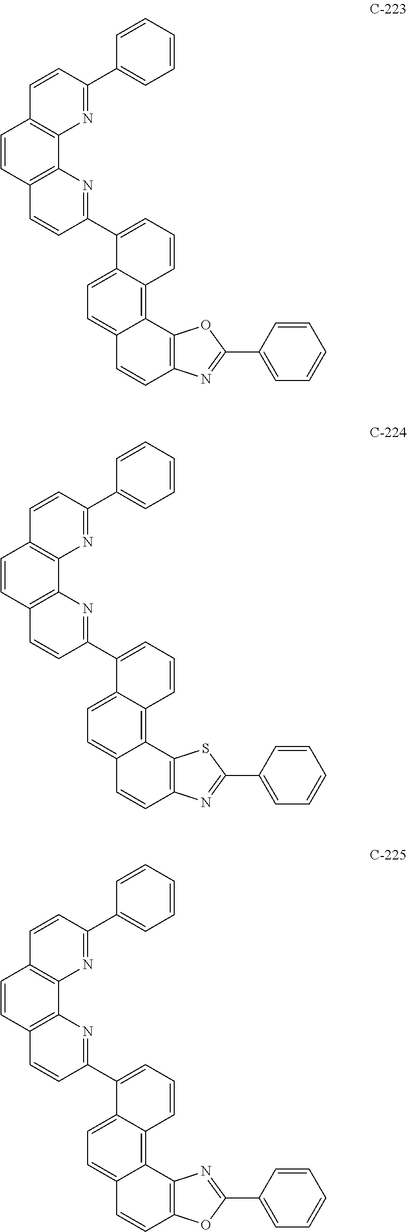 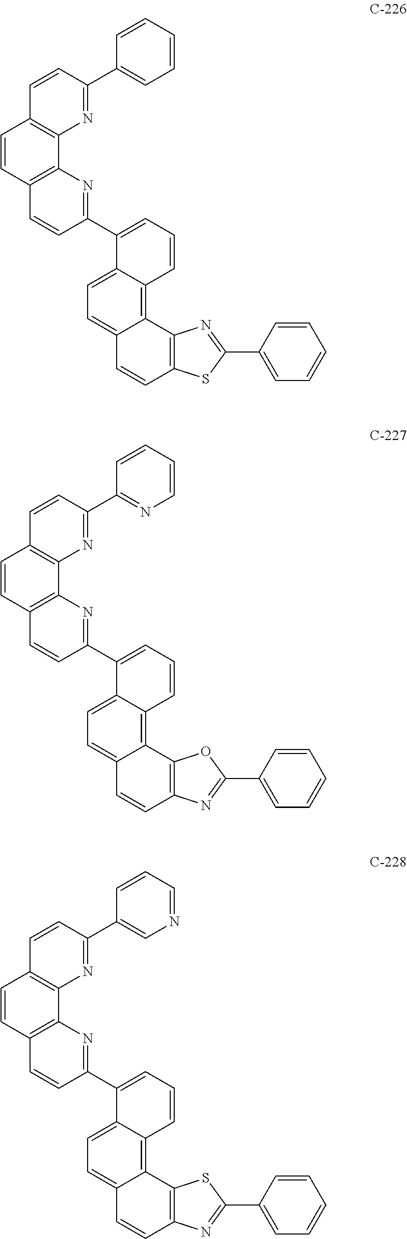 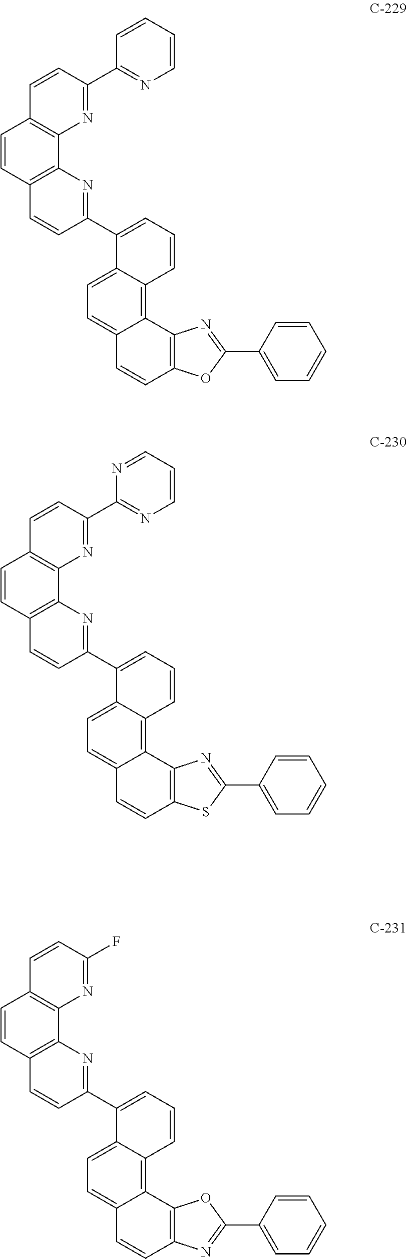 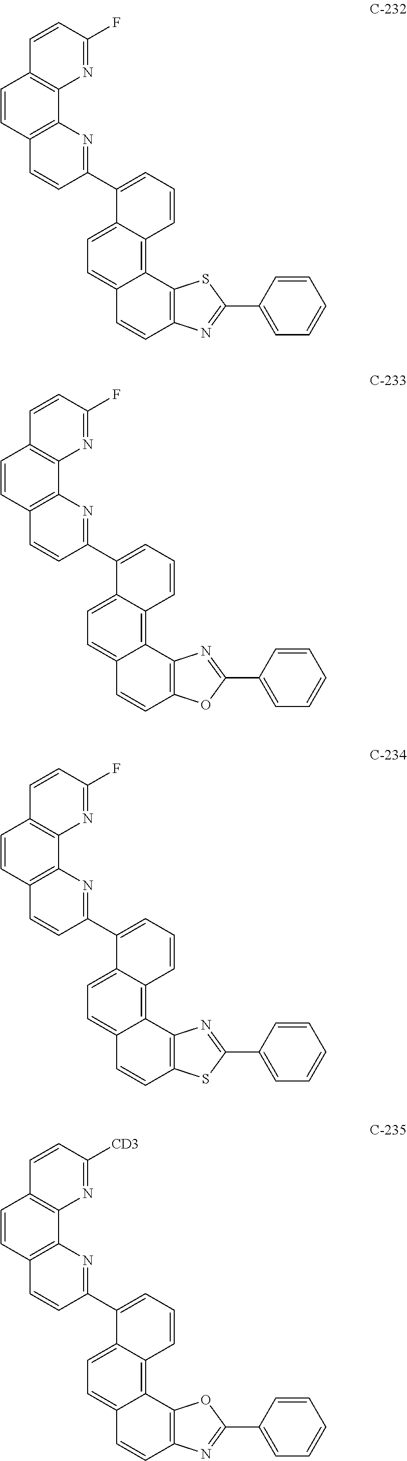 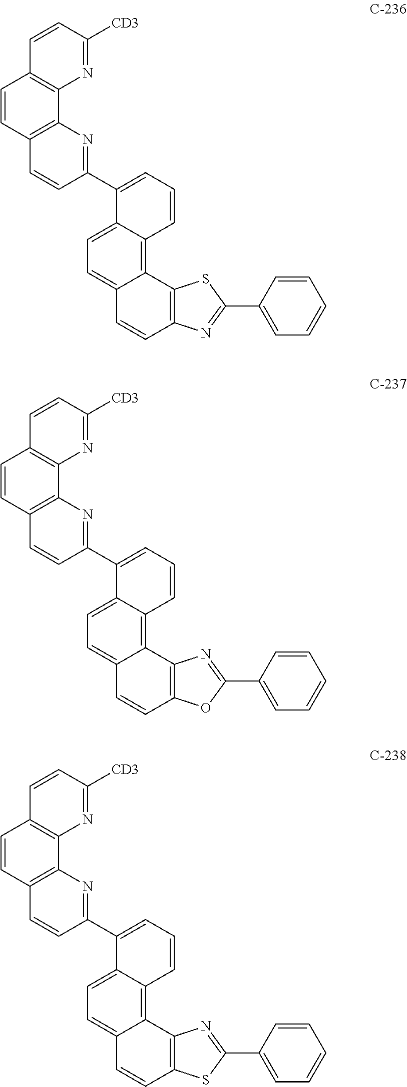 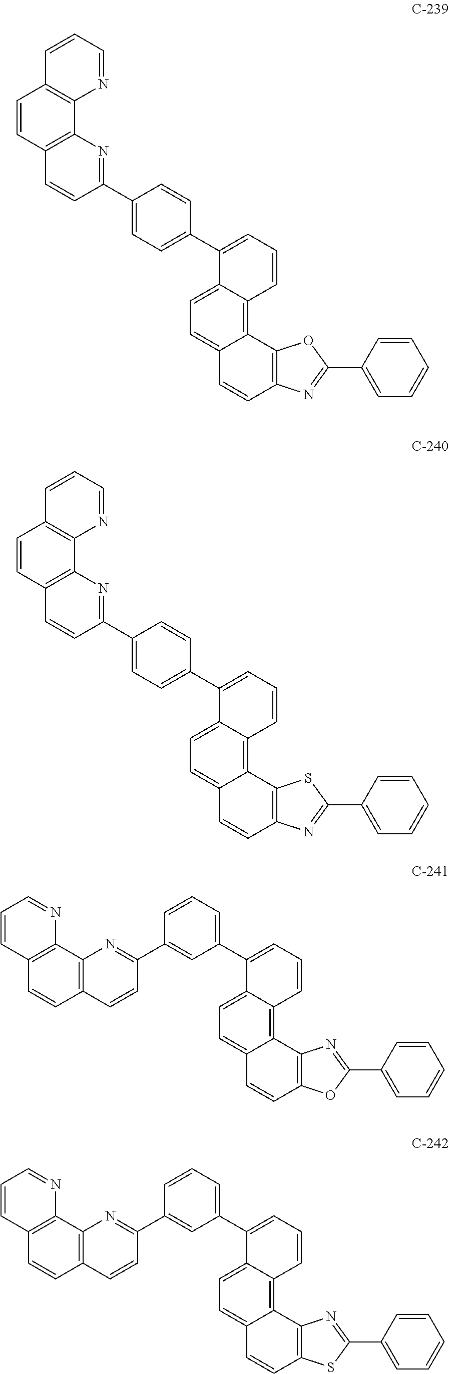 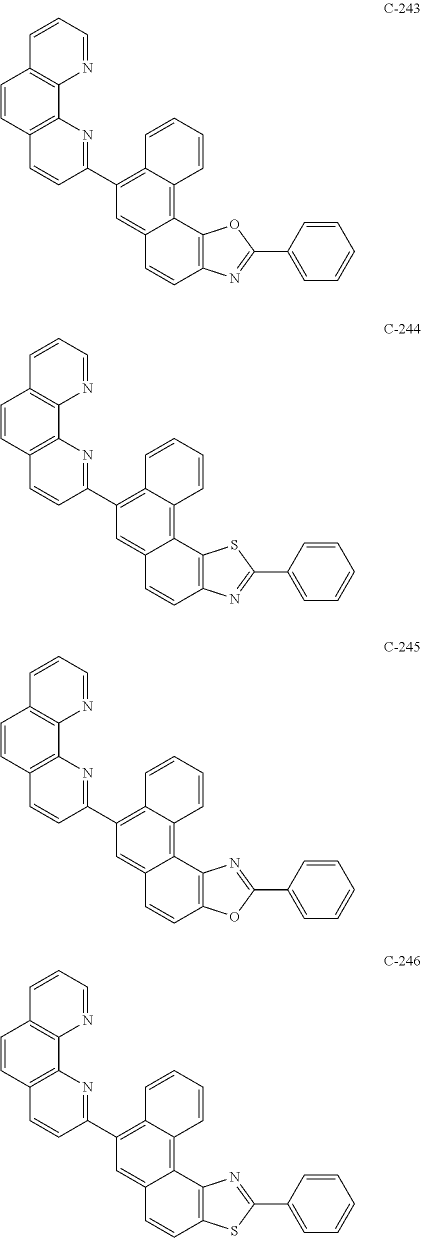 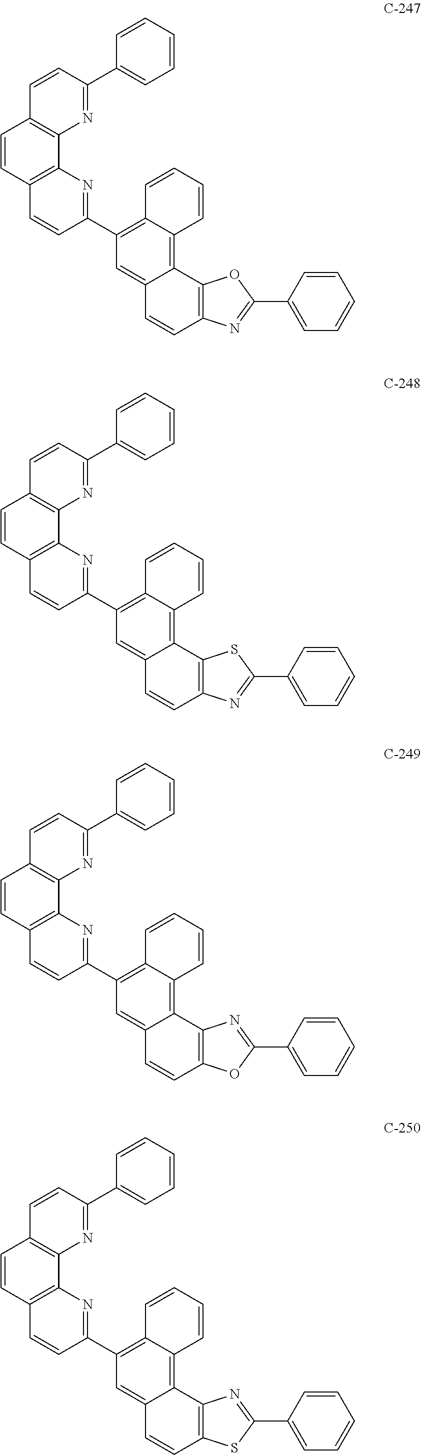 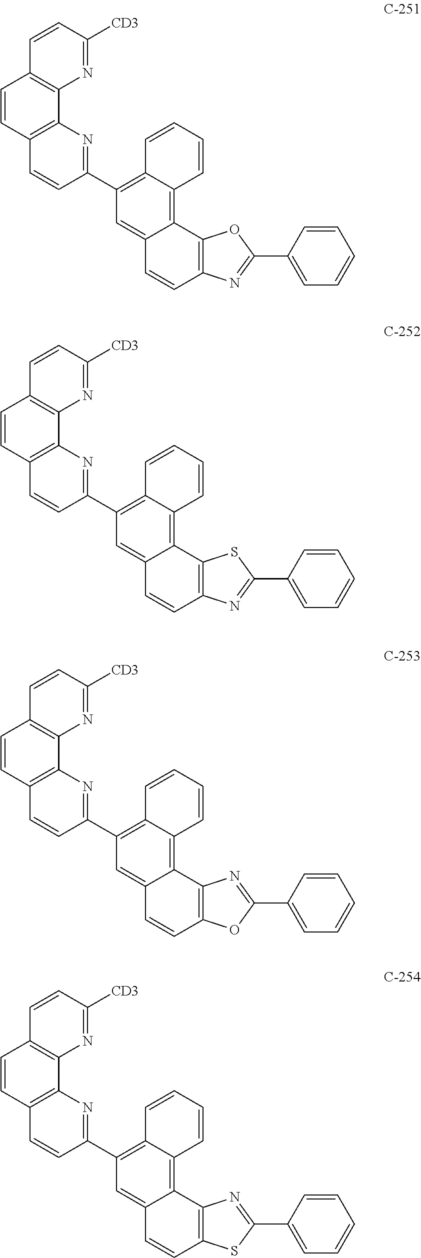 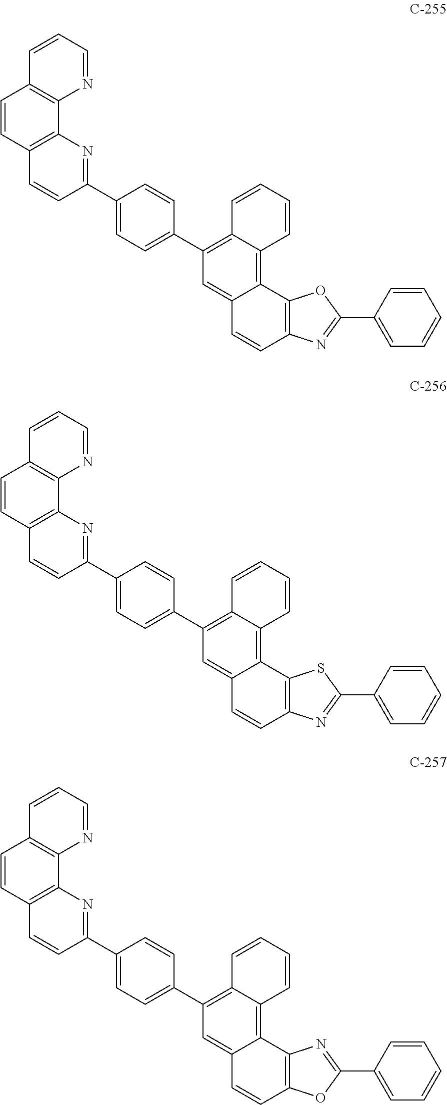 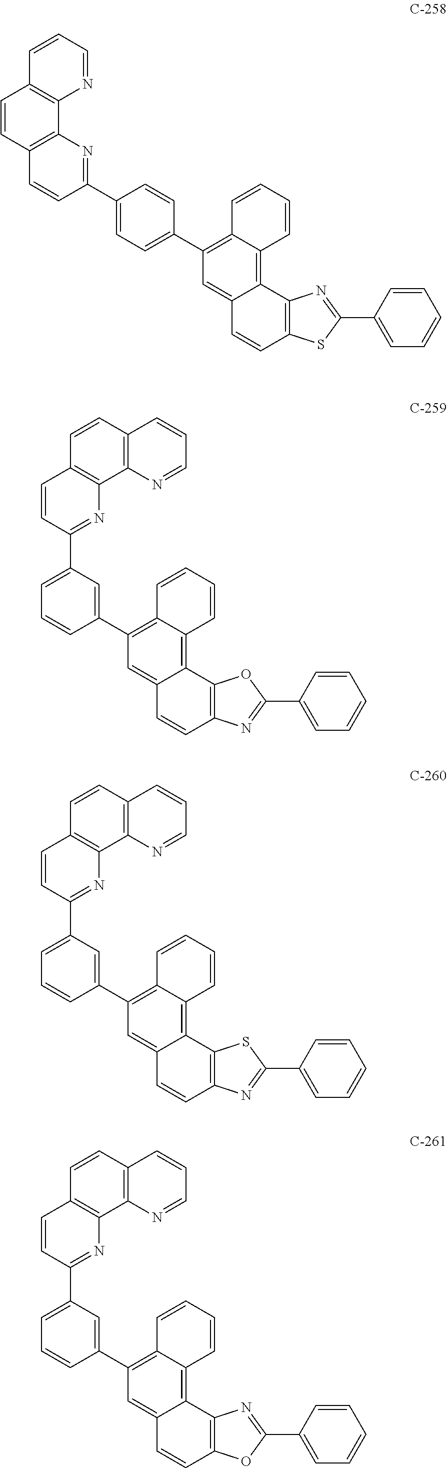 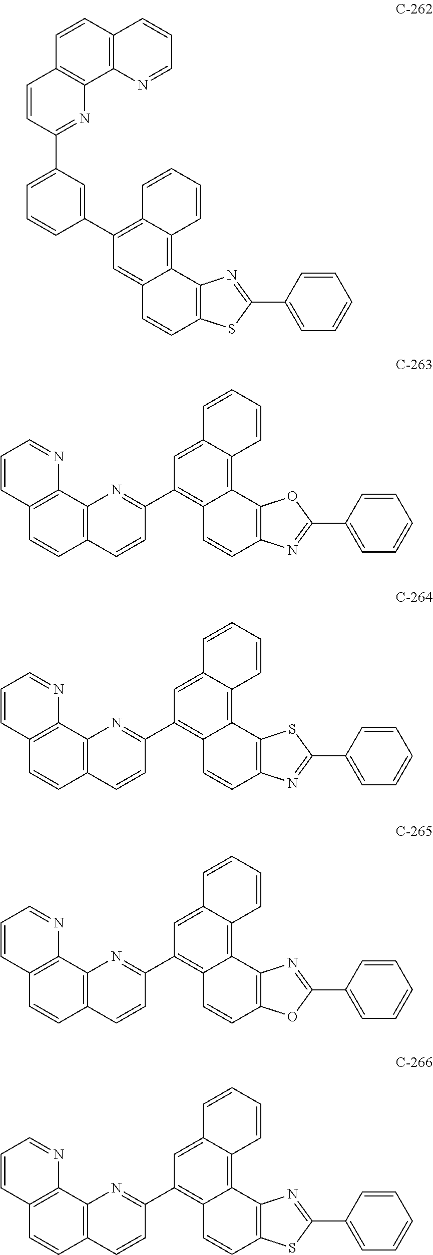 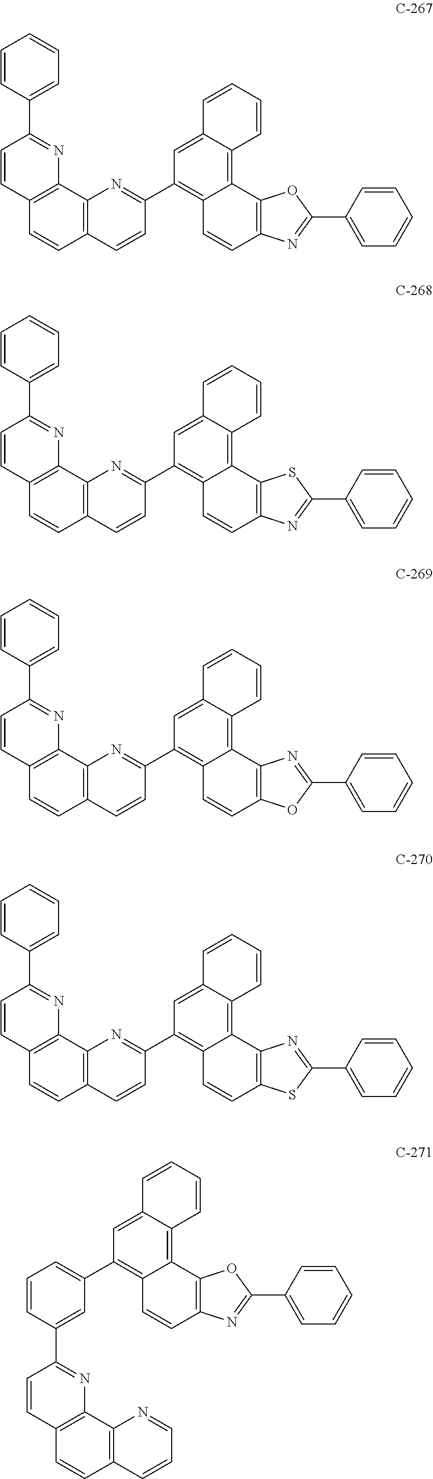 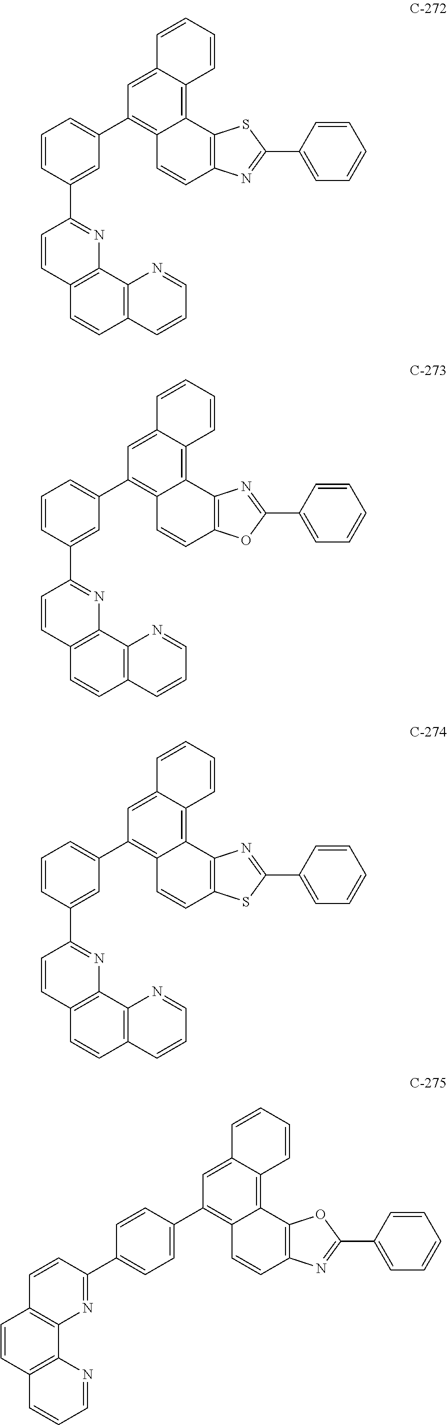 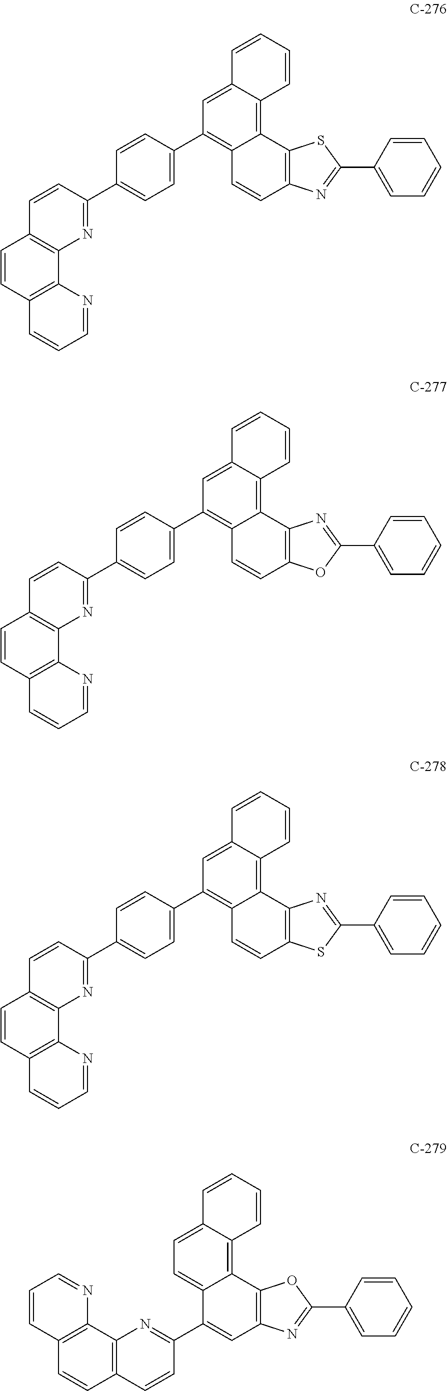 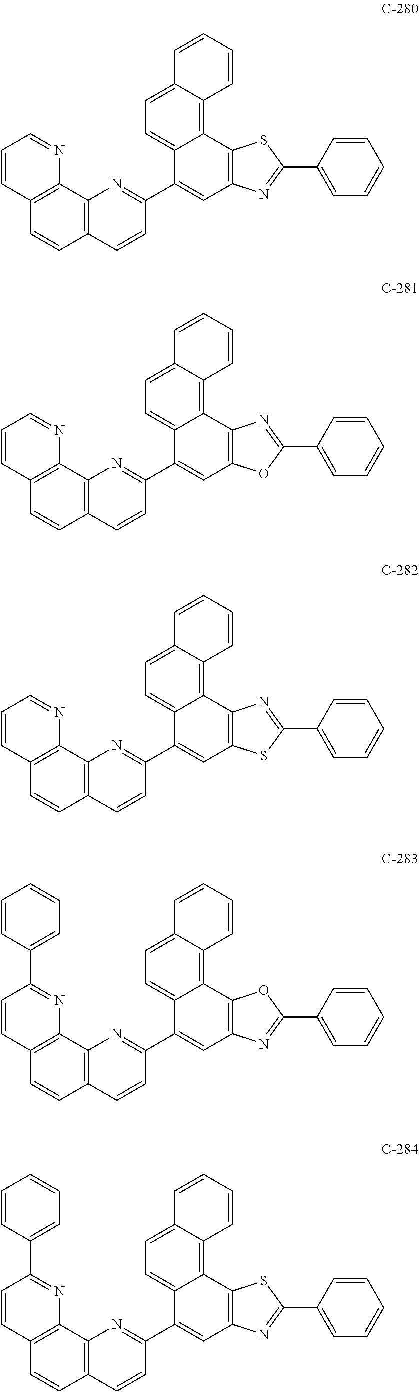 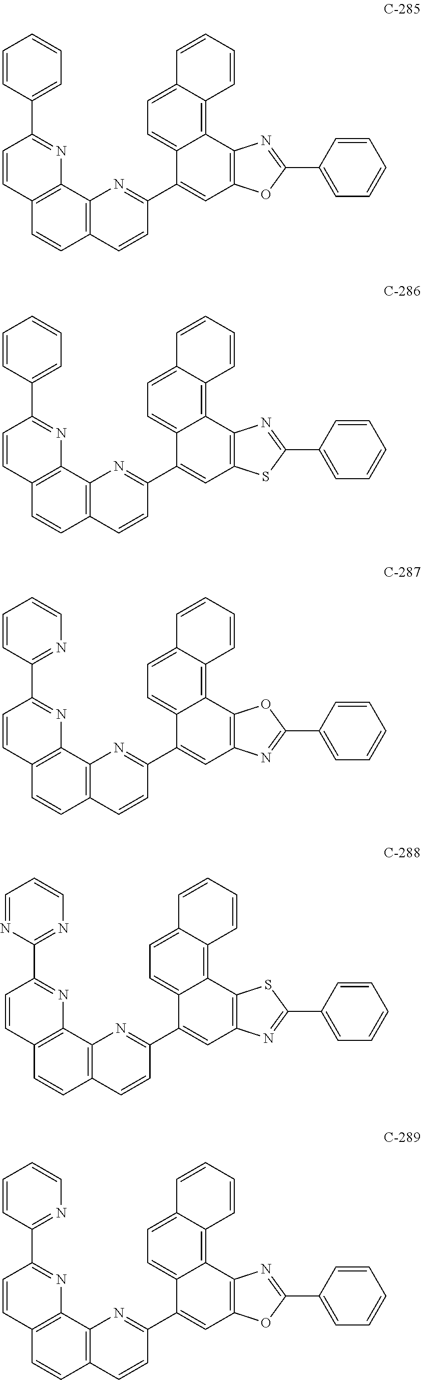 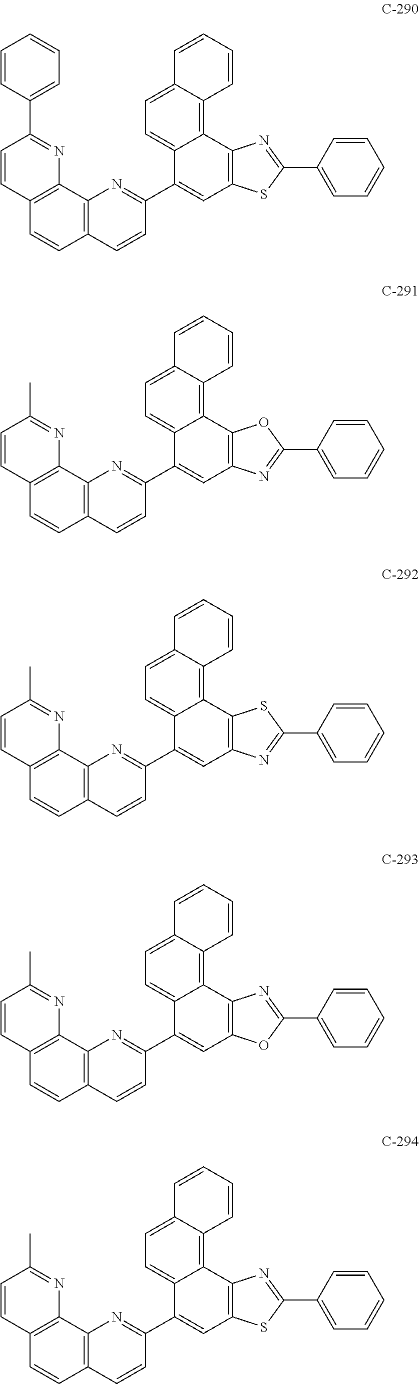 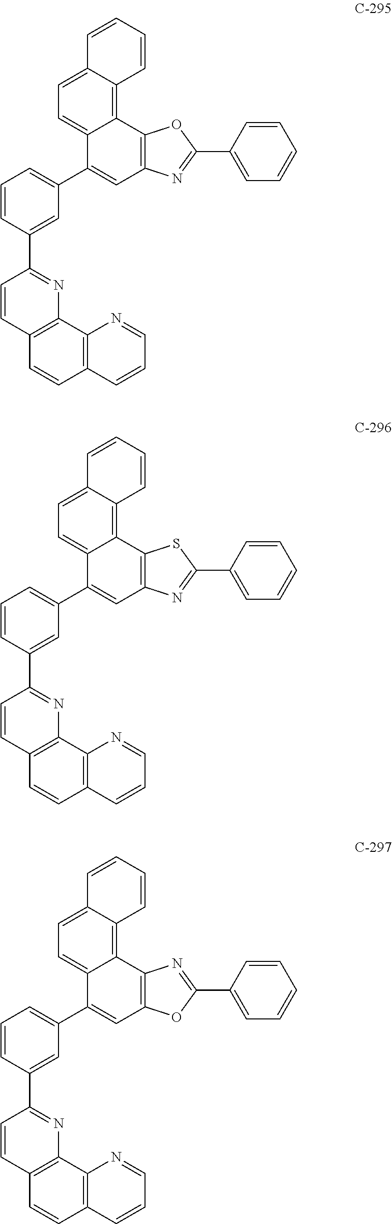 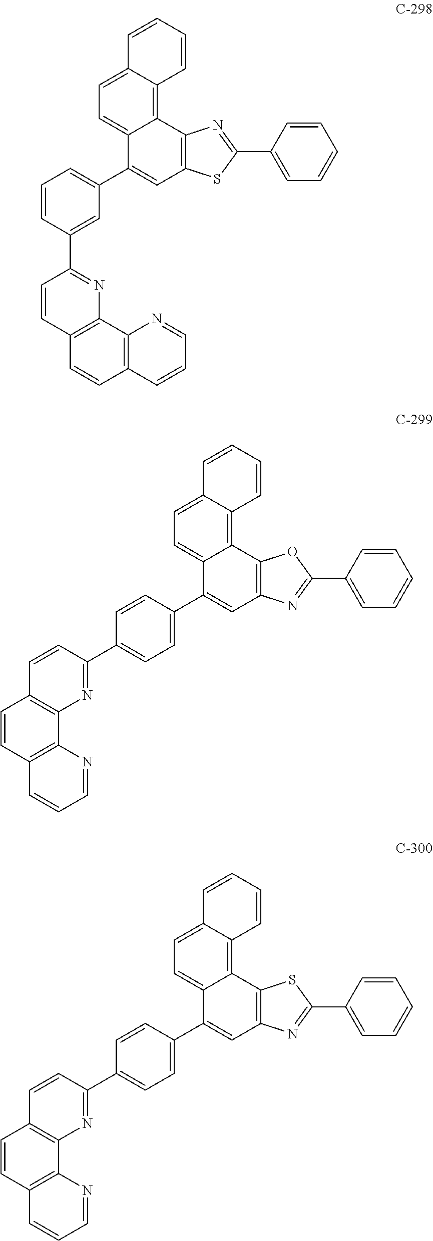 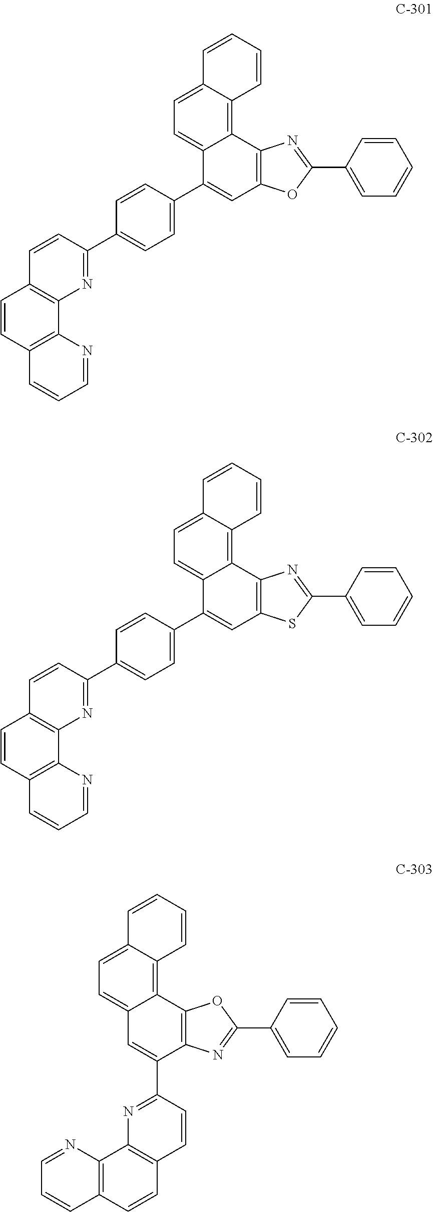 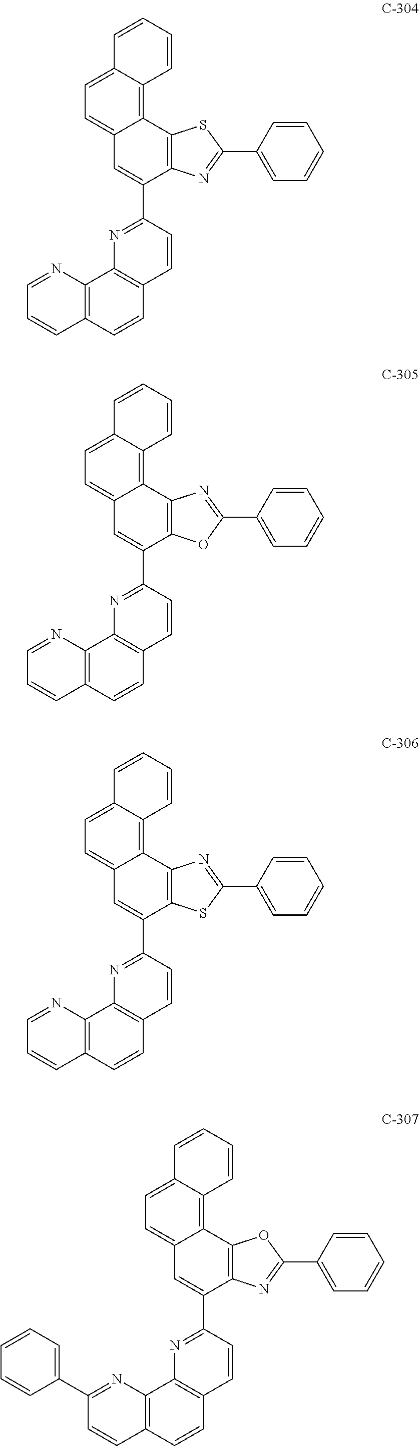 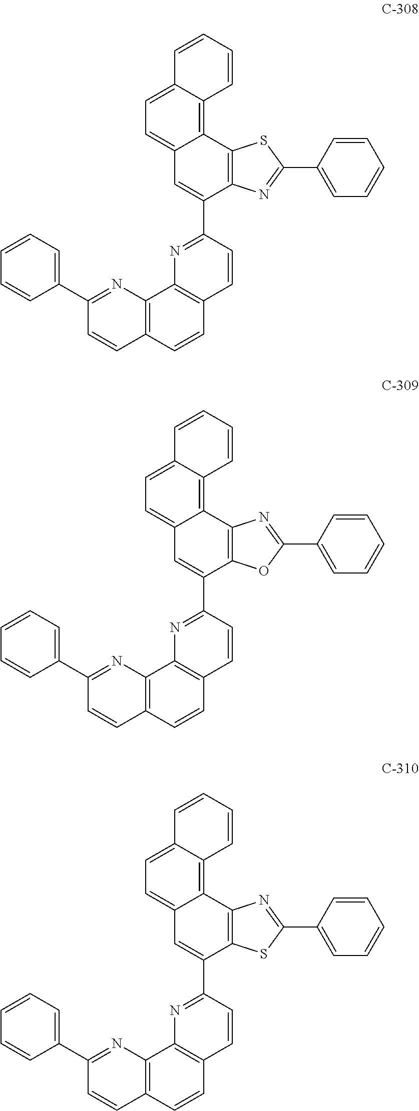 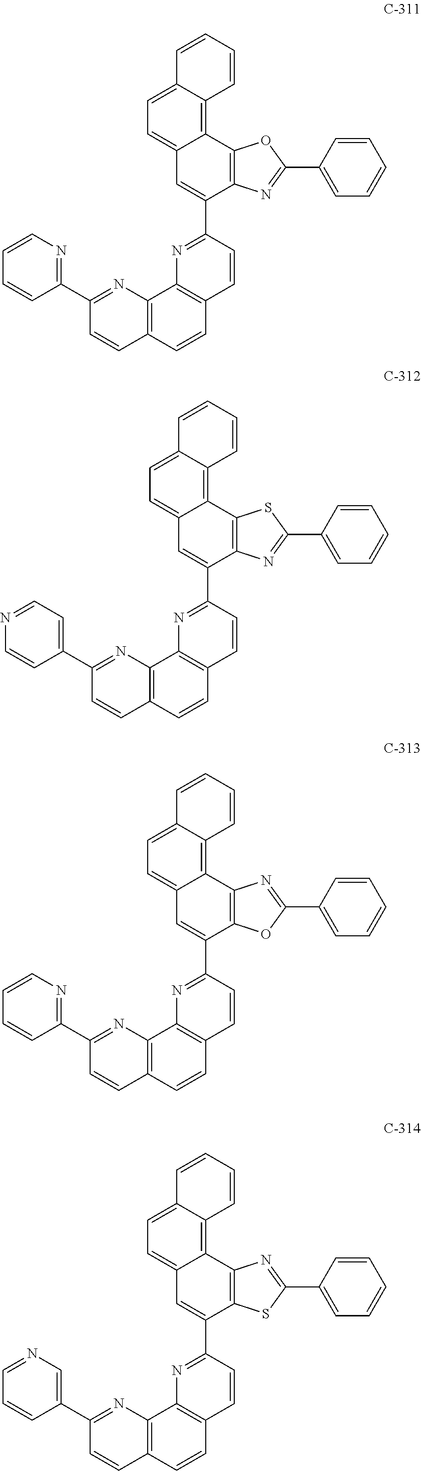 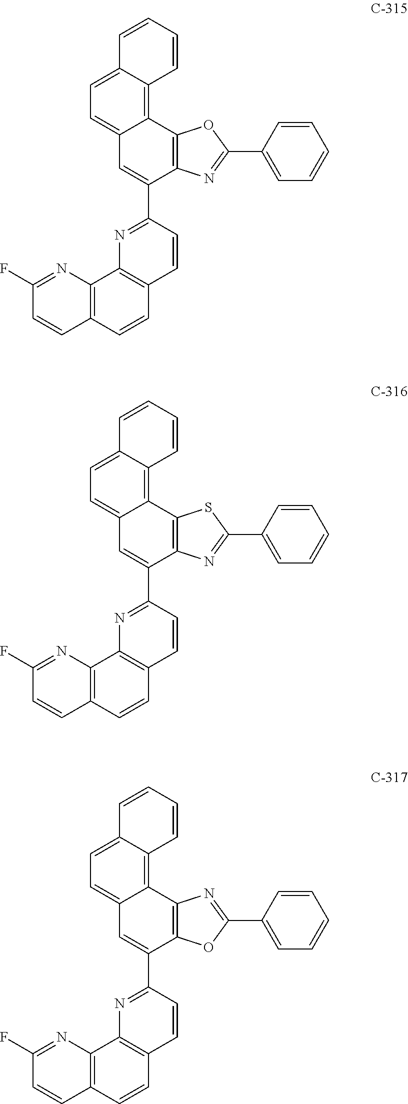 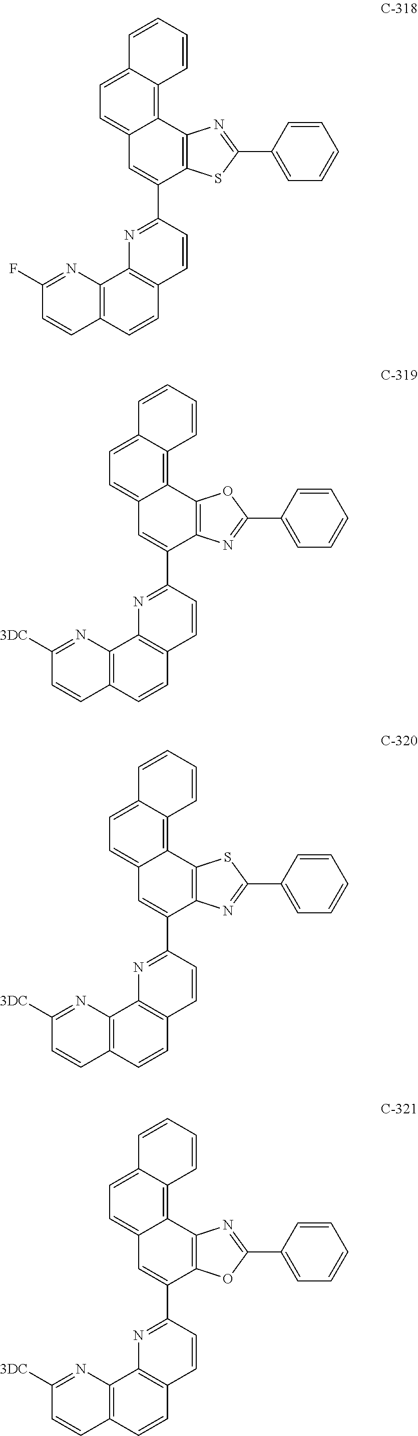 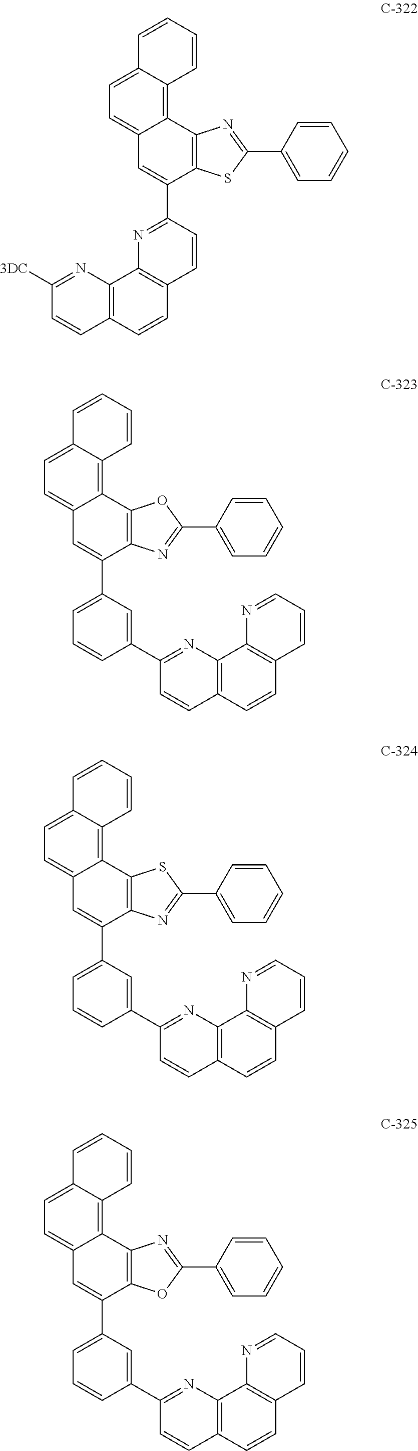 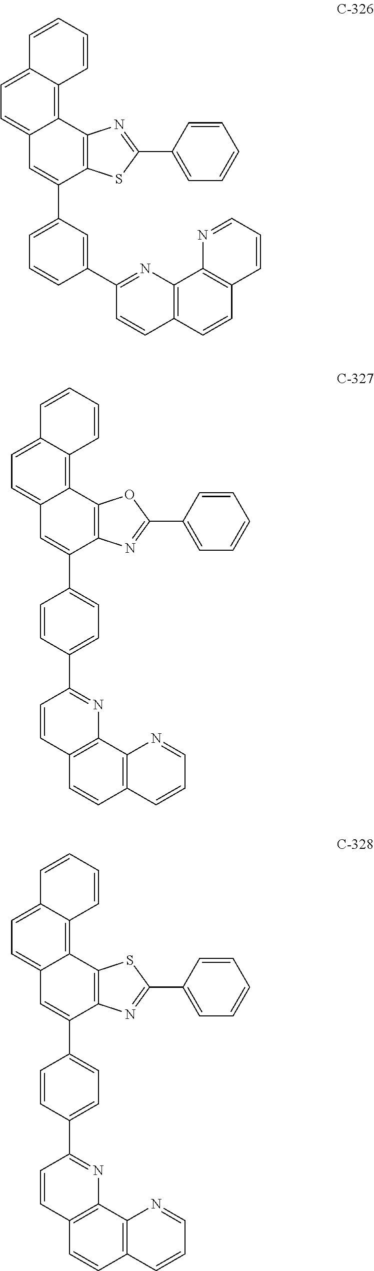 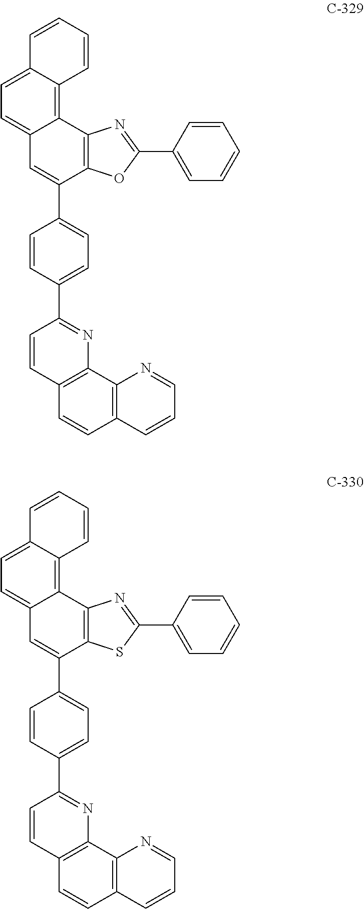 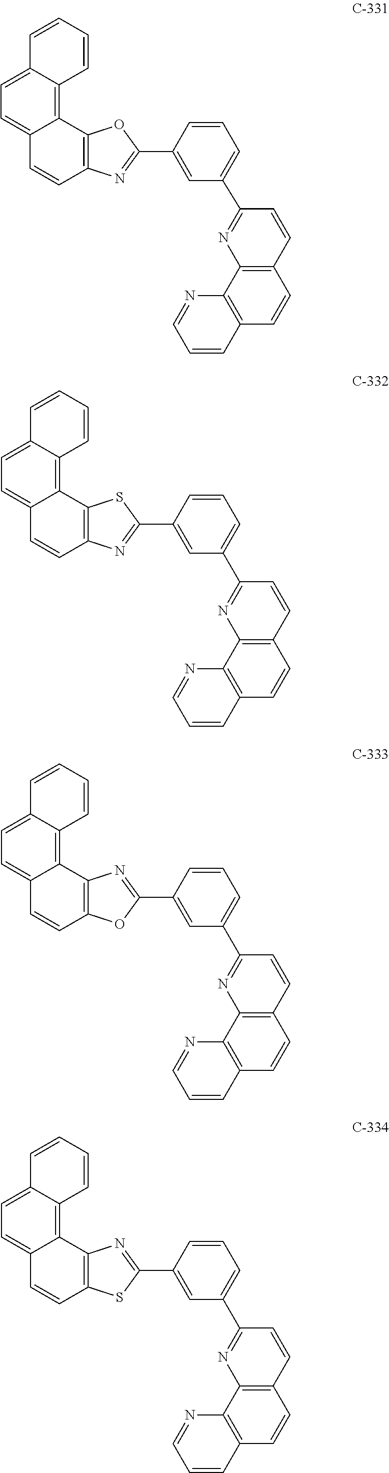 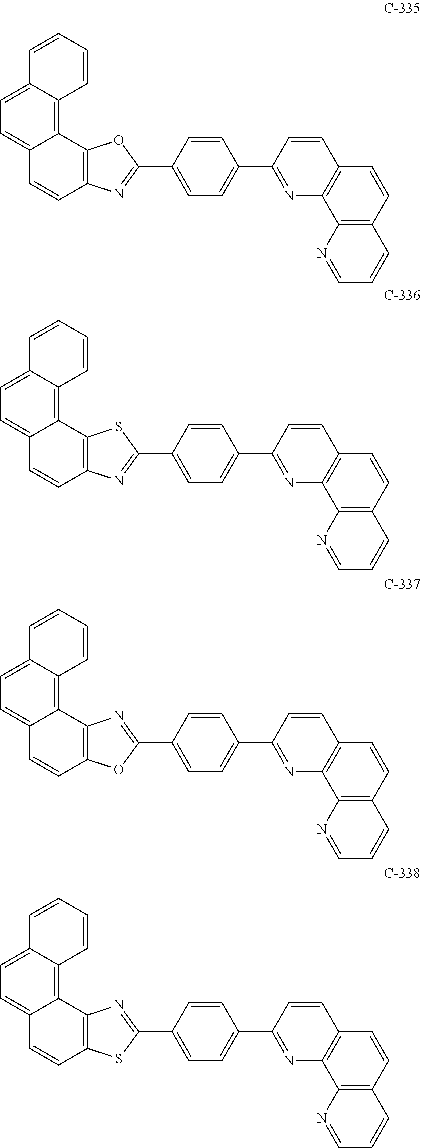 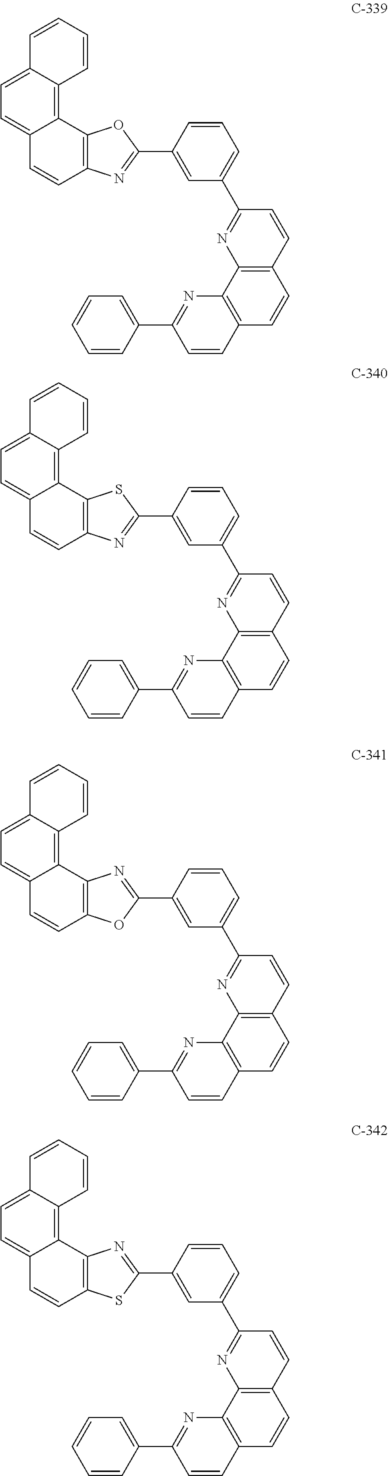 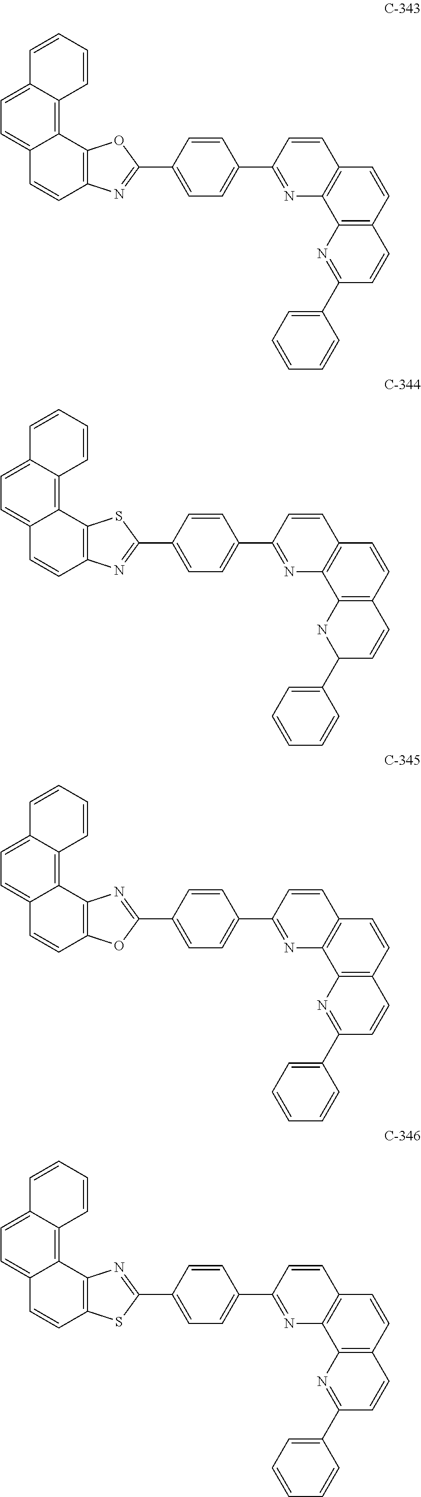 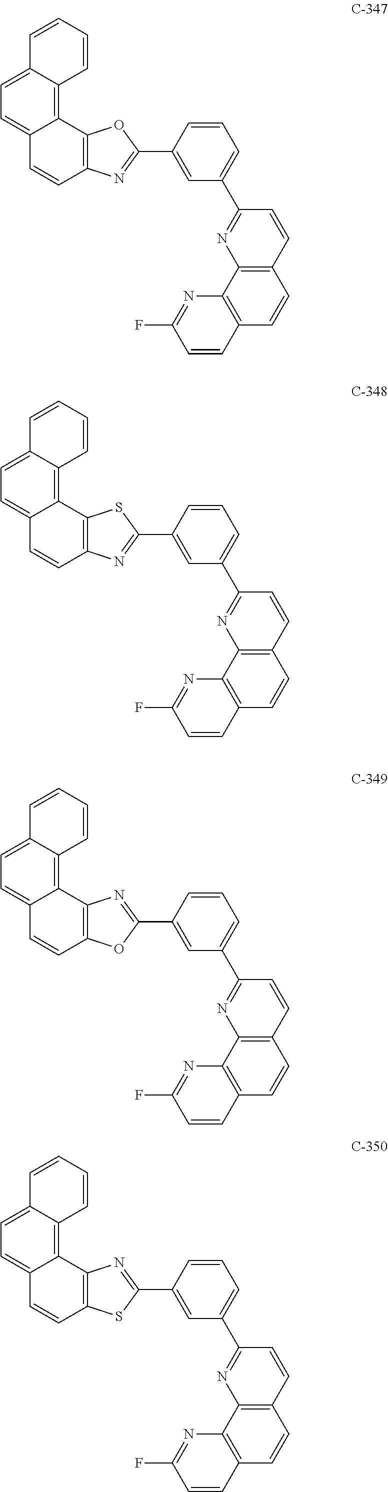 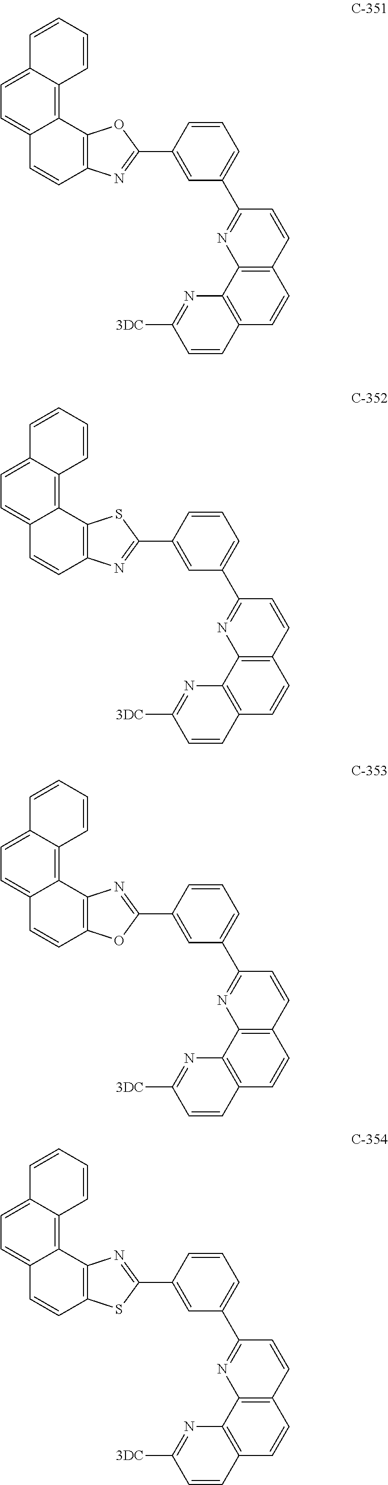 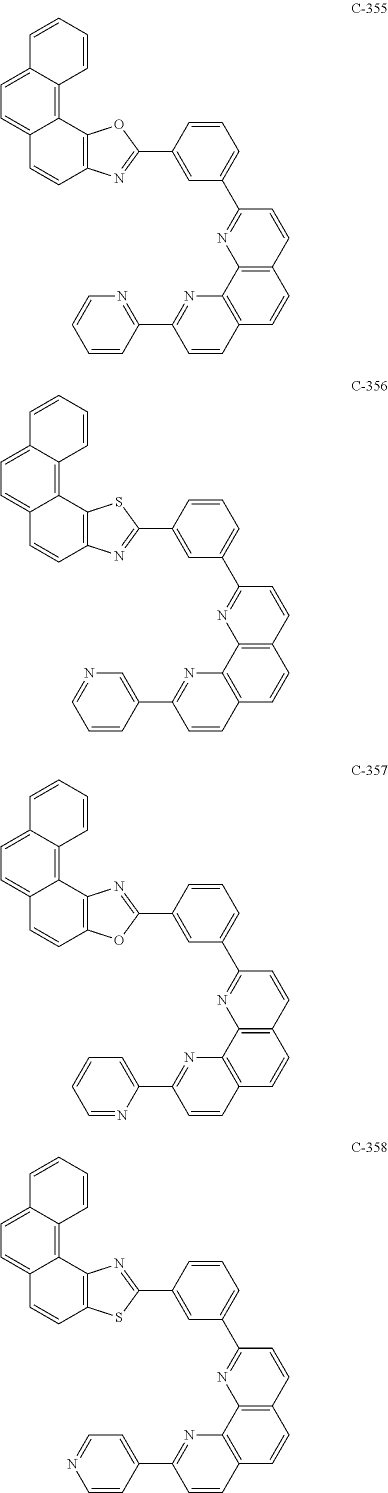 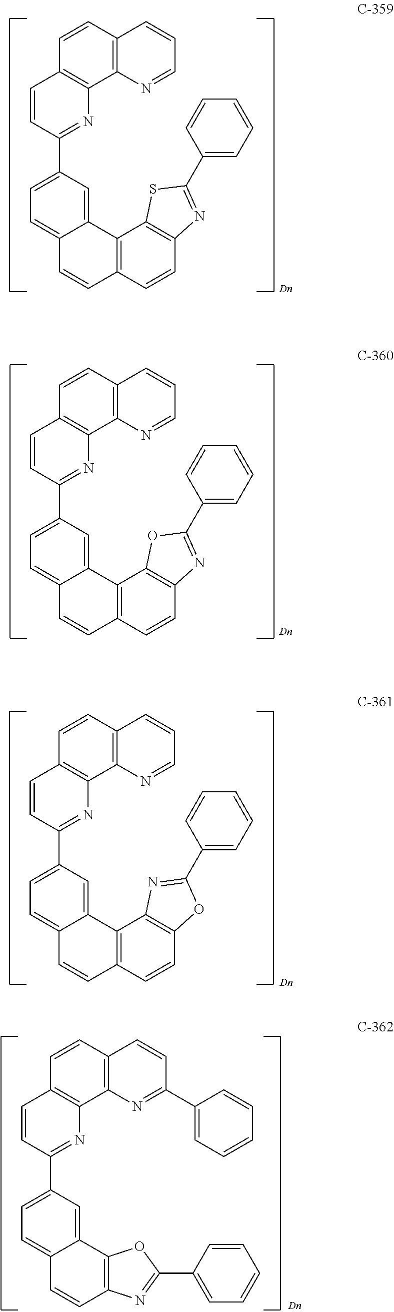 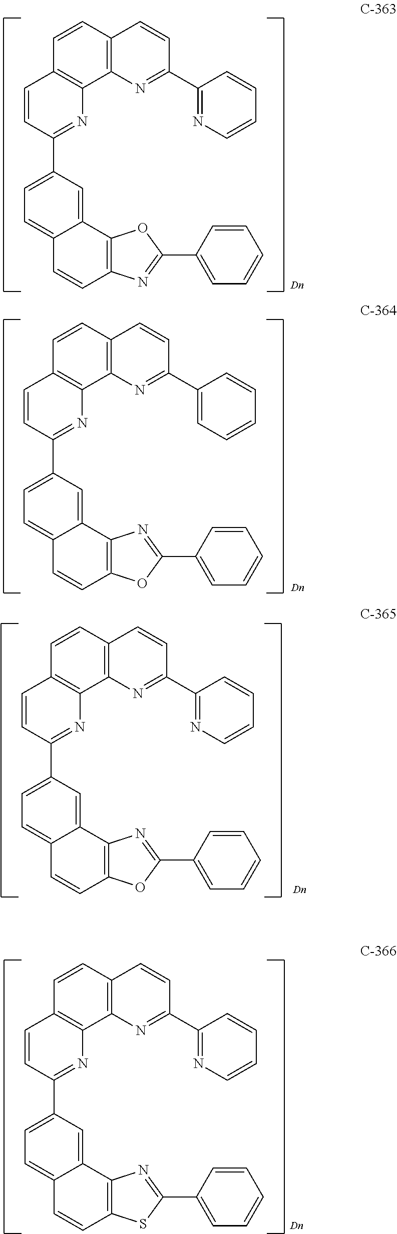 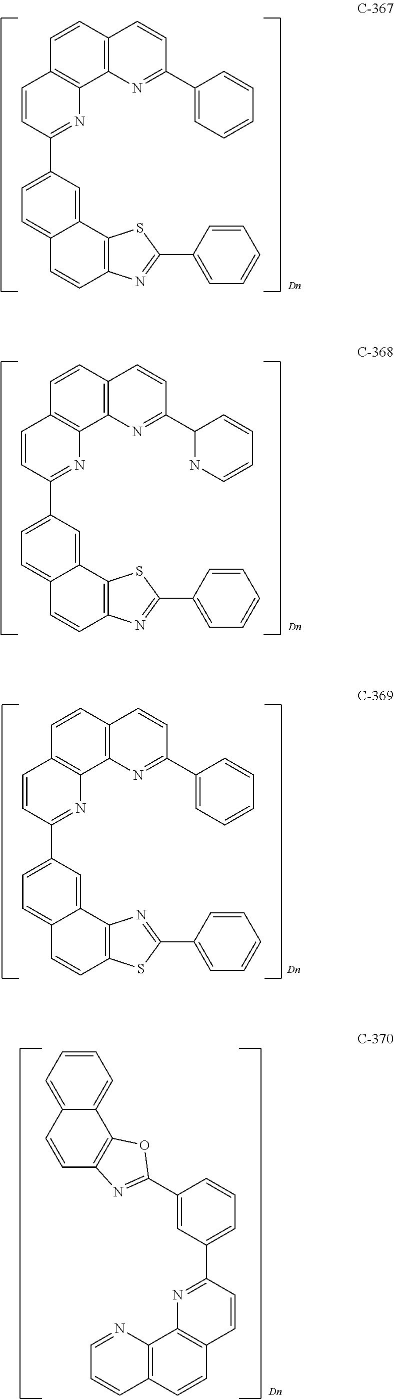 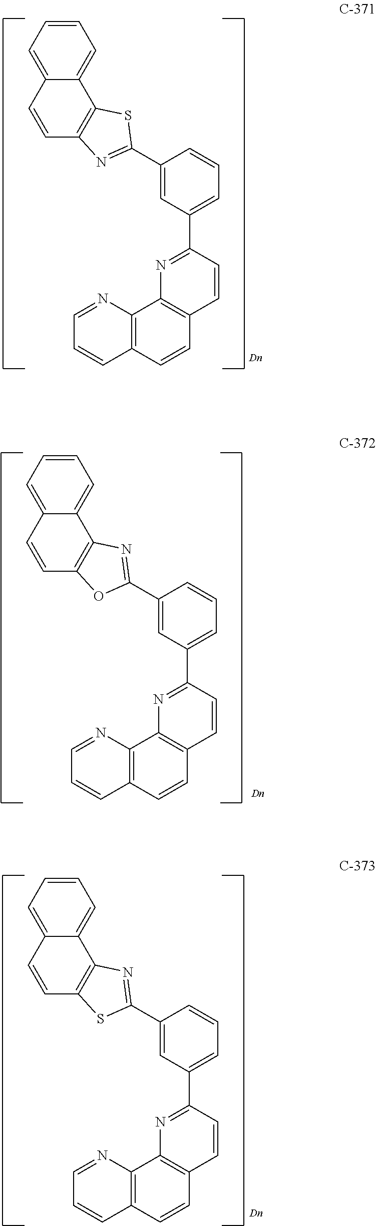 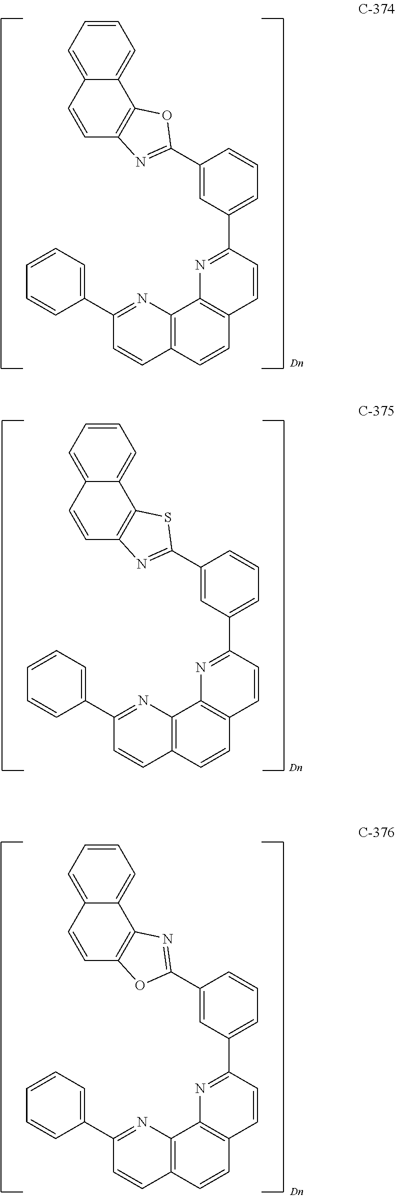 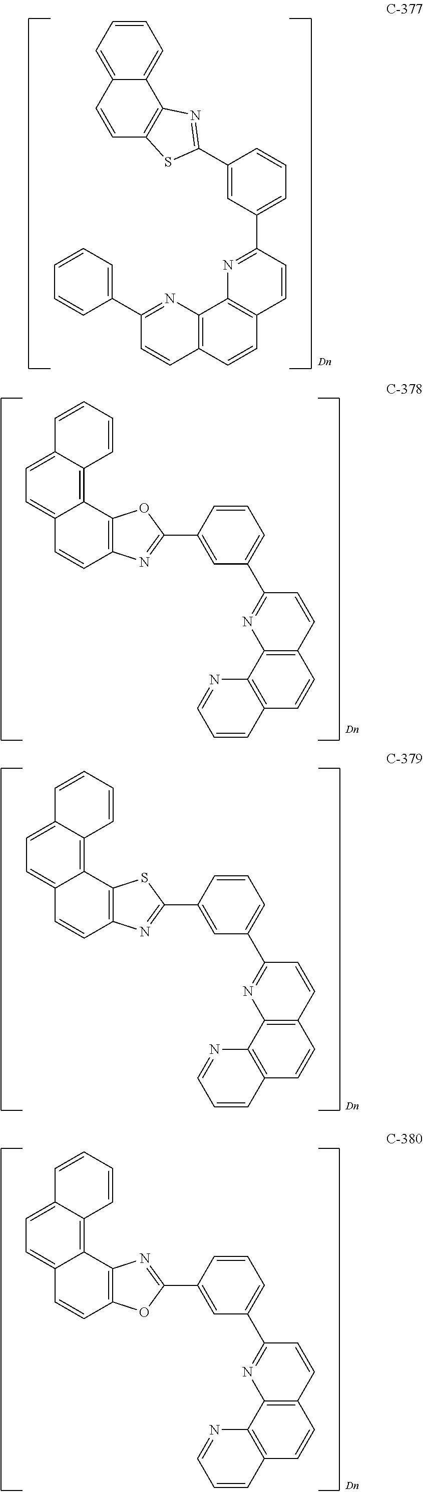 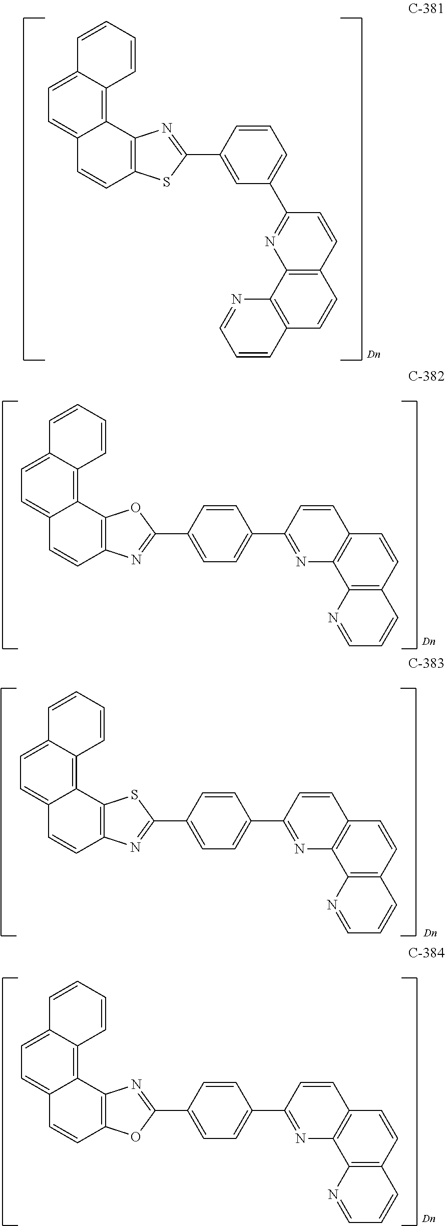 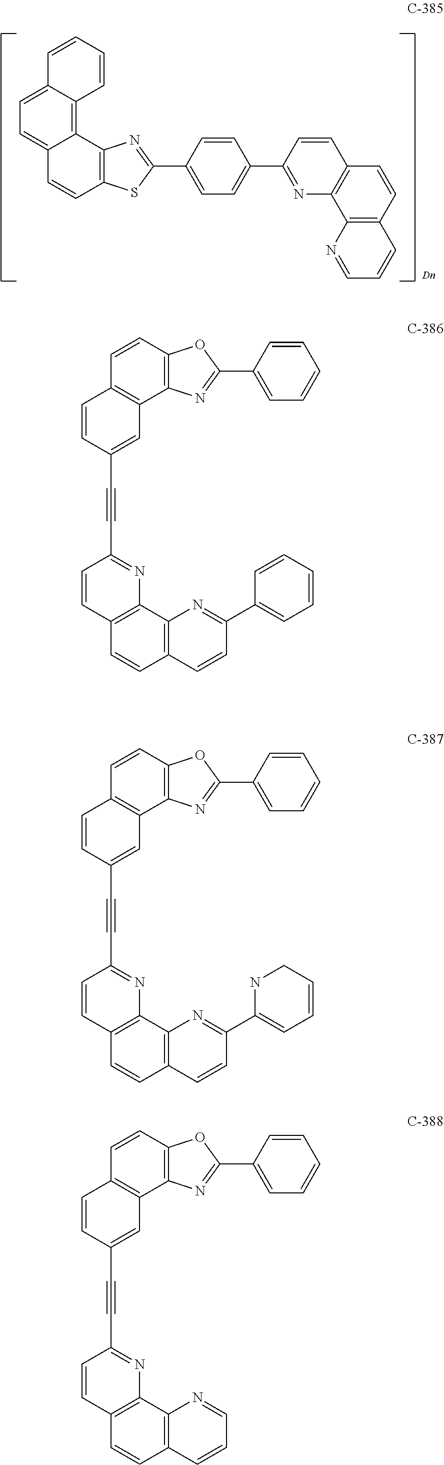 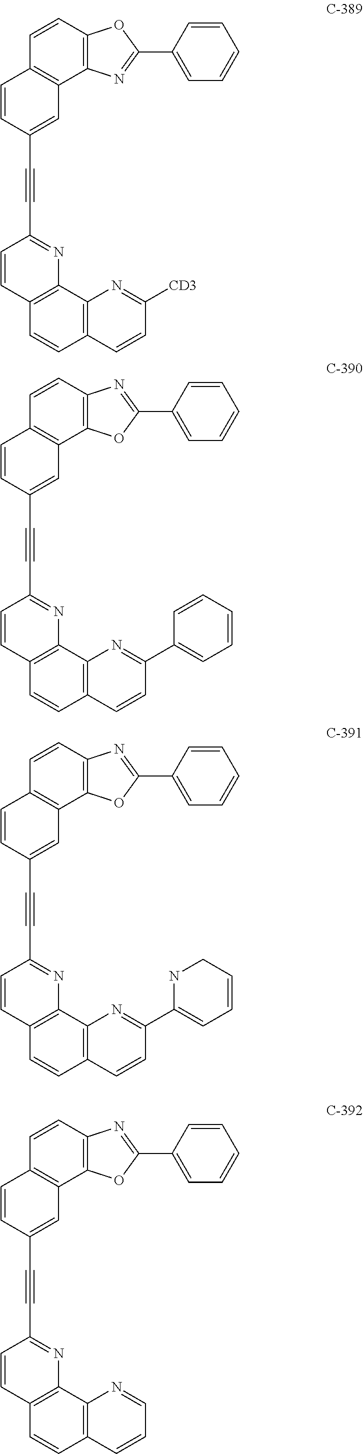 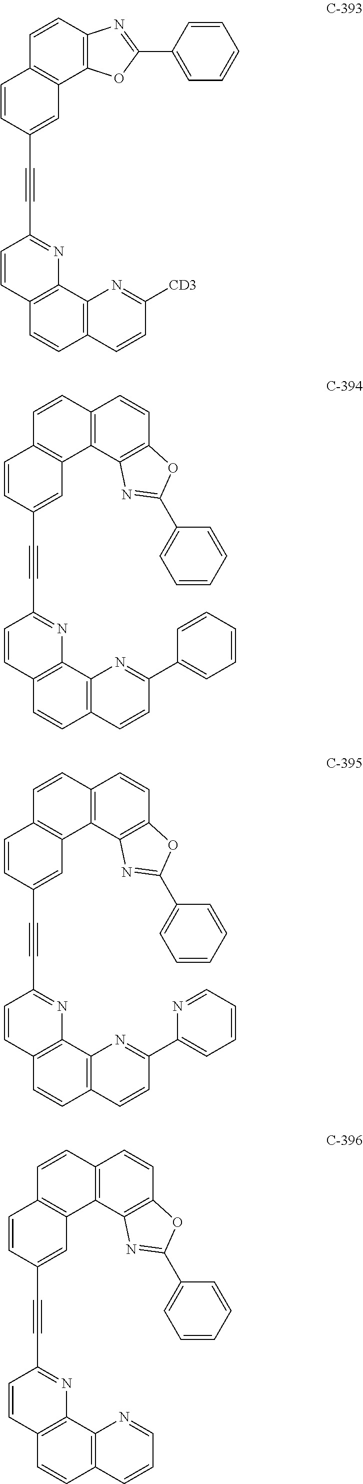 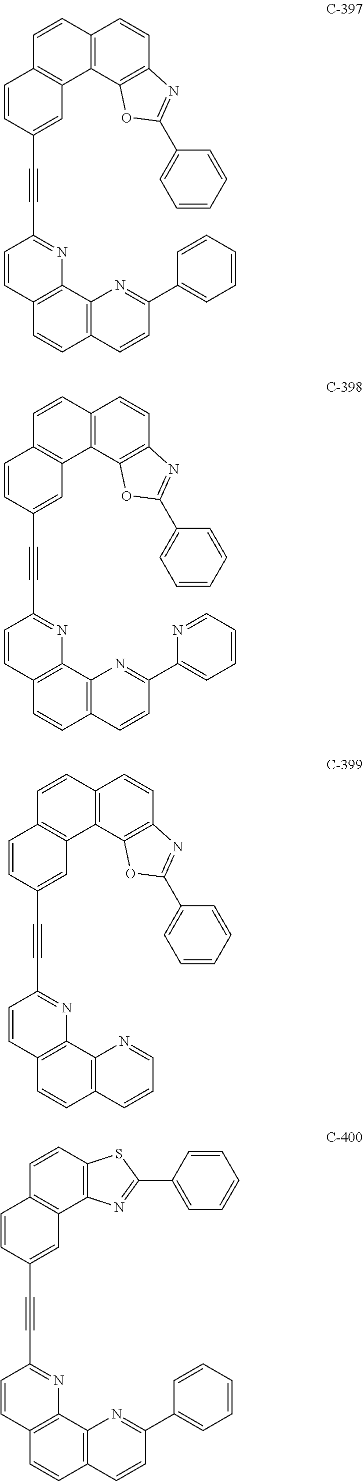 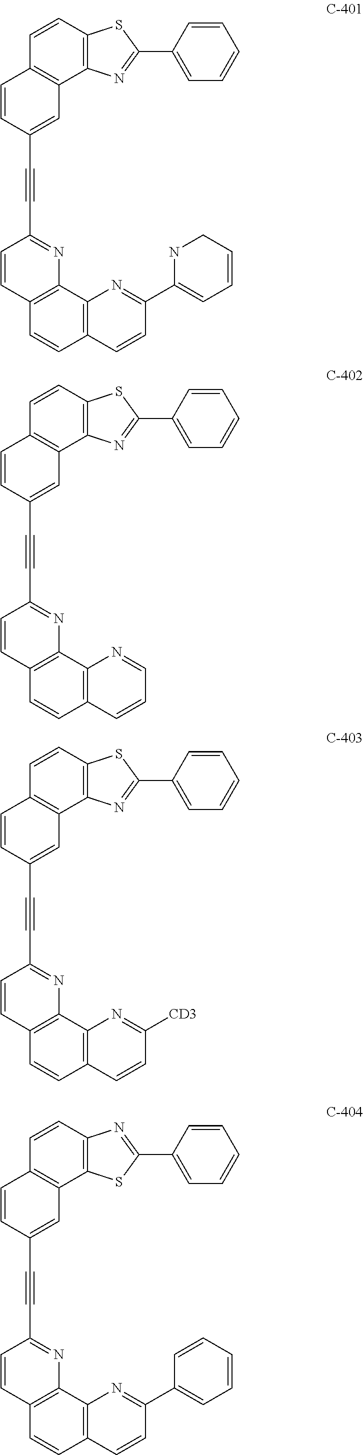 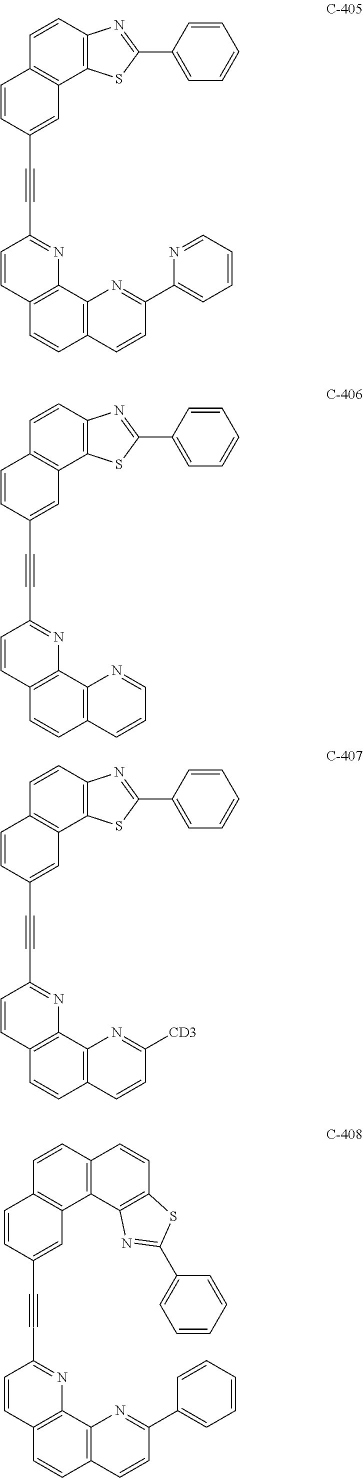 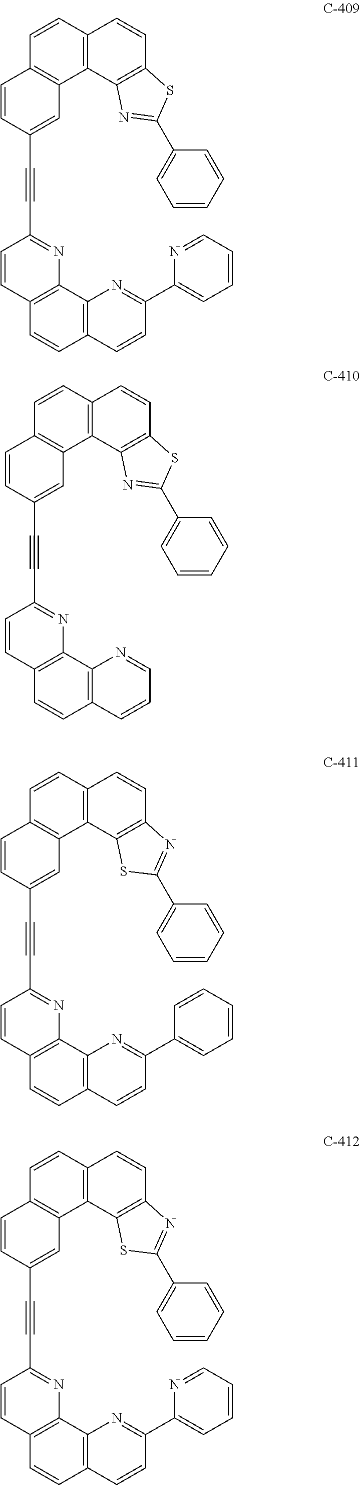 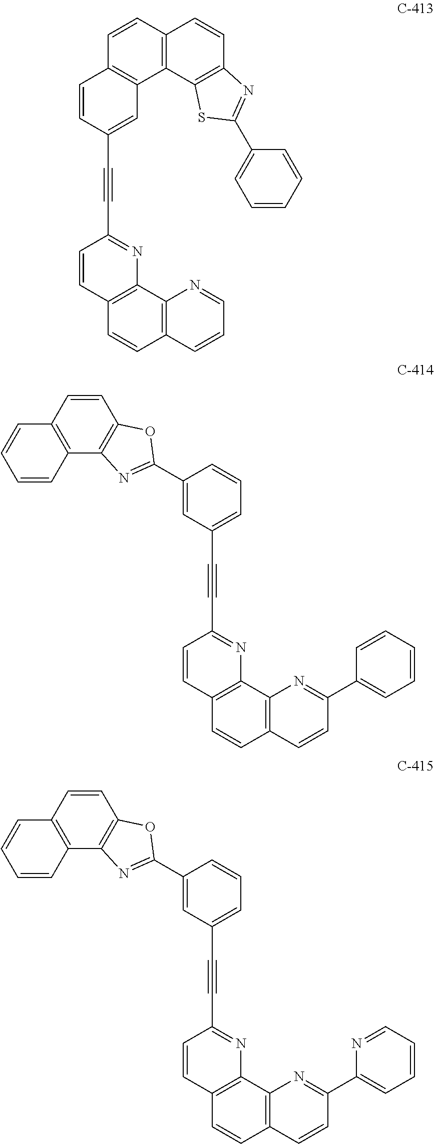 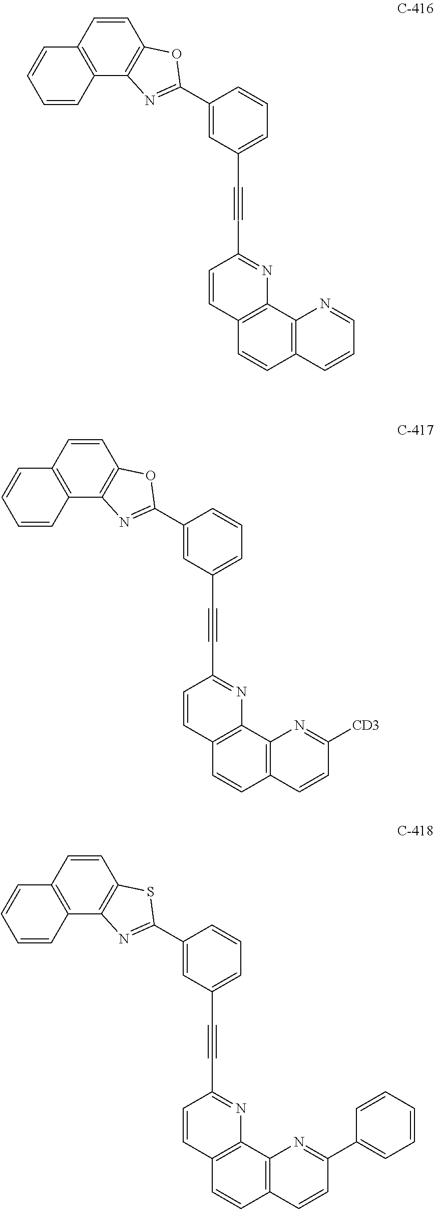 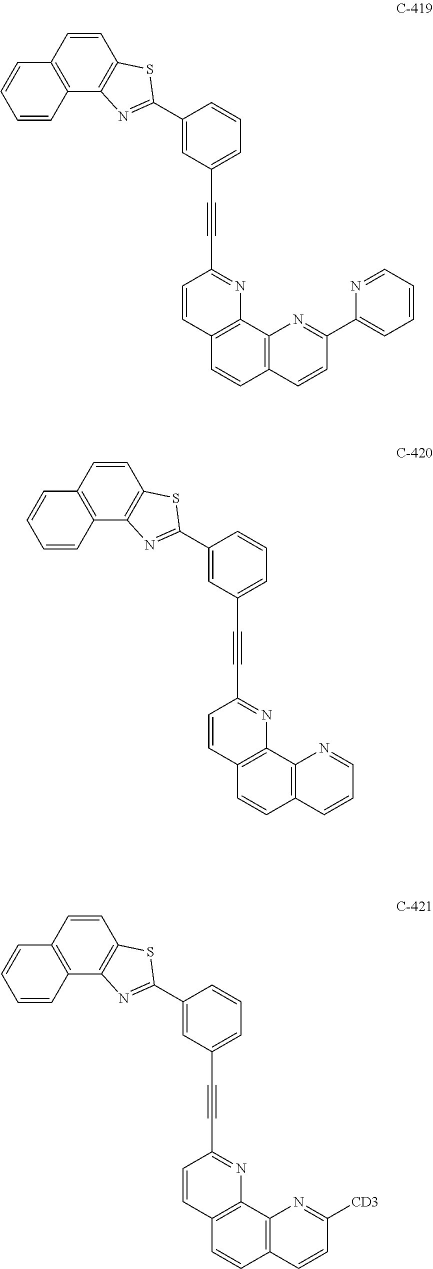 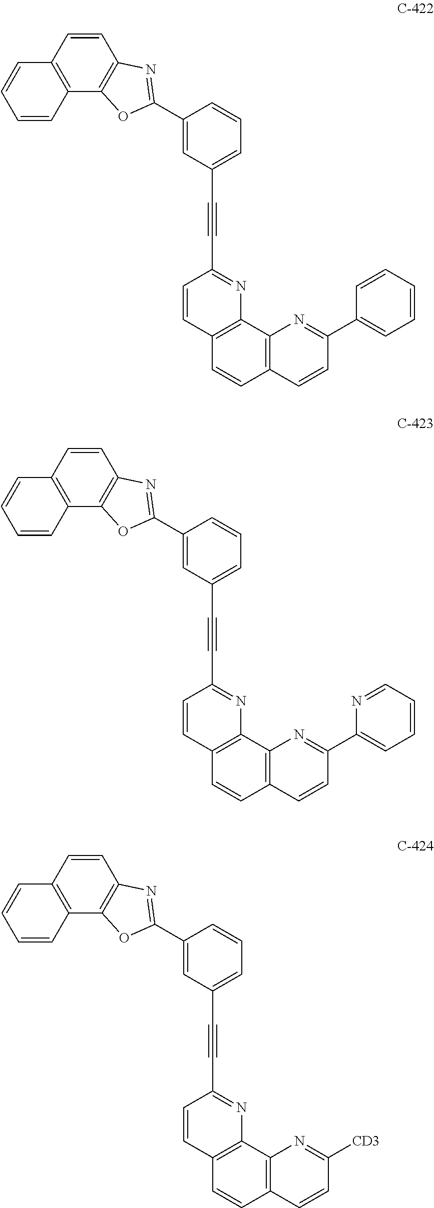 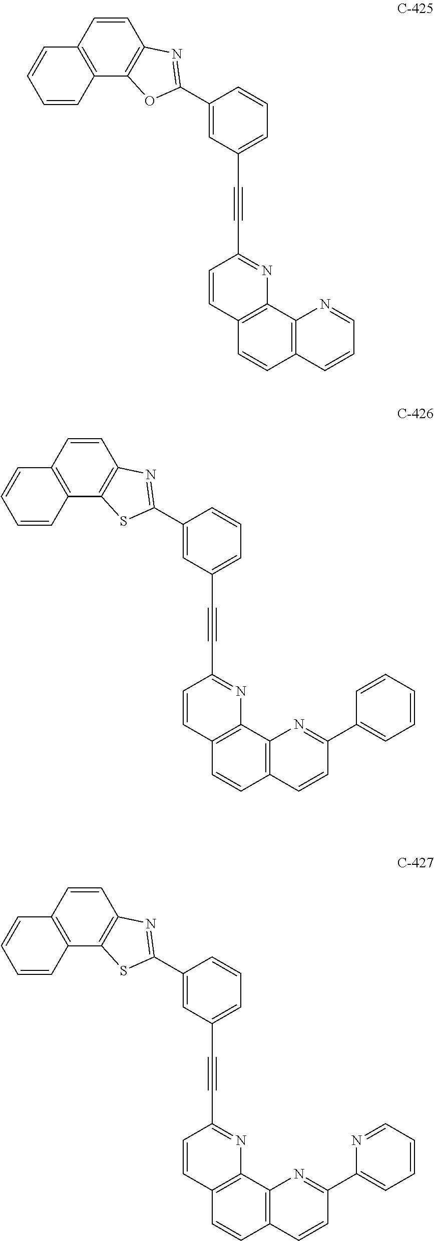 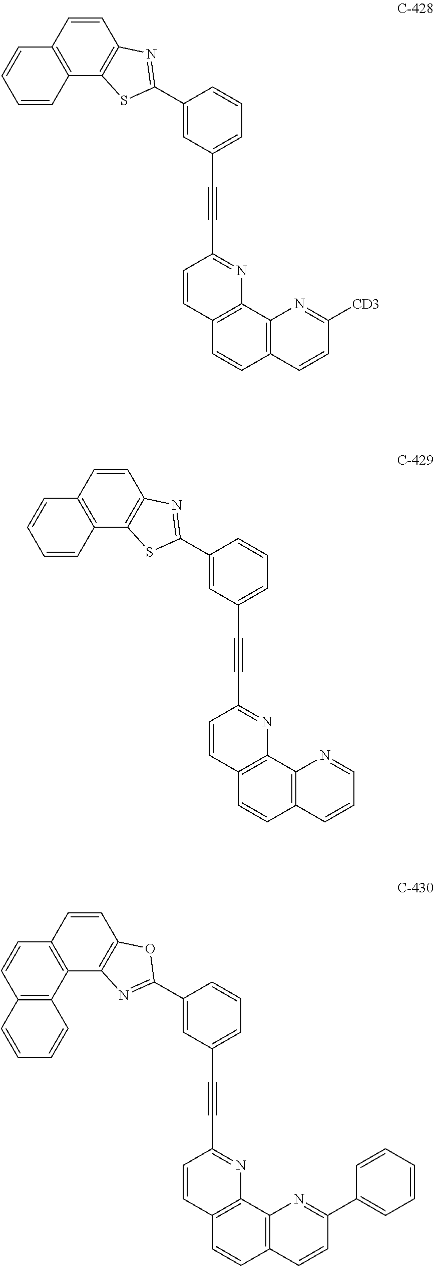 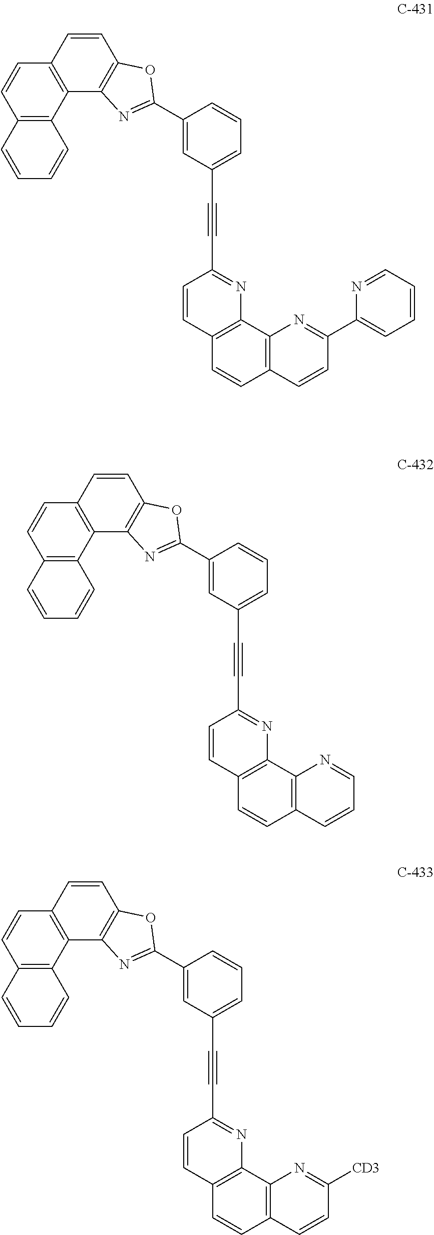 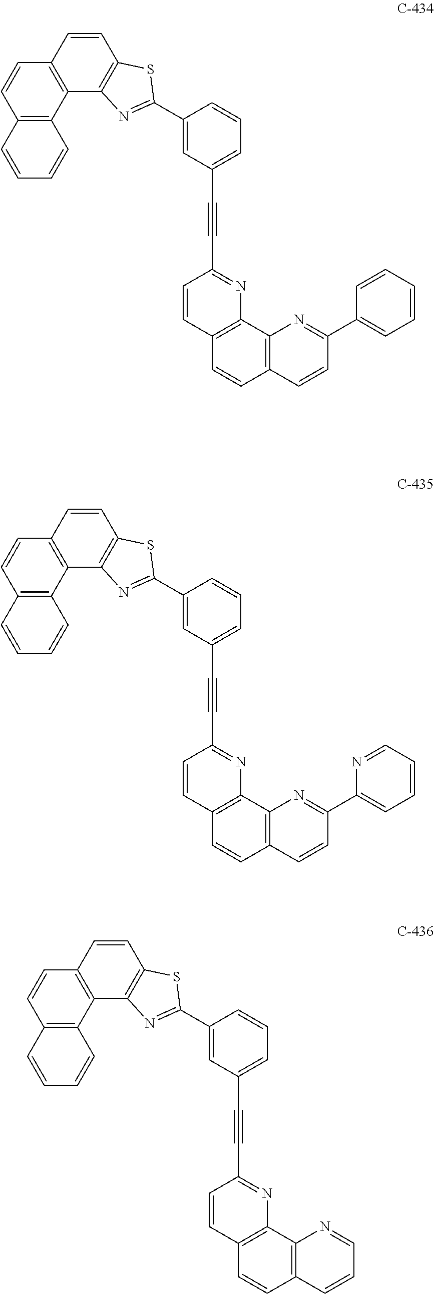 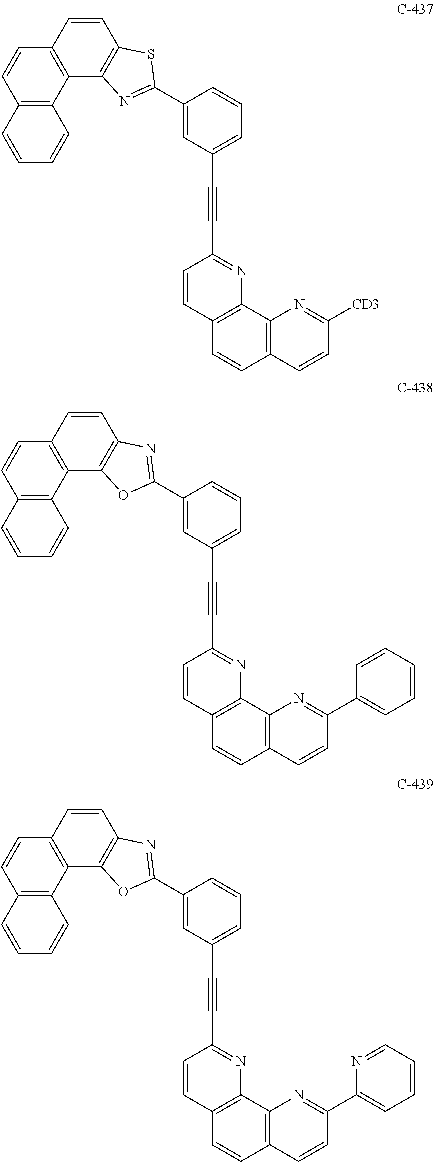 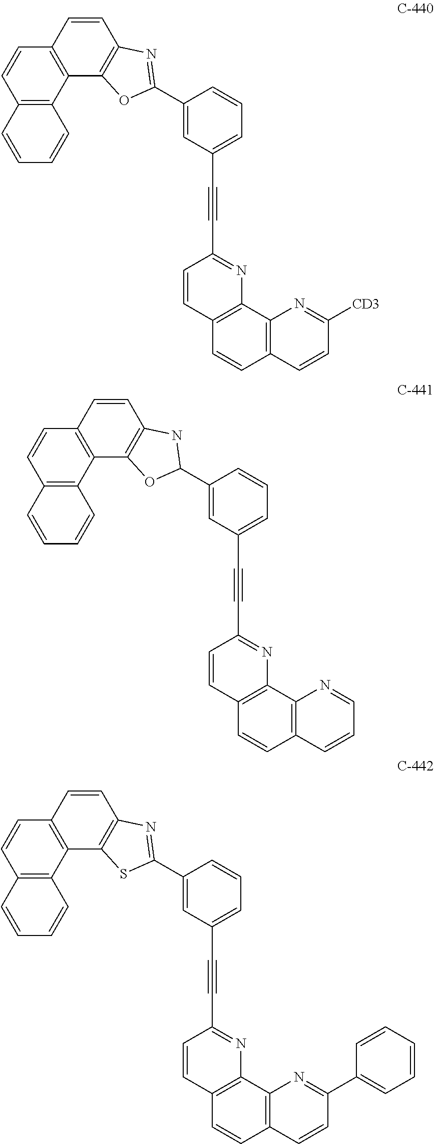 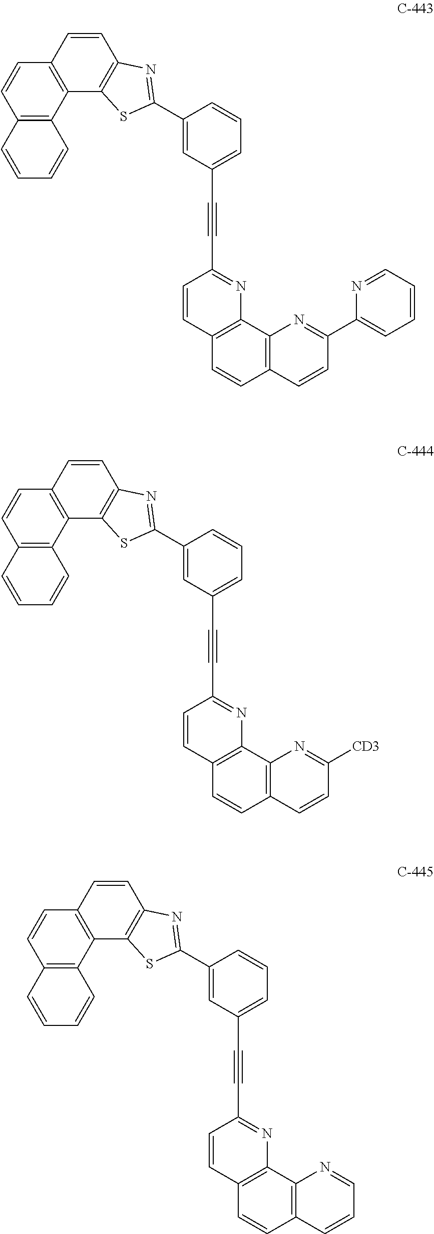 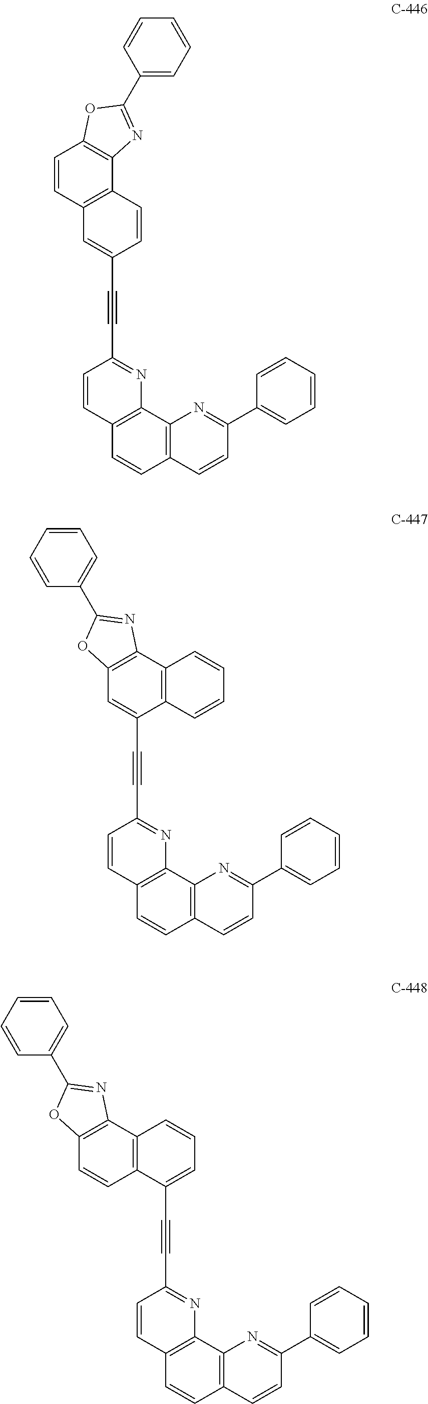 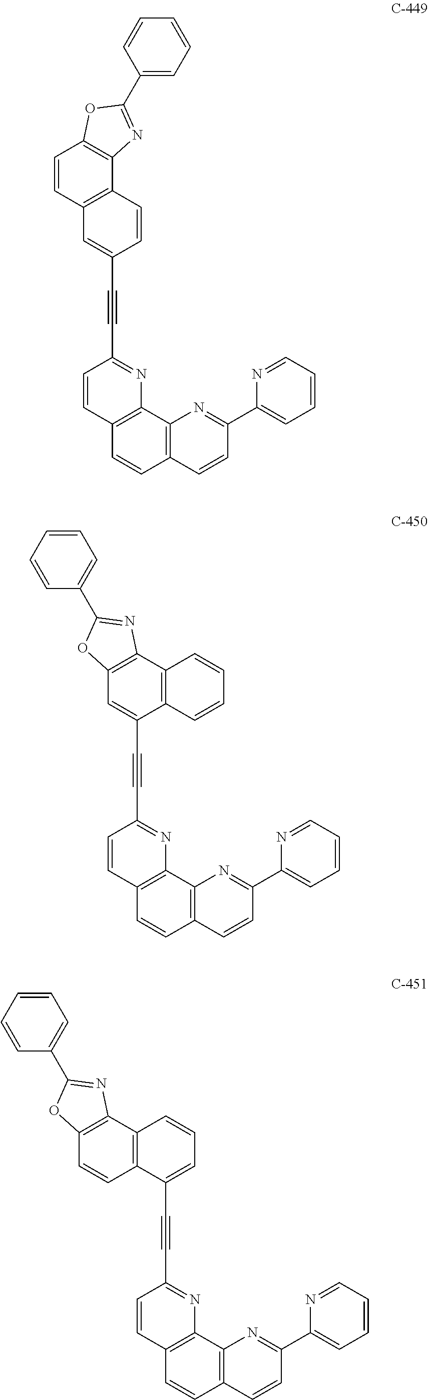 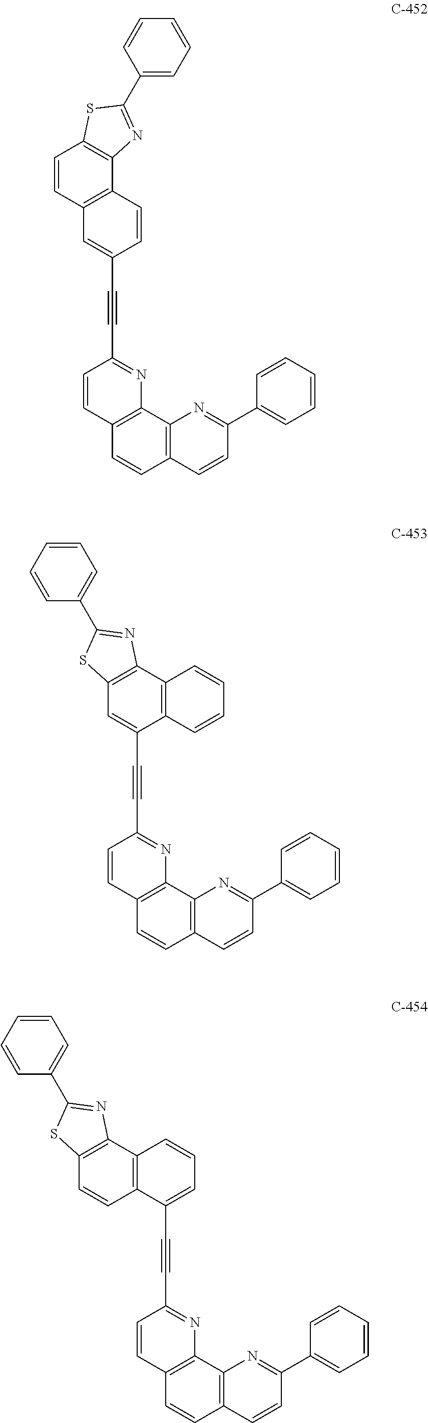 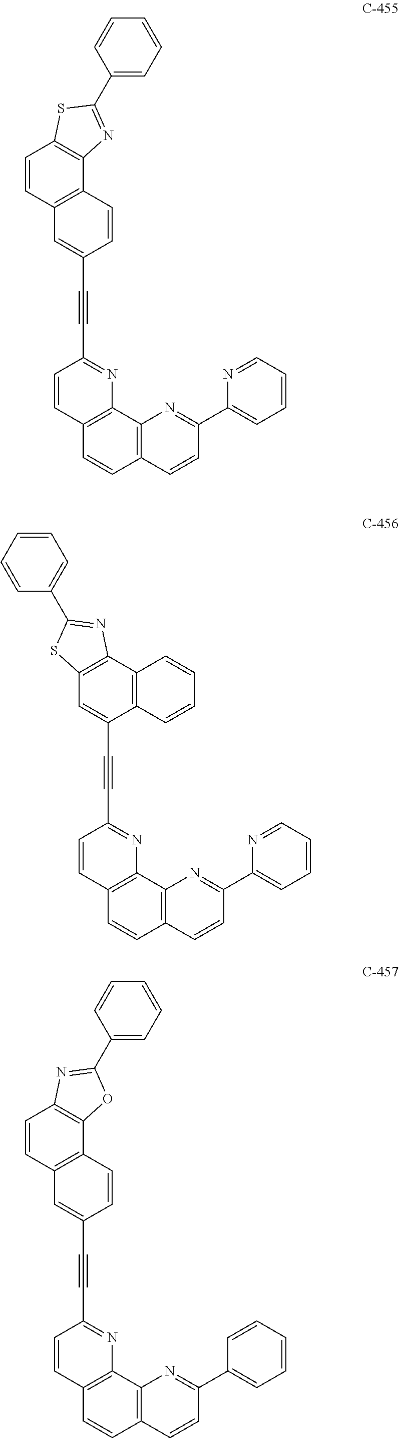 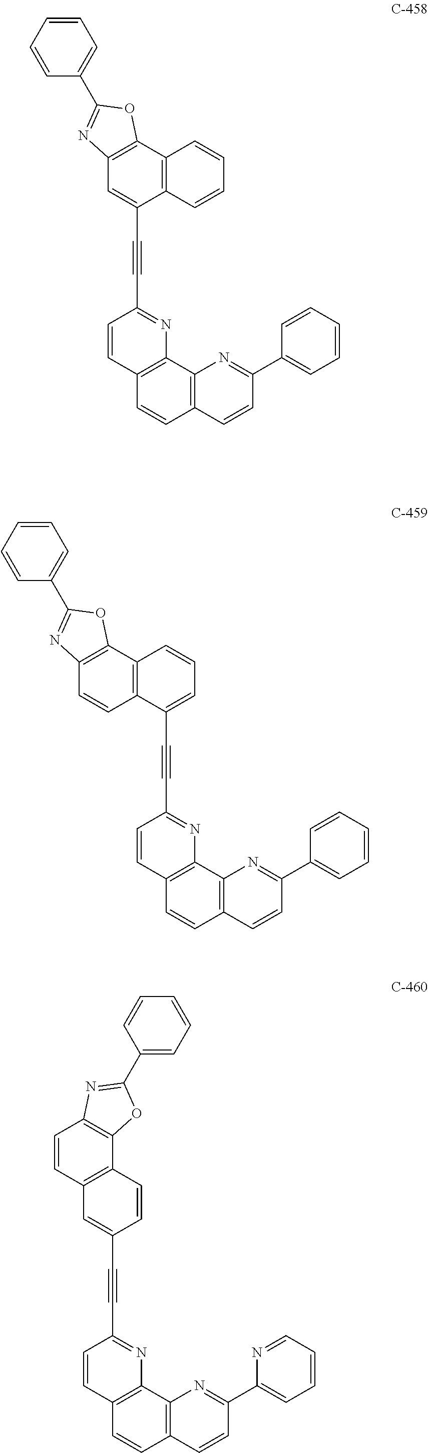 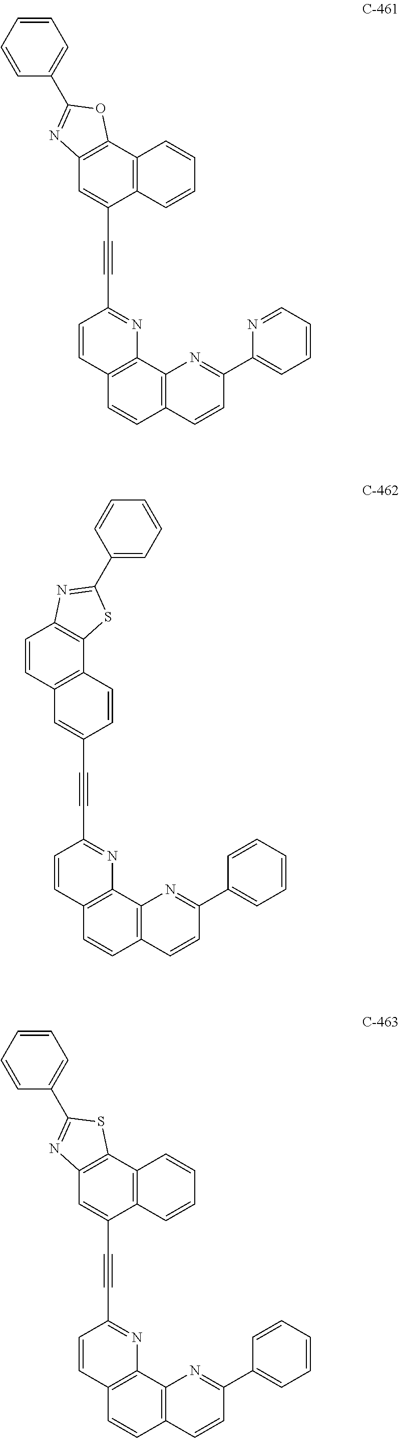 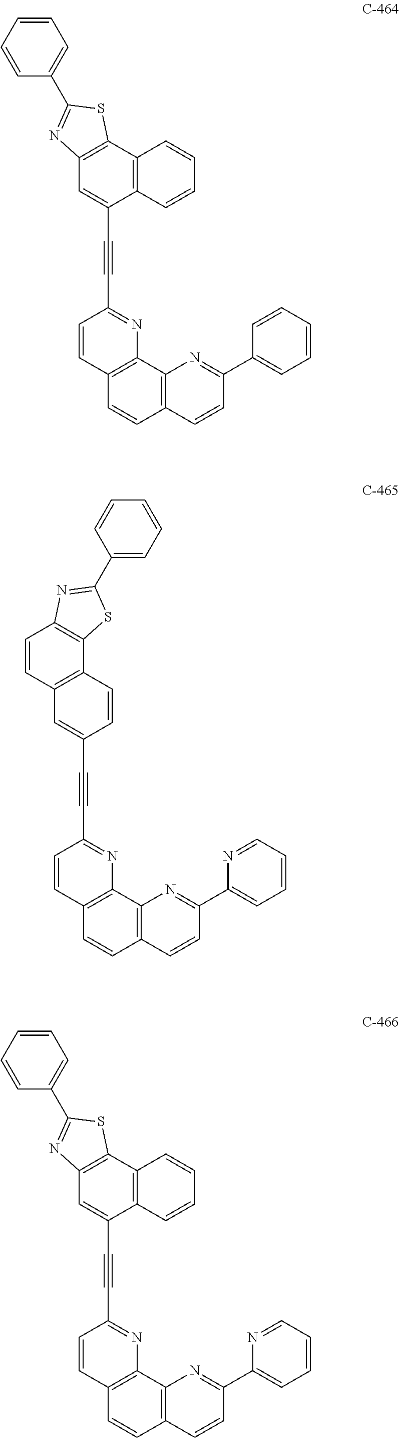 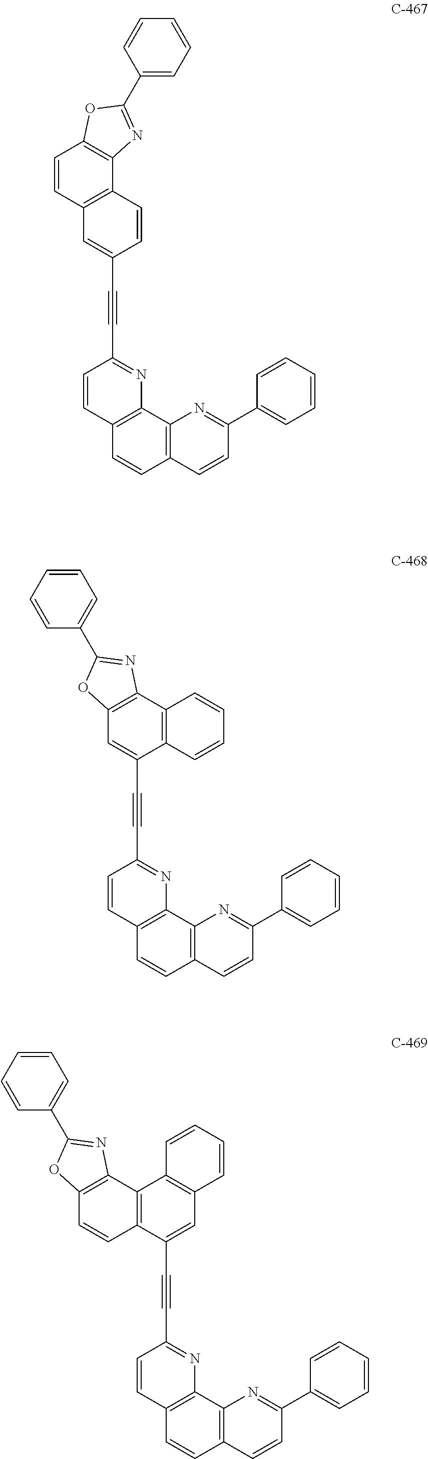 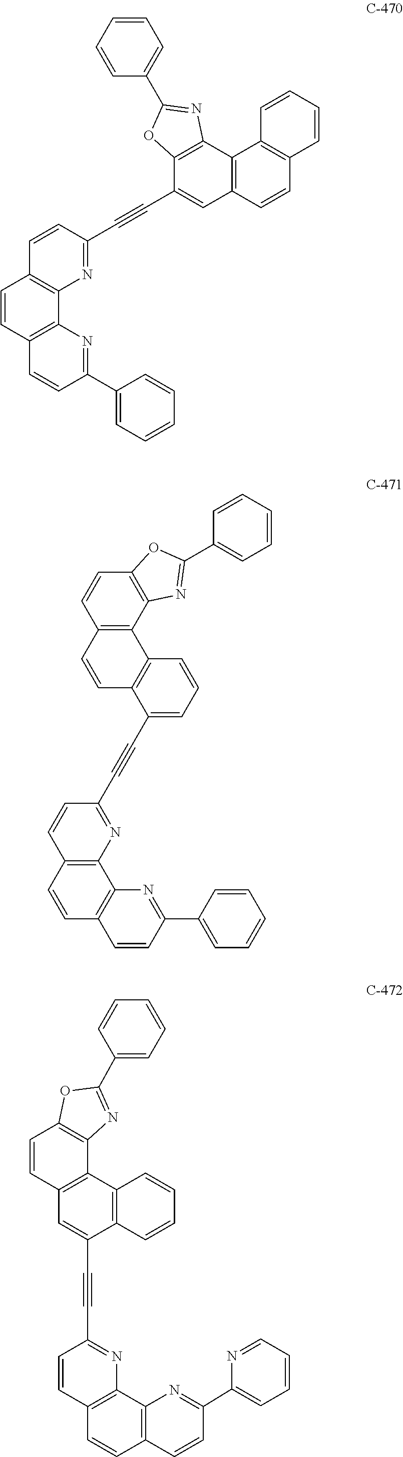 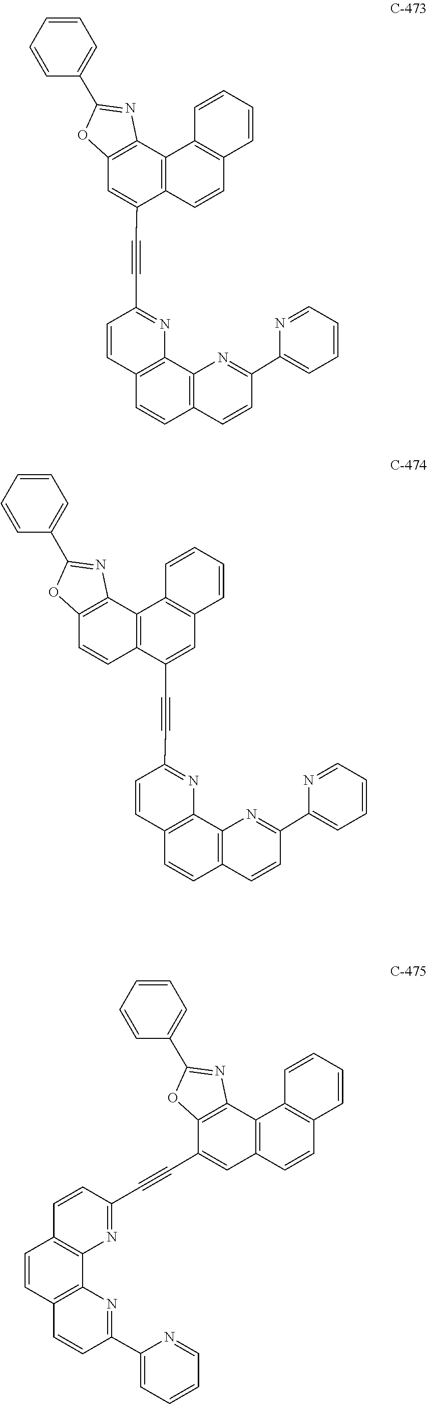 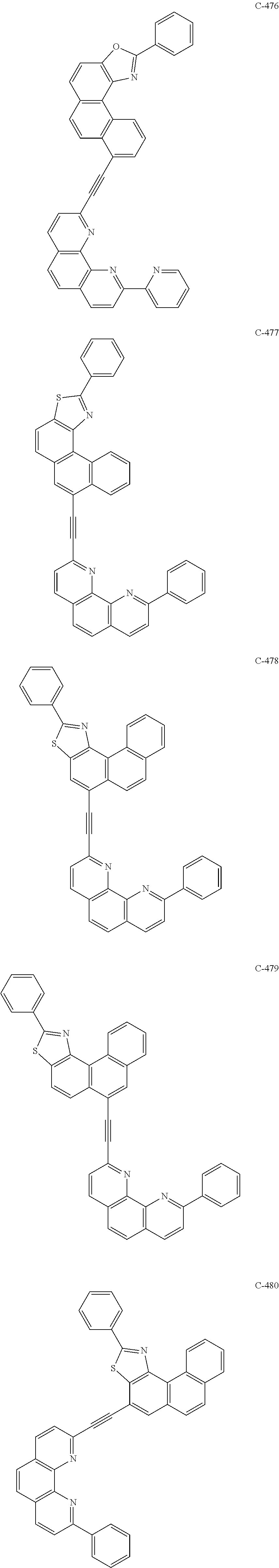 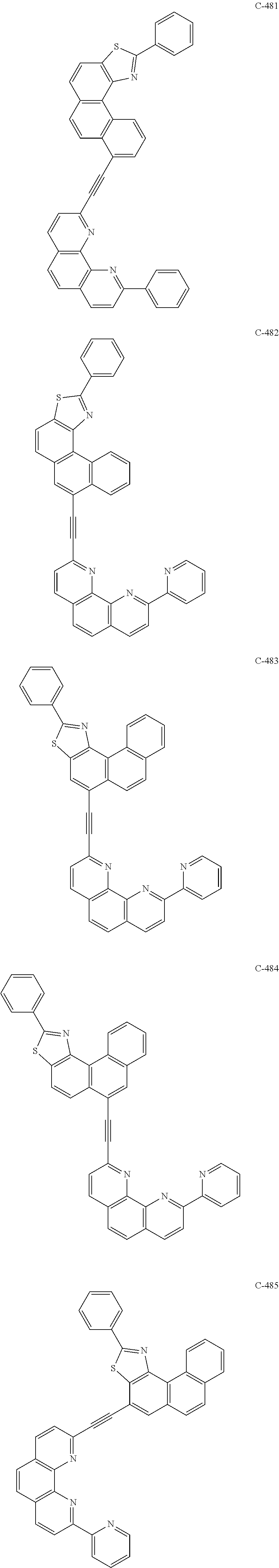 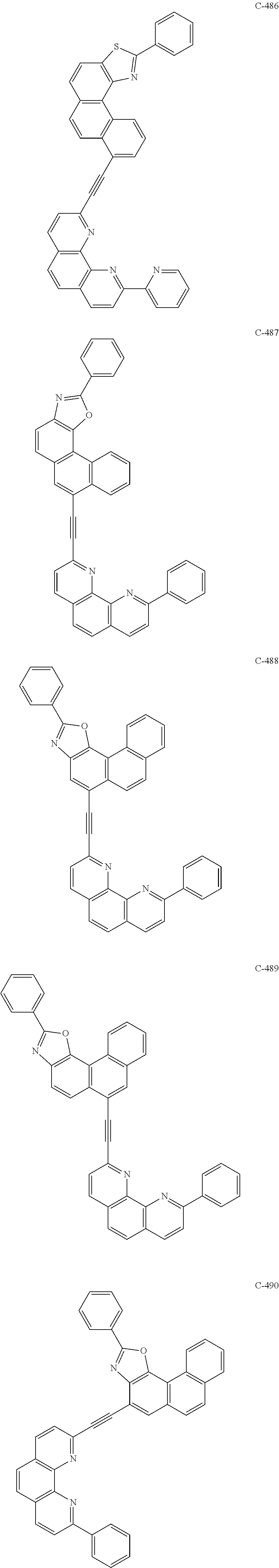 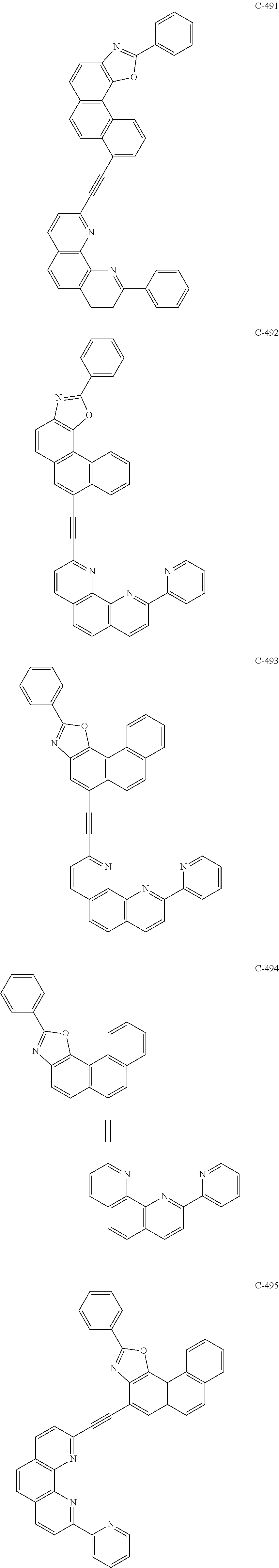 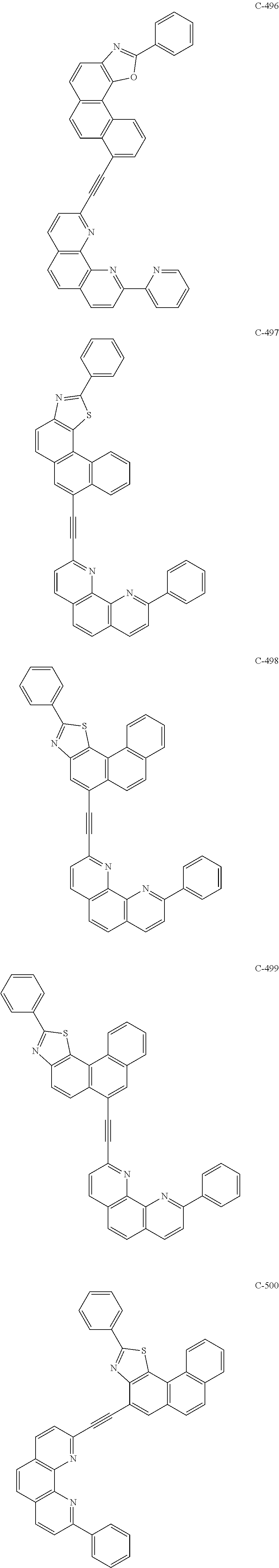 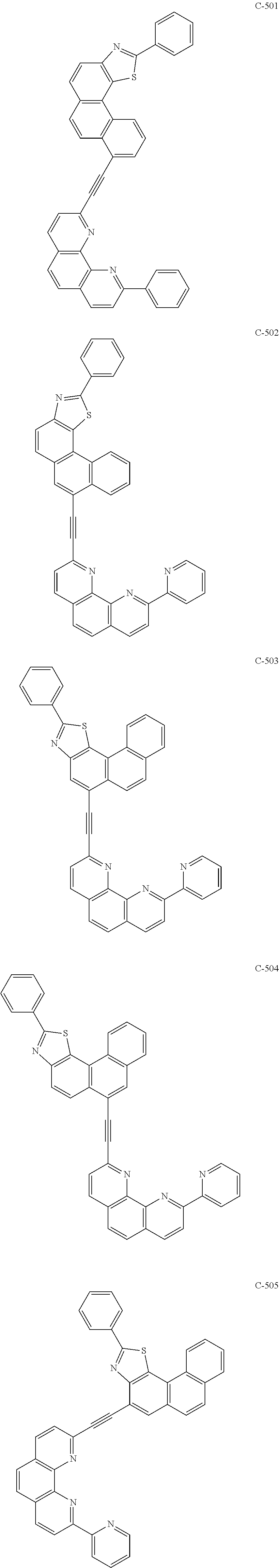 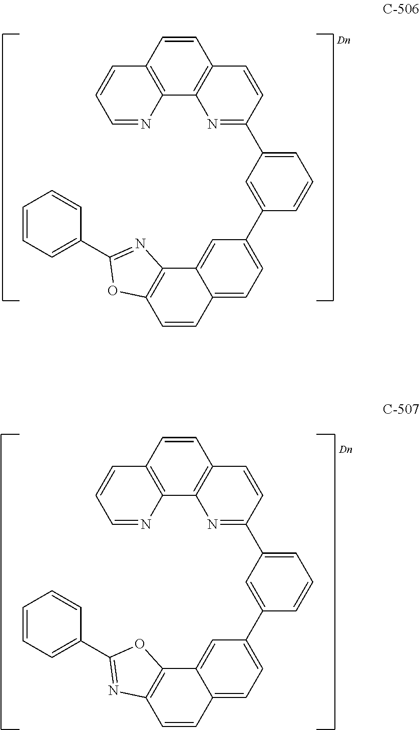 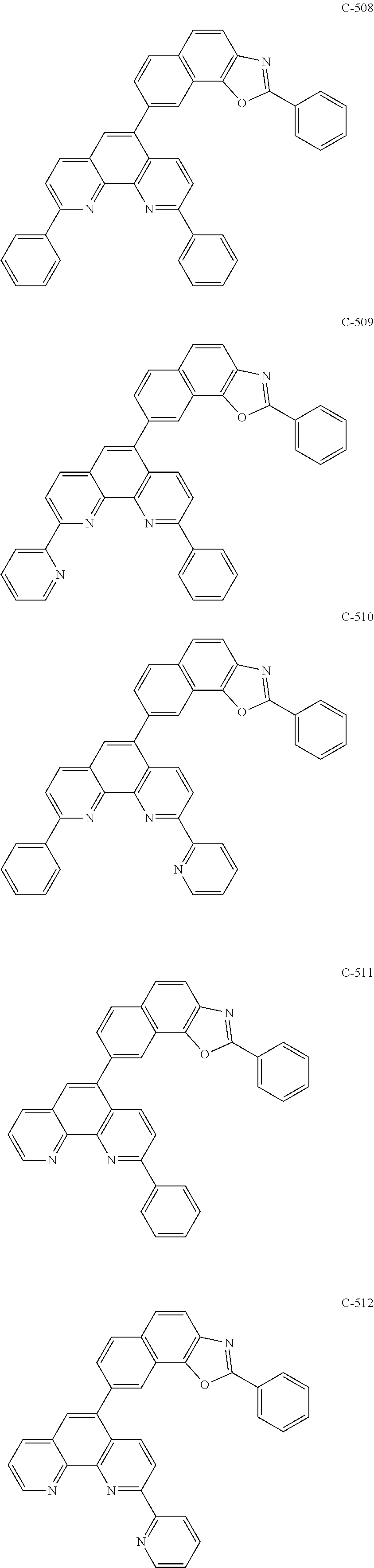 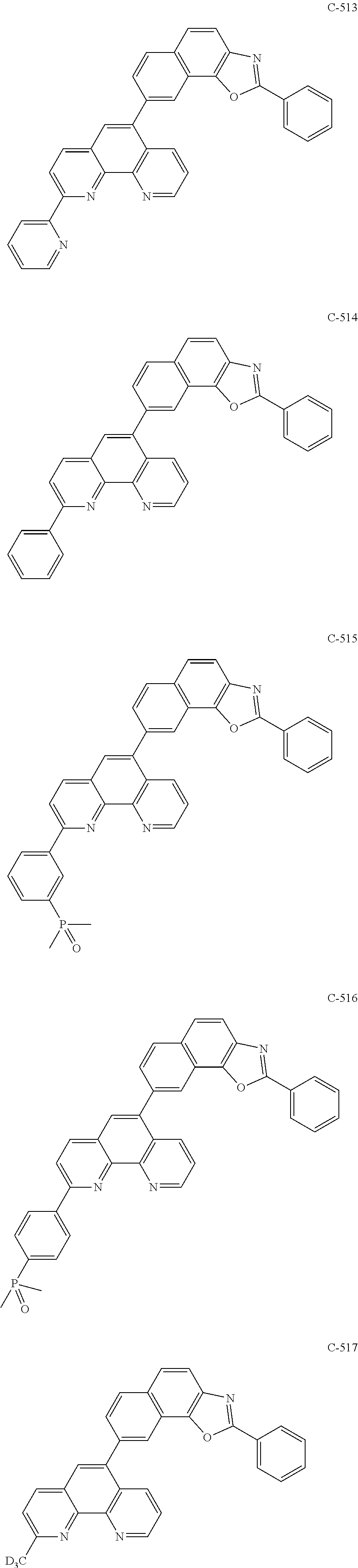 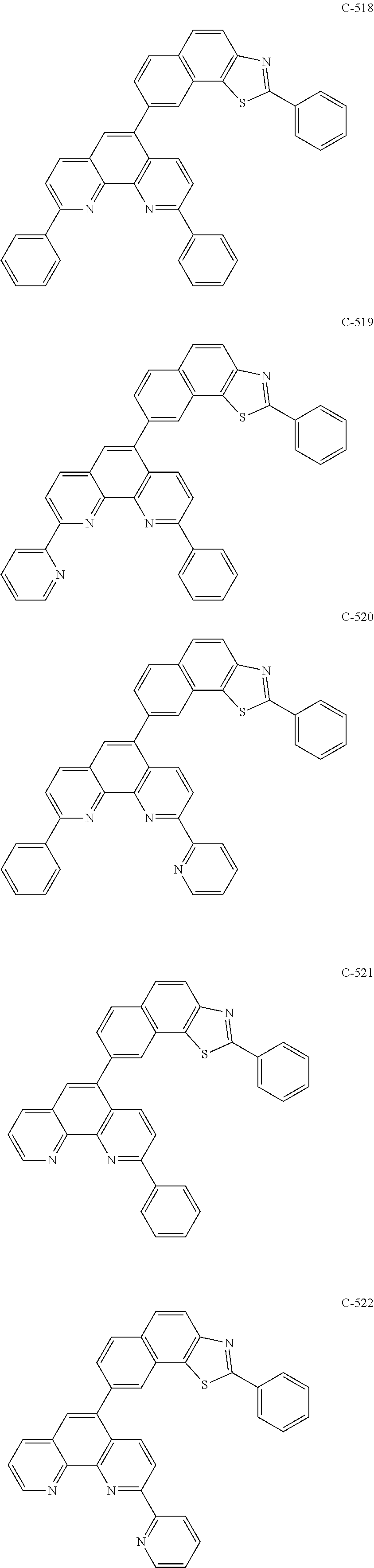 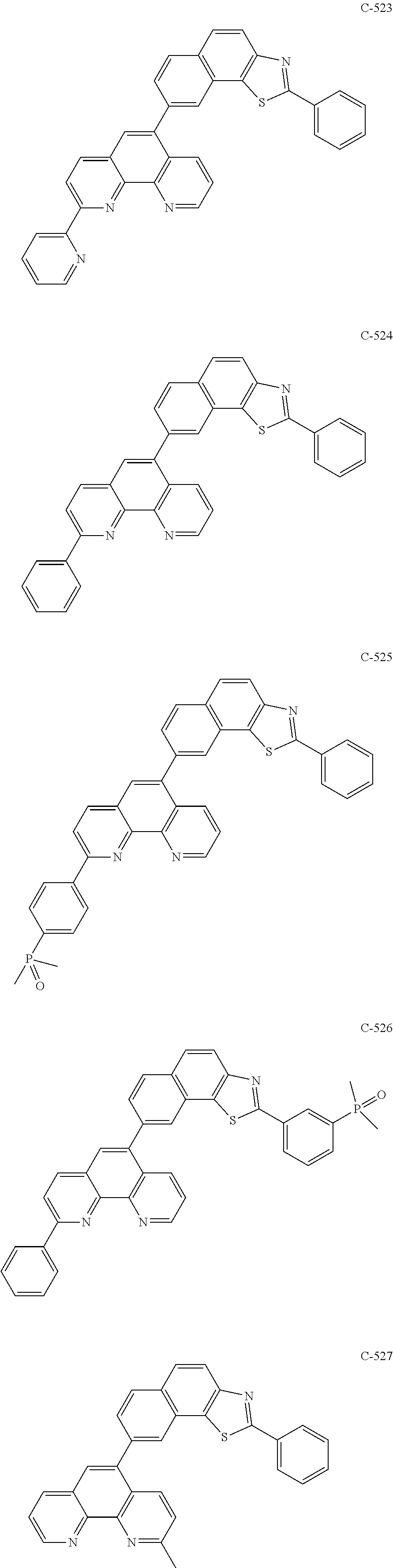 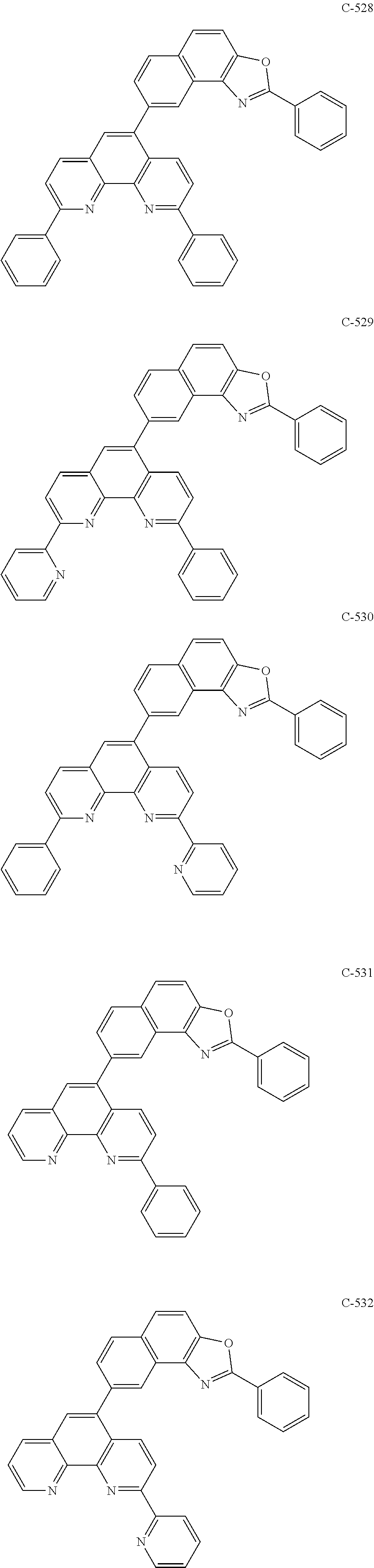 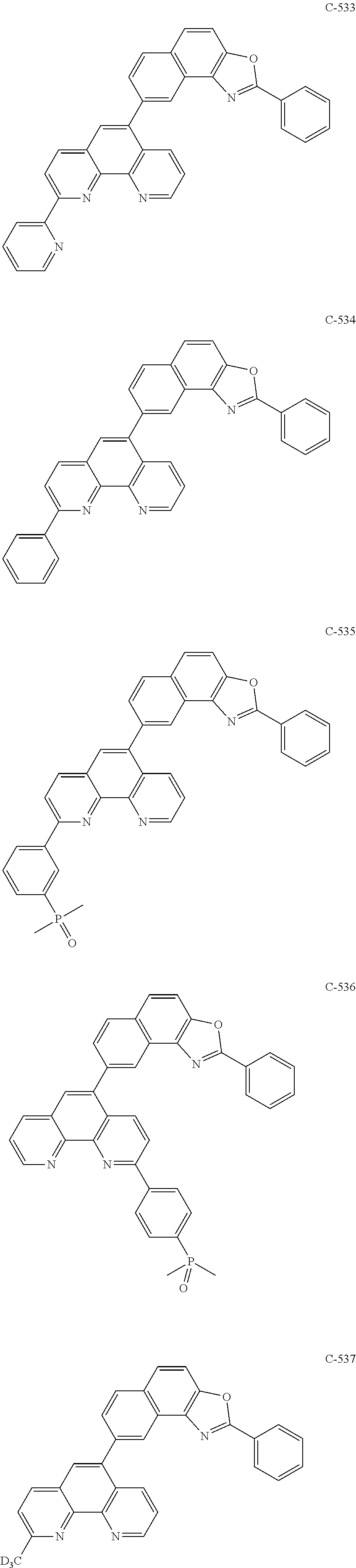 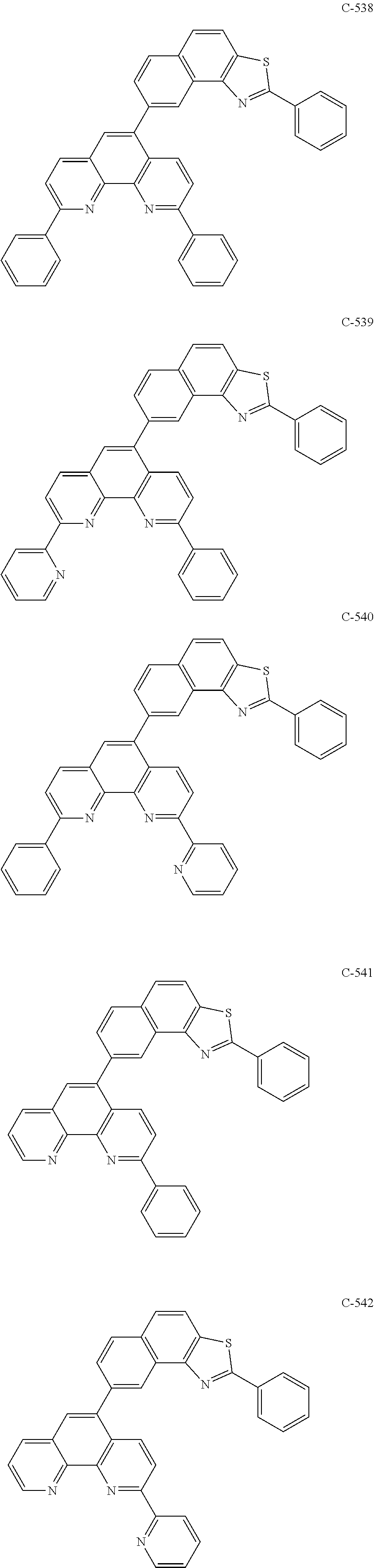 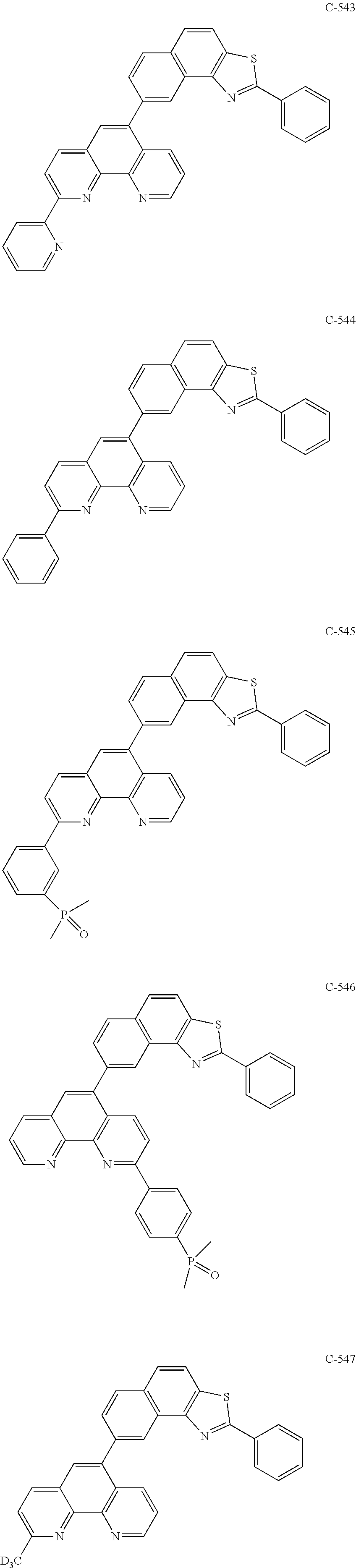 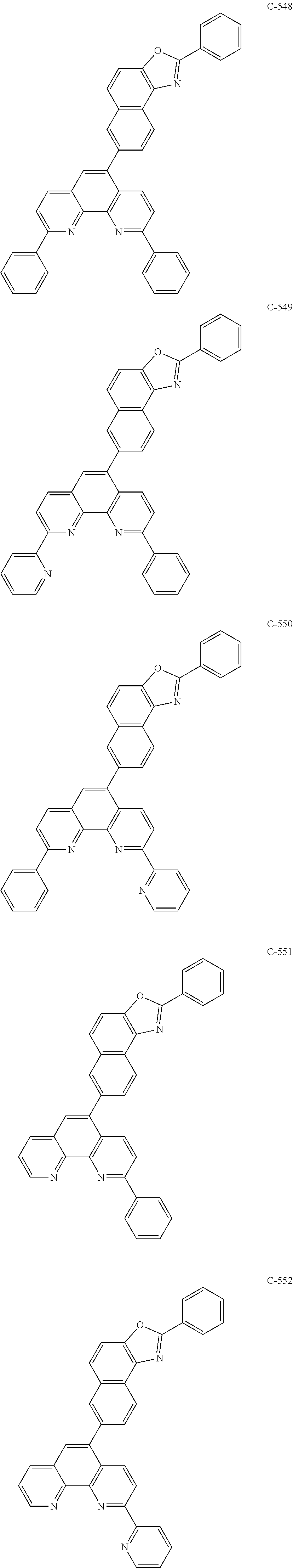 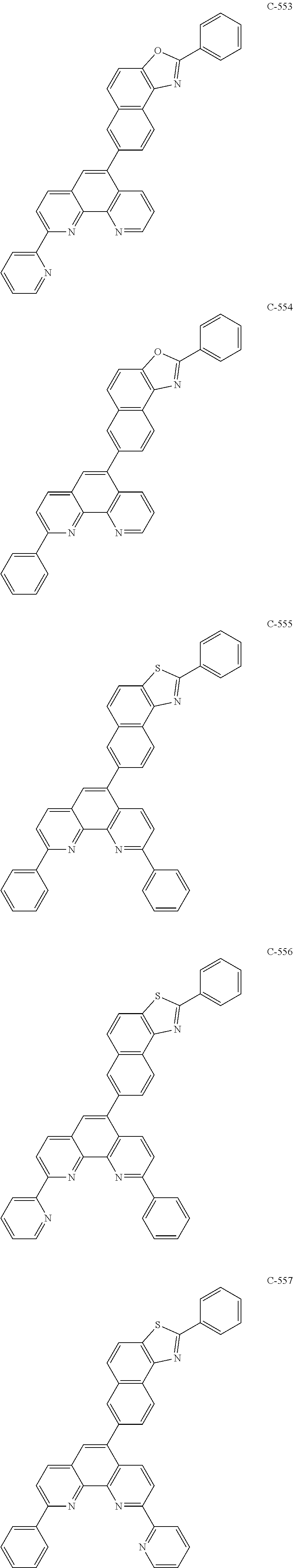 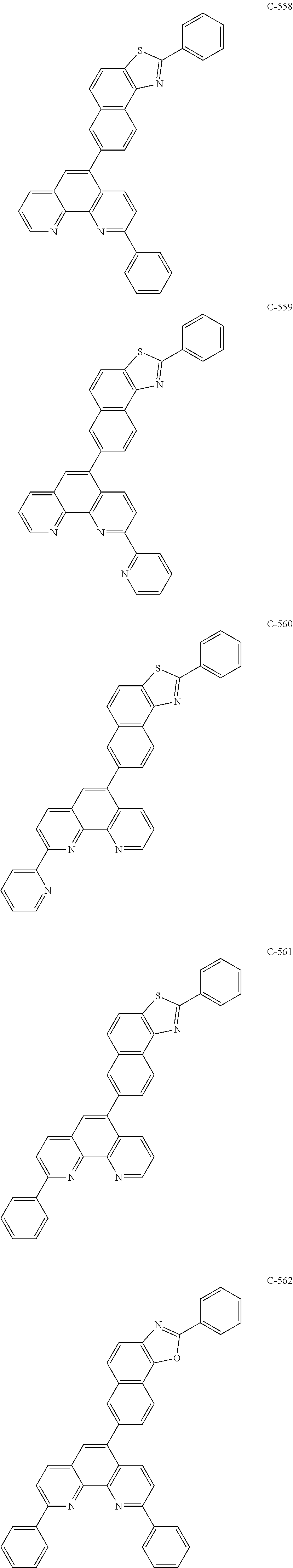 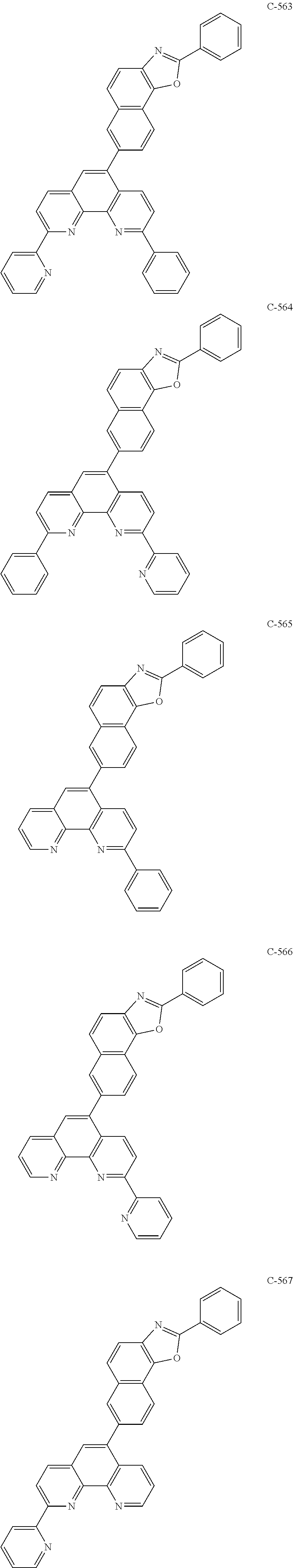 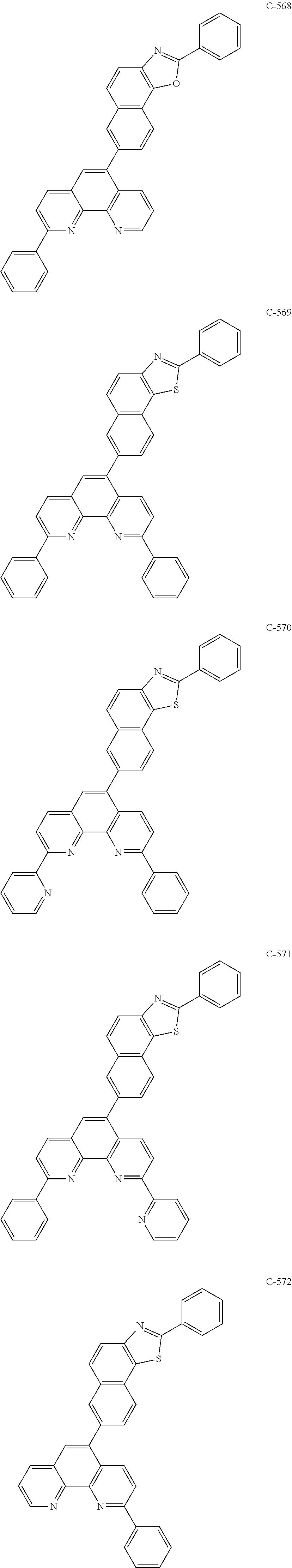 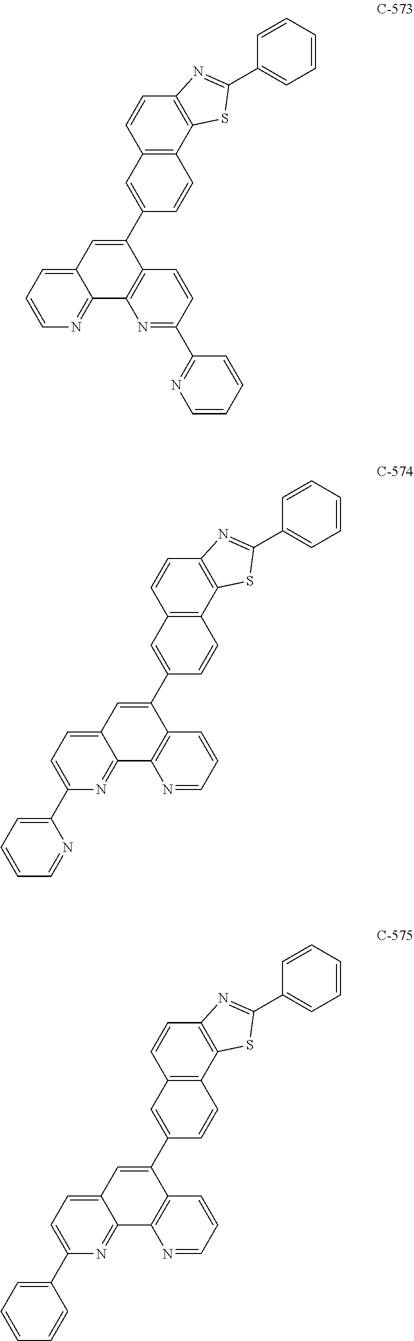 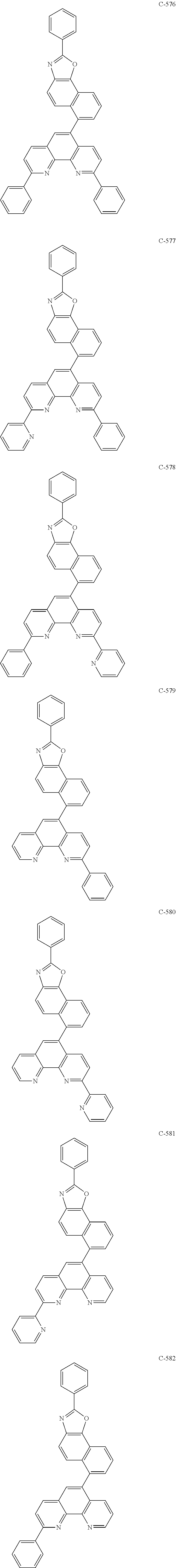 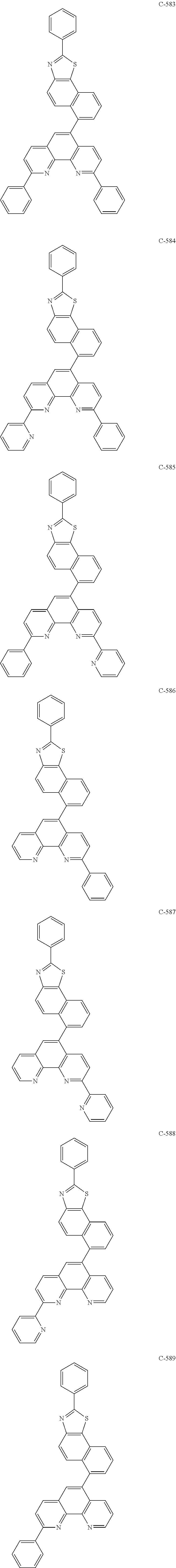 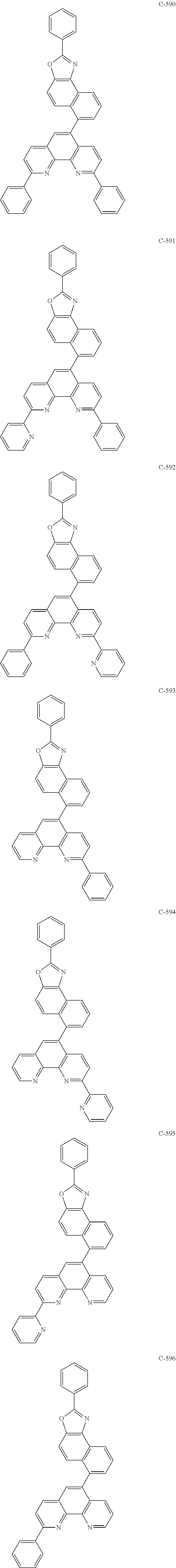 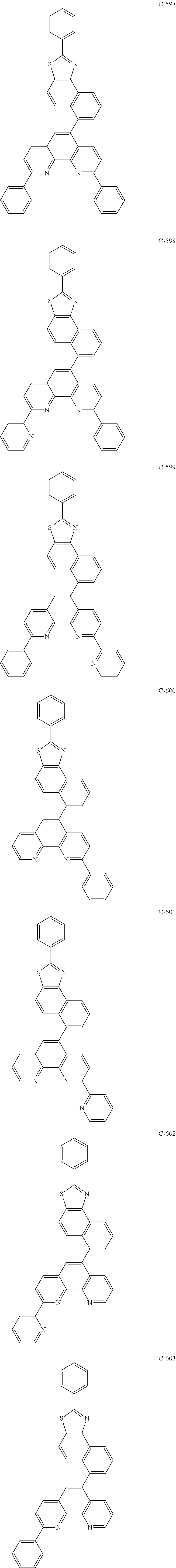 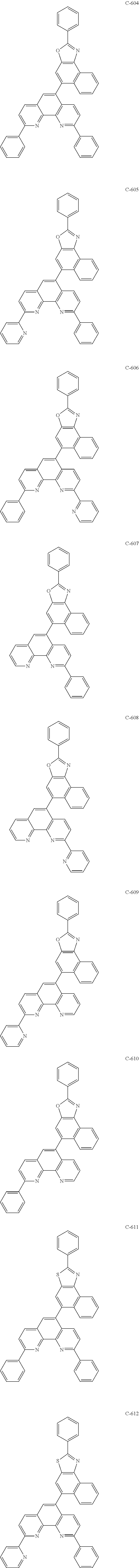 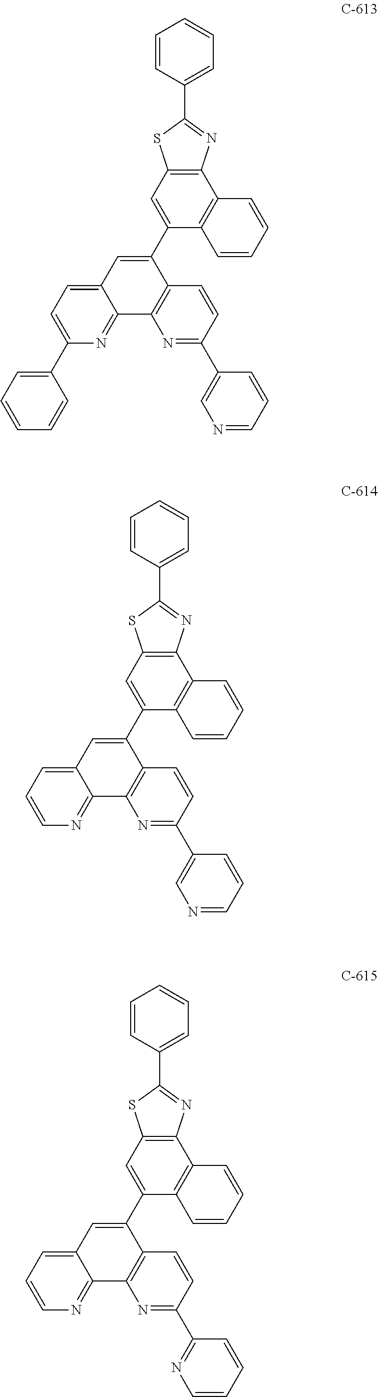 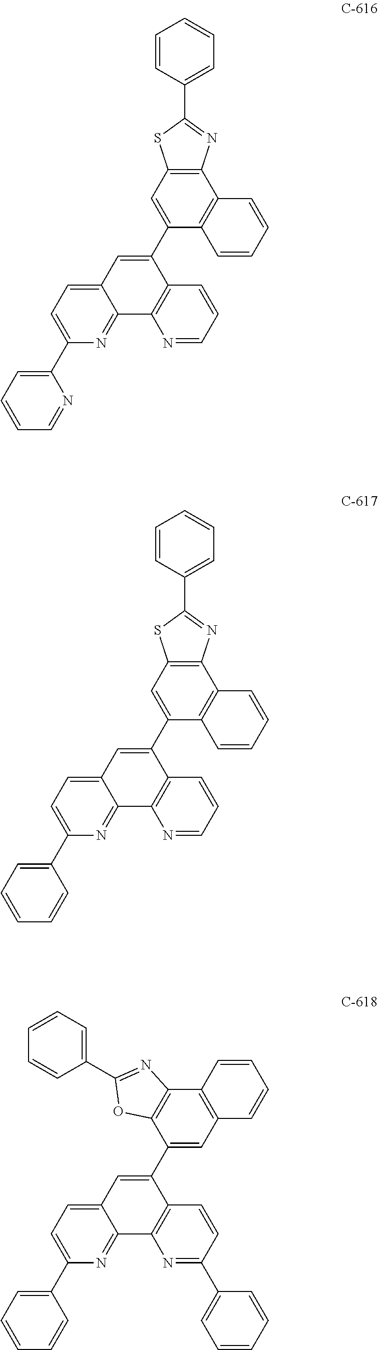 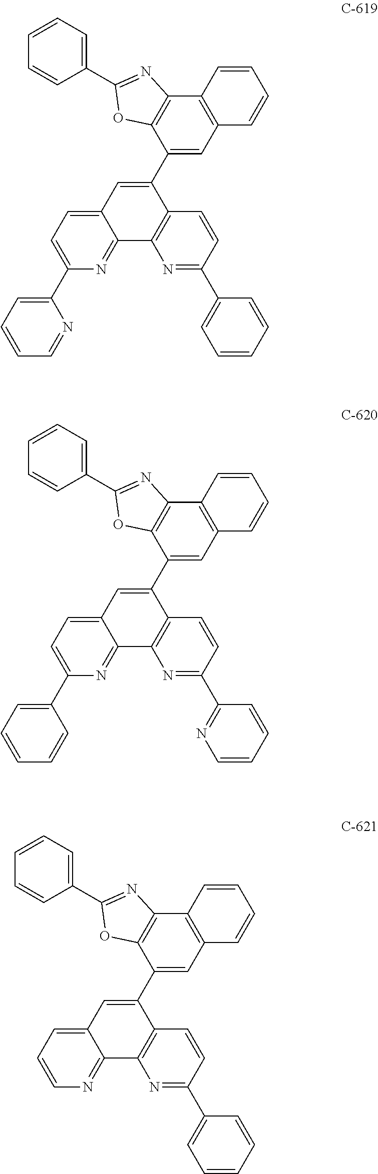 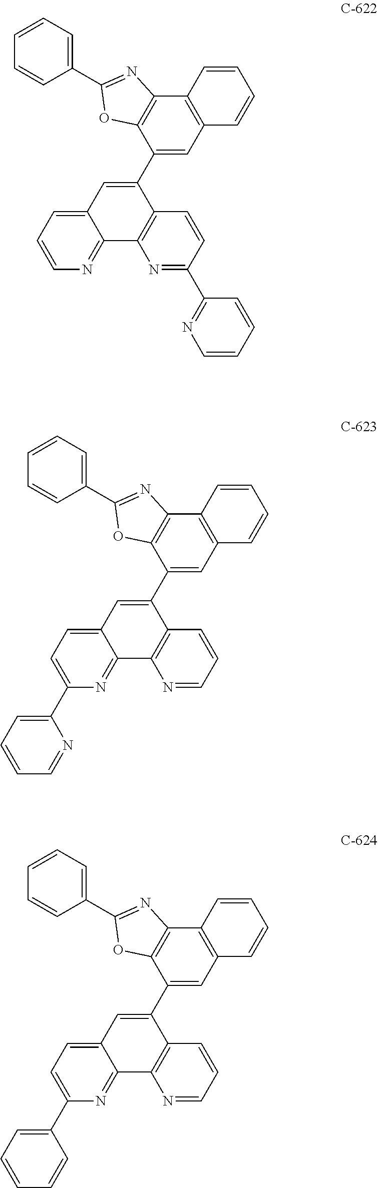 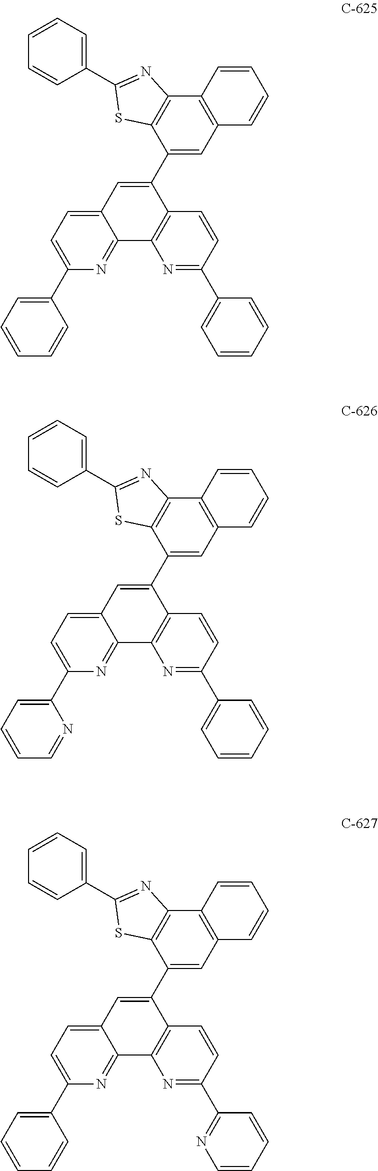 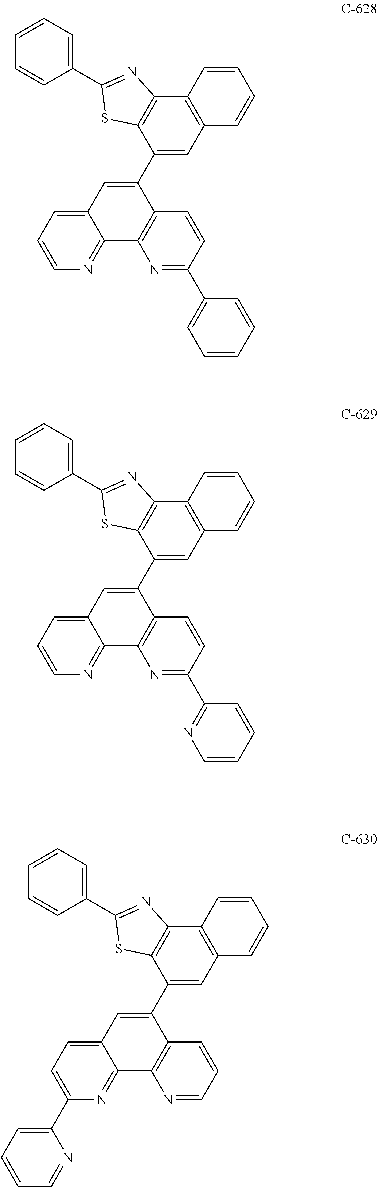 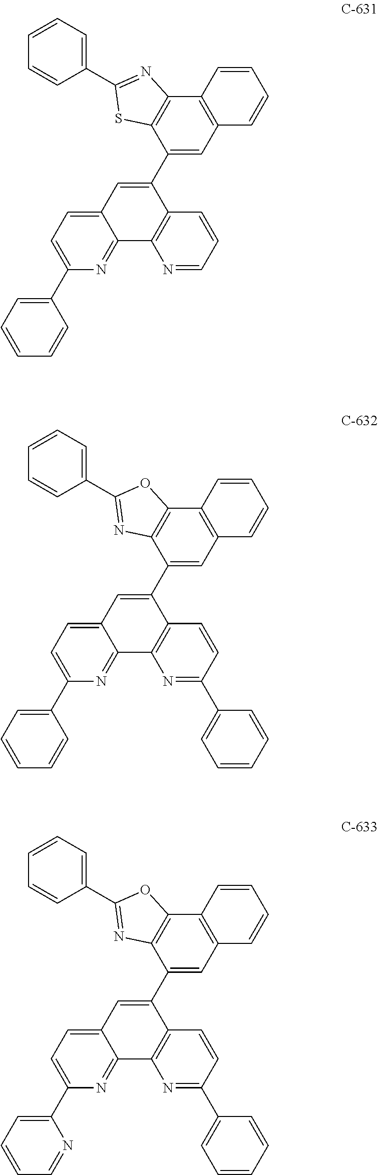 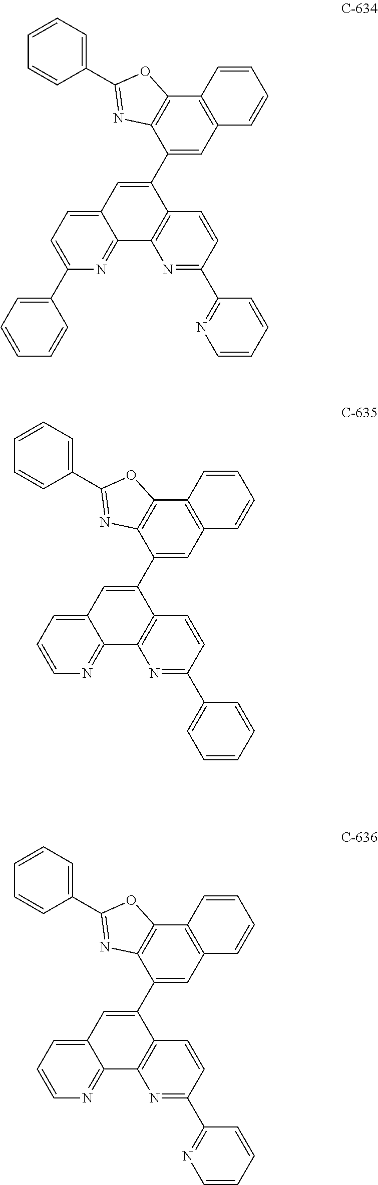 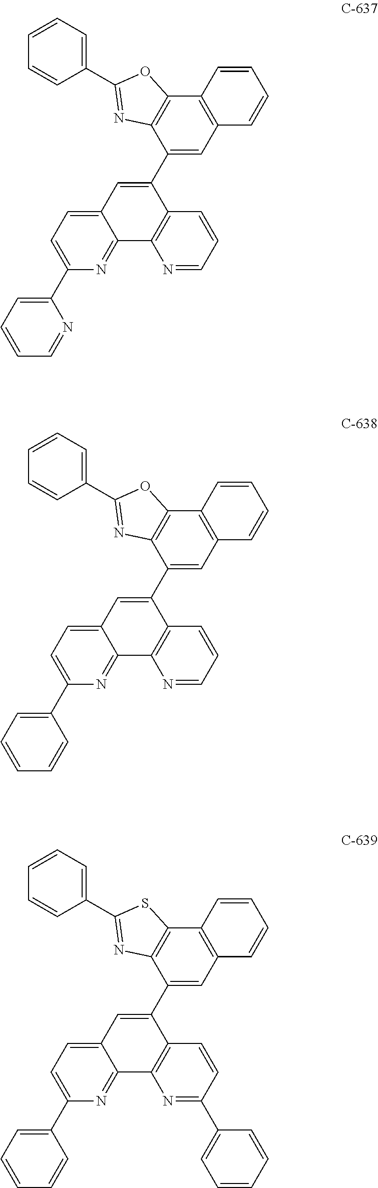 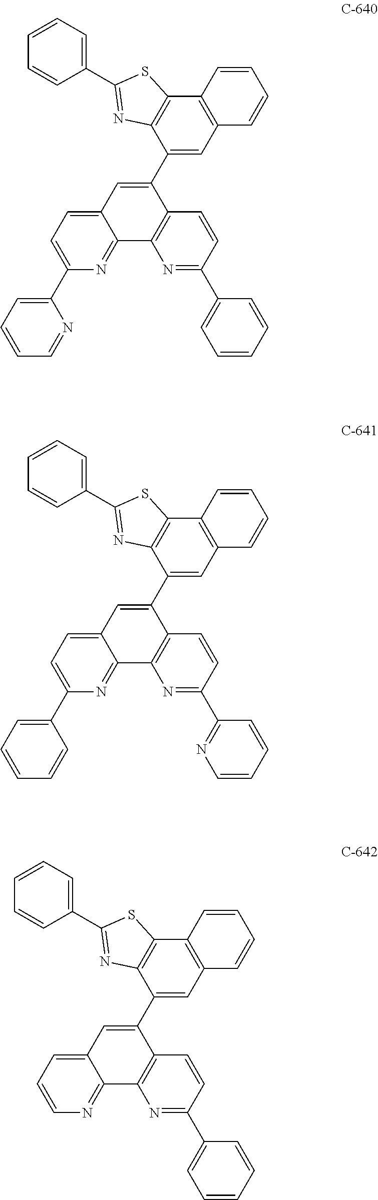 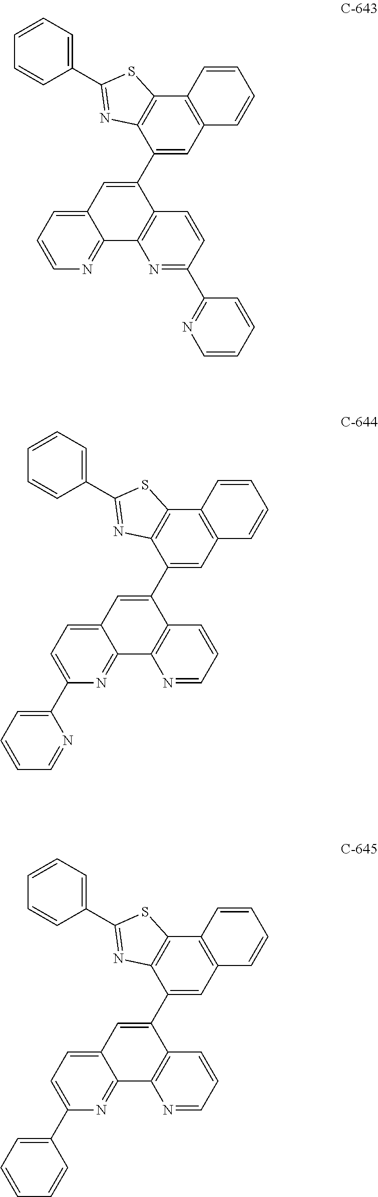 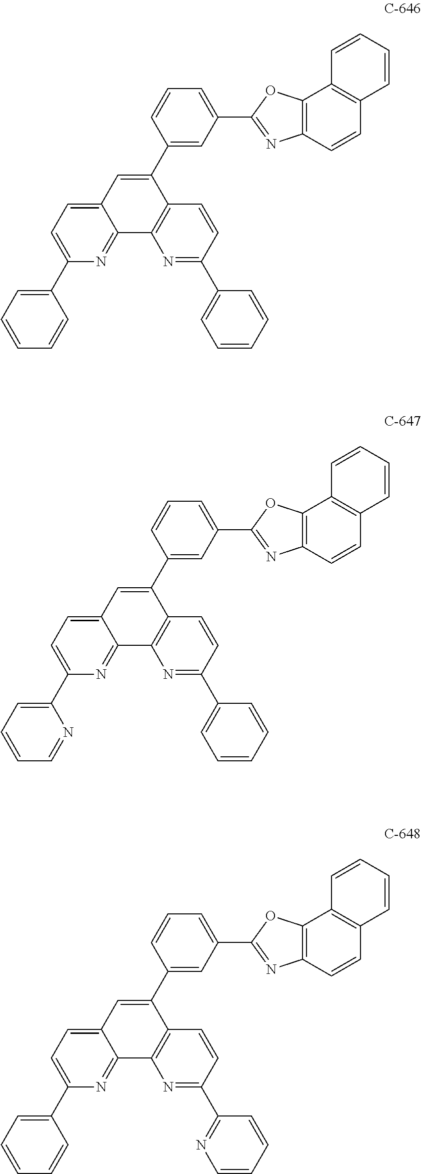 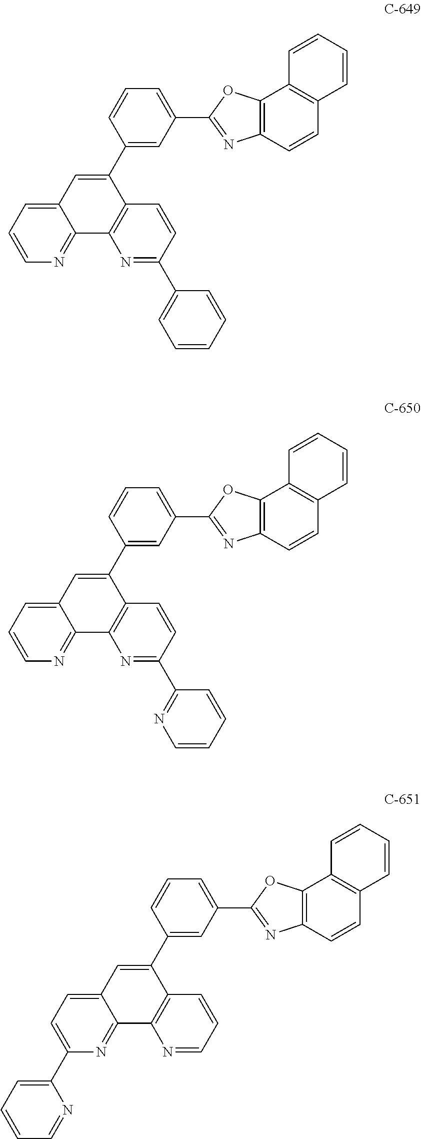 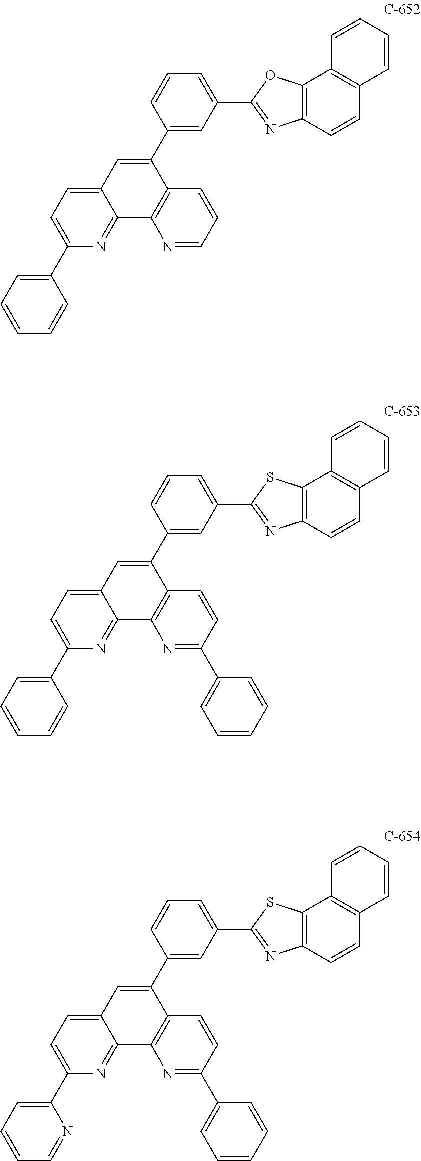 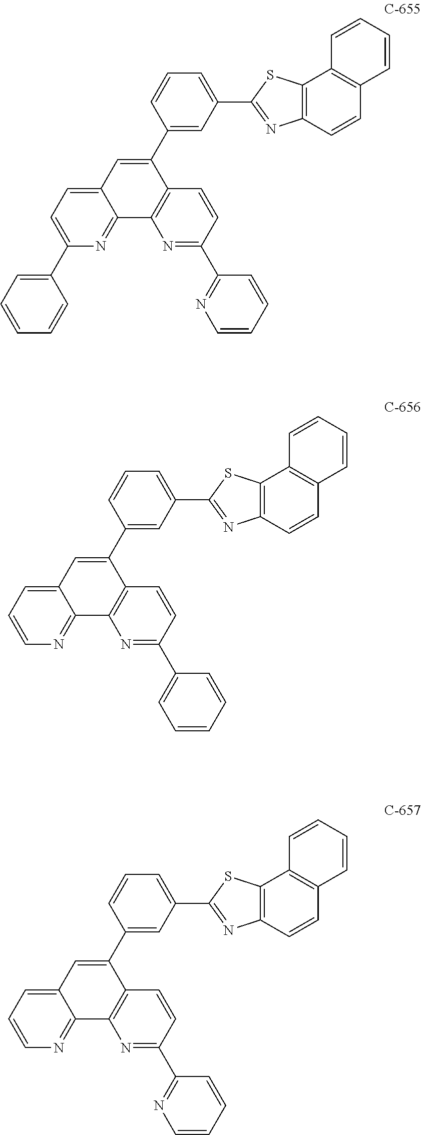 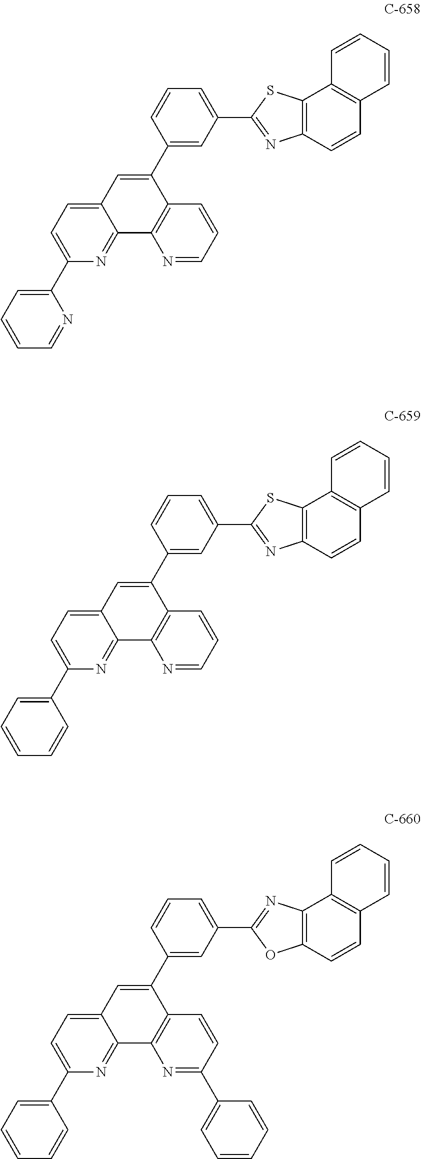 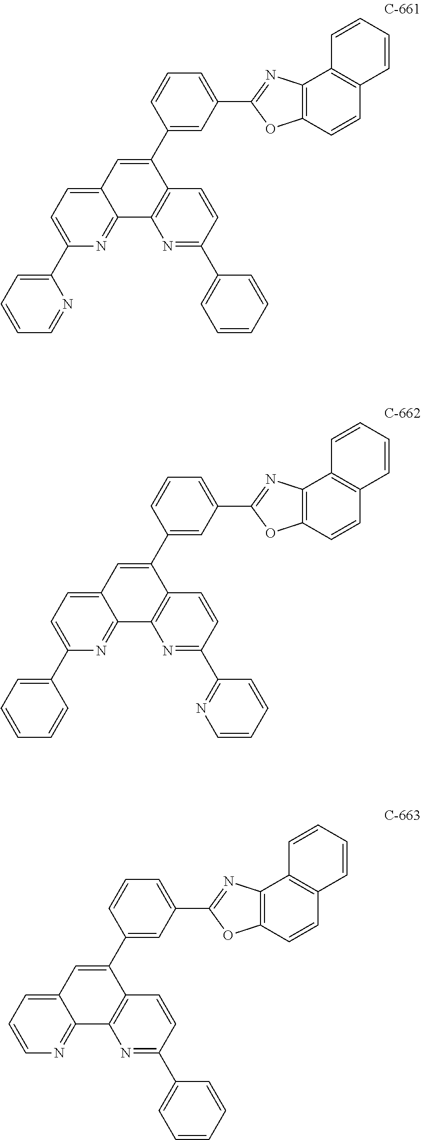 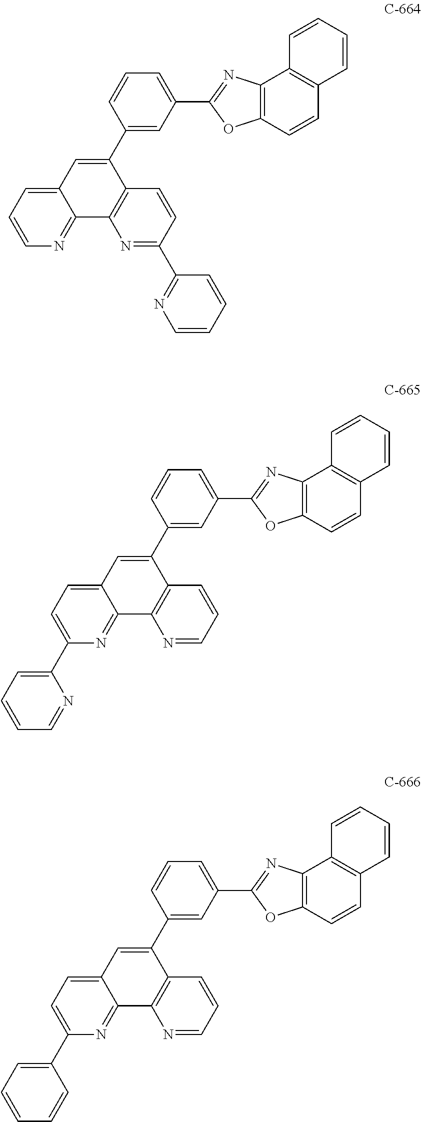 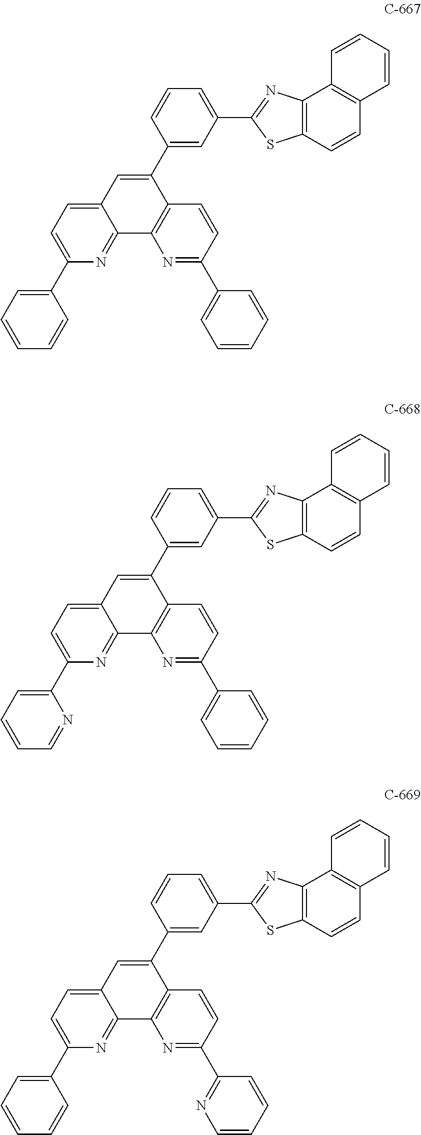 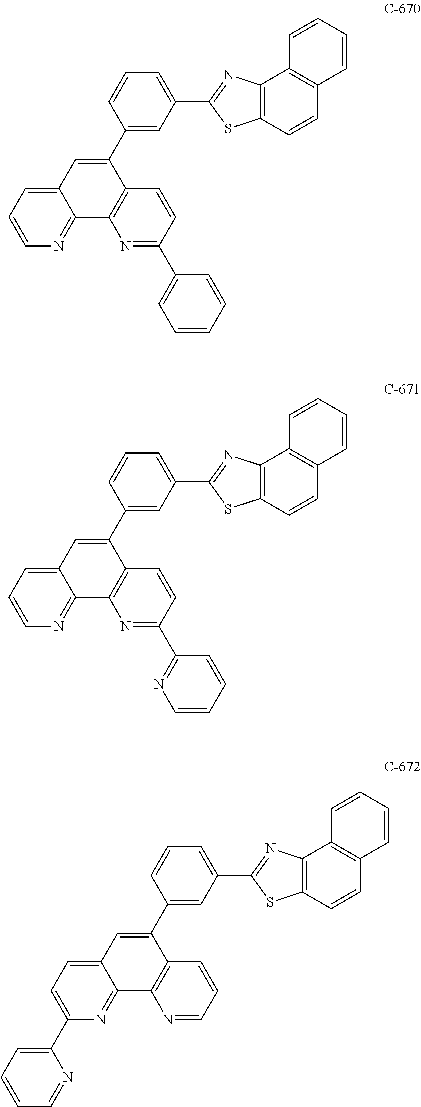 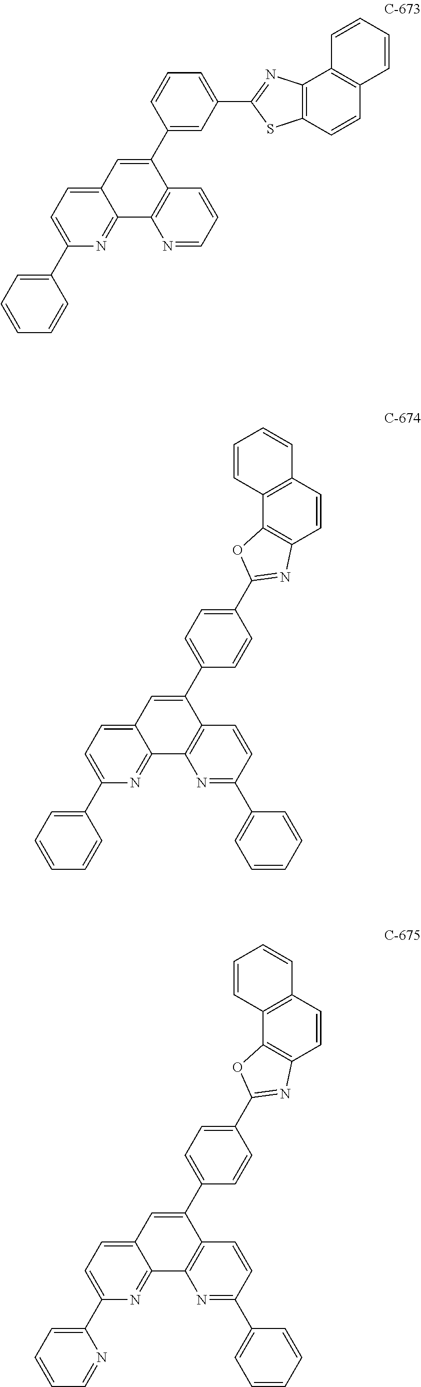 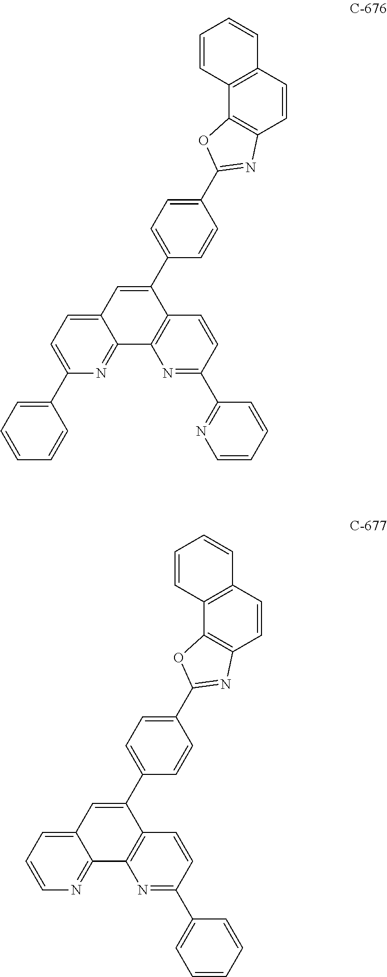 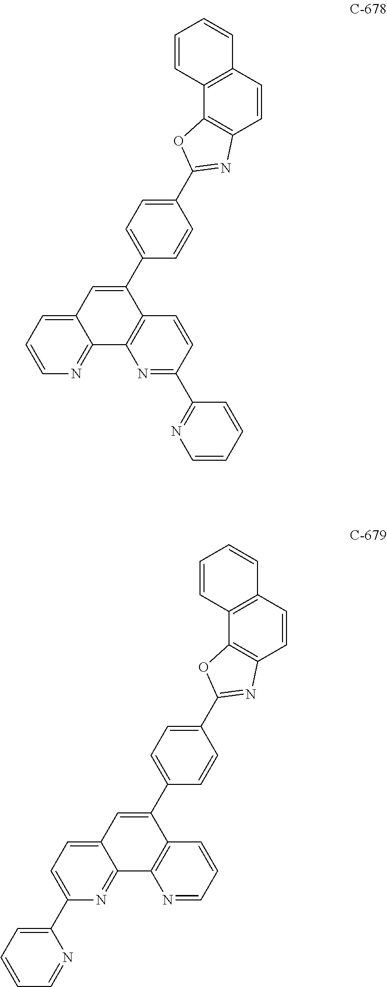 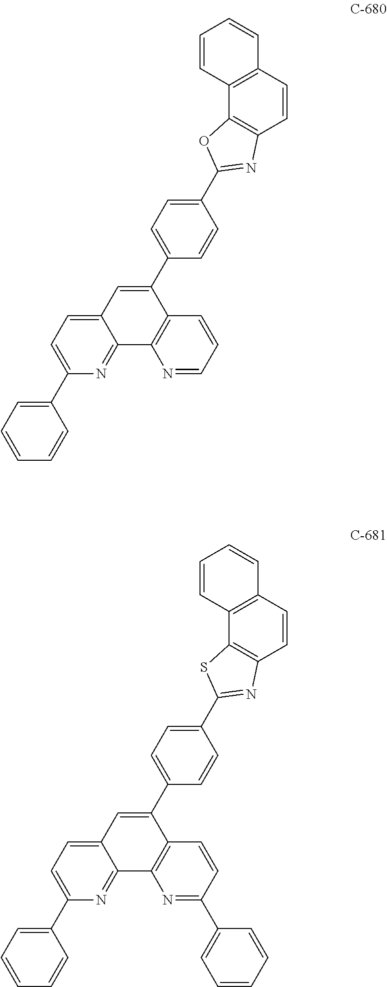 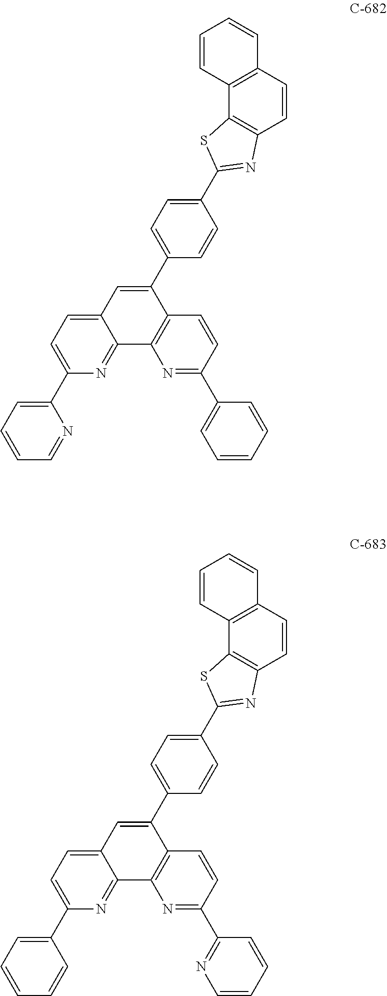 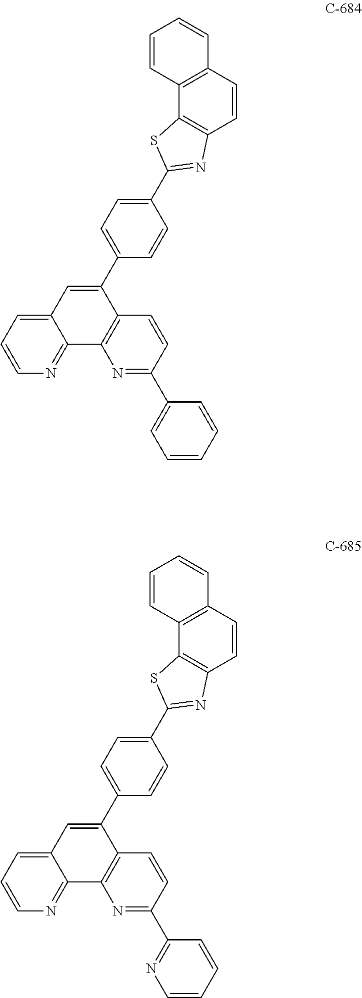 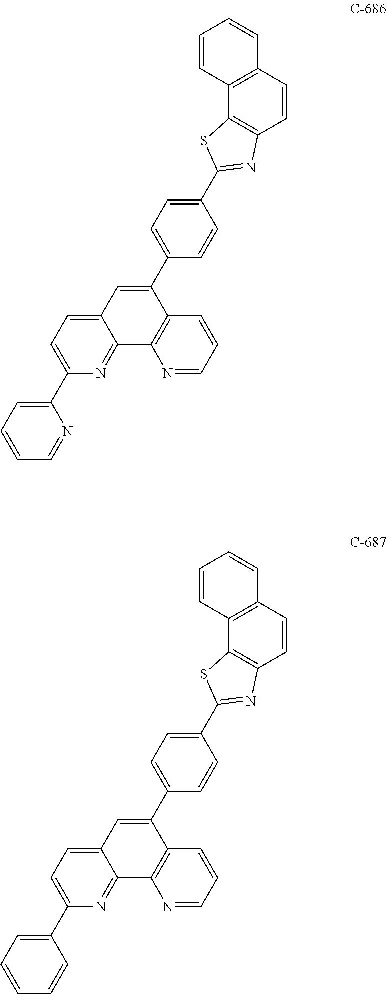 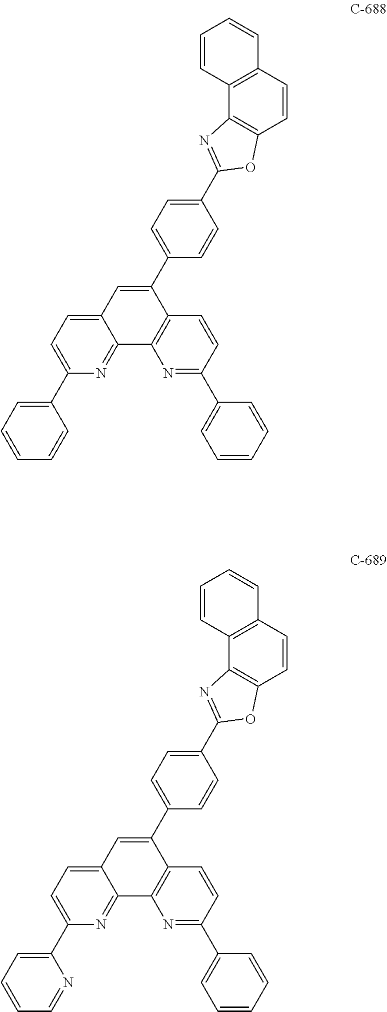 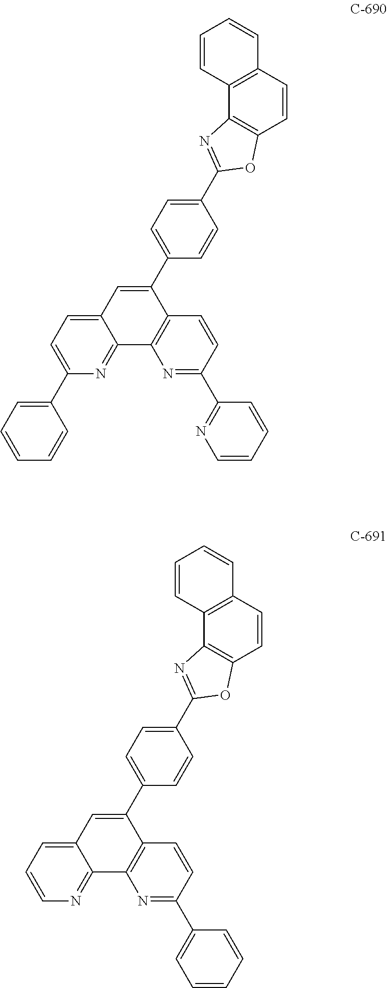 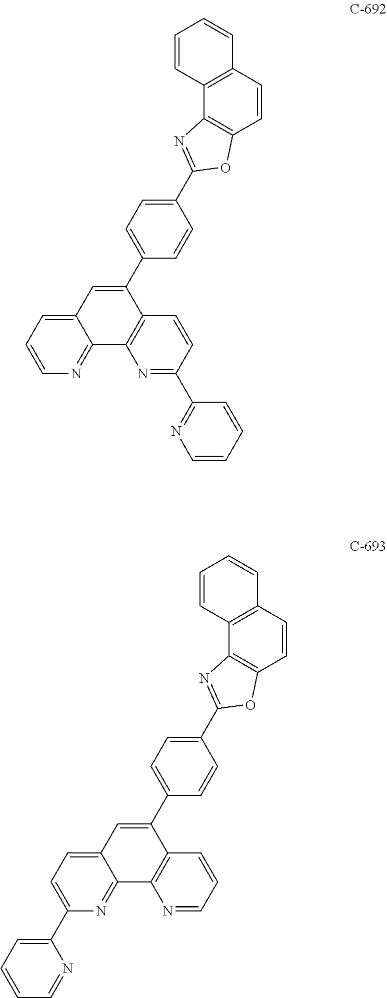 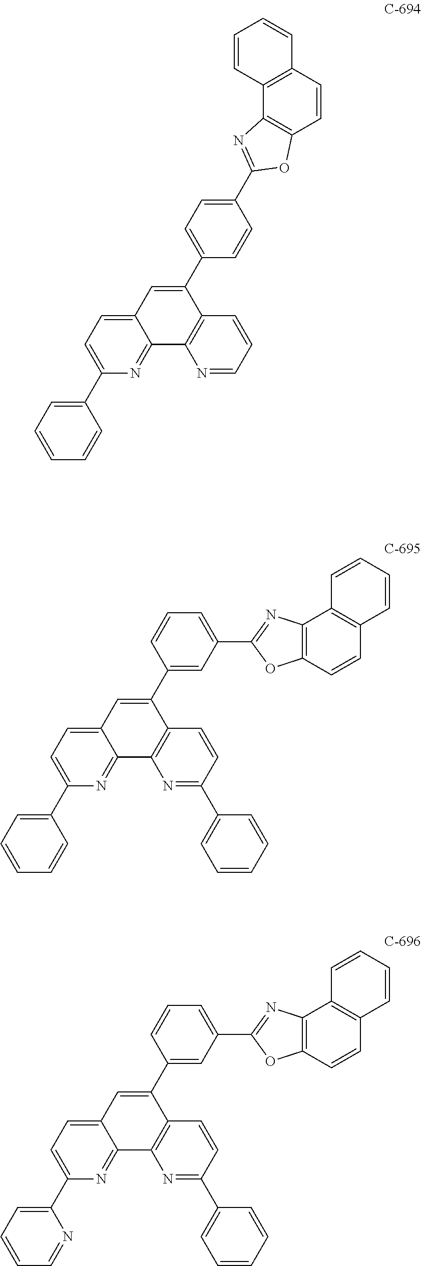 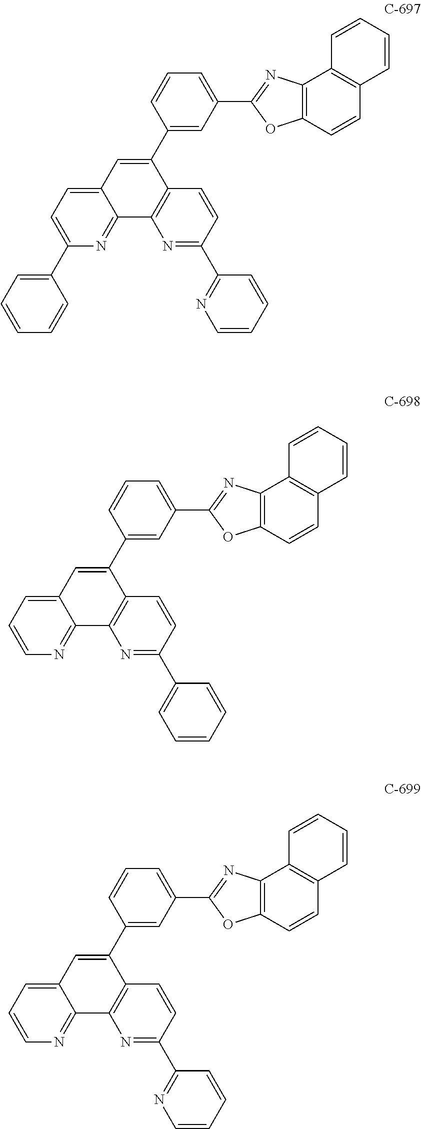 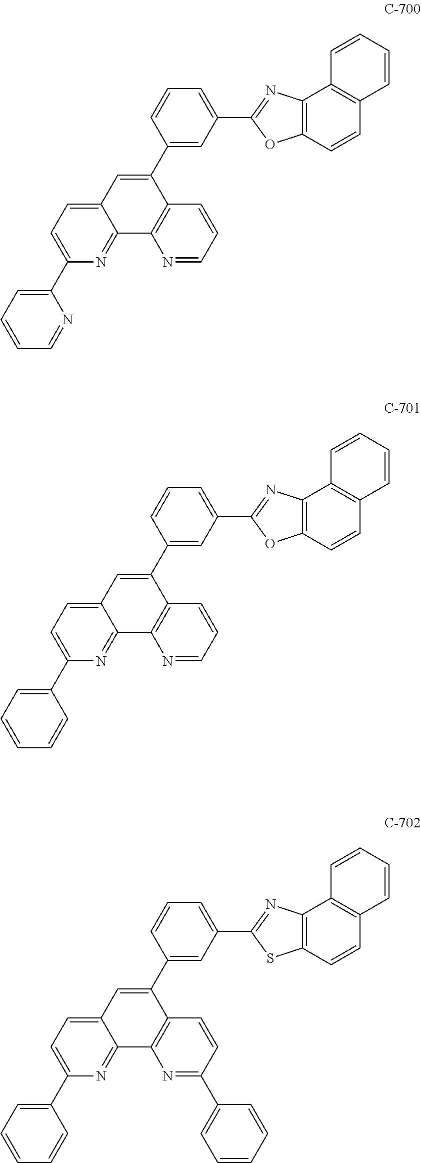 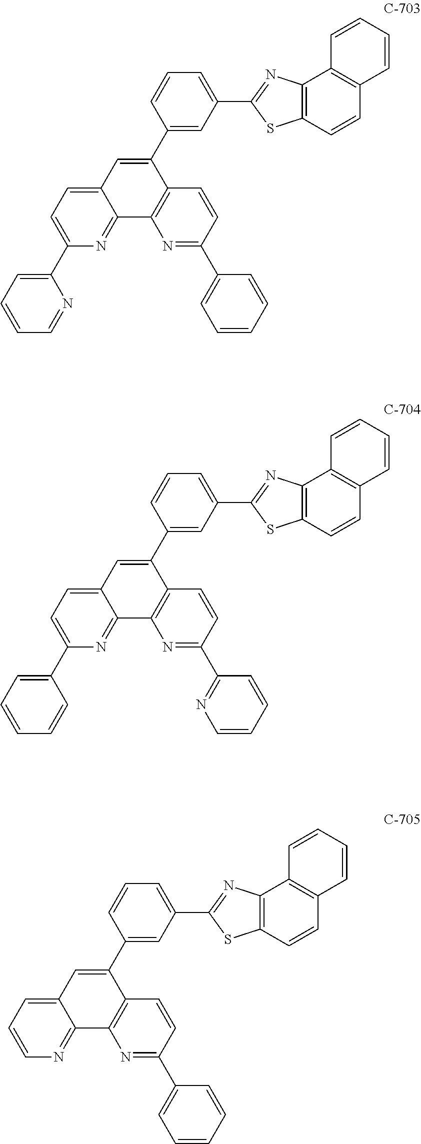 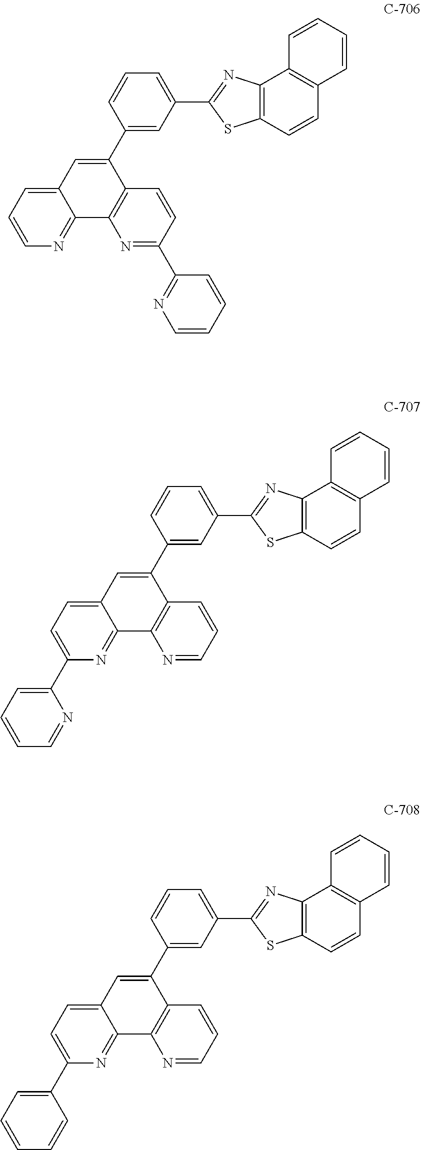 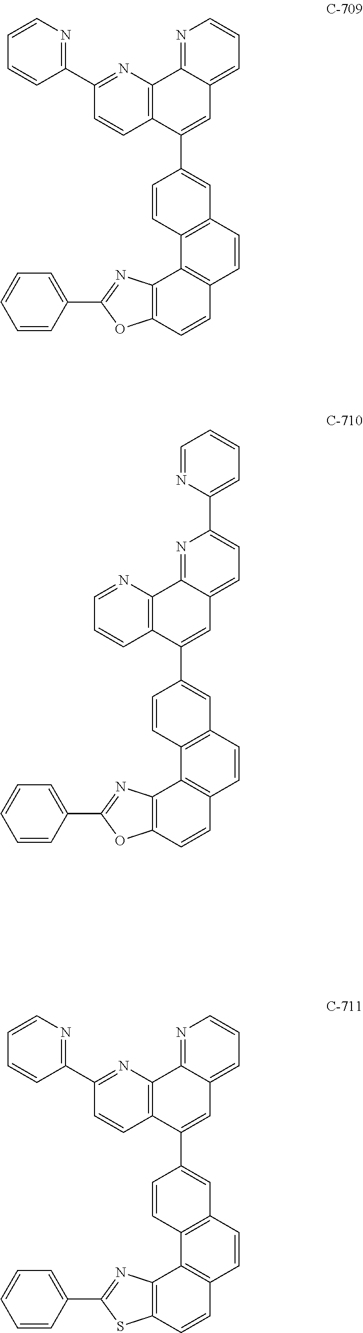 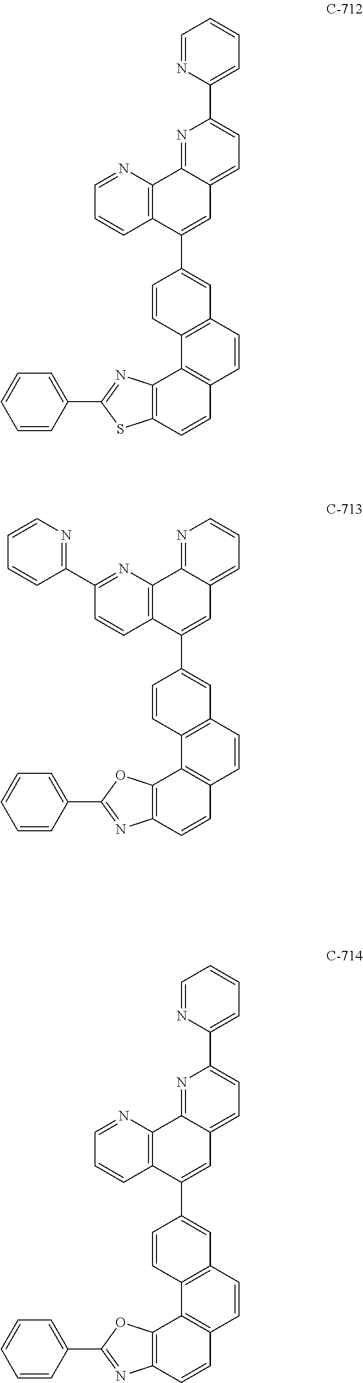 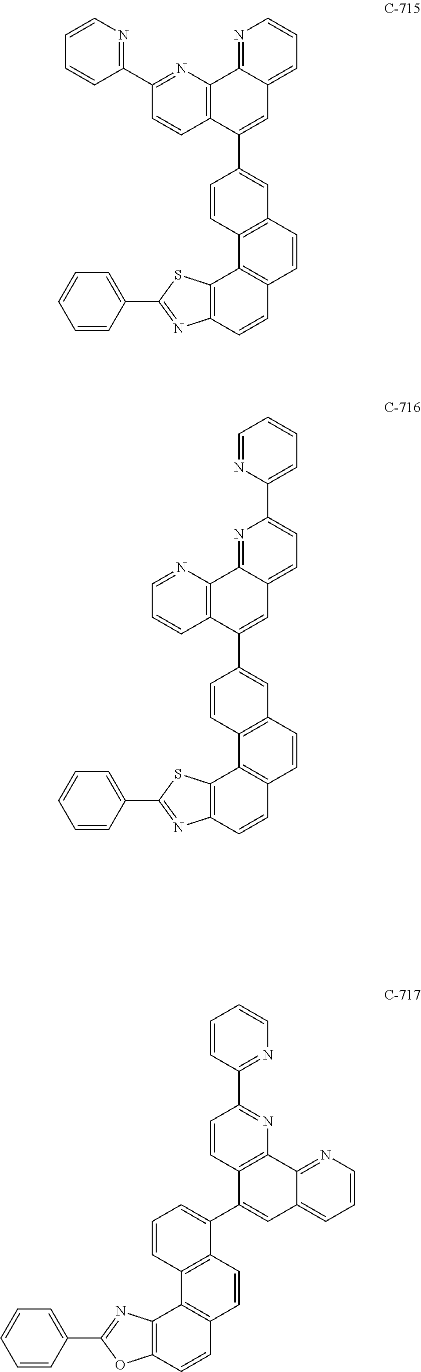 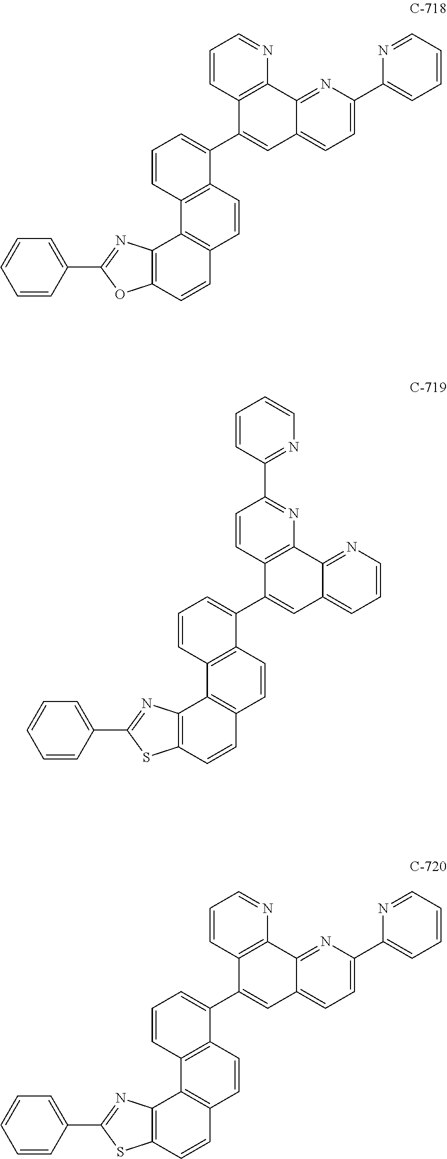 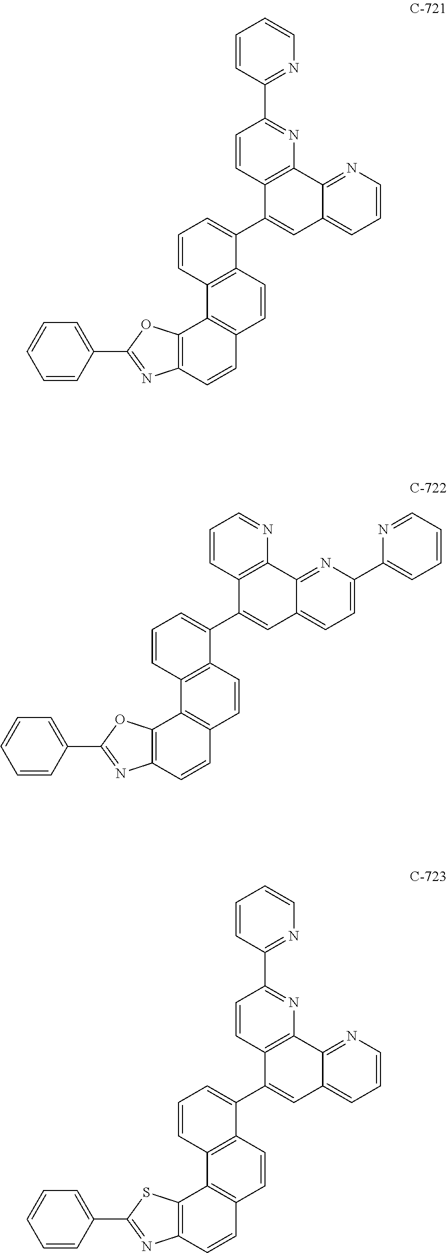 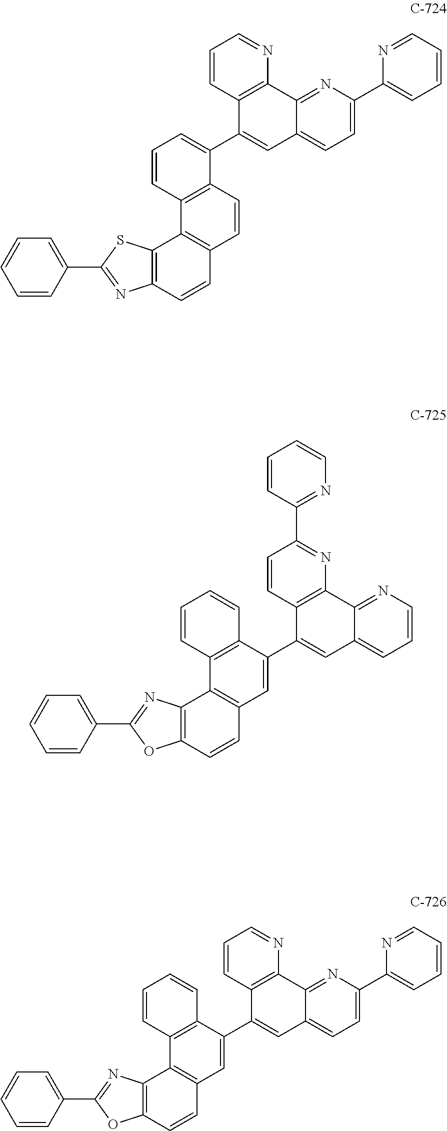 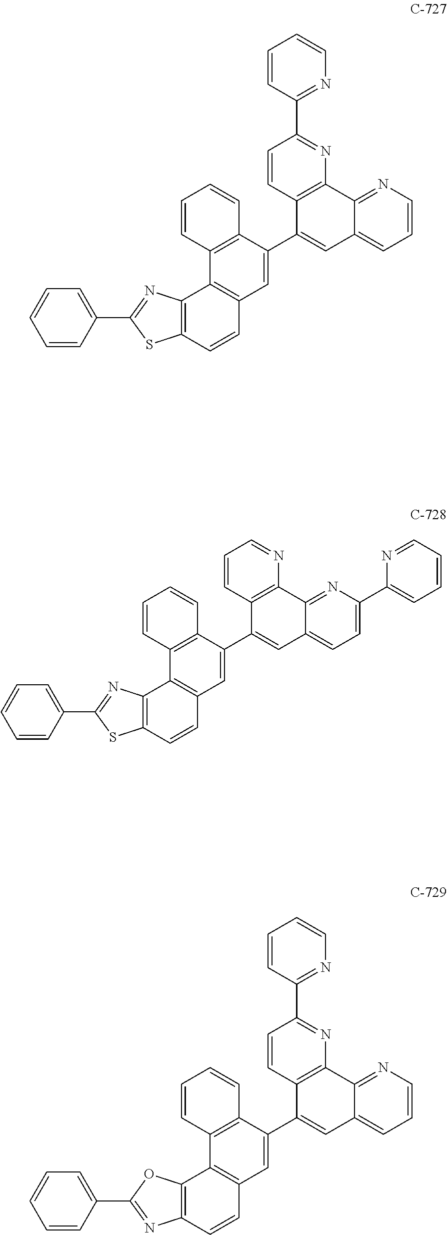 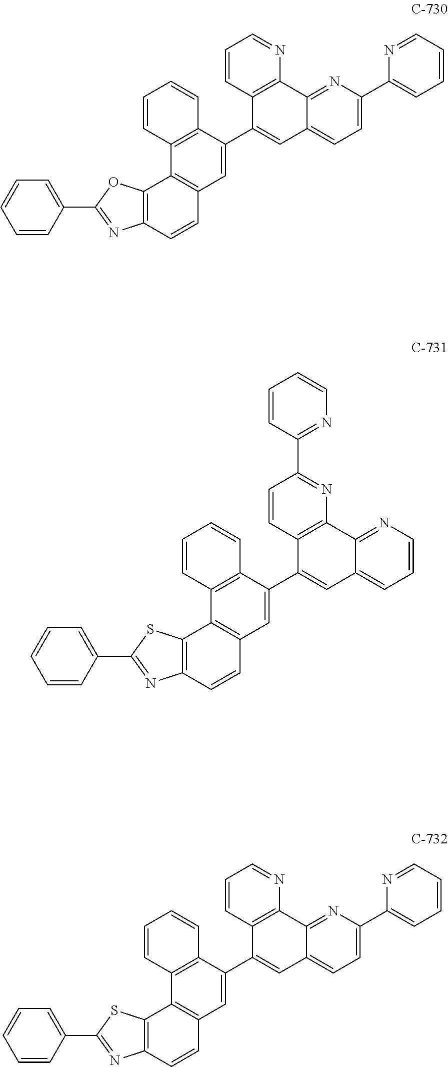 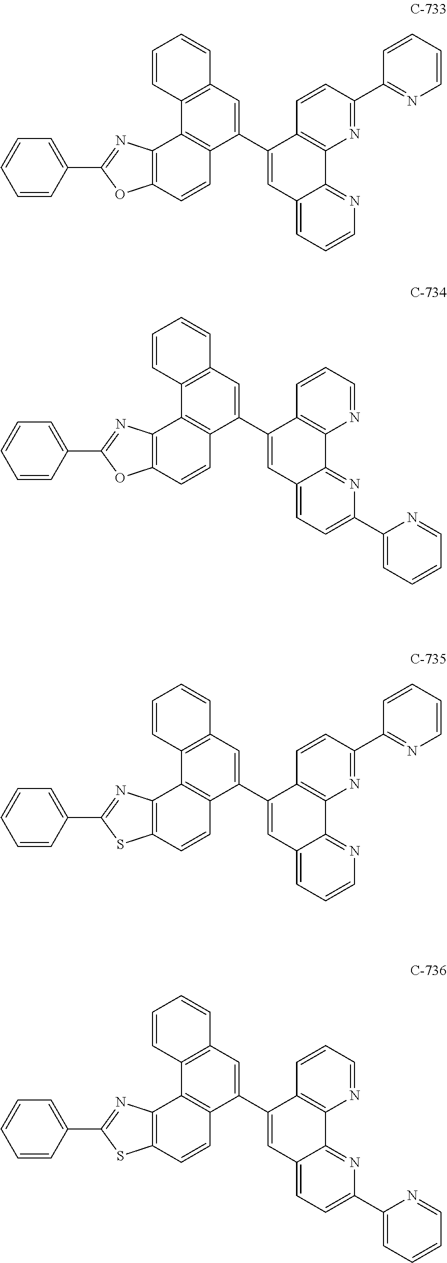 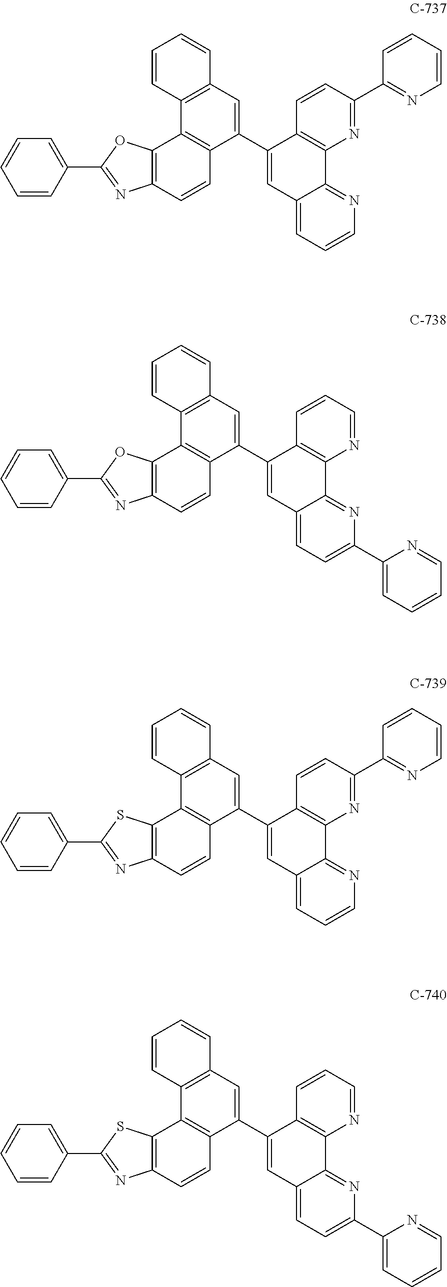 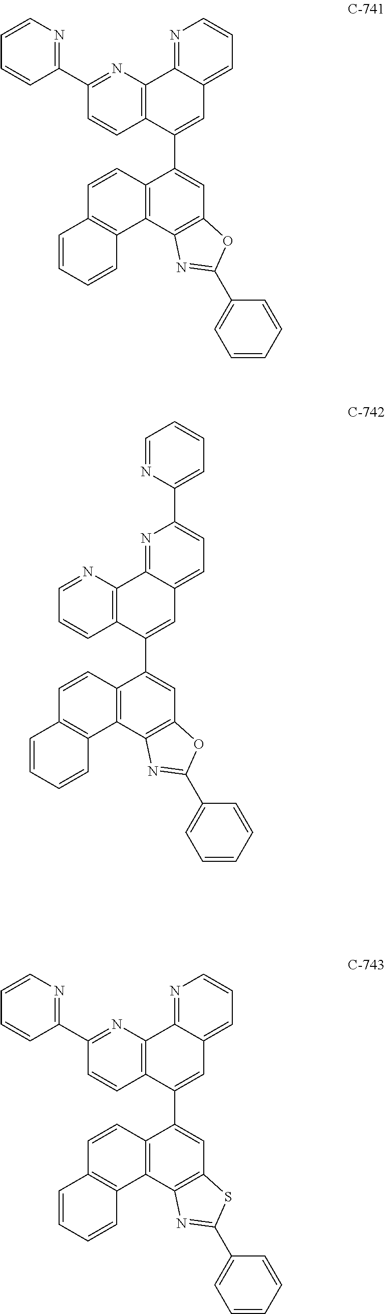 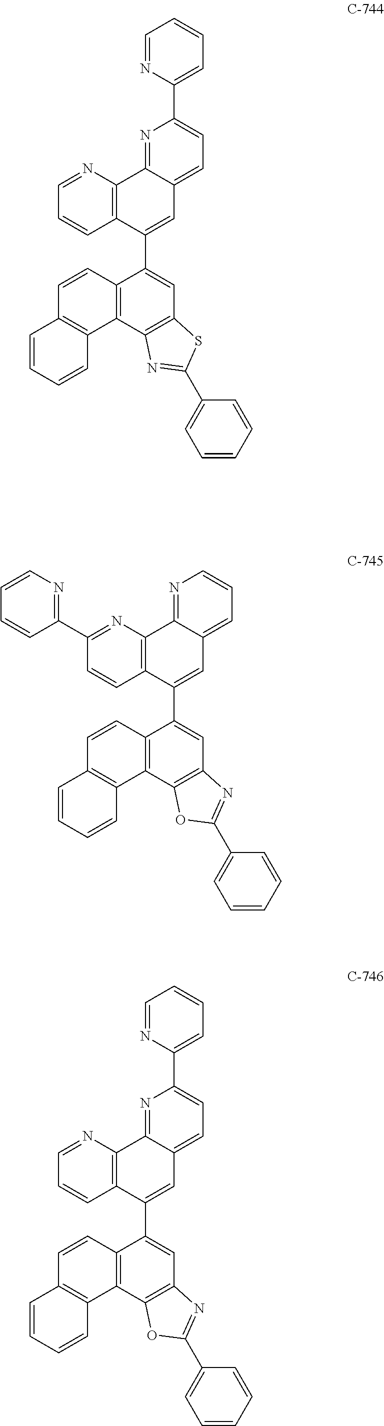 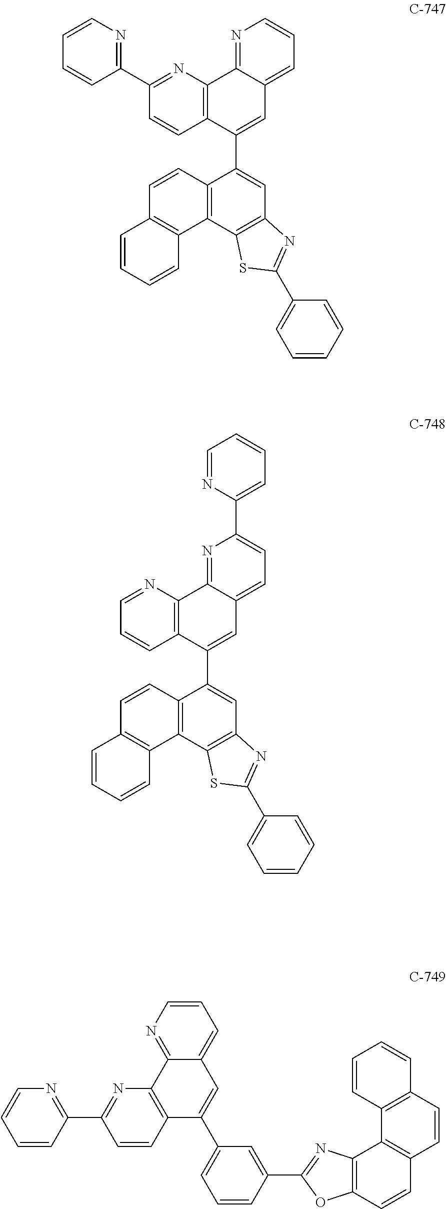 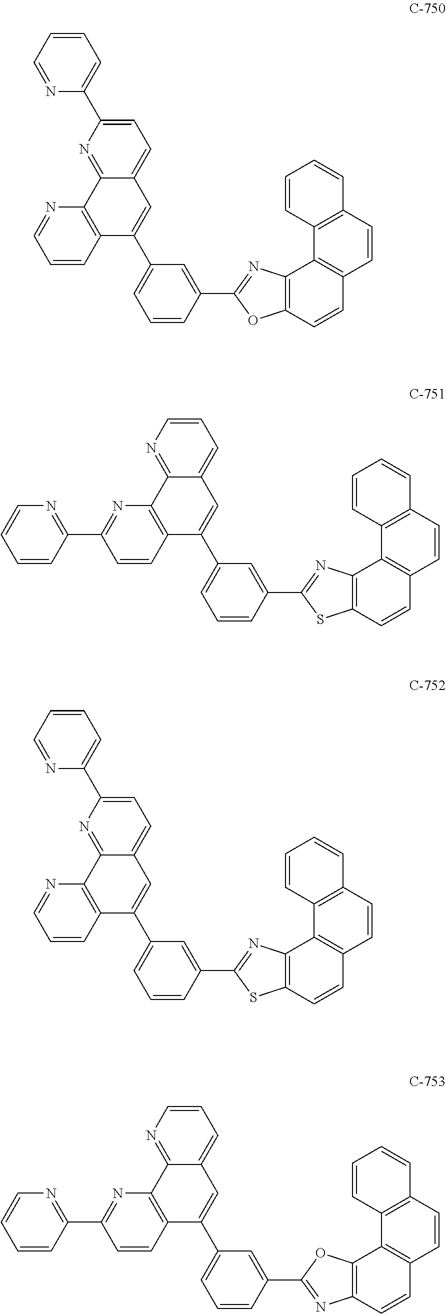 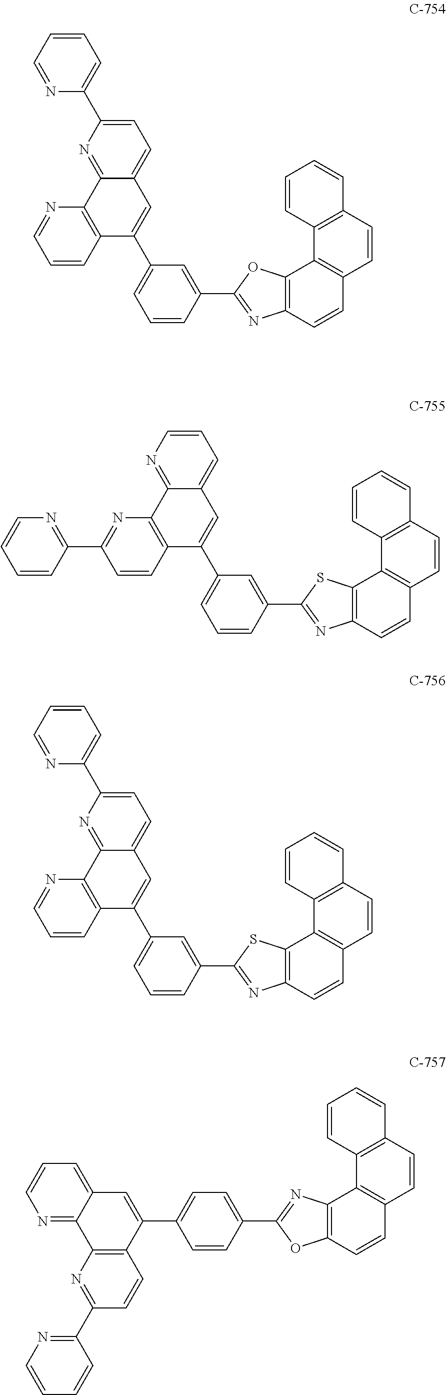 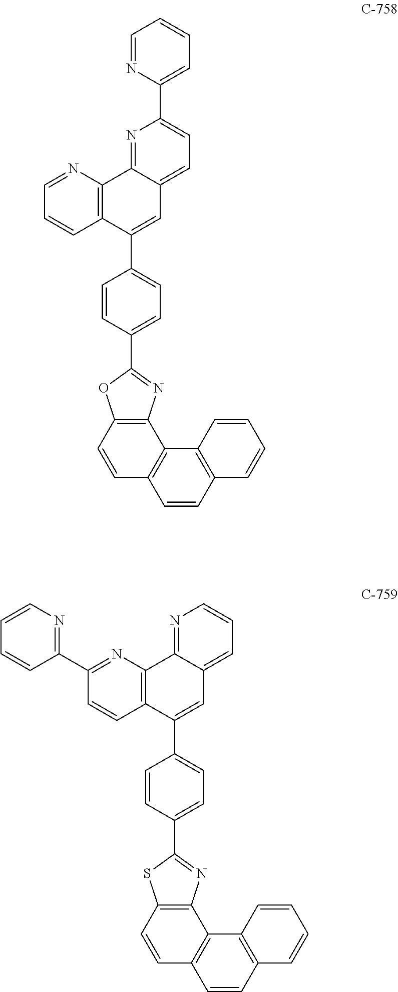 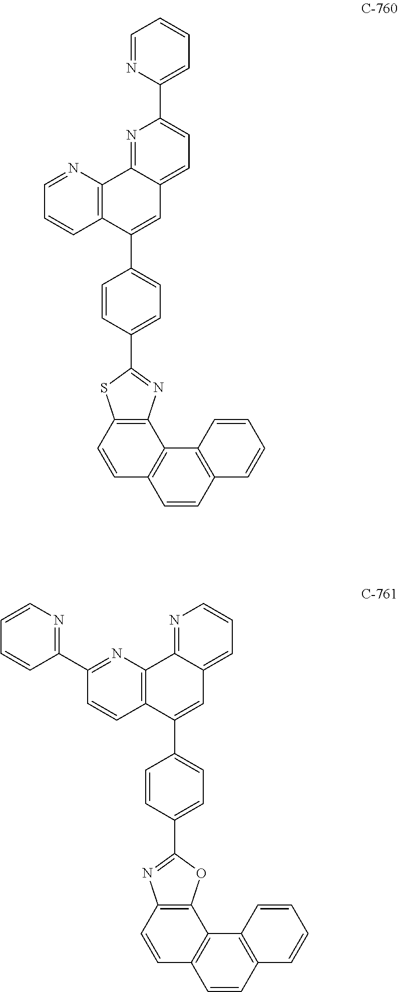 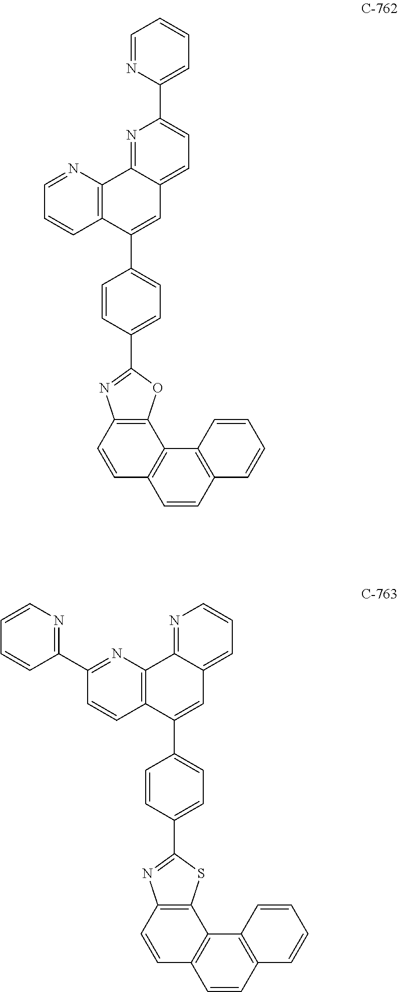 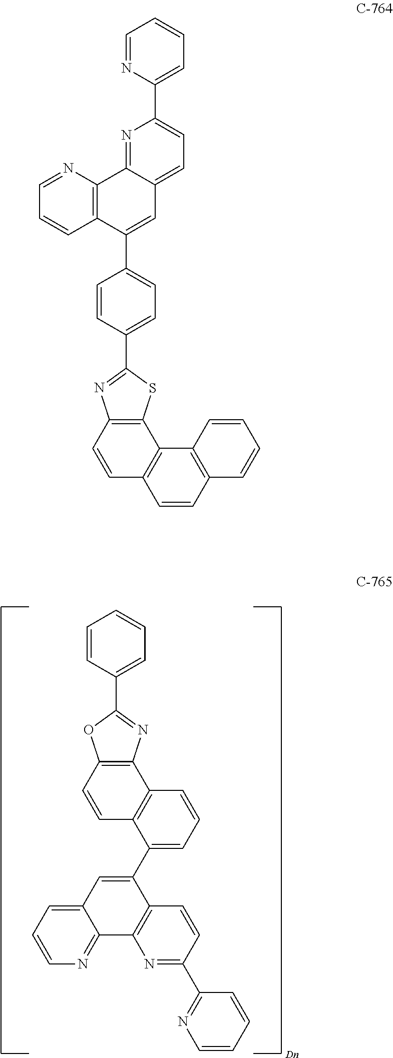 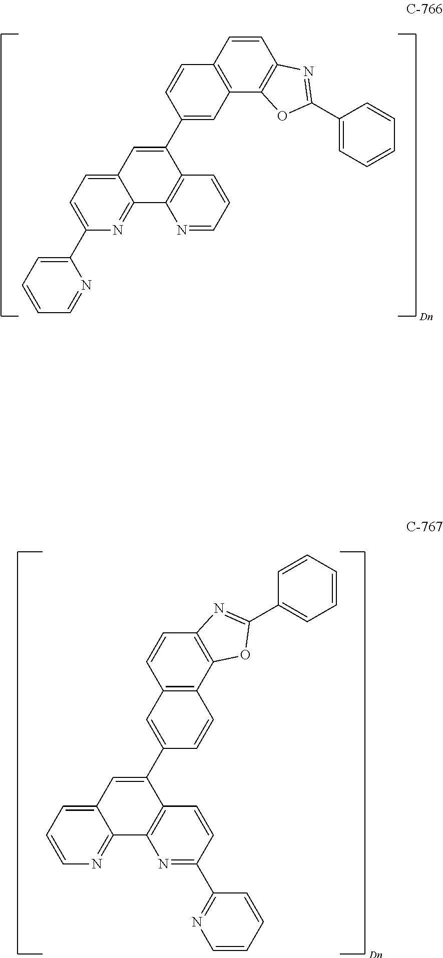 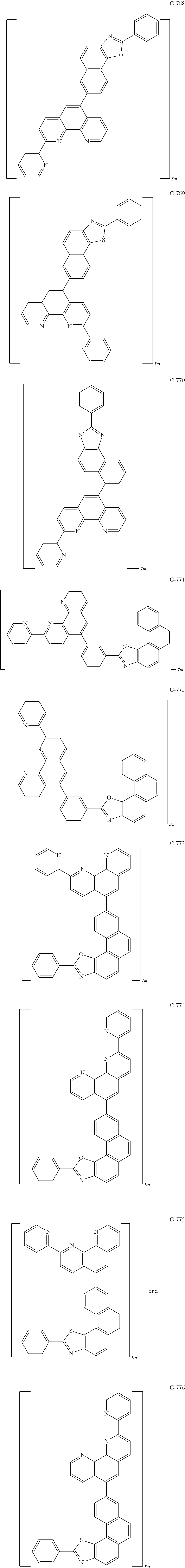
wherein Dₙ means that n number of hydrogens is replaced by deuterium, wherein n represents an integer of 1 or more, up to the total number of hydrogens of the compound.

7. An organic electroluminescent material comprising the organic electroluminescent compound according to claim 1.

8. An organic electroluminescent device comprising the organic electroluminescent compound according to claim 1.

9. An N-type charge generation material comprising the organic electroluminescent compound according to claim 1.

10. An organic electroluminescent compound that is a compound represented by Formula 1 of claim 1 that is coordinated to an alkali metal or an alkaline earth metal; or a complex compound comprising the compound represented by Formula 1 and a metal selected from an alkali metal, an alkaline earth metal, a lanthanoid, an actinoid, or a transition metal.

11. The organic electroluminescent device according to claim 8, comprising a first electrode; a second electrode; a plurality of light-emitting units disposed between the first electrode and the second electrode; and at least one charge generation layer disposed between adjacent light-emitting units among the plurality of light-emitting units; wherein the charge generation layer comprises the organic electroluminescent compound.
